(19) United States
(12) Patent Application Publication
(10) Pub. No.: US 2025/0108080 A1
(43) Pub. Date: Apr. 3, 2025

(54) EXTRACELLULAR VESICLES FROM GENETICALLY-MODIFIED MICROALGAE CONTAINING ENDOGENOUSLY-LOADED CARGO, THEIR PREPARATION, AND USES

(71) Applicant: AGS THERAPEUTICS SAS, Paris (FR)

(72) Inventors: Lila DRITTANTI, Paris (FR); Manuel VEGA, Paris (FR)

(21) Appl. No.: 18/959,314

(22) Filed: Nov. 25, 2024

Related U.S. Application Data
(63) Continuation of application No. PCT/EP2023/064751, filed on Jun. 1, 2023.
Application No. PCT/EP2023/064751 is a continuation-in-part of application No. PCT/EP2023/051650, filed on Jan. 24, 2023.
(60) Provisional application No. 63/349,006, filed on Jun. 3, 2022.

(51) Int. Cl. A61K 36/05; C12N 15/79

(52) U.S. Cl. CPC A61K 36/05; C12N 15/79; C12N 2800/10

(57) ABSTRACT

Provided are compositions containing extracellular vesicles (MEVs) produced in and isolated from genetically-engineered microalgae, which encode cargo, such as nucleic acids and polypeptides. The MEVs are endogenously loaded by the microalgae with the cargo. The MEVs have a variety of applications as a delivery system for therapeutics, including vaccines, anti-cancer therapeutics, diagnostics, and other such uses. The fate of MEVs upon administration depends upon the route of administration.

### RELATED APPLICATIONS

This application is a continuation of International PCT application No. PCT/EP2023/064751, filed Jun. 1, 2023, entitled “Extracellular Vesicles from Genetically-Modified Microalgae Containing Endogenously-Loaded Cargo, their Preparation, and Uses,” to inventors Lila Drittanti and Manuel Vega, and to Applicant AGS Therapeutics SAS. International PCT application No. PCT/EP2023/064751, claims benefit of priority to of U.S. provisional application Ser. No. 63/349,006, filed Jun. 3, 2022, entitled “Extracellular Vesicles from Genetically-Modified Microalgae Containing Endogenously Loaded Cargo, their Preparation, and Uses,” to Applicant AGS Therapeutics SAS, and inventors Lila Drittanti and Manuel Vega. International PCT application No. PCT/EP2023/064751, claims benefit of priority to of International PCT application No. PCT/EP2023/051650, filed Jan. 24, 2023, entitled “Extracellular Vesicles from Microalgae, Their Biodistribution Upon Administration, and Uses,” to Applicant AGS Therapeutics SAS, and inventors Lila Drittanti, and Manuel Vega for subject matter only entitled to the Jan. 24, 2023 date. International PCT application No. PCT/EP2023/064751 is a continuation-in-part of International PCT application No. PCT/EP2023/051650.

This application claims the benefit of priority to, as a continuation-in-part, of International PCT Application No. PCT/EP2023/051650, filed Jan. 24, 2023, entitled “Extracellular Vesicles from Microalgae, Their Biodistribution Upon Administration, and Uses,” to Applicant AGS Therapeutics SAS, and inventors Lila Drittanti, and Manuel Vega for subject matter only entitled to the Jan. 24, 2023 date.

Benefit of priority is claimed to U.S. provisional application Ser. No. 63/349,006, filed Jun. 3, 2022, entitled “Extracellular Vesicles from Genetically-Modified Microalgae Containing Endogenously Loaded Cargo, their Preparation, and Uses,” to Applicant AGS Therapeutics SAS, and inventors Lila Drittanti and Manuel Vega.

The subject matter of each of these applications is incorporated by reference in its entirety.

### Incorporation by Reference of Sequence Listing Provided Electronically

An electronic version of the Sequence Listing is filed herewith, the contents of which are incorporated by reference in their entirety. The electronic file was created Nov. 15, 2024, is 1,357,270 bytes in size, and is titled 5504SEQ001.XML.

### FIELD

Provided are compositions, methods, and uses, containing extracellular vesicles produced by genetically-modified microalgae. Extracellular vesicles can serve as drug delivery systems. The extracellular vesicles provided herein contain endogenously-loaded cargo, and are produced by the genetically-modified microalgae, which produce microalgae extracellular vesicles (MEVs) that are endogenously loaded with bioactive cargo. The MEVs have a variety of applications as therapeutics, including for delivery of therapeutics, such as proteins, polypeptides, peptide therapeutics, and RNA. They, thus, serve as drug delivery systems. Also provided are the genetically-modified microalgae and methods of producing the endogenous cargo-loaded MEVs.

### BACKGROUND

Extracellular vesicles (EVs) are natural particles produced by most cells. EVs include exosomes (about 30-120 nm in size), which are released to the extracellular environment upon fusion of multivesicular endosomes with the plasma membrane and include microvesicles (about 50-1000 nm), which are produced by the outward budding of membrane vesicles from the cell surface. Exosomes and microvesicles have similar properties, and in general are referred to as EVs.

EVs function in intercellular communication via cell-cell transfer of proteins, and nucleic acids, such as microRNAs (miRNAs), long noncoding RNAs (lncRNAs), and mRNAs. By virtue of this, EVs derived from mammals and plants have been used as carriers for short interfering RNA (siRNA) delivery, microRNA (miRNA), and small molecule drugs. There is a need for conveniently produced EVs that are readily delivered to cells and tissues. It is an object herein to provide such EVs.

### SUMMARY

Provided are endogenously-loaded extracellular vesicles (MEVs) from genetically engineered microalgae. Microalgae are unicellular green algae, and include those that belong to the order Chlorellales, in particular the Chlorellaceae family, and in particular those that belong to the Chlorella genus, such as Chlorella vulgaris. The MEVs are loaded with cargo that includes bioactive molecules, long non-coding (Inc) RNA encoding small open reading frames (sORFs), or its translated small peptides (less than 100 amino acids); mRNA encoding open reading frames (ORFs) or its translated peptides (more than 100 amino acids), and proteins; and protein/protein or protein/RNA complexes. The MEVs herein are endogenously loaded with cargo by genetically-engineered microalgae cells. The genetically-engineered microalgae cells encode the cargo, which is loaded in vivo into the MEVs.

MEVs are endogenously loaded by genetically engineered microalgae cells with bioactive molecule cargo heterologous to the MEVs or the microalgae in that the cargo is encoded by the generically-modified microalgae for packaging in the MEVs. The resulting compositions, thus, contain MEVs that have the same endogenously-loaded heterologous cargo because the cargo is encoded by the microalgae and packaged into the MEVs by the microalgae.

Provided are compositions that contain endogenously-loaded MEVs, particularly those produced by engineered microalgae cells from the order Chlorellales, in particular from the Chlorellaceae family, and in particular from the Chlorella genus, such as Chlorella vulgaris. Methods for obtaining endogenously loaded MEVs are provided. The cargos are bioactive molecules or combinations thereof, including nucleic acids, sORFs, peptides and proteins. The cargos include, for example, biomolecules, including biopolymers, such as RNA, peptides, proteins, protein complexes, and protein-nucleic acid complexes. The bioactive molecules include therapeutics, such as anti-cancer compounds and biomolecules, such as RNAi, proteins, and complexes; vaccine molecules, such as proteins, peptides, nucleic acid, protein complexes and protein-nucleic acid complexes; diagnostic molecules, such as detectable markers; and molecules that are cosmetics. Methods of treatment of diseases, disorders, and conditions, including pathogen infections and cancers, and uses for the endogenously loaded MEVs for treatment for the diseases and disorders are provided, as are methods of diagnosis, and uses of MEVs for prevention, reduction of risk, prophylaxis of pathogen infections and cancers. The compositions can be employed as drug delivery systems, and formulated for particular routes of administration.

The resulting endogenously loaded MEVs, loaded by genetically engineered microalgae, have applications in a variety of fields, including diagnosis, prophylaxis, treatment of human diseases, industrial uses, and cosmetics. The MEVs, with appropriate cargo for each application, can be used as vaccines, as gene therapy delivery vectors, for therapeutic prophylaxis, for a variety of purposes, such as gene silencing, gene editing, gene modulation, in the industry and for research, analytical methods, cell-based assays, and other uses and applications.

Provided are microalgae cell cultures, comprising genetically-modified microalgae, where the microalgae or microalgae cell culture comprise microalgae extracellular vesicles (MEVs) containing endogenous cargo; the MEVs are produced by the microalgae cells; the endogenous cargo is produced by microalgae in which the MEVs were produced. The endogenous cargo comprises bioactive molecules that are heterologous to the microalgae; and the heterologous bioactive molecules comprise RNA molecules, small peptides, peptides, polypeptides, and/or proteins, with the proviso that the RNA molecules do not comprise inhibitory RNA (RNAi) or particularly siRNA, and more particularly siRNA that targets pathogens. In all embodiments herein, RNAi includes small inhibiting RNA (siRNA), micro-RNA (miRNA), and/or short-hairpin RNA (shRNA).

Also provided are microalgae cell cultures, comprising genetically-modified microalgae, where: the microalgae or microalgae cell culture comprise microalgae extracellular vesicles (MEVs) containing endogenous cargo; the MEVs are produced by the microalgae cells; the endogenous cargo is produced by microalgae in which the MEVs were produced; the endogenous cargo comprises bioactive molecules that are heterologous to the microalgae; and the heterologous bioactive molecules comprise inhibitory RNA (RNAi), with the proviso that the RNAi does not comprise heterologous RNAi designed to target pathogen genes and/or host pathogen-susceptibility factors.

Also provided is cell culture medium from microalgae cell cultures. The cell culture medium, which is medium from a cell culture, with cell debris removed, comprises microalgae extracellular vesicles (MEVs) containing endogenously-loaded (endo-loaded) cargo. The MEVs are produced by genetically-modified microalgae cells in the cell culture; the microalgae cells are genetically modified to produce the endogenous cargo; the endogenous cargo is produced by the microalgae cells in which the MEVs are produced; the endogenous cargo comprises bioactive molecules that are heterologous to the microalgae; and the heterologous bioactive molecules comprise RNA molecules, small peptides, peptides, polypeptides, and/or proteins, with the proviso that the RNA molecules do not comprise RNAi, for packaging in MEVs. Provided is microalgae cell culture medium, comprising microalgae extracellular vesicles (MEVs) containing endogenous cargo, where the MEVs are produced by genetically-modified microalgae cells in the cell culture; the endogenous cargo is produced by microalgae in which the MEVs were produced; the endogenous cargo comprises bioactive molecules that are heterologous to the microalgae; and the heterologous bioactive molecules comprise RNAi, with the proviso that the RNAi does not target pathogen genes and/or host pathogen-susceptibility factors.

Provided are genetically-modified microalgae cells. The genetically-modified microalgae comprise genome modifications that improve or enhance or modulate or control production of EVs. The genetically-modified microalgae also or alternatively comprises heterologous nucleic acid that encodes cargo for endogenous loading.

Provided are genetically-modified microalgae cells that comprise microalgae extracellular vesicles (MEVs) containing endogenous cargo, where: the MEVs are produced by the genetically-modified microalgae cell; the endogenous cargo is produced by microalgae in which the MEVs were produced; the endogenous cargo comprises bioactive molecules that are heterologous to the microalgae; the endogenous cargo is produced by the microalgae cell via natural or modified biosynthetic pathway(s); and the heterologous bioactive molecules comprise RNA molecules, small peptides, peptides, polypeptides, and/or proteins, with the proviso that the RNA molecules do not comprise RNAi.

Provided are genetically-modified microalgae cells comprising microalgae extracellular vesicles (MEVs) containing endogenous cargo, where: the MEV is produced by the genetically-modified microalgae cell; the endogenous cargo is produced by microalgae in which the MEVs were produced; the endogenous cargo comprises bioactive molecules that are heterologous to the microalgae; the endogenous cargo is produced by the microalgae cell via natural or modified biosynthetic pathway(s); and the heterologous bioactive molecules comprise RNAi, with the proviso that the RNAi does not target pathogen genes and/or host pathogen-susceptibility factors. The genetically-modified microalgae cells can comprise a producer cell line for producing MEVs with endogenously-loaded (endo-loaded) cargo.

Provided are microalgae extracellular vesicles (MEVs) isolated from the cell culture or cell culture medium, and/or from or produced by the genetically-modified microalgae.

Compositions containing the microalgae extracellular vesicles (MEVs) containing endogenous cargo are provided. Compositions include pharmaceutical compositions containing an MEV or MEVs in a pharmaceutically acceptable vehicle or carrier. The compositions can be formulated for particular routes of administration. As shown herein, MEVs can be directed to different organs and tissues by selecting a route of administration. For example, the MEVs provided herein can be administered orally and survive the stomach and enter the intestines and traffic to gut-associated lymphoid tissues (GALT). Other routes of administration, as shown herein, result, for example in trafficking to mucosa-associated lymphoid tissue (MALT), bronchus-associated lymphoid tissues (BALT), nasal-associated lymphoid tissues (NALT), conjunctival-associated lymphoid tissues (CALT), larynx-associated lymphoid tissues (LALT), skin-associated lymphoid tissues (SALT), vulval-vaginal-associated lymphoid tissues (VALT), and testis associated lymphoid tissues (TALT).

Endogenous cargo for treatment of a disease, disorder, or condition that involves a tissue or organ can be endogenously loaded into the MEVs, which can then be formulated for administration by a route that targets the tissue or organ. The MEVs and compositions containing the MEVs function as drug delivery systems in which a therapeutic is produced in the microalgae, loaded into the MEVs, which are isolated and formulated for a route of administration that targets a tissue or organ that is a target for the therapeutic. Hence provided are drug delivery systems (or compositions) that comprise the MEVs provided herein.

Provided are compositions that comprise an MEV or MEVs, where: the endogenous cargo is produced by microalgae in which the MEVs were produced; the endogenous cargo comprises bioactive molecules that are heterologous to the microalgae; the endogenous cargo is produced by the microalgae via natural or modified biosynthetic pathway(s); the heterologous bioactive molecules comprise RNA molecules, small peptides, peptides, polypeptides, and/or proteins, with the proviso that the RNA molecules do not comprise RNAi. Provided are compositions comprising the MEVs containing endogenously-loaded cargo. The MEVs are produced by the cell cultures, from the cell culture medium, produced by the genetically-modified microalgae cells.

Also provided are compositions that comprise microalgae extracellular vesicles (MEVs) containing endogenous cargo, where: the endogenous cargo is produced by microalgae in which the MEVs were produced; the endogenous cargo comprises bioactive molecules that are heterologous to the microalgae; the endogenous cargo is produced by the microalgae via natural or modified biosynthetic pathway(s); the heterologous bioactive molecules comprise RNAi, with the proviso that the RNAi does not target pathogen genes and/or host pathogen-susceptibility factors.

Provided are microalgae extracellular vesicles (MEVs), comprising endogenous cargo, where: the endogenous cargo is produced by microalgae in which the MEVs were produced; the endogenous cargo comprises bioactive molecules that are heterologous to the microalgae; the endogenous cargo is produced by the microalgae via natural or modified biosynthetic pathway(s); the heterologous bioactive molecules comprise RNA molecules, small peptides, peptides, polypeptides, and/or proteins, with the proviso that the RNA molecules do not comprise RNAi Provided is a microalgae extracellular vesicle (MEV), comprising endogenous cargo, where: the endogenous cargo is produced by microalgae in which the MEVs were produced; the endogenous cargo comprises bioactive molecules that are heterologous to the microalgae; the endogenous cargo is produced by the microalgae via natural or modified biosynthetic pathway(s); the heterologous bioactive molecules comprise RNAi, with the proviso that the RNAi does not target pathogen genes and/or host pathogen-susceptibility factors.

Provided is the cell culture, cell culture medium, genetically-modified microalgae cell, composition, or MEVs, where the endogenous cargo a comprises a peptide, small peptide, polypeptide, and/or a protein. In other embodiments, the endogenous cargo comprises RNA, such as coding RNA and non-coding RNA, with the proviso that the non-coding RNA molecules do not comprise inhibitory RNA

(RNAi), particularly with the proviso that the RNAi does not comprise RNAi designed to target pathogen genes and/or host pathogen-susceptibility factors. Coding RNA includes, but is not limited to, messenger RNA (mRNA), and non-coding RNA comprising a small open reading frame (sORF); and the non-coding RNA includes long non-coding RNA (lncRNA), short hairpin RNA (shRNA), siRNA, self-amplifying RNA, and small activating RNA (saRNA). RNAi can include siRNA and/or miRNA.

In any and all embodiments herein, the genetically-modified microalgae that produce the endo-loaded MEVs can be a species of Chlorella in a pharmaceutically acceptable carrier. Among the species are Chlorella ellipsoidea, Chlorella pyrenoidosa, Chlorella sorokiniana, Chlorella vulgaris, and Chlorella variabilis. In some embodiment the Chlorella is Chlorella vulgaris.

The MEVs and compositions provided herein can be used in any of the methods and uses as described herein or known to those of skill in the art for which the endogenous cargo can be used as described above and below.

The endogenously loaded MEVs from genetically engineered microalgae can be used for a variety of uses, including treatment of diseases, disorders, and conditions, industrial, and cosmetic uses. Diseases, disorders, and conditions, include, but not limited to: genetic disorders, disorders of the digestive tract, disorders of the respiratory tract, disorders of the central nervous system (CNS), disorders of the skin, including natural disorders, and disorders induced by trauma, disorders of the urogenital tract, disorders of the naso-buccal cavity, disorders of the cardiovascular system, immune and immunomodulatory disorders, cancers, ocular disorders, disorders of the liver, systemic disorders, and diseases, disorders, and conditions caused by or involving a pathogen, such as a bacterium, virus, or parasite.

Target tissues for treatment/delivery include, for example, epithelia and mucosa cells (any kind of either external or internal mucosa: mouth, gut, uterus, trachea, bladder, and others), endothelial cells, sensory cells (visual, auditory), cancer cells, tumor cells, blood cells, blood cell precursors, neural system cells (neurons, glial cells and other CNS and peripheral nervous cells), cells of the immune system (lymphocytes, immuno-regulatory cells, effector cells), germ cells, secretory cells, gland cells, muscle cells, stem cells, including, for example, embryonic or tissue specific stem cells, liver cells, infected cells, such as cells infected with virus, bacteria, fungi, or other pathogens, native cells, and NS genetically engineered cells.

Provided are compositions that contain isolated endo-loaded microalgae extracellular vesicles (MEVs), where the genetically engineered microalgae are of species of the genus Chlorella; and the composition is formulated for administration to a subject. The heterologous bioactive cargo molecule, produced by the genetically engineered microalgae cells, is introduced into the MEVs by the same microalgae cells before release of the MEVs to the culture medium. These microalgae cells produce the MEVs and at the same time microalgae load the MEVs before release to the medium. The cargo molecule is heterologous to Chlorella; and the bioactive cargo is a biomolecule.

For all embodiments, the Chlorella is any species of Chlorella, such as, but not limited to, Chlorella selected from among Chlorella ellipsoidea, Chlorella pyrenoidosa, Chlorella sorokiniana, Chlorella vulgaris, and Chlorella variabilis. In particular embodiments, the Chlorella is Chlorella vulgaris.

Provided are compositions that contain isolated endo-loaded microalgae extracellular vesicles (MEVs), where the genetically-modified microalgae is a species of Chlorella; the MEVs in the composition contain bioactive cargo molecule that has been introduced into the MEVs by the genetically-modified microalgae by cellular pathways, existing natural or modified cellular pathways, whereby the vesicles in the composition that contain the heterologous bioactive molecule cargo contain substantially the same cargo, where: the cargo is produced by genetically engineered Chlorella itself; and the cargo is a biomolecule. Each of the MEVs that contain cargo can comprise a plurality of different heterologous cargos. The MEVs can contain endogenously-loaded cargo, and can further contain exogenously-loaded cargo.

The endogenous cargo in the MEV comprises a heterologous peptide, or heterologous small peptide, or heterologous polypeptide, or heterologous protein. The cargo is heterologous to the microalgae in that it is not naturally produced by the microalgae and/or is produced in different amounts or from a different promoter. Cargo includes heterologous nucleic acid that is RNA, with the proviso that the RNA molecules do not comprise inhibitory RNA (RNAi) that targets pathogen genes and/or host pathogen susceptibility factors. Endogenous cargo includes mRNA or modified mRNA. The mRNA as synthesized by the microalgae comprises one or more modifications that inhibit or reduce translation by the microalgae ribosomes, but do not inhibit or reduce translation by ribosomes in animals, such as a human. The endogenous cargo can comprise mRNA that comprises a sequence of linked nucleosides, a 5′ UTR, a 3′ UTR, and at least one 5′ cap structure, optionally one or more regulatory sequences. As noted above, the cargo can comprise heterologous RNAi, with the proviso that the RNAi does not target pathogen genes and/or host pathogen-susceptibility factors. The endogenous cargo can comprise a gene editing system and/or a nucleic acid encoding a gene editing system, such as, for example, clustered regularly interspaced short palindromic repeats (CRISPR)-CRISPR-associated protein 9 (CRISPR-CAS) system, where, for example, the Cas9 is encoded by the nucleic acid molecule of SEQ ID NO:70 or a sequence comprising one or more degenerate codons or a sequence having at least 80%, 85%, 90%, or 95% sequence identity to the nucleic acid molecule of SEQ ID NO:70, or comprising the sequence of amino acids set forth in SEQ ID NO:71, or a sequence of amino acids having at least 95% sequence identity to the sequence of amino acids set forth in SEQ ID NO:71.

The microalgae cell can comprise DNA encoding the endogenous cargo, which is then packaged in the MEVs. The DNA can comprise a plasmid, which can be episomal or can integrate, in whole or part, into the genome of the microalgae. The plasmid can encode a therapeutic product, or a diagnostic product, or a pathway for production of a product by the microalgae and/or production by a subject to whom MEVs containing the endogenous cargo are administered. Endogenous cargo includes, therapeutic products, which include peptides, small peptides, polypeptides, proteins and RNA as described herein, and/or known to those of skill in the art. Therapeutic products include, for example, vaccines, which can be prophylactic or for treatment, anti-cancer products, treatments for infectious agents, and any therapeutic product. Endogenous cargo also includes products with cosmetic activity and products that have industrial uses. The plasmid, for example, can encode a protein product or mRNA for delivery to an animal following administration to the animal, such as a human. For example the endogenous cargo is heterologous to the microalgae, and comprises a small peptide, peptide, polypeptide and/or protein that is packaged in the MEV. Endogenous cargo also includes RNA as described above and below. RNA includes, for example, heterologous mRNA that is packaged in an MEV. The plasmid can be the endogenous cargo product under control of a eukaryotic promoter that is recognized by the microalgae RNA polymerase. Such promoters are known to those of skill in the art, and exemplary promoters, which include microalgae promoters, plant promoters and plant virus, such as those listed in the table in the detailed description, including those set forth or contained in any of SEQ ID NOs: 86-294, modified forms and variants thereof having at least 95%, 96%, 97%, 98%, 99% or more sequence identity with any of the promoter sequences set forth in SEQ ID NOs: 86-206 and with which a eukaryotic RNA polymerase interacts to initiate transcription. Included are inducible and constitutive promoters. The plasmid can further comprise other eukaryotic transcription sequences and eukaryotic translation sequences, such as, for example, one or more of an enhancer, a poly A sequence, and/or encodes an internal ribosome entry site (IRES) sequence. For example, the endogenous cargo in the MEVs can comprise mRNA that comprises an IRES, where the IRES is for translation in an animal, such as a human, and optionally is modified, whereby translation by microalgae ribosomes is reduced, and/or translation by an animal is facilitated or occurs. The plasmid can encode two or more cargo products. The cargo includes a therapeutic product, diagnostic product, and/or biosynthetic pathway that is operably linked to regulatory sequences recognized by a eukaryotic cell. Exemplary products include, an antibody or antigen, a vaccine, an anti-cancer product, and/or an immunomodulatory product. See detailed description and claims for other exemplary products. The vaccine can be prophylactic for preventing, reducing the risk of, or reducing the severity of a disease, disorder, or condition, or can be for use as a therapeutic to treat a disease, disorder, or condition. Endogenous cargo can comprise a nucleic acid, or a small peptide, or a peptide, or a polypeptide, or protein; and/or the endogenous cargo comprises a wild-type nucleic acid, or small peptide, or peptide, or polypeptide, or protein; or a nucleic acid, peptide, or polypeptide or protein that is modified by replacements, insertions, deletions, and/or transpositions of amino acid residues or nucleotide residues; and/or, if nucleic acid, the nucleic acid comprises optimized codon for expression in the microalgae cell, or for expression in the host to whom the MEVs are administered. The endogenous cargo can comprise or encode a protein that is an enzyme, or a hormone, or a cytokine, or a transport protein, or a receptor, or a growth factor, or a member of a signaling pathway, or a member of a protein-protein or protein-nucleic acid complex, or a member of a gene-editing complex or a fragment thereof, and an antibody or antigen-binding fragment thereof, such as an scFv, a bi-specific antibody, or an antigen-binding fragment thereof. Exemplary of antibodies and antigen-binding fragments include, but are not limited to: a checkpoint inhibitor antibody or antigen-binding fragment thereof, or is a tumor antigen-specific antibody or antigen-binding fragment thereof, or is an anti-oncogene specific antibody or antigen-binding fragment thereof, or is a tumor-specific receptor or signaling molecule antibody or antigen-binding fragment thereof. Therapeutic products that can be endo-loaded include vaccines, where the vaccine comprises nucleic acid, a peptide, a small peptide, a polypeptide and/or a protein.

The cargo, thus, includes any suitable heterologous bioactive molecules that are intended for delivery to animals, including human, and that can be introduced into the MEVs in vivo by the genetically engineered microalgae. In general, the cargo is bioactive. Bioactive cargo includes, for example, any molecules, such as biomolecules, including biopolymers, that can have an effect on an animal, including human, when administered. Cargo includes, for example, proteins, peptides, and nucleic acids. Included are any molecules that have been used as drugs or therapeutics or vaccines or diagnostics or cosmetics or in industry. The cargo can be, but is not limited to, a therapeutic for treating or preventing a disease or condition or treating or preventing a symptom thereof. The cargo can be a nucleic acid molecule, a polypeptide, and/or a protein, or other molecule that is produced in vivo in the genetically-modified microalgae and loaded (endo-loaded) into or produced in the MEVs by the microalgae.

The heterologous cargo present in the endo-loaded MEVs in the compositions can comprise a biopolymer. Biopolymers include a naturally occurring biopolymer, or a modified biopolymer, or a synthetic biopolymer, such as synthetic or modified protein or polypeptide, or encoding nucleic acid. The biopolymer can be a nucleic acid or peptide, small polypeptide, polypeptide or protein that includes modifications, where the modifications comprise insertions, deletions, replacements, and transpositions of nucleotides or amino acid residues, and/or, where the biopolymer is a protein, the modifications also can comprise post-translational modifications. Post-translational modifications include, but are not limited to, glycosylation, hyper-glycosylation, and other modifications that improve or alter pharmacological dynamic or kinetic properties of the protein. In all cases, heterologous cargos are endo loaded into the MEVs by microalgae cells that have been genetically modified to become producer cells (or producer cell lines), producers of the heterologous cargos.

The cargo includes therapeutic or diagnostic or theragnostic proteins or peptides, small peptides, polypeptides, and protein complexes, such as complexes that contain two or more proteins or a protein and nucleic acid, or a protein and aptamer, or combinations of proteins, nucleic acids, and other molecules. The cargo can be a protein that is an antibody or antigen-binding fragment thereof. Antibodies can be of any form, including single chain forms, nanobodies, camelids, and other forms, such as an scFv, a bi-specific antibody, or an antigen-binding fragment thereof. Antibodies and antigen-binding fragment thereof include a checkpoint inhibitor antibody or antigen-binding fragment thereof, or a tumor antigen-specific antibody or antigen-binding fragment thereof, or an anti-oncogene specific antibody or antigen-binding fragment thereof or is a tumor-specific receptor or signaling molecule antibody or antigen-binding fragment thereof. Exemplary antibodies and antigen-binding fragment thereof specifically binds to and inhibits one or more of CTLA-4, PD-1, PD-L1, PD-L2, the PD-1/PDL1 pathway, the PD-1/PDL2 pathway, HER2, EGFR, TIM-3, LAG-3, BTLA-4, HHLA-2, CD28, and other checkpoints or immune suppressors, immunomodulators, or tumor antigens. In all cases, heterologous cargos are endo loaded into the MEVs by microalgae cells that have been genetically modified to become producer cells (or producer cell lines), producers of the heterologous cargos. The heterologous cargo in the MEVs in the compositions can include immune stimulating products, or antigens, and can be used as a vaccine to induce an immunoprotective response upon administration. The heterologous cargo can be RNA, protein, or small peptide (sOFR). The heterologous cargo can contain nucleic acid or protein or small peptide (sOFR) that is a therapeutic product for treatment of cancer, or an infectious disease, or a neurodegenerative disease or other CNS disorder, or aging, or aging associated disease, or ophthalmic disorders, or immunological disorders, or genetic disorders or chronical or metabolic diseases. The heterologous cargo can be a cosmeceutical or a cosmetic or cosmetically active product. The heterologous cargo in the MEVs in the compositions can be or comprise a diagnostic marker or detectable product, such as, but not limited to, luciferase or a fluorescent protein. In all cases, heterologous cargos are endo loaded into the MEVs by microalgae cells that have been genetically modified to become producer cells (or producer cell lines), producers of the heterologous cargos.

Methods of preparing the endo loaded MEVs are provided. The methods include obtaining the genetically engineered microalgae cells producers of the heterologous cargos. The methods also include obtaining MEVs endogenously loaded with the heterologous cargo, produced, and loaded by the genetically engineered producer cells. The cargo includes any molecule for whom delivery into or onto an animal, including man is desired. Generally, the cargo is or contains or provides a bioactive molecule product, including biopolymers. The biopolymers are naturally occurring, or modified. The heterologous cargo includes a small peptide, a protein, or a nucleic acid. In some embodiments the MEVs are produced by genetically engineered Chlorella, such as, but not limited to a species of Chlorella selected from among Chlorella ellipsoidea, Chlorella pyrenoidosa, Chlorella sorokiniana, Chlorella vulgaris, and Chlorella variabilis. The endo-loaded MEVs produced by the methods and any of the endo-loaded MEVs provided herein, including the compositions containing the MEVs can be used in one or more of a method of diagnosis, or as vaccine, or therapy for treatment, or diagnosis of a disease, or treatment of a disease or condition, or for cosmetic uses, or for industrial uses, or any use known to those of skill in the art.

The endo-loaded MEVs can be used in any such method, which include methods of treatment of a disease, disorder, or conditions. Exemplary of diseases, disorders, and conditions is cancer, such as a cancer comprising a solid tumor or a hematological malignancy, or metastases thereof. Other diseases, disorders, and conditions include those of or involving the respiratory system, of or involving the central nervous system or the nervous system, of or involving the skin and exposed epithelia or mucosa, of or involving the digestive tract, of or involving an infectious agent. Infectious agents include bacteria, viruses, parasites, prions, oomycetes, and fungi.

The heterologous cargo can provide therapeutic molecules for treatment or can induce an immune response to serve as a vaccine for treating or preventing (reducing the risk of developing or reducing the symptoms, sequela, and/or consequences of) a disease, disorder, or condition. For example, the endo-loaded MEVs can contain a cargo that comprises an immunostimulatory protein or an antigen or encodes an immunostimulatory protein or antigen, whereby the endo-loaded MEVs, upon administration are immune-stimulating and elicit an innate or adaptive immune response, or the endo-loaded MEVs and/or the cargo can elicit an immune-protective response to prevent or treat a disease or condition. The endo-loaded MEVs can be used to treat a disease, disorder, or condition resulting from trauma. Trauma includes, but is not limited to, trauma from or involving wounds, burns, surgery, skin cuts, broken bones, hair loss, dermis exposure, mucosal exposure, fibrosis, lacerations, and ulcerations. The endo-loaded MEVs can be used to elicit an effect to treat a condition resulting from natural aging, or pathogenic or disease or otherwise induced aging. Other diseases, disorders, and conditions that can be treated by the endo-loaded MEVs, are diseases, disorders, and conditions of the skin or the eye. These include dermatitis, wrinkles and/or other age-related changes in the skin, macular degeneration, glaucoma, diabetic retinopathies, cataracts, or conditions resulting from diabetic retinopathy.

The compositions containing the endo-loaded MEVs can be formulated for administration by any route of administration. Routes, include, but are not limited to, local, systemic, topical, parenteral, enteral, mucosal, aerosols for inhalation into the lung or intranasal, parenteral, enteral, vaginal, rectal, aural, oral, nasal, and other routes of administration. The endo-loaded MEVs can be formulated in any form, including as a tablet, as a liquid, such as an emulsion, as a powder, as a cream, as a gel, for oral administration, for nebulization, or for inhalation.

The compositions or endo-loaded MEVs can be used for any of the methods and treatments described herein or known to those of skill in the art. Methods include, for example, any described herein, for use for one or more of gene silencing, gene interference, gene therapy, gene/protein overexpression, gene editing, inhibition or stimulation of protein activity, and pathway signaling. The compositions and endo-loaded MEVs can be used for prophylaxis and/or vaccination. They can be used for dermatological applications, and for cosmetic applications. They can be used for industrial purposes, for example for manufacturing, characterization, and calibration.

The MEVs provided herein have unique biodistribution patterns, which are a function of the route of administration. Biodistribution of the MEVs is different from mammalian EVs and other EVs and/or nanoparticles. For example, systemically delivered mammalian EVs accumulate in the liver, kidneys and spleen. Some mammalian-derived secreted EVs have limited pharmaceutical acceptability (see, e.g., International PCT Publication No. WO2021/122880). While others have shown that certain photosynthetic microalgae release EVs into growth medium, there is no description or understanding of the use of such EVs as drugs or as drug delivery vehicles; there is no description of or understanding of their fate upon administration. It is shown herein that MEVs upon administration via various routes are distributed to organs and tissues differently from mammalian EVs. As one example, while mammalian EVs, with the exception of bovine milk EVs, cannot be administered orally because they do not survive the harsh environment of the stomach, MEVs can be orally administered and delivered to the intestine, from where they traffic to the spleen, including the white spleen. It is shown herein, for example, that intranasally administered MEVs follow unique trafficking patterns and traffic to specific areas of the brain.

As shown and described herein, MEVs, upon intranasal (IN) administration, traverse unique pathways to the brain. Upon IN administration, the MEVs are internalized by olfactory sensory neurons (OSN) from where they travel to the glomeruli. MEVs arriving to the glomeruli from the olfactory sensory neurons (OSN) enter the mitral neurons and tufted neurons and travel intracellularly following a clear pathway with clear kinetics throughout the lateral olfactory tract (LOT). LOTs are composed of the long axons of mitral and tufted neurons that travel from the olfactory bulb (OB) to various anterior-posterior brain regions directly involved in the olfactory network of connections, which include the: anterior olfactory nucleus, olfactory tubercle, tenia tecta, piriform cortex, amygdala, and entorhinal cortex. Lateral ramifications of the main long axons of the mitral/tufted neurons enter and colonize each of the brain regions, the anterior olfactory nucleus, olfactory tubercle, tenia tecta, piriform cortex, amygdala, and entorhinal cortex. Inside these regions, the mitral/tufted axons are connected (via synapses) with neurons from other regions (having a more secondary olfactory role), including the frontal cortex, the hypothalamus, the thalamus, and the hippocampus.

Regions reached by MEVs via IN administration reach all and each of the brain regions connected to the olfactory nerve and the lateral olfactory tract (LOT) in both hemispheres; ventral, lateral and dorsal regions; external and internal regions; along the antero-posterior axis. These regions are: the anterior olfactory nucleus, the olfactory tubercle, the tenia tecta, the piriform cortex, the amygdala, the entorhinal cortex, the primary motor cortex, the frontal cortex, the agranular insular cortex, the primary somatosensory cortex, the auditory cortex, the retrosplenial granular cortex, the temporal association cortex, the basolateral amygdaloid nucleus, the arcuated hypothalamic, the corpus callosum, the internal capsule, the thalamus, and the hippocampus (fimbria, dentata gyrus).

The MEVs are endogenously loaded with a variety of cargos (also referred to as “payloads”), including, but not limited to, DNA, RNA, such as inhibitory RNAs and other RNA products, oligonucleotides, plasmids, peptides, and proteins as detailed herein. As shown and described herein, the MEVs can deliver the cargo to organs, tissues, and cells, and can be targeted by the route of delivery, where they can be delivered. It is shown herein that the MEVs, including the Chlorella MEVs, have a striking capacity to pass through stringent natural barriers, such as the digestive tract, and olfactory neurons, that are not shared by other extracellular vesicles (EVs) from other sources, including mammalian EVs.

The MEVs can be considered or used as drug delivery systems that comprise the MEVs or compositions containing the MEVs formulated for delivery by a particular route of administration. As shown herein, following administration, MEVs traffic by different paths ending up in different organs and/or tissues depending upon the route of administration. The MEVs, thus, are drug delivery systems formulated for administration that targets or delivers to tissue or organs involved in a disease, disorder, or condition, such that the cargo can treat the disease, disorder, or condition. The drug delivery systems thus link a tissues and organs, cargo, route of administration, and formulation. Details, described below and in the claims, of such systems are discussed below.

All of the claims are incorporated by reference into this section.

### BRIEF DESCRIPTION OF THE DRAWINGS

FIGS. 1A and 1B. FIG. 1A depicts the profile of light intensity (in μmol/m²/s) across time (in days) used in the HECTOR photobioreactor cultures of Chlorella; FIG. 1B shows an elution profile for purification of MEVs by size exclusion chromatography (SEC).

FIG. 2 depicts the in vivo full body imaging of a representative animal after intravenous administration as described in Example 10A.

FIG. 3 depicts the in vivo full body imaging of a representative animal per os (oral) administration as described in Example 10A.

FIG. 4 depicts the in vivo full body imaging of a representative animal after intranasal administration as described in Example 10A. As discussed in the Example, and shown in subsequent Examples, MEVs, upon intranasal administration, traffic to the brain. The high volume administered intranasally went to other passages in addition to nasal.

FIG. 5 depicts the in vivo full body imaging of a representative animal after intratracheal administration as described in Example 10A.

FIG. 6 depicts the kinetics of accumulation in liver, spleen, brain, and kidneys (average of 6 animals) after intravenous administration, as described in Example 10A.

FIG. 7 depicts the kinetics of accumulation in liver, spleen, intestine, and kidneys (average of 6 animals) per os administration, as described in Example 10A.

FIG. 8 depicts the kinetics of accumulation in the lungs and kidneys (average of 4 animals) after intranasal administration, as described in Example 10A.

FIG. 9 depicts the kinetics of accumulation in the lungs and kidneys (average of 3 animals) after intratracheal administration, as described in Example 10A.

FIGS. 10A-10D depict ex vivo fluorescence analysis (total radiant efficiency) in organs [10A) liver; 10B) spleen; 10C) lungs; and 10D) brain]isolated 3 days after intravenous (IV), intranasal (IN), per os (PO), and intratracheal (IT) administration.

FIG. 11 depicts the structure of the T-DNA (transferred DNA) portion of the Ti plasmids provided herein. Plasmid sequences, as depicted in the figure, are written from LB (left border of the T-DNA (transferred DNA) element in the Ti plasmid) to RB (right border of the T-DNA element).

FIGS. 12A and 12B depict the blood-brain barrier.

FIG. 13 is a schematic that depicts routes for passage through the olfactory epithelium.

FIG. 14 shows a positive control Dir-MEV on DAPI-stained brain slice: a drop of MEV suspension deposited on top of a brain tissue slide. Puncta are Dir-labeled MEV.

FIG. 15 is a schematic of the Insula and its connections (reproduced from Gogolla (2017) “The insular cortex,” Current Biology 27 (12): R580-R586.

FIG. 16 is a schematic diagram of brain neuronal pathway from the olfactory sensory neurons (OSN) through the olfactory bulb (OB) to the mitral and tufted neurons, to the olfactory tract (OT).

FIG. 17 is a schematic showing the pathways and approximate average distances from the olfactory and respiratory epithelium to CNS targets (reproduced from Lochhead et al. (2019). “Perivascular and Perineural Pathways Involved in Brain Delivery and Distribution of Drugs after Intranasal Administration” Pharmaceutics 11 (11): 598, doi.org/10.3390/pharmaceutics11110598).

FIG. 18 is a schematic of a cortical projection of mitral and tufted cells showing a ventrolateral view of the brain (reproduced from Imai (2014) “Construction of functional neuronal circuitry in the olfactory bulb,” Seminars in Cell and Developmental Biology 35, DOI: 10.1016/j.semcdb.2014.07.012).

FIG. 19 shows transport of MEVs via olfactory pathway. After IN administration, MEVs are taken by the olfactory epithelium transported by axonal transport by olfactory neurons to the olfactory bulb then by mitral and tufted neurons to the primary olfactory regions that process the olfactory signal (reproduced from Selvaraj et al. (2018) Artificial Cells, Nanomedicine, and Biotechnology An International Journal 46:2088-2095, doi.org/10.1080/21691401.2017.1420073).

FIG. 20 depicts the olfactive pathway used by MEVs after IN administration (schematic of the general pathway reproduced from “What-when-how in Depth tutorials and information, Olfaction and Taste, Sensory system, part 1” (what-when-how.com)).

FIGS. 21A-21G: FIG. 21A depicts a general overview of the experimental design of brain biodistribution studies. FIG. 21B depicts the position of the 5 brain sections studied; FIG. 21C depicts the regions analyzed to determine the PK and biodistribution of MEVs in each of the 5 brain sections studied. FIGS. 21D-21G depict and identify regions of the brain for reference with the following figures that show MEVs in the brain following IN administration.

FIGS. 22A-22D show the pharmacokinetic (PK) and biodistribution of MEVs in different regions of section 1 from FIG. 21. Images of labelled-MEVs with DiR are the black dots.

FIG. 23 shows the PK and biodistribution of MEVs in different regions of section 1, providing a graphical representation of the total number of labelled MEVs with DiR spots per surface of regions of section 1, normalized by total analyzed area.

FIGS. 24A-24D show the PK and biodistribution of MEVs in different regions of section 2 (showing images of labelled-MEVs with DiR).

FIGS. 25A-25D show the PK and biodistribution of MEVs in different regions of section 2 as a graphical representation of the total number of labelled MEVs with DiR spots per surface of regions of section 2, normalized by total analyzed area.

FIGS. 26A-26D show the PK and biodistribution of MEVs in different regions of section 3; images of DiR-labelled MEVs.

FIGS. 27A-27F show the PK and biodistribution of MEVs in different regions of section 3 in a graphical representation of total number of labelled MEVs with DiR spots per surface of regions of section 3, normalized by total analyzed area.

FIGS. 28A-28D show the PK and biodistribution of MEVs in different regions of section 4 as images of MEVs labelled with DiR.

FIGS. 29A-29F show the PK and biodistribution of MEVs in different regions of section 4, providing a graphical representation of total number of labelled MEVs with DiR spots per surface of regions of section 4, normalized by total analyzed area.

FIGS. 30A-30D show the PK and biodistribution of MEVs in different regions of section 5 as images of DiR-labelled MEVs.

FIGS. 31A and 31B show the kinetics of brain penetration by the MEVs, from the rostral to the distal parts of the brain.

FIGS. 32A-32E show the biodistribution of MEVs in vivo following per os (PO) administration. FIG. 32A depicts Hematoxylin & Eosin staining of intestine (G=GALT tissue). FIG. 32B depicts DAPI (nuclei) staining and MEV-PKH26 fluorescence (see ro) showing the MEVs in GALT cells (macrophages and dendritic cells). FIG. 32C depicts the INTESTINAL MUCOSA stained with DAPI (for nuclei) and MEV-PKH26 (fluorescence/lighter gray bright puncta) showing the MEVs in the ENTEROCYTES. FIG. 32D depicts the SPLEEN pulp stained with DAPI (for nuclei) and MEV-PKH26 (fluorescence/lighter gray bright puncta). FIG. 32E is a diagram showing the migration of MEVs from the GALT to the spleen.

FIG. 33 shows whole-body bioluminescence imaging of a representative animal treated with MEVs loaded with luciferase mRNA.

FIG. 34 depicts whole-body bioluminescence imaging of a representative animal treated with MEVs loaded with luciferase enzyme.

FIG. 35 depicts image analysis using the Incucyte® live cell analyzer.

FIGS. 36A-36I show results of assessment of toxicity of MEVs in a mouse model after oral (PO) or intratracheal (IT) administration at different doses in 4 groups of mice for each parameter. MEV toxicity was evaluated by chemistry parameters: ALAT, ASAT, urea, and creatine (FIGS. 36A-D, respectively); and 2) by hematology parameters: red blood cells, hemoglobin, hematocrit, MCV and eosinophils (FIGS. 36E-I, respectively). The groups were: Group 1 mice administered 100 μl of PBS (white bars) by PO delivery; Group 2 mice were administered 100 μl of 4*10¹¹ MEV/mouse by PO delivery (white bar with black dots); Group 3 mice were administered 100 μl of 4*10¹² MEV/mouse by PO delivery (white bars with vertical lines); Group 4 mice were administered 100 μl of 4*10¹¹ MEV/mouse by IT delivery (squared bars). Data were measured for six mice per group for each parameter. FIG. 36A shows ALAT: Alanine Aminotransferase; FIG. 36B shows ASAT: Aspartate Aminotransferase; FIG. 36C shows urea; FIG. 36D shows creatine; FIG. 36E shows red blood cells; FIG. 36F shows hemoglobin; FIG. 36G shows hematocrit; FIG. 36H shows MCV (Mean Corpuscular Volume); and FIG. 36I shows eosinophils. PO designates per os (oral delivery) and IT designates Intratracheal administration.

FIG. 37 shows confocal microscopy of Hep-G2 cells including GFP protein expression in Hep-G2 cells after 24 h incubation with MEVs loaded with GFP-protein (MEV-GFP) or MEVs loaded with mRNA-eGFP (MEV-mRNA).

FIG. 38 shows confocal microscopy of Huh7 cells including GFP protein expression in Huh7 cells after 24 h incubation with MEVs loaded with GFP-protein (MEV-GFP) or MEVs loaded with mRNA-eGFP (MEV-mRNA).

FIG. 39 depicts in vivo delivery and expression of mRNA after topical instillation of MEVs into the eyes in rabbits.

FIG. 40 provides representative patterns of biodistribution according to the route of administration, for the Intravenous (IV), Intratracheal (IT) and Per os (PO) routes as described in Example 10A.

### DETAILED DESCRIPTION

### Outline

A. DEFINITIONS
B. MICROALGAE AND OVERVIEW
C. EXTRACELLULAR VESICLES
   1. Types of Extracellular Vesicles (EVs)
      a. Exosomes
      b. Microvesicles
      c. Apoptotic Bodies
   2. Uptake of EVs
   3. General Methods for Isolating EVs
      a. Ultracentrifugation (UC)
      b. Size-Based Techniques
      c. Immunoaffinity Capture-Based Techniques
      d. Exosome Precipitation
      e. Microfluidic Based Isolation Techniques
   4. Microalgae-Derived Extracellular Vesicles (MEVs)
   5. Green algae-Chlorella species
      a. Life Cycle
      b. Genomic Analyses of Chlorella Species
      c. Commercial and Biotechnological Uses of Chlorella
      d. Chlorella MEVs
D. ENDOGENOUSLY LOADED (ENDO-LOADED) MICROALGAE EXTRACELLULAR VESICLES (MEVS), HETEROLOGOUS CARGO, AND TARGETS
   1. Choice and preparation of Cargo
   2. Genetic engineering of producer cells
   3. Isolation of MEVs
   4. Exemplary Heterologous Cargo and Exemplary Uses of the Endogenously Loaded MEVs
      a. Heterologous Cargo
         1) RNA Cargo and nucleic acid cargo
         2) Protein Cargo
      b. Diseases and Methods of Treatment
      c. Cosmetic and Dermatological Applications
E. PHARMACEUTICAL COMPOSITIONS, FORMULATIONS, KITS, ARTICLES OF MANUFACTURE, AND COMBINATIONS, AND DRUG DELIVERY SYSTEMS
   1. Pharmaceutical Compositions and Formulations
   2. Articles of Manufacture/Kits and Combinations
   3. Administration of Endogenously Loaded MEVs and Routes of Administration
   4. Drug Delivery Systems
   5. Combination Therapies
F. BIODISTRIBUTION OF MEVs FOLLOWING ADMINISTRATION VIA VARIOUS ROUTES
   1. Biodistribution of mammalian EVs
   2. Microalgae EVs Biodistribution
      a. Oral Administration
         1) Components of the Lymphatic System
         2) Targeting GALT
   3. Diseases and conditions treated by MEVs
G. FORMULATIONS, ROUTES OF ADMINISTRATION, AND DISEASE AND DISORDERS
H. BIODISTRIBUTION AND DELIVERY OF MEVs TO THE BRAIN VIA INTRANASAL (IN) ADMINISTRATION FOR TREATING DISEASES, DISORDERS, AND CONDITIONS OF THE BRAIN AND CNS
   1. Brain structure
      a. Anterior Olfactory Nucleus
      b. Tenia Tecta
      c. Olfactory Tubercle
      d. Piriform Cortex
      e. Amygdala
      f. Entorhinal Cortex
      g. Frontal Cortex
      h. Striatum: caudate nucleus and putamen
      i. Nucleus accumbens
      j. Thalamus
      k. Hypothalamus
      l. Substantia nigra pars compacta
      m. Hippocampus
      n. Colliculus
      o. Pontine Raphe nuclei
   2. The Blood-Brain Barrier
   3. Brain and target cells
   4. Differences between Biodistribution of MEVs and other delivery vehicles
   5. Intranasal administration
   6. MEVs and delivery to the brain following intranasal administration
   7. Trafficking and biodistribution of MEVs following intranasal (IN) administration
   8. Primary and secondary circuitry of the olfactory system and regions reached by the MEVs upon IN administration
   9. Delivery of MEVs via IN administration to the brain-exemplary bioactive cargo and uses thereof
I. MEV-MEDIATED INTRACELLULAR SIGNALING
J. EXAMPLES

### A. Definitions

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, cargo refers to any heterologous molecules, such as bioactive molecules, including biomolecules, that are either (1) endogenously loaded (by the microalgae) into the extracellular vesicles (MEVs) produced by the genetically-modified microalgae, engineered to express the heterologous cargo and to load it into the MEVs prior to the secretion of the MEVs by the producer cell; or (2) exogenously loaded (by man) into the extracellular vesicles (MEVs) produced by the microalgae, following the isolation and purification of said MEVs.

As used herein, microalgae producer cells are genetically-modified (used interchangeably with genetically engineered) microalgae cells that have been engineered, such as by introduction of a plasmid, to encode a heterologous product. The genetically-modified microalgae then can stably produce the product and package it in MEVs generated in the microalgae.

As used herein, endogenous cargo, refers to cargo in the endogenously-loaded (endo-loaded) MEVs that is loaded into the microalgae extracellular vesicles (MEVs) provided herein inside the producing cell by the microalgae natural or modified pathways and before the MEVs have been isolated. For purposes herein, the MEVs that are loaded with endogenous cargo are referred to as “endo-loaded or “endo loaded.” The cargo is referred to as endogenous cargo since it is endogenously-loaded by the microalgae that produce the cargo and the MEVs loaded with the cargo. Endo-loaded MEVs, are MEVs that contain heterologous cargo that is produced in (and loaded by) the genetically-modified microalgae cell that produces the MEVs. Endogenous cargo is cargo that, generally is heterologous, to the microalgae. The endo-loaded MEVs can contain molecules that are naturally produced in the microalgae, but the cargo generally is heterologous (to the microalgae) but produced by the microalgae upon genetic engineering to create a producer cell or producer cell line. The MEVs can contain endo-loaded and exogenously loaded (exo-loaded) cargo.

As used herein, cargo that is heterologous to the microalgae refers to cargo that is not produced by the microalgae in nature or prior to introduction of nucleic acid encoding the product, or to product produced by genetically modifying the microalgae to produce more of the produce, such as by introducing an additional copy of nucleic acid encoding a product or linking its expression to regulatory sequences that result in higher levels of expression of the product. The microalgae is modified, such as by introducing a plasmid into the microalgae that encodes a product, or by mutation of the genome of the microalgae, or other method by which the microalgae produce a product of interest in sufficient amounts to be packaged in endo-loaded MEVs produced from the microalgae.

As used herein, exogenous cargo refers to cargo loaded after the MEVs are produced and isolated or partially isolated sufficiently for introduction of exogenous cargo.

As used herein, a bioactive molecule refers to any molecule that can have a biological activity or that can act in vivo on a subject. Bioactive molecules include biomolecules, such as RNA, proteins, and any suitable molecules that would be delivered to a subject, such as a human or other animal or a plant or a microorganism (bacteria or other), in connection with a therapy, a diagnostic, or other such uses, such as a cosmetic or a vaccine. The biomolecule can function as or have an activity as, for example, a therapeutic, an immunogen, a diagnostic, a detectable marker, or a cosmetic. The bioactive molecules for use herein are any that can be endogenously loaded by the genetically-modified microalgae itself into the microalgae extracellular vesicles (MEV).

As used herein, a biomolecule refers to any biologically active biopolymer or molecule that occurs, or can occur, in a living organism or virus or that is a modified form of such biopolymer or molecule. Biomolecules, thus, include modified naturally-occurring biomolecules, such as, for example proteins that include a modified primary sequence, such as by deletions, insertions, and/or replacements of amino acids to alter the primary sequence, and or by modification, such as post-translational modifications of the protein.

As used herein, a therapeutic refers to any product that, when administered, results in treating, preventing, reducing the risk of, or ameliorating the symptoms of, or etiology of, a disease, disorder, or condition.

As used herein, a microalgae producer cell is a genetically-modified microalgae that contains genome modifications or a plasmid, generally stably present, so that the microalgae cell is modified, such as a modification to its membranes, modification to express a selectable marker or a detectable marker or a heterologous bioactive molecule, to the constituents of its MEVs, to the products it produces. Endogenously-loaded (endo-loaded) MEVs are generated in producer cells, which are modified to express the endo-loaded product, and/or to alter the microalgae cell. Producer cells produce the MEVs, which can be endo-loaded (by the producer microalgae), or exogenously loaded (exo-loaded) (by man) or both endo- and exo-loaded.

As used herein, a subject is any organism, generally an animal, particularly humans, into which or on which the composition containing the endo-loaded MEV is administered.

As used herein, disease or disorder refers to a pathological or undesirable or undesired condition in an organism resulting from a cause or condition including, but not limited to, infections, acquired conditions, and genetic conditions, and that is characterized by identifiable symptoms.

As used herein, treating a subject with a disease or condition means that the subject's symptoms or manifestations of the disease or conditions are partially or totally alleviated, or remain static following treatment.

As used herein, treatment refers to any effects that ameliorate symptoms of a disease or disorder. Treatment encompasses prophylaxis, therapy and/or cure. Treatment also encompasses any pharmaceutical use of any endo-loaded or exo-loaded MEVs and composition provided herein. Treatment refers to any effects that ameliorate or prevent or other reduce or eliminate any symptom or manifestation of a disease or disorder. Treatment also encompasses any pharmaceutical use of any endo-loaded or exo-loaded MEV or composition provided herein.

As used herein, prophylaxis refers to prevention of a potential disease and/or a prevention of worsening of symptoms or progression of a disease. Prevention or prophylaxis, and grammatically equivalent forms thereof, refer to methods in which the risk or probability of developing a disease or condition is reduced or eliminated and products that reduce or eliminate the risk or probability of developing a disease or condition. Prevention also includes reducing the severity of the disease, disorder, and/or condition, or consequences, sequelae, or other effects of disease, disorder, and/or condition. The vaccine can be used for prophylaxis or prevention of a disease, disorder, and/or condition, and also to treat a disease, disorder, and/or condition. Such vaccines eliciting a humoral immune response, or a cellular immune response, or a combination of both, against pathogens, cancer cells, or other.

As used herein, a vaccine refers to compositions comprising the MEVs, such as the MEVs endo-loaded with a heterologous antigen (such as a small peptide, peptide, polypeptide, and protein) or antigen-producer molecule (such as a mRNA). The vaccine can be used to treat a disease or prevent (reduce the risk of getting a disease), such as for prophylaxis. Vaccines can elicit a humoral immune response, or a cellular immune response, or a combination thereof, such as against pathogens, cancer cells, immune checkpoints, and other therapeutic targets for intervention.

As used herein, a modification with reference to modification of a sequence of amino acids of a polypeptide or a sequence of nucleotides in a nucleic acid molecule refers to and includes deletions, insertions, and replacements of amino acids or nucleotides, respectively. These include modifications of the primary sequence of a polypeptide or protein. Methods of modifying a polypeptide and nucleic acid molecule are routine to those of skill in the art, such as by using recombinant DNA methodologies. Modifications, when referring to polypeptide or protein, not to a sequence, refer to post-translational changes, such as glycosylation or adding purification tags, detectable reporters, and other such moieties.

As used herein, small activating RNA (saRNA) is a small double-stranded RNA that targets a gene promoter to activate a gene. They are generally small, generally 21 nucleotides long. They are structurally similar to siRNA, but the activity of the saRNA is to activate expression; whereas siRNA inhibits expression of its target.

As used herein, self-amplifying RNA refers to RNA that encodes a viral replicase. Upon entry into a host cell, the replicase is translated, which makes a complementary negative copy of the mRNA, which then is used by the replicase as a template to synthesize more mRNA.

As used herein, non-coding RNA is RNA that does not encode a protein. Classes of non-coding RNA, include, but are not limited to, RNAi, such as small interfering RNA (siRNA) and microRNA (miRNA). Also included long noncoding RNAs (lncRNAs).

As used herein, an open reading frame (ORF) is sequence of nucleotides that encodes a start codon followed by a downstream in-frame stop codon. Eukaryotic mRNA predominantly have a single primary ORF and comprises the protein coding sequence (CDS). Shorter ORFs also can occur in the transcript. These are called small ORFs (sORFs).

As used herein, small ORF-encoded peptides (sORFs) occur in non-coding RNA, including in long non-coding RNA (lncRNA), circular RNA, and ribosomal RNA. sORFs encode proteins, small peptides and peptides that are generally less than 100 amino acids. sORFs can include an AUG canonical start codon, but also can include near-cognate codons, such CUG, GUG, UUG, and ACG, that differ from AUG by one nucleotide; sORFs generally terminate with a stop codon (UAA, UGA, and UAG). Many of the products encoded by sORFs have regulatory functions, such as modulating ribosomal RNA.

As used herein, RNA interference (RNAi) is a biological process in which RNA molecules inhibit gene expression or translation, by neutralizing targeted mRNA molecules to inhibit translation and thereby expression of a targeted gene.

As used herein, RNA molecules that act via RNAi are referred to as inhibitory by virtue of their silencing of expression of a targeted gene. Silencing expression means that expression of the targeted gene is reduced or suppressed or inhibited.

As used herein, gene silencing via inhibitor RNA, RNAi, inhibits, suppresses, disrupts, and/or silence expression of a gene with which the RNAi interacts (i.e., a targeted gene). A targeted gene contains sequences of nucleotides that correspond to or are complementary to sequences in the inhibitory RNA, whereby the inhibitory RNA silences expression of encoded mRNA. RNAi includes small interfering RNA (siRNA).

As used herein, long RNA is an RNA molecule that can be enzymatically processed into shorter RNAs, such as short-hairpin RNA (shRNA), micro-RNA, and other RNAs that inhibit or silence expression of mRNA or is long noncoding RNA (lncRNA). For example, small interfering RNAs (siRNAs) are small pieces of double-stranded (ds) RNA, usually about 18-23 nucleotides long, with 3′ overhangs (2 nucleotides) at each end that can interfere with the translation of proteins by binding to and promoting the degradation of messenger RNA (mRNA) at specific sequences. In doing so, siRNAs prevent the production of specific proteins based on the nucleotide sequences of their corresponding mRNAs. The process is called RNA interference (RNAi), and also is referred to as siRNA silencing or siRNA knockdown. A short-hairpin RNA or small-hairpin RNA (shRNA) is an artificial RNA molecule with a tight hairpin turn that can be used to silence target gene expression via RNA interference (RNAi). Expression of shRNA in cells can be accomplished by delivery of plasmids or through viral or bacterial vectors.

As used herein, inhibiting, suppressing, disrupting or silencing a targeted gene refers to processes that alter expression, such as translation, of the targeted gene, whereby activity or expression of the product encoded by the targeted gene is reduced. Reduction includes a complete knock-out or a partial knockout, whereby, with reference to the MEVs provided herein and administration herein, treatment is effected.

As used herein, coding RNA is RNA that encodes a protein, polypeptide, peptide or small Open Reading Frame (sORFs) or a small peptide, generally 10 or fewer amino acids, or peptides (less than hundred amino acids). Classes of coding RNA, include, but are not limited to, mRNA or messenger RNAs that code for proteins and peptides, and long noncoding RNAs (lncRNAs) that encode a sORF encoding a small peptides or peptides.

As used herein, a tumor microenvironment (TME) is the cellular environment in which the tumor exists, including surrounding blood vessels, immune cells, fibroblasts, bone marrow-derived inflammatory cells, lymphocytes, signaling molecules and the extracellular matrix (ECM). Conditions that exist include, but are not limited to, increased vascularization, hypoxia, low pH, increased lactate concentration, increased pyruvate concentration, increased interstitial fluid pressure and altered metabolites or metabolism, such as higher levels of adenosine, indicative of a tumor.

As used herein, recitation that a nucleic acid or encoded RNA targets a gene means that it inhibits or suppresses or silences expression of the gene by any mechanism. Generally, such nucleic acid includes at least a portion complementary to the targeted gene, where the portion is sufficient to form a hybrid with the complementary portion.

As used herein, deletion, when referring to a nucleic acid or polypeptide sequence, refers to the deletion of one or more nucleotides or amino acids compared to a sequence, such as a target polynucleotide or polypeptide or a native or wild-type sequence.

As used herein, insertion, when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence.

As used herein, additions to nucleic acid and amino acid sequences describe addition of nucleotides or amino acids onto either termini compared to another sequence.

As used herein, substitution or replacement refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Amino acid replacements compared to a particular polypeptide can be expressed in terms of the number of the amino acid residue along the length of the polypeptide sequence.

As used herein, at a position corresponding to, or a recitation that nucleotides or amino acid positions correspond to nucleotides or amino acid positions in a disclosed sequence, such as set forth in the Sequence Listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carrillo et al. (1988) SIAM J Applied Math 48:1073).

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other CDNAs, aligned with genomic DNA sequence. Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, a property of a polypeptide, such as an antibody, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an activity of the polypeptide. For example, a change in the binding specificity of the antibody polypeptide can alter the ability to bind an antigen, and/or various binding activities, such as affinity or avidity, or in vivo activities of the polypeptide.

As used herein, an activity or a functional activity of a polypeptide, such as an antibody, refers to any activity exhibited by the polypeptide. Such activities can be empirically determined. Exemplary activities include, but are not limited to, ability to interact with a biomolecule, for example, through antigen-binding, DNA binding, ligand binding, or dimerization, or enzymatic activity, for example, kinase activity or proteolytic activity. For an antibody (including antibody fragments), activities include, but are not limited to, the ability to specifically bind a particular antigen, affinity of antigen-binding (e.g., high or low affinity), avidity of antigen-binding (e.g., high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g., panning assays).

As used herein, bind, bound, and grammatical variations thereof refers to the participation of a molecule in any interaction with another molecule or among molecules, resulting in a stable association in which the molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

As used herein, antibody refers to immunoglobulins and immunoglobulin fragments, whether natural or partially or wholly synthetically, such as recombinantly produced, including any fragment thereof containing at least a portion of the variable heavy chain and light region of the immunoglobulin molecule that is sufficient to form an antigen binding site and, when assembled, to specifically bind an antigen. Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen binding site (e.g., heavy chains include, but are not limited to, VH chains, VH-CHI chains and VH-CHI-CH2-CH3 chains), and each light chain can be a full-length light chain or a portion thereof sufficient to form an antigen binding site (e.g., light chains include, but are not limited to, VL chains and VL-CL chains). Each heavy chain (H and H′) pairs with one light chain (L and L′, respectively). Typically, antibodies minimally include all or at least a portion of the variable heavy (VH) chain and/or the variable light (VL) chain. The antibody also can include all or a portion of the constant region.

For purposes herein, the term antibody includes full-length antibodies and portions thereof including antibody fragments, such as anti-tumor antibody or anti-pathogen or gene silencing fragments. Antibody fragments, include, but are not limited to, Fab fragments, Fab′ fragments, F(ab′) 2 fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibody also includes synthetic antibodies, recombinantly produced antibodies, multispecific antibodies (e.g., bispecific antibodies), human antibodies, non-human antibodies, humanized antibodies, chimeric antibodies, and intrabodies. Antibodies provided herein include members of any immunoglobulin class (e.g., IgG, IgM, IgD, IgE, IgA and IgY), any subclass (e.g., IgG1, IgG2, IgG3, IgG4, IgA1 and IgA2) or sub-subclass (e.g., IgG2a and IgG2b).

As used herein, sORF refers to a small open reading frame encoding a small polypeptide fewer than 100 amino acids. sORFs include longer sORF of around 80 amino acid long that resemble canonical proteins, and dwarf sORFs that, in general, are about 20 amino acids or as few as 3 or 4 amino acids.

As used herein, nucleic acid refers to at least two linked nucleotides or nucleotide derivatives, including a deoxyribonucleic acid (DNA) and a ribonucleic acid (RNA), joined together, typically by phosphodiester linkages. Also included in the term nucleic acid are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a backbone bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single-(sense or antisense) and double-stranded nucleic acids. Deoxyribonucleotides include deoxyadenosine, deoxycytidine, deoxyguanosine and deoxythymidine. For RNA, the uracil base is uridine.

As used herein, an isolated nucleic acid molecule is one which is separated from other nucleic acid molecules which are present in the natural source of the nucleic acid molecule. An isolated nucleic acid molecule, such as a cDNA molecule, can be substantially free of other cellular material, or culture medium when produced by recombinant techniques, or substantially free of chemical precursors or other chemicals when chemically synthesized. Exemplary isolated nucleic acid molecules provided herein include isolated nucleic acid molecules encoding RNAi or a therapeutic protein.

As used herein, operably linked with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to a nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, the residues of naturally occurring α-amino acids are the residues of those 20 α-amino acids found in nature which are incorporated into protein by the specific recognition of the charged tRNA molecule with its cognate mRNA codon in humans. As used herein, an amino acid is an organic compound containing an amino group and a carboxylic acid group. A polypeptide contains two or more amino acids. For purposes herein, amino acids contained in the antibodies provided include the twenty naturally-occurring amino acids (see Table below), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table below). The nucleotides, which occur in the various nucleic acid molecules and fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are generally in the L isomeric form. Residues in the D isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§ 1.821-1.822, abbreviations for amino acid residues are shown in the following Table:

Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. The phrase amino acid residue is defined to include the amino acids listed in the above Table of Correspondence, modified, non-natural and unusual amino acids. A dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in the art and generally can be made without altering a biological activity of a resulting molecule. Those of skill in the art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224).

Such substitutions can be made in accordance with the exemplary substitutions set forth in the following Table:

Exemplary conservative amino acid substitutions

| Original residue | Exemplary Conservative substitution(s) |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, naturally occurring amino acids refer to the 20 L-amino acids that occur in polypeptides.

As used herein, the term non-natural amino acid refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-stereoisomers of amino acids. Exemplary non-natural amino acids are known to those of skill in the art, and include, but are not limited to, 2-Aminoadipic acid (Aad), 3-Aminoadipic acid (bAad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (Melle), 6-N-Methyllysine (MeLys), N-Methylvaline (MeVal), Norvaline (Nva), Norleucine (Nle), and Ornithine (Orn).

As used herein, polypeptide, peptide, and protein refer to polymers of amino acids of any length. Where not used interchangeably other characteristics, such as size, and structure, are contemplated, as defined below. The polymer can be linear or branched, and can contain amino acids, including modified amino acids, and it can be interrupted by non-amino acids. Also included are amino acid polymers that include sequence modifications including, replacements, insertions, deletions, and transpositions. Also included are amino acid polymers that contain post-translational modifications, such as disulfide bonds, glycosylation, sialylation, conjugation to other proteins, peptides, and polypeptides, such, but not limited to, conjugation to a detectable marker, or reporter.

As used herein the term “amino acid” includes natural and/or unnatural or synthetic amino acids, including glycine and the D or L optical isomers, and amino acid analogs and peptidomimetics.

As used herein, a peptide contains at least 2 amino acids, and generally fewer than 100 amino acids. A small peptide is a peptide that contains fewer than 10 amino acids, typically 2 to 6 amino acids.

As used herein, a polypeptide is an amino acid chain that contains a plurality of peptides, and is generally 100 amino acids or longer.

As used herein, a protein is a polypeptide that has a three-dimensional structure and can include bonds in addition to peptide bonds, such as disulfide bonds and other interactions, that participate in forming the two- and three-dimensional structure.

As used herein, cell culture medium that contains MEVs refers to medium harvested from a cell culture that produce MEVs from which the MEVs can be purified. Culture medium is as isolated from the cell culture with cells, cellular, and other debris removed.

As used herein, gene editing technology includes clustered regularly interspaced short palindromic repeats (CRISPR)-CRISPR-associated protein 9 (Cas9), and also other technologies that are used to edit genomes. These include, for example: transcription activator-like effector nucleases (TALENs), zinc-finger nucleases (ZFNs), and homing endonucleases or meganucleases, and other “CRISPR-like” and CRISPR-associated systems, such as the systems developed by Metagenomi.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated nt) or base pairs (abbreviated bp). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule cannot be paired. Such unpaired ends will, in general, not exceed 20 nucleotides in length.

As used herein, production by recombinant methods refers to the use of the well-known methods of molecular biology for expressing proteins encoded by cloned DNA

As used herein, heterologous nucleic acid is nucleic acid that encodes products (i.e., RNA and/or proteins) that are not normally produced in vivo by the cell in which it is expressed, or nucleic acid that is in a locus in which it does not normally occur, or that mediates or encodes mediators that alter expression of endogenous nucleic acid, such as DNA, by affecting transcription, translation, or other regulatable biochemical processes. Heterologous nucleic acid, such as DNA, also is referred to as foreign nucleic acid. Any nucleic acid, such as DNA, that one of skill in the art would recognize or consider as heterologous or foreign to the cell in which it is expressed, is herein encompassed by heterologous nucleic acid; heterologous nucleic acid includes endogenously added nucleic acid that is also expressed endogenously. Heterologous nucleic acid is generally not endogenous to the cell into which it is introduced, but has been obtained from another cell or prepared synthetically or is introduced into a genomic locus in which it does not occur naturally, or its expression is under the control of regulatory sequences or a sequence that differs from the natural regulatory sequence or sequences.

As used herein, MEV therapy involves the administration of an MEV, such as the endo-loaded MEVs provided herein, to a subject to treat a disease, disorder, or condition. The MEVs are loaded with a heterologous cargo, so that they deliver or express products when introduced to a subject.

As used herein, expression refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantification of the polypeptide in the cell lysate by ELISA, Coomassie blue staining following gel electrophoresis, Lowry protein assay and Bradford protein assay.

As used herein, a host cell is a cell that is used to receive, maintain, reproduce and/or amplify a vector. A host cell also can be used to express the polypeptide encoded by the vector. The nucleic acid contained in the vector is replicated when the host cell divides, thereby amplifying the nucleic acids.

As used herein, a vector is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide or RNA. The vector is used to introduce the nucleic acid encoding the polypeptide into the host cell for amplification of the nucleic acid or for expression/display of the polypeptide encoded by the nucleic acid. The vectors can remain episomal, but can be designed to effect integration of a gene or portion thereof into a chromosome of the genome. A vector also includes virus vectors or viral vectors, and bacterial vectors.

As used herein, an expression vector includes vectors capable of expressing DNA that is operatively linked with regulatory sequences, such as promoter regions, that are capable of affecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well-known to those of skill in the art and include those that are replicable in eukaryotic cells and/or prokaryotic cells and those that remain episomal or those which integrate into the host cell genome.

As used herein, primary sequence refers to the sequence of amino acid residues in a polypeptide or the sequence of nucleotides in a nucleic acid molecule.

As used herein, sequence identity refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the no penalty for end gaps is selected. Generally, a global alignment is used on sequences that share significant similarity over most of their length. Exemplary algorithms for performing global alignment include the Needleman-Wunsch algorithm (Needleman et al. (1970) J. Mol. Biol. 48:443). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a local alignment is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2:482 (1981)). For example, 50% sequence identity based on local alignment means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% identical, or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Typically, the full-length sequence of each of the compared polypeptides or nucleotides is aligned across the full-length of each sequence in a global alignment. Local alignment also can be used when the sequences being compared are substantially the same length.

Therefore, as used herein, the term identity represents a comparison or alignment between a test and a reference polypeptide or polynucleotide. In one non-limiting example, at least 90% identical to refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differ from those of the reference polypeptide. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, a therapeutic effect means an effect resulting from treatment of a subject that alters, typically improves or ameliorates, the symptoms of a disease or condition or that cures a disease or condition.

As used herein, a therapeutically effective amount or a therapeutically effective dose refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, therapeutic efficacy refers to the ability of an agent, compound, material, or composition containing a compound to produce a therapeutic effect in a subject to whom the agent, compound, material, or composition containing a compound has been administered.

As used herein, a prophylactically effective amount or a prophylactically effective dose refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose, and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, an anti-cancer agent refers to any agent that is destructive or toxic to malignant cells and tissues. For example, anti-cancer agents include agents that kill cancer cells or otherwise inhibit or impair the growth of tumors or cancer cells.

As used herein therapeutic activity refers to the in vivo activity of a therapeutic polypeptide. Generally, the therapeutic activity is the activity that is associated with treatment of a disease or condition.

As used herein, the term subject refers to an animal, including a mammal, such as a human being.

As used herein, a patient refers to a human subject.

As used herein, animal includes any animal, such as, but not limited to, primates including humans, gorillas and monkeys; rodents, such as mice and rats; fowl, such as chickens; ruminants, such as goats, cows, deer, and sheep; and pigs and other animals. Non-human animals exclude humans as the contemplated animal.

As used herein, a composition refers to any mixture. It can be a solution, suspension, liquid, powder, paste, aqueous, non-aqueous, or any combination thereof.

As used herein, a drug delivery system is a composition or combination of associated compositions or devices or other components for delivery of a bioactive molecule or other cargo packaged in an MEV, such as the endogenously-loaded MEVs provided herein. The delivery system comprises an MEV or MEVs, such as the MEVs that contain endogenous cargo as detailed herein. The delivery systems contain the MEVs in compositions, particularly those formulated for a particular route of administration. Different tissues and organs can be targeted by virtue of the route of administration. The delivery system can include additional components, such as a bioactive agent for combination therapy, or a delivery device. For example, a drug delivery system comprises MEVs produced as described herein. The MEVs can be formulated for a particular route of administration, and the system optionally can include a device for administration of the MEVs.

As used herein, a combination refers to any association between or among two or more items. The combination can be two or more separate items, such as two compositions or two collections, a mixture thereof, such as a single mixture of the two or more items, or any variation thereof. The elements of a combination are generally functionally associated or related.

As used herein, combination therapy refers to administration of two or more different therapeutics. The different therapeutic agents can be provided and administered separately, sequentially, intermittently, or can be provided in a single composition.

As used herein, a kit is a packaged combination that optionally includes other elements, such as additional reagents and instructions for use of the combination or elements thereof, for a purpose including, but not limited to, activation, administration, diagnosis, and assessment of a biological activity or property.

As used herein, a unit dose form refers to physically discrete units suitable for human and animal subjects and packaged individually as is known in the art.

As used herein, a single dosage formulation refers to a formulation for direct administration.

As used herein, a multi-dose formulation refers to a formulation that contains multiple doses of a therapeutic agent and that can be directly administered to provide several single doses of the therapeutic agent. The doses can be administered over the course of minutes, hours, weeks, days or months. Multi-dose formulations can allow dose adjustment, dose-pooling and/or dose-splitting. Because multi-dose formulations are used over time, they generally contain one or more preservatives to prevent microbial growth.

As used herein, an article of manufacture is a product that is made and sold. As used throughout this application, the term is intended to encompass any of the compositions provided herein contained in articles of packaging.

As used herein, a fluid refers to any composition that can flow. Fluids thus encompass compositions that are in the form of semi-solids, pastes, solutions, aqueous mixtures, gels, lotions, creams and other such compositions.

As used herein, an isolated or purified polypeptide or protein (e.g., an isolated antibody or antigen-binding fragment thereof) or biologically-active portion thereof (e.g., an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound. As used herein, a cellular extract or lysate refers to a preparation or fraction which is made from a lysed or disrupted cell.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms a, an and the include plural referents unless the context clearly dictates otherwise. Thus, for example, reference to a polypeptide, comprising an immunoglobulin domain includes polypeptides with one or a plurality of immunoglobulin domains.

As used herein, the term or is used to mean and/or unless explicitly indicated to refer to alternatives only or the alternatives are mutually exclusive.

As used herein, ranges and amounts can be expressed as about a particular value or range. About also includes the exact amount. Hence about 5 amino acids means about 5 amino acids and also 5 amino acids.

As used herein, optional or optionally means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

For clarity of disclosure, and not by way of limitation, the detailed description is divided into the subsections that follow.

### B. Microalgae and Overview

### Microalgae

Algae are a complex, polyphyletic collection of predominantly photosynthetic organisms. These organisms include micro- and macroscopic forms. Macroalgae (seaweed) are multicellular, large-size algae, visible with the naked eye. Microalgae are microscopic single cells and include prokaryotes (e.g., cyanobacteria), and eukaryotes, such as green algae.

Compared to photosynthetic crops, microalgae have a higher growth rate and can be cultivated on non-arable land, and also in bioreactors. Many species of microalgae can be grown year-round in industrial scale photobioreactors under controlled cultivation conditions (Adamo et al. (2021) Journal of Extracellular Vesicles 10: e12081).

Algae generally are classified into eleven major phyla: Cyanophyta, Chlorophyta, Rhodophyta, Glaucophyta, Euglenophyta, Chlorarachniophyta, Charophyta, Cryptophyta, Haptophyta, Heterokontophyta, and Dinophyta (Barkia et al. (2019) Mar. Drugs 17 (5): 304). Different pigments occur in each algae group. Cyanobacteria (or Cyanophyta) contain chlorophyll-a,-d, and -f, in addition to the phycobiliproteins (proteins that capture light energy), phycocyanin, allophycocyanin, and phycoerythrin. Glaucophytes contain chlorophyll-a and harvest light via phycobiliproteins. Chlorophytes have chlorophyll-a and -b, as well as carotenoids, including β-carotene and various xanthophylls (e.g., astaxanthin, canthaxanthin, lutein, and zeaxanthin). The primary pigments of Rhodophyta (red algae) are phycoerythrin and phycocyanin, which can mask chlorophyll-a; red algae also produce a broad spectrum of carotenes and xanthophyll light-harvesting pigments (Barkia et al. (2019) Mar. Drugs 17 (5): 304).

Provided herein are extracellular vesicles produced by microalgae, particularly unicellular green algae, such as species of Chlorella, for use for delivery of endogenously loaded heterologous cargo to animals, including humans. The microalgae (Chlorella) are unicellular eukaryotes that typically are haploid but can have a diploid stage of the life cycle. The microalgae can be cultured in bioreactors and the extracellular vesicles isolated therefrom. The resulting extracellular vesicles can be endogenously loaded inside the genetically engineered producing cell by the microalgae itself with a heterologous cargo produced by the engineered producer cell, generally a cargo of bioactive molecules to produce compositions that contain the extracellular vesicles for administration to animals, including human. The compositions can be formulated for any desired route of administration, including topical, local, systemic, parenteral, intranasal, inhalation, by any type of nebulizer and oral. These routes include oral, intravenous, subcutaneous, inhalation, mucosal, rectal, vaginal, intranasal and other suitable routes. The heterologous cargo includes biomolecules, such as DNA, RNA, proteins, protein complexes, and protein-nucleic acid complexes. The extracellular vesicles can be formulated as liquids, powders, including lyophilized powders, tablets, capsules, emulsions, particles, sprays, gels, ointments, creams, and other formulations. They can be used for therapeutic, diagnostic, theragnostic, cosmetic, and other uses. The extracellular vesicles can be used to treat diseases and conditions, that include cancers, inflammatory diseases and conditions in which the immune system plays a role in the etiology or symptoms, nervous system disorders, and pathogen infections, including viral and bacterial and other pathogens. They can be used to treat dermatological diseases and conditions, lung diseases and conditions, and gastric diseases and conditions. The extracellular vesicles can be targeted to specific organs or tissues or can be locally administered.

### Microalgae and Microalgae Extracellular Vesicles (MEVs)

The following discussion provides an overview of the disclosure herein. Provided herein are MEVs, and genetically-modified microalgae cells that produce the MEVs, including heterologous cargo-loaded MEVs, cargo that can be loaded into the MEVs, routes of administration of MEVs, in vivo distribution of MEVs upon administration to animals.

As described and shown herein, MEVs have a number of advantages over the use of existing drug delivery systems, such as, exosomes derived from mesenchymal stem cells, gold nanoparticles, liposomes and other plant and animal-derived EVs. Mesenchymal stem cells are a commonly used source of exosomes, and exosomes derived from mesenchymal stem cells are used in drug delivery, for example as anti-cancer vaccines, because they have enhanced passive targeting (a method of preparing a drug carrier system so that it remains circulating in the blood stream), as a result of their small size, indigenous nature, and ability to cross biological barriers. Mesenchymal stem cells, however, have limited secretion of exosomes, and scaling up production of exosomes is difficult due to the need to optimize purification, increase the homogeneity of exosomes, and establish efficient transfection strategies. Nanoparticles can lead to toxicity and techniques for synthesizing nanoparticles are limited in their ability to scale for manufacturing purposes. Nanoparticle and liposome-based drug delivery methods also can lead to the formation of a teratoma (a tumor comprised of several different types of tissue). Liposome-based drug delivery methods have been shown to be less efficient for internalization into a specific cell, tissue or organ, compared to exosomes. Plant-derived EVs, such as those from curcumin, ginger, grapefruit, and lemon, have been used for drug delivery, but their extraction process and use in treatment has not been optimized nor exploited. The production of EVs from agricultural products, such as fruits and milk, is economically impractical since such products require 3-4 months to grow.

Algae cells can be grown anywhere and within a few days. Algal EVs avoid phagocytosis or degradation by macrophages and circulate for prolonged times in vivo, and have low immunogenicity. Algal EVs also have a lower risk of teratoma formation. Algae, thus, provide a source from which pure, well-characterized EVs of high quality can be obtained (Kuruvinashetti et al. (2020) 20th International Conference on Nanotechnology 354-357). Kuruvinashetti et al. does not describe the use of Chlorella species as a source of EVs, nor its advantages as a source. Prior art does not describe the biodistribution of MEVs per se, nor the implications thereof for administration of MEVs with drugs directed to particular organs, tissues, or systems.

A focus herein are MEVs that are endogenously loaded (endo-loaded; loaded by the microalgae), and the genetically-modified microalgae producer cells that express the heterologous cargo and endo-load it into the MEVs before the MEVs are secreted into the culture medium. The MEVs and compositions containing them can serve as drug delivery systems.

Extracellular vesicles (EV) mediate interaction between cells, mediate non-classical protein secretion, facilitating processes such as antigen presentation, in trans signaling to neighboring cells and transfer of RNAs, proteins and metabolites. These vesicles are secreted by different cell types/tissues and harbor a common set of molecules that are essential for their structure and trafficking apart from distinct subsets of proteins/RNA, reflecting the biological function of the producer cell (Mathivanan et al. (2009) Proteomics 9:4997-5000, doi.org/10.1002/pmic.200900351). MEVs are naturally internalized by human cells cultured in vitro as well as by bacteria (see, International PCT Publication No: WO2022/053687, and in vivo, by mouse cells, as a mammalian model, as shown and described herein.

Native MEVs contain proteins, lipids, including fatty acids and glycerolipids, carbohydrates, including sugars, oligosaccharides, and polysaccharides, and nucleotides. They have a membrane that forms a particle that has a diameter of 50-250 nm. MEVs do not have a cell wall. The lumen of the MEVs contain macromolecules that originate with the producer microalgae cell. These macromolecules include, for example, mRNAs, sRNAs, peptides and proteins, including surface proteins and glycoproteins can be surface markers. The contents of the MEV lumen are protected from degradation by proteases and RNases.

Extracellular vesicles from microalgae (MEVs) also mediate interactions between cells and among cells, facilitating processes such as trans-signaling to neighboring cells and transfer of bioactive molecules, such as siRNA. These vesicles are secreted by the microalgae cell and harbor a common set of molecules that are essential for their structure and trafficking. The microalgae cells can be genetically modified to encode heterologous siRNA to provide producer cells that express the siRNA and package the siRNA in MEVs (see, International PCT Publication No.: WO 2022/053687). This finding is extended herein to provide genetically-modified producer microalgae cells that generate endo-loaded MEVs that contain other heterologous bioactive molecules, such as peptides and proteins, mRNA, other types of RNAi, including siRNA to different targets, shRNA, saRNA, and products produced by the genetically-modified microalgae producer cells.

MEVs are generated by a producer cell of a microalgae species, such as Chlorella vulgaris. They can be generated in a suspension cell culture system in volumes up to at least 170 liters. The MEVs can be generated in a semi-continuous culture system in volumes up to at least 600 liters. They are isolated by methods including steps of centrifugation, filtration (such as 1.2 μm) and membrane concentration (such as with a molecular weight cut-off of Da) and can be further purified. For example, they can be further purified in a multiple-step process that includes tangential flow filtration (TFF), diafiltration, size exclusion chromatography (SEC), and ultracentrifugation. They can be stored at −50° C./−80° C. (for at least 2 years), at +4° C. for at least 1-2 weeks, until use. MEVs can be subjected to at least 3 cycles of freezing/thawing without losing their integrity. MEVs can be heated up to 60° C. without losing their integrity.

MEVs can be modified by introducing heterologous cargo either exogenously (by man, after isolation and purification of the MEV), and/or endogenously (by producer cells genetically modified to express and load the heterologous cargo, prior to the secretion of the MEVs). MEVs thereby can comprise heterologous cargo, such as a polypeptide, a nucleic acid (such as DNA or RNA) or other polynucleotide, large biologicals, or small molecules. The cargo can be introduced before (endo-loading) of after (exo-loading) separation of MEVs from the producer cell or by endo-loading and then exo-loading to add second heterologous cargo, such as for combination therapy. EVs can be isolated from the producer cell and then modified, thereby generating exo-loaded MEVs. The microalgae cell can be genetically modified with a nucleic acid (genetic coding regions for siRNA, mRNA; miRNA, proteins, polypeptides, SORF (small open reading frames), lncRNAs (long non-coding RNA) to generate a producer cell line; producer of MEVs with modified composition and/or behavior.

As described herein, the cells can be modified to express heterologous proteins or produce heterologous mRNA product, or to produce other biomolecules, such as by introduction of nucleic acid encoding a biosynthetic pathway. Appropriate selection of regulatory sequences can result in producer cells that produce a lot of a particular product that is packaged in MEVs.

The producer cells also can be genetically modified, for example, to facilitate or increase production of MEVs or result in MEVs with particular structure. For example, microalgae producer cells can be genome modified to have modified cellular or membrane content, such as changed cytoplasm content and/or changed content of the cell membrane. In another example, a producer microalgae cell can contain a nucleic acid that can be transcribed (e.g., mRNA), and when mRNA is made, it can be translated into a polypeptide, by either the MEV producer cell or by the cells targeted by the MEVs. The producer cell can also be modified to generate non-translatable RNA (e.g., siRNA, miRNA or long non-coding RNA), with regulatory roles, and also to produce mRNA that is not translated or not translated to a large extent by the microalgae cell, but is translated by a mammalian, such as human cell. This can be effected by modifying portions of the mRNA responsible for interaction with ribosomes, and/or for interaction with aminoacyl-tRNAs complexes and/or other activities related to translation. MEVs derived from the producer cell can carry the non-translatable RNA, the transcribed RNA, or the translated polypeptide as a cargo. MEVs generated from the modified producer cell comprise the modifications of the producer cell, which can include a heterologous cargo. Those MEVs are endo-loaded with the heterologous cargo. MEVs can also be endo-loaded and exo-loaded with different molecular cargo(s).

EVs, including MEVs can interact with the target cell and deliver a cargo (e.g., a therapeutic agent or other) to a target cell. The target cell can be a cell from any organism, including an animal, such as a mammal, including a human, a plant, a bacterium, or a fungus. The heterologous cargo, which is a bioactive agent, such as a therapeutic agent, acts on the target cell that is contacted with the cargo-loaded MEV. Contacting can occur in vitro or in vivo in a subject. It is shown herein that MEVs can be internalized and distributed to various tissues and organs; biodistribution depends upon the route of administration. As shown and described herein, MEVs can be detectably labelled to track and visualize them inside cells, tissues and organisms. Labeling agents, include, but are not limited to, fluorescent membrane markers, such as PKH26, PKH 67, DiR, and others.

As described and shown herein, MEVs can be internalized by cells of the human respiratory system in vitro in a dose-dependent manner. As shown herein, MEVs can be internalized by cells, in tissues and organs in animal models. As shown herein, the MEVs have distribution patterns that differ from animal EVs. For example, MEV absorption in vitro by human alveolar epithelial cells is in the order of 90% of the cells at 4 hours post-treatment, with a maximum at 24 hours. Exposure of human alveolar epithelial cells to MEVs is not toxic at a MEV-to-cell ratio up to 10,000,000. MEVs can be administered in vivo to a mouse model, at doses of up to 4×10¹⁰ MEVs per animal, with no signs of toxicity.

As shown herein, the MEVs target specific organs upon administration by several routes, as shown in a rodent model in the Examples. The routes include intravenous, intranasal, inhalation (intratracheal) and oral (per os). It is shown herein that specific organs targeted by MEVs depend on the route of administration and include the lungs, the spleen, the liver, the brain, the intestine, and the GALT (gut-associated lymphoid tissue). Upon intranasal administration, MEVs are transported from the olfactory bulb to different regions of the brain in vivo. Upon oral administration, MEVs are transported to the gut-associated lymphoid tissue (GALT), and internalized by GALT cells in vivo. Upon oral administration, as shown here, MEVs are transported from the intestine to the spleen in vivo. Upon intravenous administration, MEVs are transported to the liver and to the spleen, in vivo. Upon intratracheal administration, MEVs are transported to the lungs, in vivo.

### C. Extracellular Vesicles

Extracellular vesicles (EVs), in general, are biomolecular structures released from plant and animal cells that play a role in cell-to-cell communication.

Structurally, EVs are negatively charged lipid bilayer vesicles with a density of 1.13 to 1.19 g/mL. EVs are able to cross barriers such as the plasma (or cytoplasmic) membrane and the blood/brain barrier, and enable the horizontal transfer of their functional contents (i.e., proteins, lipids, RNA molecules, and circulating DNA) from a donor to a recipient cell (Kuruvinashetti et al. (2020) 20th International Conference on Nanotechnology 354-357). EVs also are naturally stable in various biological fluids, immunologically inert, and can exhibit organ-specific targeting abilities (Picciotto et al. (2021) Biomater. Sci. doi: 10.1039/d0bm01696a).

EVs contain endogenous lipids, nucleic acids, and proteins. Although results differ due to variations in isolation techniques and methods of analyzing the data, EVs generally contain proteins associated with the plasma membrane, cytosol and those involved in lipid metabolism (see, e.g., Doyle and Wang (2019) Cells 8 (7): 727). Proteins involved in the biogenesis of EVs (e.g., components of the ESCRTs), EV formation and release (e.g., RAB27A, RAB11B, and ARF6), signal transduction, and antigen presentation, as well as tetraspanins, commonly occur in EVs (Abels et al. (2016) Mol. Neurobiol. 36 (3): 301-312). EVs are enriched for cholesterol, sphingomyelin, glycosphingolipids, and phosphatidylserine (Kuruvinashetti et al. (2020) 20th International Conference on Nanotechnology 354-357).

Although a small number of studies have identified genomic and mitochondrial DNA in EVs, EVs are primarily enriched with endogenous small RNAs. Studies have identified mRNAs, miRNAs, rRNAs, long and short non-coding RNA, RNA fragments, piwi-interacting RNA, vault RNA, and Y RNA in EVs. Most of the RNA that naturally occurs in EVs is ˜200 nucleotides long (with a small portion up to 4 kb) and thus it is fragmented, although circular RNAs also have been shown to be enriched and stable in EVs. RNA in EVs is protected from RNase digestion in the extracellular environment by the lipid bilayer (Abels et al. (2016) Mol. Neurobiol. 36 (3): 301-312). The Exocarta, Vesiclepedia, and EVpedia databases are publicly available and provide data on the protein, nucleic acid, and lipid content of EVs (generally EVs from mammalian, such as human origin), as well as the isolation and purification procedures used, from EV studies (Abels et al. (2016) Mol. Neurobiol. 36 (3): 301-312).

In mammals, EVs are used by cells to mediate several physiological processes or affect various pathological conditions associated with the activation of an immune response or the spread of disease or infection. EVs, in general, also can mediate cross-species communication and they occur in all kingdoms of life. Sources of EVs include mammalian cells, bacteria, bovine milk and plants (Adamo et al. (2021) J. Extracell. Vesicles 10: e12081). Although plants and algae possess a cell wall outside their plasma membrane, which could be a physical barrier for the release of EVs, plants and algae release EVs (Picciotto et al. (2021) Biomater. Sci. doi: 10.1039/d0bm01696a; see also Khamsi (2020) Nature 582 (7812): S19-S19, DOI: 10.1038/d41586-020-01771-1; and International PCT Publication No. WO2022/053687, discussed above).

### 1. Types of Extracellular Vesicles (EVs)

### a. Exosomes

There are three primary subtypes of EVs; they are classified based on their biogenesis, mode of release, size, content, and function: microvesicles (MVs), exosomes, and apoptotic bodies (Doyle and Wang (2019) Cells 8 (7): 727). Exosomes, or intraluminal vesicles (ILVs), are 30-150 nm in diameter and are released through multivesicular bodies (MVBs) in the endosomal pathway. In the endosomal pathway, early endosomes form by inward budding of the plasma membrane and can transform into late endosomes, which accumulate ILVs by inward budding of the endosomal membrane. Late endosomes which contain a number of small vesicles are called MVBs. MVBs either fuse with the lysosome and are degraded, or the plasma membrane which releases the IL Vs as exosomes into the extracellular space. The endosomal sorting complexes required for transport (ESCRT) pathway regulates MVB transportation and exosome formation and is reported to be the primary driver of exosome biogenesis, although other mechanisms of exosome biogenesis exist, including those mediated by the sphingolipid ceramide, which can facilitate membrane invagination, or proteins in the tetraspanin family. The ESCRT accessory proteins Alix, TSG101, HSC70 and HSP90β have been referred to as exosomal marker proteins (Doyle et al. (2019) Cells 8 (7): 727).

Exosomes are released into the extracellular space by the fusion of the MVB limiting membrane with the plasma membrane. A number of proteins are involved in the release of exosomes, including Rab GTPases, diacylglycerol kinase a, and SNARE proteins (Abels et al. (2016) Cell Mol. Neurobiol. 36 (3): 301-312).

Exosomes are candidates for drug delivery systems: they have a long circulating half-life; exosomes are tolerated by the human body and can penetrate cell membranes and target specific cell types; and they can be loaded with genetic material, a protein, or a small molecule (Doyle and Wang (2019) Cells 8 (7): 727).

### b. Microvesicles

Microvesicles (MVs, or ectosomes) form by outward budding, or pinching, of the cell's plasma membrane, and have a diameter of 100 nm to 1 μm. The formation of MVs involves cytoskeleton components, such as actin and microtubules, molecular motors such as kinesins and myosins, and fusion machinery such as soluble N-ethylmaleimide-sensitive factor attachment protein receptors (SNAREs), and tethering factors. The physiological state and microenvironment of the donor cell effects the number of MVs produced, and the physiological state and microenvironment of the recipient cell effects the number of MVs consumed. MVs also have a number of marker proteins, including cytosolic and plasma membrane associated proteins, as well as cytoskeletal proteins, heat shock proteins, integrins, and proteins containing post-translational modifications, although there are no known specific markers to distinguish MVs from exosomes. Like exosomes, MVs can be loaded with cargo (such as proteins, nucleic acids, and lipids) for delivery to another cell, thereby altering the recipient cell's functions (Doyle and Wang (2019) Cells 8 (7): 727).

### c. Apoptotic Bodies

Apoptotic bodies are released by dying cells into the extracellular space, and have a diameter from 50 nm to 5000 nm. Apoptotic bodies are formed when the cell's plasma membrane separates from the cytoskeleton due to increased hydrostatic pressure after the cell contracts. Unlike exosomes and MVs, apoptotic bodies contain intact organelles, chromatin, and small amounts of glycosylated proteins (Doyle and Wang (2019) Cells 8 (7): 727).

### 2. Uptake of EVs

Cells internalize EVs by fusion with the plasma membrane, or more commonly by endocytosis (Abels et al. (2016) Cell Mol. Neurobiol. 36 (3): 301-312). Uptake via endocytosis can be through several types of endocytotic processes, and different processes have been described in different cell types: clathrin-dependent endocytosis and phagocytosis have been described in neurons, macropinocytosis in microglia, phagocytosis and receptor-mediated endocytosis in dendritic cells, caveolin-mediated endocytosis in epithelial cells, and cholesterol- and lipid raft-dependent endocytosis in tumor cells. Blocking heparin sulfate proteoglycans (HSPGs) on the plasma membrane with heparin reduces the uptake of EVs in cell culture, as does blocking the scavenger receptor type B-1 (SR-B1) with a synthetic nanoparticle mimic of HDL, which suggests a role for HSPGs and SR-B1 in EV uptake (Abels et al. (2016) Cell Mol. Neurobiol. 36 (3): 301-312). Fusion of EVs with the plasma membrane also is a method of uptake, and requires low pH conditions; treatment of EVs with the combination of a pH-sensitive fusogenic peptide with cationic lipids resulted in increased cellular uptake of exosomes and the cytosolic release of cargo within the exosomes (Nakase et al. (2015) Sci. Rep. 5:10112). Low pH conditions occur in tumors (Abels et al. (2016) Cell Mol. Neurobiol. 36 (3): 301-312), so that EVs for delivering therapeutic cargos to tumor cells can enter cells through fusion with the plasma membrane.

Like cells, EVs have extracellular receptors and ligands on the outside and cytoplasmic proteins and nucleic acids on the inside. They, thus, can communicate with cells in a variety of ways. EVs bind to the cell surface, undergo endocytosis, and/or fuse with the plasma membrane, and release their cargos in the extracellular space. If entering by endocytosis, the EV cargo must escape the degradative pathway; late endosomes can fuse with lysosomes or the plasma membrane, so cargo must exit before it is degraded in a lysosome or re-released through the fusion of MVBs with the plasma membrane. EVs that contain cargo, including mRNAs and non-coding RNAs, can be transferred to recipient cells in culture and in vivo (Abels et al. (2016) Cell Mol. Neurobiol. 36 (3): 301-312; Maas et al. (2017) Trends Cell Biol. 27 (3): 172-188).

### 3. General Methods for Isolating EVs

### a. Ultracentrifugation (UC)

Ultracentrifugation methods are used to isolate exosomes; alternative methods also have been developed. Due to the complex nature of the biological fluids from which exosomes are isolated, the overlap in physiochemical and biochemical properties between exosomes and other types of EVs, and the heterogeneity among exosomes, isolation methods can result in complex mixtures of EVs and other components of the extracellular space. Differential ultracentrifugation depends on the initial sedimentation of larger and denser particles from the extracellular matrix, and results in an enrichment of exosomes, but not a complete separation of exosomes from other components in the extracellular space. Density gradient centrifugation is another ultracentrifugation method and is based on separation by size and density in the presence of a density gradient (typically made of sucrose or iodoxinol) in the centrifuge tube. Density gradient centrifugation effectively separates EVs from protein aggregates and non-membranous particles but has low exosome recovery, although purity can be improved by coupling differential ultracentrifugation with types of density gradient centrifugation, such as rate-zonal centrifugation or isopycnic centrifugation (Doyle and Wang (2019) Cells 8 (7): 727).

### b. Size-Based Techniques

There are a number of size-based techniques for isolating exosomes (Doyle and Wang (2019) Cells 8 (7): 727). Ultrafiltration separates particles based on the size and molecular weight cut off of the membrane, whereby particles larger than the molecular weight cut off of the membrane are retained, and particles smaller than the molecular weight cut off of the membrane are passed through into the filtrate; low isolation efficiency can occur however if the filter becomes clogged and vesicles become trapped. The ExoMir Kit (Bioo Scientific; Austin, TX) is a commercially available kit in which two membranes (200 nm and 20 nm) are placed into a syringe and a sample (typically pre-treated with centrifugation and proteinase K) is passed through the syringe; the larger vesicles remain above the first 200 nm filter, the smallest vesicles are passed through the syringe and discarded, and the vesicles between 20 and 200 nm remain between the two filters in the syringe. Sequential filtration also relies on a series of filtration steps to isolate exosomes (Doyle and Wang (2019) Cells 8 (7): 727).

Tangential Flow Filtration (TFF) often is employed before UC or to replace UC for EV isolation and concentration of EVs to optimize the recovery of intact EV. TFF methods use to streams flow tangentially to a tubular filter membrane, which allows the passage of particles smaller than the pore size from the feed stream into the permeate stream and retains larger particles in the retentate stream. Depending on the choice of pore size the TFF can be used to purify and concentrate EVs and eliminate smaller contaminants. TFF can be applied to buffer exchange or for the product concentration in the retentate stream. TFF is a flexible and rapid methods to purify and concentrate EVs. TFF is scalable, industrialize The TFF does not alter the integrity of EVs or liposomes thus offer a gentler purification of EVs in comparison to UC and despite high purification yield of intact EVs but provides EVs with lower purity than UC. A process using TFF coupled with SEC enable more efficient removal of contaminant and similar yield of EVs compared to UC. (Paganini et al. (2019) Biotechnol. J. 14:1800528).

Size Exclusion Chromatography (SEC), often used in parallel with ultracentrifugation methods (in which the exosome pellet obtained from ultracentrifugation is resuspended and further purified using SEC), of exosomes is similar to using SEC to separate proteins. SEC can be used in parallel with TFF methods. In SEC, a column is packed with a porous stationary phase in which small particles can penetrate and thus elute after larger particles. Typically SEC methods require several hours of run time; however, the qEV Exosome Isolation Kit (iZON Science, New Zealand) allows for rapid and precise exosome isolation by SEC within 15 minutes (Doyle and Wang (2019) Cells 8 (7): 727).

In Flow Field-Flow Fractionation (FFFF), a sample injected into a chamber is subjected to parabolic flow as it is pushed down the chamber, in addition to a flow perpendicular to the parabolic flow, a crossflow, to separate particles in the sample. Larger particles are more affected by the crossflow and are pushed toward the walls of the chamber, which have a slower parabolic flow, and smaller particles remain in the center. Smaller particles elute earlier, and larger particles later, in FFFF (Doyle and Wang (2019) Cells 8 (7): 727).

In Hydrostatic Filtration Dialysis (HFD), hydrostatic pressure forces a sample through a dialysis tube with a membrane having a molecular weight cut off of 1000 kDa. The result is that small solutes are able to pass through the tube, but larger particles, including exosomes and EVs, remain in the tube and can then be further separated using, for example, ultracentrifugation (Doyle and Wang (2019) Cells 8 (7): 727).

### c. Immunoaffinity Capture-Based Techniques

Immunoaffinity capture-based techniques can isolate exosomes based on expression of an antigen on the surface of the exosome, and allow for the isolation of exosomes derived from a particular source. In these methods, an antibody specific for a target antigen can be attached to a plate (e.g., in Enzyme-Linked Immunosorbent Assay, ELISA), magnetic beads (e.g., in magneto-immunoprecipitation), resins and microfluidic devices; these surfaces are then exposed to the exosome sample, resulting in the immobilization of the exosomes expressing the antigen. This assay requires that the protein/antigen for isolating the exosomes be expressed on the surface of the exosomes, and its specificity is limited by the specificity of the antibody that is used, often resulting in a lower yield but higher purity of isolated exosomes. These methods also can be used to separate exosomes within mixed populations of EVs. Immunoaffinity capture-based techniques often are used after ultracentrifugation or ultrafiltration (Doyle and Wang (2019) Cells 8 (7): 727).

### d. Exosome Precipitation

Methods for precipitation of exomes include precipitation by polyethylene glycol (PEG) and lectin. In PEG precipitation, the PEG polymer ties-up the water molecules, allowing the other particles, including exosomes, to precipitate out of solution. PEG precipitation is quick and is not limited to the starting volume of solution, but lacks selectivity, as other EVs, extracellular proteins, and protein aggregates are precipitated with EVs. Sample pretreatment using filtration and/or ultracentrifugation can improve exosome yield. Commercially available kits for isolating exosomes using precipitation include, for example, ExoQuick (System Biosciences, Palo Alto, CA) and Total Exosome Isolation Kit (Thermo Fisher Scientific, Waltham, MA). Alternatively, lectin precipitation can be used, typically after ultracentrifugation, whereby lectins bind to carbohydrates on the surface of exosomes, altering their solubility and leading to their precipitation out of solution (Doyle and Wang (2019) Cells 8 (7): 727).

### e. Microfluidic Isolation Techniques

Microfluidic based techniques isolate exosomes based on their physical and biochemical properties simultaneously, and are rapid, efficient, and require small starting volumes. In acoustic nanofilter, a matrix containing EVs and other cellular components is injected into a chamber and exposed to ultrasound waves. The particles respond differently to the radiation forces exerted by the waves, depending on their size and density; large particles experience stronger forces and migrate faster toward the pressure nodes. The immuno-based microfluidic isolation technique is similar to that of an ELISA, although, unlike ELISAs, it does not require prior ultrafiltration or ultracentrifugation of exosomes (Doyle and Wang (2019) Cells 8 (7): 727). The ExoChip (Kanwar et al. (2014) Lab Chip. 14 (11): 1891-1900) and ExoSearch Chip (Zhao et al. (2016) Lab Chip. 16 (3): 489-496) have been developed to isolate exosomes using microfluidic technology.

### 4. Microalgae-Derived Extracellular Vesicles (MEVs)

Microalgae are bioresources for the production of EVs for use in nanomedicine and other fields. The mechanism of secretion of EVs from microalgae is known in relation to primary and motile cilia/flagella (Picciotto et al. (2021) Biomater. Sci. doi: 10.1039/d0bm01696a). EVs can be isolated from different microalgae strains, and the microalgae isolated EVs exhibit the key features of EVs. For example, eighteen (18) microalgae strains (Ankistrodesmus sp., Brachiomonas sp., Chlamydomonas reinhardtii, Dunaliella tertiolecta, Tetraselmis chuii, Chloromonas sp., Rhodella violacea, Kirchneriella sp., Pediastrum sp., Nannochloropsis sp., Cyanophora paradoxa, Cryptomonas pyrenoidifera, Phaeodactylum tricornutum, Phaeothamnion sp., Diacronema sp., Isochrysis galbana, Stauroneis sp., and Amphidinium sp.) from the main microalgae lineages were studied. These include strains with a variety of features such as saltwater and freshwater inhabitants, small and large sized cells, colonial and single cells, and species with sequenced genomes. EVs were isolated using a differential ultracentrifugation protocol and characterized following the International Society for Extracellular Vesicles (ISEV) guidelines, and all strains tested showed the presence of EVs in the culture medium. EV-producing microalgae strains were established based on the EV protein content, the expression of EV protein markers (e.g., Alix, Hsp70, enolase, and β-actin), the total scattering signal (measured by dynamic light scattering, DLS) or total particle number (measured by NTA), and the microalgae small EVs (sEVs, designated nanoalgosomes) average size and size range. Chlorella strains were not considered or tested nor considered for future study. The strains identified for future study were Cyanophora paradoxa, Tetraselmis chuii, Amphidinium sp., Rhodella violacea, Diacronema sp., Dunaliella tertiolecta, Phaeodactylum tricornutum, Pediastrum sp., and Phaeothamnion sp. the data for Cyanophora paradoxa showed ˜2×10⁹ sEV particles per mL of microalgae-conditioned media, with strong positive signals for EV markers, and a size distribution with a mode of 130±5 nm, in agreement with data from plant-derived vesicles. Cytotoxicity and genotoxicity studies showed that sEVs isolated from Cyanophora paradoxa, a freshwater Glaucophyte, did not show toxicity on the tumorigenic MDA-MB 231 breast cancer or C2C12 myoblast cell lines, neither over time nor at different concentrations, nor did MDA MB 231 cells treated with the sEVs show morphological nuclear changes associated with apoptotic events (Picciotto et al. (2021) Biomater. Sci. doi: 10.1039/d0bm01696a).

The studies did show that, in general, microalgae produce EVs that can be isolated using traditional methods; microalgae-derived EVs are similar in size and concentration, and exhibit similar markers compared to EVs isolated from other species; EVs isolated from microalgae do not show cytotoxic or genotoxic effects in vitro; and that microalgae-derived EVs can be taken up by cells. Such studies have not considered Chlorella species, nor assessed routes of administration, nor the fate of administered microalgae-derived EVs following administration.

EVs have been extracted from algal cells using ultra-centrifugation (Kuruvinashetti et al. (2020) 20th International Conference on Nanotechnology 354-357). This method is well established for the isolation of EVs. Algae cells are cultured; the cultured algal cells are collected and centrifuged; the supernatant is collected (and further centrifuged); a sucrose solution is added to the supernatant; and the algal supernatant with the sucrose solution is ultra-centrifuged; because of the sucrose solution, the high-density EVs settle at the bottom of the ultra-centrifugation tube and can be collected using a pipette. Extracted algal EVs can be characterized in size and concentration using Nanoparticle Tracking Analysis (NTA). Studies using this method have isolated green algal EVs that range in size from 25-200 nm, with a concentration of 0.89E8 to 0.94E8 particles/mL (Kuruvinashetti et al. (2020) 20th International Conference on Nanotechnology 354-357).

An ultra-centrifugation protocol also can be used to isolate EVs from marine microalgae grown under various conditions; NTA showed that the nano-particles have a size distribution between 100 and 200 nm, and western blotting of proteins confirmed the presence of EV markers (VES4US, Extracellular vesicles from a natural source for tailor-made nanomaterials, 2020). Subsequent studies have identified sEVs (nanoalgosomes), isolated from the marine photosynthetic microalgae chlorophyte Tetraselmis chuii. The production of nanoalgosomes is an evolutionarily conserved trait within microalgae strains as similar results were obtained using sEVs isolated from batch cultures of two other microalgae species, the chlorophyte Dunaliella tertiolecta, and the dinoflagellate Amphidinium sp. The nanoalgosomes were isolated using differential centrifugation (dUC) and tangential flow filtration (TFF), as well as gradient ultracentrifugation, which was used to further purify samples enriched for sEVs by TFF or dUC. The isolated nanoalgosomes were shown to share characteristics of EVs from other sources. The EV yield (measured by sEV protein content and sEV number) from dUC and TFF was consistent with reported numbers of isolated EVs, around 10⁹ EV particles/μg EV proteins. Biophysical analysis of particle size using multi-angle dynamic light scattering (DLS), nanoparticle tracking analysis (NTA), fluorescence nanoparticle tracking analysis (F-NTA), and fluorescence correlation spectroscopy (FCS) yielded consistent size distributions, with the size that appeared the most frequently from DLS (DLS mode) around 70 nm. Compared to exosomes derived from mammalian cells, which have a density of 1.15-1.19 g/mol, nanoalgosomes had a slightly lower density of 1.13 g/mol. Electron microscopy reveals that the nanoalgosomes are spherical, heterogeneous in size and shape, and possess a lipid-bilayer structure. Compared to the microvesicles (or large EVs, 1EVs) and lysates, the sEVs were enriched for three of the four target protein biomarkers (Alix, enolase, HSP70 and β-actin). DLS measurements indicated that the nanoalgosomes were resistant to changes in pH and stable in human blood plasma. The tumorigenic MDA-MB 231 breast cancer cell line, the non-tumorigenic 1-7 HB2 cell line, and the human hepatocarcinoma Hep G2 cell line did not show cytotoxic or genotoxic effects after nanoalgosome treatment. The nanoalgosome were taken up by the MDA-MB 231 and 1-7 HB2 cell lines (Adamo et al. (2021) J. Extracell. Vesicles 10: e12081).

It has been shown that EVs from mammalian origin can deliver cargo to a target cell and can have therapeutic use for delivery of a variety of cargos for treating a number of diseases or conditions. For example, macromolecular proteins and nucleic acids can be embedded into the exosomes. The nucleic acids can include those encoding a gene of interest. Specific targeting ligands, imaging probes, and covalent linkage could be attached to the exosome surface and tracked using NTA, fluorescence, or by bioluminescence. Other than speculation that microalgae EVs possibly might have a use for drug delivery (see, Kuruvinashetti et al. (2020) 20th International Conference on Nanotechnology 354-357), there is no published evidence nor technical descriptions that the uses described for mammalian EVs can be applied to or developed for microalgae-derived extracellular vesicles (MEVs). It is shown herein that MEVs can deliver cargo to target cells in animals, and that the biodistribution pathways are unique and appear to be different from the mammalian EV pathways.

As described and shown herein, however, MEVs have a number of advantages compared to existing drug delivery systems, such as, exosomes derived from mesenchymal stem cells, gold nanoparticles, liposomes and other plant- and animal-derived EVs. Mesenchymal stem cells are a commonly used source of exosomes, and exosomes derived from mesenchymal stem cells are used in drug delivery, for example anti-cancer vaccines, because they have enhanced passive targeting (a method of preparing a drug carrier system so that it remains circulating in the blood stream), as a result of their small size, indigenous nature, and ability to cross biological barriers. Mesenchymal stem cells, however, have limited secretion of exosomes, and scaling up production of exosomes is difficult due to the need to optimize purification, increase the homogeneity of exosomes, and establish efficient transfection strategies. Nanoparticles can lead to toxicity and current techniques for synthesizing nanoparticles are limited in their ability to scale for manufacturing purposes. Nanoparticle and liposome-based drug delivery methods also can lead to the formation of a teratoma (a tumor comprised of several different types of tissue). Liposome-based drug delivery methods have been further shown to be less efficient for internalization into a specific cell, tissue or organ, compared to exosomes. Plant-derived EVs, such as those from curcumin, ginger, grapefruit, and lemon, have been used for drug delivery, but their extraction process and use in treatment has not yet been optimized. The production of EVs from agricultural products, such as fruits and milk, is economically impractical and need 3-4 months to grow, compared to algal EVs, which can be grown anywhere and within a few days. Kuruvinashetti et al. (2020) 20th International Conference on Nanotechnology 354-357, discussed above does not describe the use of Chlorella species as a source of EVs, nor its advantages as a source nor the production of and use of genetically-modified microalgae to produce EVs that are endogenously loaded with heterologous bioactive molecules.

### 5. Green Algae-Chlorella Species

Previous studies and consideration of EVs have not focused on nor assessed Chlorella species as sources of EVs. Chlorella and the resulting EVs have advantages for growth, manipulation, and administration of drugs that other species and EVs do not provide. Green algae belong to phylum Chlorophyta, and encompass a diverse group of photosynthetic eukaryotes. Green algae include unicellular and multicellular organisms. Algae originally included in the genus Chlorella are among the most widely distributed and frequently encountered algae in freshwater. These algae exist in aqueous environments and on land. They are typically small (˜2 to 10 μm in diameter), unicellular, spherical in shape, non-motile, and contain a single chloroplast, and some have a rigid cell wall (Blanc et al. (2010) Plant Cell 22 (9): 2943-2955).

Molecular analyses have separated Chlorella species into two classes of chlorophytes: the Trebouxiophyceae, which contains the true Chlorella, and the Chlorophyceae. For use herein, Chlorella species include any that can be or that are used as food complement or that can be consumed by humans or other animals, such as livestock. Exemplary species include, but are not limited to, the species: Chlorella ellipsoidea, Chlorella pyrenoidosa, Chlorella sorokiniana, Chlorella vulgaris, and Chlorella variabilis.

True Chlorella species are characterized by glucosamine as a major component of their rigid cell walls. Although most Chlorella species are naturally free-living, the Trebouxiophyceae include most of the known green algal endosymbionts, living in lichens, unicellular eukaryotes, plants, and animals (for example mussels and hydra). For example, Chlorella variabilis NC64A is a hereditary photosynthetic endosymbiont (or photobiont) of Paramecium bursaria, a unicellular protozoan, and NC64A also is a host for a family of large double-stranded DNA viruses that are occur in freshwater (Blanc et al. (2010) Plant Cell 22 (9): 2943-2955).

### a. Life Cycle

In unicellular organisms, such as microalgae, life cycle is the same as the cell cycle. Chlorella is a haploid organism that reproduces asexually by autosporulation. The cell cycle and proliferation of Chlorella vulgaris has been investigated using flow cytometric analysis of 5-,6-carboxyfluorescein diacetate succinimidyl ester (CFSE)-stained algal cells (see, Rioboo et al. (2009) doi: 10.1016/j.aquatox.2009.07.009). The results indicate that, as generally described for microalgae, the growth of C. vulgaris mother cells takes place during light periods, whereas cytoplasmic division and liberation of daughter cells takes place during dark periods. C. vulgaris also shows a distinct light/dark cycle, marked by an increase in cell size, cell complexity, and autofluorescence during periods of light, measured over a 96-hour period. A monoparametric histogram of CFSE-stained C. vulgaris cells showing only one peak of daughter cells indicates that each mother cell undergoes only one division cycle in 96 hours; the cytoplasmic division was further shown to take place during periods of darkness. Thus, the strain of C. vulgaris used exhibits three life cycle phases: 1) growth of mother cells, 2) cell division, and 3) liberation of daughter cells. C. vulgaris cells grew during 2 light periods and began to divide during following dark period; cell division occurs once the mother cells are double the size of daughter cells. Furthermore, C. vulgaris cells exposed to the herbicide terbutryn need a longer growth period in order to reach a large enough cell size to divide. This suggests there is a critical threshold size needed for C. vulgaris to complete the growth phase and begin the division phase, and that this critical threshold can control the progression of the G1 phase of the C. vulgaris cell cycle. Finally, this study demonstrates that the intensity of the peak of CFSE-fluorescence of mother cells is four times greater than that of the daughter cells, indicating that 4 daughter cells are produced from each mother cell. Thus, C. vulgaris cells undergo a first mitosis followed by cytoplasmic division, and then two other simultaneous mitoses, which result in the liberation of 4 daughter cells (Rioboo et al. (2009) doi: 10.1016/j.aquatox.2009.07.009).

### b. Genomic Analyses of Chlorella Species

Although species of Chlorella are reported to be non-motile and lack a sexual cycle, genomic analyses of Chlorella variabilis NC64A (NC64A) and Chlorella vulgaris 211/11P (211/11P) reveal the presence of genes involved in sexual reproduction and motility (Blanc et al. (2010) Plant Cell 22 (9): 2943-2955; Cecchin et al. (2019) Plant J. 100 (6): 1289-1305). The NC64A nuclear genome (GenBank Accession No. ADIC00000000.1) is 46.2 Mb, and composed of 12 chromosomes. The meiosis-specific proteins dosage suppressor of MCK1 DMC1, homologous-pairing proteins HOP1 and HOP2, meiotic recombination protein MER3, meiotic nuclear division protein MND1, and mutS homolog protein MSH4 are encoded in NC64A; these genes also occur in most of the other sequenced chlorophyte algal species. Nineteen homologs of the microalgae Chlamydomonas gametolysin proteins, which promote disassembly of the gametic cells walls and allow gamete fusion, also were identified in NC64A. Additionally, an ortholog of the Chlamydomonas GCS1 protein, which is essential for cell fusion, occurs in NC64A (Blanc et al. (2010) Plant Cell 22 (9): 2943-2955). The primary genes involved in meiosis also occur in the Chlorella vulgaris 211/11P 40 Mb genome (GenBank Accession No. SIDB00000000), in addition to the gene encoding gametolysin (g3347), and a gene encoding a protein that contains a domain with a putative GCS1/HAP2 function (Cecchin et al. (2019) Plant J. 100 (6): 1289-1305). Thus, although Chlorella species have been observed only in the haploid phase, the presence of meiosis genes indicates that the life cycle of Chlorella could include a diploid phase.

Similarly, while flagella have not been observed in NC64A, orthologs of the Chlamydomonas flagellar proteins were identified in the NC64A genome, including orthologs to the intraflagellar transport (IFT) proteins IFT52, IFT57, and IFT88, kinesin-2 motor protein FLA8, the kinesin-associated protein KAP, and proteins involved in the axonemal outer dynein arm (Blanc et al. (2010) Plant Cell 22 (9): 2943-2955).

Sequencing of three Chlorella sorokiniana strains, strain 1228, UTEX 1230, and DOE1412, reveals the presence of sex- and flagella-related genes (Hovde et al. (2018) Algal Research 35:449-461). The genome of several other Chlorella species has been sequenced: Chlorella protothecoides sp. 0710 (Gao et al. (2014) BMC Genomics 15 (1): 582; GenBank Accession No. APJO00000000); Chlorella sorokiniana UTEX 1602 (GenBank Accession No. LHPG00000000) and Chlorella sp. strain SAG 241.80 (Micractinium conductrix; GenBank Accession No. LHPF00000000) (Arriola et al. (2018) Plant J. 93 (3): 566-586); and the Chlorella vulgaris strains UTEX 395 (Guarnieri et al. (2018) Front. Bioeng. Biotechnol. 6:37; GenBank Accession No. LDKB00000000), UMT-M1 (Teh et al. (2019) Data Brief 27:104680; GenBank Accession No. VJNP00000000), UTEX 259 (GenBank Accession No. VATW00000000) and NJ-7 (Wang et al. (2020) Mol. Biol. Evol. 37 (3): 849-863; GenBank Accession No. VATV00000000).

### c. Commercial and Biotechnological Uses of Chlorella

The commercial cultivation of microalgae for food purposes began with the production of Chlorella vulgaris in Japan and Taiwan in the 1960s. Dried biomass products from Arthrospira and Chlorella are included in dietary supplements due to reports of high protein content, nutritive value, and health benefits. For example, Chlorella extracts have been shown to lower cholesterol and have antioxidant, antibacterial, and antitumor activities. Production of high yields of Chlorella is routine, and, as detailed herein, Chlorella MEVs can be isolated from the cell culture medium. For its use as a pharmaceutical, it is known that ingestion of Chlorella is non-toxic and non-immunogenic in humans.

Chlorella has been used in a variety of biotechnology applications, including biofuels, sequestering CO₂, producing molecules of high economic value, or removing heavy metals from wastewaters (Blanc et al. (2010) Plant Cell 22 (9): 2943-2955). Chlorella species show metabolic flexibility in response to environmental perturbations, and are capable of using nutrients, such as organic carbon and minerals, directly from wastewater for growth. Among microalgae, Chlorella species have higher photosynthetic efficiency over other photosynthetic organisms. Additionally, Chlorella vulgaris is able to grow either in autotrophic, heterotrophic or mixotrophic conditions (Zuñiga et al. (2016) Plant Physiol. 172 (1): 589-602).

Chlorella species also can be genetically modified by Agrobacterium-mediated transformation. A study by Cha et al. developed a method to genetically transform Chlorella vulgaris using the Agrobacterium tumefaciens strain LBA4404, and the presence of gene fragments in 30% of the transgenic lines, compared to the wild-type non-infected Chlorella, indicates the T-DNA was integrated into the Chlorella genome (Cha et al. (2012) World J. Microbiol. Biotechnol. 28:1771-1779).

### d. Chlorella MEVs

As described herein, Chlorella species, such as C. vulgaris, are advantageous species for the production of EVs, referred to herein as MEVs, for use for delivery of biomolecules for many applications, including therapeutic, diagnostic, and cosmetic uses. Of particular interest herein are MEVs produced by Chlorella species. Chlorella EVs have not been exploited as sources of MEVs for endogenous loading of heterologous biomolecular products. Chlorella, as a source of EVs for such applications, provides numerous advantages. Chlorella is a haploid organism, which means that specific and targeted variants can be produced by genetic engineering; it readily can be genetically modified to produce or contain heterologous biologically active molecules. Stable cell lines can be produced, including stable producers of encoded heterologous products. They are defined products, and, when endogenously loaded by the microalgae, the resulting compositions contain MEVs that carry the same heterologous products as a cargo.

Detailed genetic maps can be obtained, and correlations between genotype and phenotype can be established. Chlorella genomes have been fully sequenced, so the structure and function of various genes can be known. Phylogenetically, Chlorella is at the very crossroads between higher plants and microalgae. As such, Chlorella shares with higher plants a significant (and useful) number of molecular biological and metabolic features, but still is a unicellular haploid microalgae. Exemplary of molecular biological features shared with eukaryotes is the intracellular machinery that involves the dicer enzyme system for processing endogenous RNA into siRNA. Chlorella is autotrophic: unlike mammalian and other animal cells, it can therefore be cultured and reproduced without the need for nutrients or factors of animal origin.

With respect to use of its EVs as therapeutics, Chlorella species are not toxic. For example, tablets made from Chlorella vulgaris biomass (i.e. compressed whole Chlorella cells) have been consumed regularly for years by the public worldwide as a dietary supplement, without constraints related to toxicity or immunogenicity. Japan is the world leader in the consumption of Chlorella biomass. It also is used, for example, in Japan, for medical treatments because it has shown to have immunomodulatory properties and purported anti-cancer activities, for use for anti-aging applications, such as for cardiovascular diseases, hypertension and cataracts; it reduces the risk of atherosclerosis and stimulates the synthesis of collagen for the skin.

Chlorella cells naturally produce extracellular vesicles that respond to the ‘standard specifications’ of better known EVs (such as mammalian EVs). EVs from plant origin bear a number of features that make them more promising/convenient than synthetic nanoparticles or semisynthetic EVs, for use as a drug delivery system in humans. These include, for example, higher stability, lower toxicity, and lower immunogenicity. Since they are close to plants, Chlorella provides a source of MEVs with similar characteristics to plant EVs. At the same time, mass production of Chlorella in large scale is easier and cheaper than for higher plants. The glycosylation pattern of membrane proteins in Chlorella is similar/identical to the glycosylation pattern present in higher plants.

The size of the Chlorella MEVs ranges between about or between 50 nm and 200 nm, with an average size of about 130 nm. The morphology resembles plant and mammalian exosomes. For use for administration, the size distribution can be rendered more uniform by separating the MEVs by size and selecting those of a size of interest, which can vary depending upon the intended use and route of administration.

### D. Endogenously Loaded (Endo-Loaded) Microalgae Extracellular Vesicles (Mevs), Heterologous Cargo, and Targets

For endo-loaded MEVs, DNA encoding a product of interest, such as a protein, mRNA, synthetic pathway, or other product, is introduced into the microalgae cell by any suitable method. Methods for introducing DNA into a microalgae cell are known in the art (for a review see, e.g., Gutierrez et al. (2021) Biology 10:265). Heterologous DNA can be introduced into microalgae by a variety of methods, including but not limited to, mechanical agitation, surfactant permeabilization, electroporation, particle bombardment, bacterial DNA transfer, nanoparticles, liposomes, and cell penetrating peptides or cell penetrating polymers to mediate penetration into the cell, and other methods known to those of skill in the art for introducing DNA into plant cells, particularly microalgae cells. For examples, microalgae cells can be transformed by Agrobacterium tumefaciens transformation using the Ti plasmid of the agrobacterium. This process is well-known to those of skill in the art. The Ti plasmid, into which DNA of interest can be cloned, introduces DNA into the microalgae genome. The DNA of interest integrates into the microalgae genome. To prepare endo-loaded MEVs, DNA that encodes the heterologous product to be endo-loaded in the MEVs, is introduced into the microalgae and the microalgae produces the heterologous product, such as a protein, or mRNA.

Targets and cargo (see discussions below) include any known to those of skill in the art. For endo-loading, the heterologous product must be one that is produced by or loaded into the microalgae cell, and from the cell into the cell-produced MEVs.

### 1. Choice and Preparation of Cargo

The MEVs can be endogenously loaded with any suitable heterologous cargo, including, but not limited to, nucleic acid molecules, including, for example RNAi, such as siRNA, miRNA, lncRNA, and mRNA, including modified mRNA, encoding coding any protein, polypeptide and peptide, detectable marker proteins and tags or any therapeutic or prophylactic or vaccine polypeptide or peptide, gene editing systems, and others, and combinations thereof. The MEVs can deliver therapeutic molecules, can serve as vaccines, and can be used in human health, gene therapy applications, including delivery genes, modification of genes with gene editing systems, and gene silencing nucleic acids, cosmetic applications, dermatological applications, diagnostic applications, industrial uses, and others. The MEVs can deliver regulators of gene pathways to produce a beneficial product, and can be used to deliver gene editing systems, such as CRISPR/cas (see e.g., SEQ ID NOs: 70 and 71 for exemplary CRISPR/cas protein and encoding nucleic sequences, respectively) to effect gene editing.

Diseases and conditions that can be treated include any known to those of skill in the art, including but not limited to, cardiovascular diseases, metabolic diseases, infections, including respiratory infections, bladder infections and other urinary tract infections, infectious diseases, including viral diseases, such as hepatitis, HIV, corona viruses, including SARS-COV-2, CNS diseases, ocular diseases, and liver diseases. As discussed, delivered cargo includes protein products, such as, but not limited to, enzymes, regulatory factors, signaling proteins, antigens, antibodies and antigen-binding forms thereof, RNA products, such as, but not limited to, siRNA, miRNA (micro RNA), lncRNA (long non-coding RNA), saRNA (small activating RNA), shRNA, and mRNA, including modified mRNA, such as modified mRNA to increase stability for delivery.

An advantage of MEVs for delivery, is that the RNA is a labile molecule, and so, mRNAs delivered by other kinds of nanoparticles, like lipid nanoparticles (LNPs) have been modified to increase RNA stability. For delivery in MEVs, the mRNA does not necessarily have to be modified. For endo-loading, the mRNA, in general, the mRNA will be unmodified.

Modified mRNA, which in general will not be for endo-loaded MEVs, can be synthetic mRNA that comprises a translatable region that contains at least one nucleoside modification in which at least a percentage of the uridine nucleotides in the synthetic mRNA are modified (see, e.g., U.S. Pat. No. 9,464,124, which describes modified mRNA for delivery and translation). For example, at least one nucleoside modification can be pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. The mRNA can include a) a sequence of linked nucleotides, a 5′ UTR, a 3′ UTR, and at least one 5′ cap structure. The mRNA also can include other regulatory sequences for translation and trafficking in a eukaryotic, such as a mammalian, such as a human, host cell.

The MEVs can carry cargos that include reporter genes and proteins and other detectable products, such as, for example, a fluorescent protein, such as, but not limited to an enhanced green fluorescent protein (EGFP; SEQ ID NO:10), a luciferase gene (SEQ ID NO:11), luxA (SEQ ID NO:8), luxB (SEQ ID NO:9), and the Lux operon (luxCDABE and luxABCDE; SEQ ID NO:12).

Other cargos can target genes or products involved in diseases, such as, but not limited to, Peptidyl-prolyl cis-trans isomerase FKBP4 or FKBP52 (SEQ ID NO: 1); gamma-aminobutyric acid type B receptor subunit 1 (GABBR1; SEQ ID NO: 3); oncogenes such as MYCN or NMYC (SEQ ID NO:38), RAS (H-RAS, N-RAS, and K-RAS; see SEQ ID NOs: 39, 40, and 41, respectively), BCL2 (SEQ ID NO: 43), and PLK1 (SEQ ID NO:44). Genes involved in diseases, such as oncogenes, and checkpoints, can be modulated by cargo that encodes a product that inhibits or agonizes expression of a gene, or inhibits or agonizes a gene product. Exemplary of such modulators, are RNAis, such as, for example, siRNAs, miRNAs, shRNAs, peptides and/or tetratricopeptides. For example, siRNAs and ASOs targeting EGFP (SEQ ID NOs: 5 and 6), firefly luciferase (SEQ ID NO:7), MYCN (SEQ ID NOs: 13-19), RAS (SEQ ID NOs: 20-27), BCL2 (SEQ ID NOs: 29-31), and PLK1 (SEQ ID NOs: 32-35), and microRNA-34A, which targets MYC and BCL2 (SEQ ID NO:28), are exemplified herein.

Gene silencing using RNA interference, including siRNAs and miRNAs, can be used to silence developmental genes, such as, for example, adhesion molecules, cyclin kinase inhibitors, Wnt family members, Pax family members, Winged helix family members, Hox family members, cytokines/lymphokines and their receptors, growth/differentiation factors and their receptors, and neurotransmitters and their receptors; oncogenes; tumor suppressor genes; enzymes; genes associated with a pathological condition; genes associated with autoimmune diseases; anti-angiogenic genes; angiogenic genes; immunomodulator genes; genes associated with alcohol metabolism and liver function; genes associated with neurological disease; genes associated with tumorigenesis or cell transformation; and genes associated with metabolic diseases and disorders (see, e.g., WO 2009/082606, JP 2014-240428A, WO 2011/072292A2, WO 2010/141724, and WO 2020/097540). These types of heterologous products can be delivered in or encoded in MEVs to activate genes or pathways or to provide therapeutic effects. Certain cytokines can be used to treat diseases/disorders, such as certain cancers, in which immune suppression plays a role.

Endogenously-loaded MEVs can be used to transfer therapeutic agents such as nucleic acids, such as microRNA, mRNA, tRNA, IRNA, siRNA, regulatory RNA, non-coding and encoding RNA, and DNA fragments (see, e.g., CN105821081A and CN110699382A); nucleotides or amino acids comprising a detectable moiety; polypeptides (e.g., enzymes) (see, U.S. Pat. No. 10,195,290). Non-limiting examples of proteins that may be encoded for by the nucleic acid cargo molecule include, but are not limited to: antibodies, intrabodies, single chain variable fragments, affibodies, enzymes, transporters, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins, structural proteins, neurotrophic factors, membrane transporters, nucleotide binding proteins, heat shock proteins, CRISPR-associated proteins, cytokines, cytokine receptors, caspases and any combination and/or derivatives thereof (see, e.g., AU2018365299).

Cargo bioactive molecules can target central nervous system diseases, such as neurodegenerative diseases, such as Alzheimer's Disease. Exemplary of such is FKBP52 and the tetratricopeptide derivative therefrom. The full sequence of human peptidyl-prolyl cis-trans isomerase FKBP4 is (SEQ ID NO:1):

MTAEEMKATESGAQSAPLPMEGVDISPKQDEGVLKVIKREGTGTEMPMI
GDRVFVHYTGWLLDGTKFDSSLDRKDKFSFDLGKGEVIKAWDIAIATMK
VGEVCHITCKPEYAYGSAGSPPKIPPNATLVFEVELFEFKGEDLTEEED
GGIIRRIQTRGEGYAKPNEGAIVEVALEGYYKDKLFDQRELRFEIGEGE
NLDLPYGLERAIQRMEKGEHSIVYLKPSYAFGSVGKEKFQIPPNAELKY
ELHLKSFEKAKESWEMNSEEKLEQSTIVKERGTVYFKEGKYKQALLQYK
KIVSWLEYESSESNEEAQKAQALRLASHLNLAMCHLKLQAFSAAIESCN
KALELDSNNEKGLFRRGEAHLAVNDFELARADFQKVLQLYPNNKAAKTQ
LAVCQQRIRRQLAREKKLYANMFERLAEEENKAKAEASSGDHPTDTEMK
EEQKSNTAGSQSQVETEA

### The Tetratricopeptide Repeat (TPR) Domain 260-400 is (SEQ ID NO:2):

MNSEEKLEQSTIVKERGTVYFKEGKYKQALLQYKKIVSWLEYESSFSNE
EAQKAQALRLASHLNLAMCHLKLQAFSAAIESCNKALELDSNNEKGLER
RGEAHLAVNDFELARADFQKVLQLYPNNKAAKTQLAVCQQRI

For example, cargo can be delivered or encoded in MEVs for treatment of Alzheimer's Disease by preventing accumulation of Tau. An in vitro model is available and was developed by a group from Institut National de la Santé et de la Recherche Médicale, Université Paris XI (see, Chambraud et al. (2007) FASEB J. 21 (11): 2787-97; and Chambraud et al. (2010) Proc. Natl. Acad. Sci. U.S.A. 107 (6): 2658-63). The effect of depletion of FKBP52 in PC12 cultured cells was examined by introducing two different small, interfering RNA (siRNA) duplexes specific for rat FKBP52, named RNAi 1 and RNAi 2. The sense sequence of siRNAs and an oligonucleotide duplex with a scrambled sequence corresponding to RNAi 1 were used as negative control. In these experiments, the level of FKBP52 analyzed by Western blot was substantially reduced after 48 h and remained low 72 h post-transfection. Tubulin and FKBP52 stainings were performed 72 h post transfections. In cells transfected with RNAi 1 or 2, FKBP52 staining was significantly lower than that observed in control cells, and tubulin staining revealed a change in the PC12 cell phenotype—in particular, the loss of FKBP52 in PC12 cells results in these cells forming extensions. Therefore, these cells acquired a differentiated phenotype that could be compared with PC12 cells treated with NGF. No significant modification could be observed in cells transfected with controls. In another study, Chambraud et al. ((2010) Proc. Natl. Acad. Sci. U.S.A. 107 (6): 2658-63) reports that FKBP52 prevents Tau Accumulation and Neurite Outgrowth in PC12 Cells. The FKBP52-inducible expression system based on a tetracycline-responsive element was used. The system allows the generation of a stably transformed PC12 cell line to determine a cellular role for FKBP52. Among clones that were positively tested, one clone, so-called H7C2, was selected and used to study the effects of FKBP52 overexpression on PC12 cells and to further investigate the relationship between FKBP52 and Tau. Under basal conditions, H7C2 cells expressed endogenous FKBP52, and treatment with doxycycline (Dox) resulted in a marked increase of recombinant FKBP52 protein expression. FKBP52 induction in H7C2 cells was about four-fold after 5 days of Dox treatment. Next, the effect of FKBP52 on the accumulation of Tau was examined. The amount of Tau protein was determined by Western blotting of extracts from cultures of either PC12 cells or H7C2 cells, treated with or without nerve growth factor (NGF) (50 nM) for 5 days with or without Dox. In PC12 cells, FKBP52 expression was unchanged after treatment with NGF. As expected, in both PC12 and H7C2 cells an increase in Tau was observed after NGF treatment. When H7C2 cells were exposed to Dox in addition to NGF, so that they overexpress FKBP52, no additional accumulation of Tau protein occurred. An increase in Tau protein was still observed in PC12 cells treated with NGF and Dox, ruling out the possibility that Dox was responsible for the lack of decrease in Tau. The report concludes that FKBP52 prevents the accumulation of Tau induced by NGF in PC12 cells.

Because one role of Tau is to stimulate neurite outgrowth, the consequence of FKBP52 overexpression on neurite length in PC12 and H7C2 cells also was investigated. In the absence of NGF, no neurite outgrowth was observed in H7C2 cells, whether or not they were treated with Dox for a week. In H7C2 cells treated with 50 nM NGF and Dox, a 40% (+7) decrease in neurite length, compared to control (H7C2 not treated with Dox) was observed. The same effect of Dox on neurite length was observed in H7C2 cells treated with 10 or 20 nM NGF. Dox by itself was not involved in the process of neurite outgrowth because there was no difference in neurite length between Dox-treated and untreated PC12 cells observed. The inhibition of neurite outgrowth resulting from FKBP52 overexpression is in agreement with the previous report (Chambraud et al. (2010) Proc. Natl. Acad. Sci. U.S.A. 107 (6): 2658-63) showing that the loss of FKBP52 in PC12 cells results in the formation of neurite extensions. The FKBP52 effect on neurite length could be explained by the binding of Tau to FKBP52, removing Tau from microtubules. The prevention of Tau accumulation by overexpression of FKBP52 is consistent with the decrease of neurite length and is indicative of a role of this immunophilin in Tau function. Hence, the above target and sequences can be delivered or encoded in MEVs for treatment of Alzheimer's Disease by preventing accumulation of Tau.

Porphyromonas gingivalis has been identified in the brains of patients with Alzheimer's disease (AD). P. gingivalis has been identified as a risk factor for AD, and its components, gingipains and lipopolysaccharides, have been shown to cause AD-like neurodegeneration in infected neurons derived from induced pluripotent stem cells in in vitro culture system with persistent expression of active gingipains. P. gingivalis has been detected in the brain tissues of AD patients and associated with pathological changes. The MEVs herein can be loaded with agents that inhibit P. gingivalis, and/or gingipains to prevent or treat AD. It has been proposed that the amyloid pathway is an inflammatory response to the infection and the toxic products, including the gingipains. The resulting amyloid plaque and abnormal protein tau can be a source of the neuroinflammation and neurodegeneration (see, e.g., Seymour et al. (2022) J. Exploratory Res. in Pharmacology 7:45-53). The MEVs provided herein can be loaded with inhibitors of the bacterium and/or inhibitor, such as atuzaginstat, of the gingipains, and delivered via intranasal administration, to the brain, including to neurons.
Reporter Genes, Reporter Proteins, and/or Modulators Thereof can be Delivered in the MEVs.
Reporter proteins

Target sequences, in the form of siRNAs, miRNAs to modulate (inhibition or stimulation) of each of the marker genes, such as a beta-glucuronidase (GUS), green fluorescent protein (GFP) a eukaryotic luciferase, or a prokaryotic Luciferase, such as: Lux operon (luxCDABE) and lux operon (luxABCDE), can be used, for example for diagnostics and gene expression assessments (SEQ ID NOs: 72-77, 5, 6, 7, and 62-65, respectively). Exemplary siRNAs that target the encoding nucleic acid are set forth in the following table:

| Target | Type of | | SEQ ID |
|---|---|---|---|
| gene | sequence | Sequence(s) | NO: |
| GUS | siRNA¹ | GT1 sense: 5′- | 72 |
| | | TACACTGTGGAATTGATCAGCGTTCAGATGACGCTGATCA | 73 |
| | | ATTCCACAGTTTTTTTT | 74 |
| | | GT1 antisense: 5′- | |
| | | CTAGAAAAAAAACTGTGGAATTGATCAGCGTCATCTGAA | |
| | | CGCTGATCAATTCCACAGTGTA | |
| | | GT2 sense: 5′- | |
| | | TACTTGATCAGCGTTGGTGGGATTCAGATGATCCCACCAA | 75 |
| | | CGCTGATCAATTTTTTT | |
| | | GT2 antisense: 5′- | |
| | | CTAGAAAAAAATTGATCAGCGTTGGTGGGATCATCTGAA | |
| | | TCCCACCAACGCTGATCAAGTA | |
| | | GT3 sense: 5′- | 76 |
| | | TACCTGTGGAATTGATCAGCGTTTCAGATGAACGCTGATC | 77 |
| | | AATTCCACAGTTTTTTT | |
| | | GT3 antisense: 5′- | |
| | | CTAGAAAAAAACTGTGGAATTGATCAGCGTTCATCTGAA | |
| | | ACGCTGATCAATTCCACAGGTA | |
| DEGFP | siRNA | sense: 5′-GCAAGCUGACCCUGAAGUUCAUUU-3′ | 5 |
| | | antisense: 5′-AUGAACUUCAGGGUCAGCUUGCCG-3′ | 6 |
| firefly | shRNA | 5′-CTGACGCGGAATACTTCGA-3′ | 7 |
| luci- | | | |
| ferase | | | |
| luxA | siRNA | sense: 5′-CAAACAGAGGUAAUGAAAUGGUUG-3′ | 62 |
| (Lux | | antisense: 3′-CAACCAUUUCAUUACCUCUGUUUG-5′ | 63 |
| operon) | | | |
| luxB | siRNA | sense: 5′-AUGUUAAGUUGAAUAAGUUCUGCA-3′ | 64 |
| (Lux | | antisense: 3′-UGCUCUUGAAUAAGUUGAAUUGAU-5′ | 65 |
| operon) | | | |

¹Lu et al., (2004). RNA silencing in plants by the expression of siRNA duplexes. Nucleic Acids Research, 32(21): e171).

The GUS coding sequence is set forth in SEQ ID NO:78, and the encoded protein in SEQ ID NO:79 (see, doi.org/10.1093/nar/gnh170); also in SEQ ID NOs: 59 and 60.

Heterologous cargo includes, for example, anti-angiogenic agents. Anti-angiogenic agents can be a protein, such as an antibody, Fc fusion, and cytokine, that binds to a growth factor or growth factor receptor involved in promoting angiogenesis. Examples of anti-angiogenic agents include but are not limited to antibodies that bind to Vascular Endothelial Growth Factor (VEGF) or that bind to VEGF-R, RNA-based therapeutics that reduce levels of VEGF or VEGF-R expression, VEGF-toxin fusions, Regeneron's VEGF-trap, angiostatin (plasminogen fragment), antithrombin III, angiozyme, ABT-627, Bay 12-9566, BeneFin, bevacizumab, bisphosphonates, BMS-275291, cartilage-derived inhibitor (CDI), CAI, CD59 complement fragment, CEP-7055, Col 3, Combretastatin A-4, endostatin (collagen XVIII fragment), farnesyl transferase inhibitors, fibronectin fragment, GRO-beta, halofuginone, heparinases, heparin hexasaccharide fragment, HMV833, human chorionic gonadotropin (hCG), IM-862, interferon alpha, interferon beta, interferon gamma, interferon inducible protein 10 (IP-10), interleukin-12, kringle 5 (plasminogen fragment), marimastat, metalloproteinase inhibitors (e.g., TIMPs), 2-methoxyestradiol, MMI 270 (CGS 27023A), plasminogen activator inhibitor (PAI), platelet factor-4 (PF4), prinomastat, prolactin 16 kDa fragment, proliferin-related protein (PRP), PTK 787/ZK 222594, retinoids, solimastat, squalamine, SS3304, SU5416, SU6668, SU11248, tetrahydrocortisol-S, tetrathiomolybdate, thalidomide, thrombospondin-1 (TSP-1), TNP470, transforming growth factor beta (TGF-β), vasculostatin, vasostatin (calreticulin fragment), ZS6126, and ZD6474.

Heterologous cargo includes immunomodulatory agents that increase or decrease production of one or more cytokines, up- or down-regulate self-antigen presentation, mask MHC antigens, or promote the proliferation, differentiation, migration, or activation state of one or more types of immune cells. Examples of immunomodulatory agents include but are not limited to cytokines such as TGFβ, IFNα, IFNβ, IFNγ, IL-2, IL-4, IL-10; cytokine, chemokine, or receptor antagonists including antibodies, soluble receptors, and receptor-Fc fusions, such as those against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D), CTLA4, eotaxin, Fas, ICAM, IFNα, IFNβ, IFNγ, IFNAR, IgE, IL-1, IL-2, IL-2R, IL-4, IL-5R, IL-6, IL-8, IL-9, IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as anti-idiotypic antibodies for MHC binding peptides and MHC fragments.

Other heterologous cargo includes cytokines which include, but are not limited to lymphokines, monokines, and polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; Müllerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha,-beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

Other exemplary heterologous cargo includes cytokines and other agents that stimulate cells of the immune system and enhance desired effector function. For example, agents that stimulate NK cells include IL-2; agents that stimulate macrophages include but are not limited to C5a, formyl peptides such as N-formyl-methionyl-leucyl-phenylalanine. Heterologous cargo includes agents that stimulate neutrophils, such as, for example, G-CSF and GM-CSF. Additional agents include, but are not limited to, interferon gamma, IL-3 and IL-7.

In all instances, the cargo includes peptides, small peptides, polypeptides and proteins, nucleic acid encoding the proteins, including various forms of RNA, such as mRNA. The nucleic acids can be operably linked to regulatory elements that are recognized in the particular subject, such as a mammal, in which they are to be delivered.

| Target | | Exemplary route of |
|---|---|---|
| organ/tissue | Exemplary indication | administration |
| Lung | Cystic Fibrosis | Inhalation |
| Lung | Cystic Fibrosis | Inhalation |
| Lung | Idiopathic Pulmonary Fibrosis | Inhalation |
| Lung | Primary Ciliary Dyskinesia | Inhalation |
| Lung | Pulmonary Arterial Hypertension | Inhalation |
| Liver | Inborn error of metabolism | Intravenous or direct |
| | | injection into the liver |
| Lung | Covid-19 (preventive or | Intranasal or Inhalation |
| | therapeutic) | |
| Lymphatic | Covid-19 (vaccine) | Intravenous or |
| | | intramuscular |
| Lung | Influenza (preventive or | Intranasal or |
| | therapeutic) | Inhalation |
| Lymphatic | Influenza (vaccine) | Intravenous or |
| | | intramuscular |
| Lymphatic | Viral pathogen | Intravenous or |
| | | intramuscular |
| Lymphatic | Bacterial pathogen | Intravenous or |
| | | intramuscular |

### 2. Genetic Engineering of Producer Cells

As shown herein, the endogenously loaded MEVs from Chlorella can be used for delivery to humans by any suitable route, including but not limited to intravenous, oral, topical, mucosal, intratracheal, inhalation, intranasal, and any other routes known to those of skill in the art for delivery of vehicles, such as lipid nanoparticles, vectors, therapeutic bacteria, and therapeutic viruses. Upon administration, the MEVs are taken up by cells. Any heterologous cargo suitable to be obtained in the producer cells can be loaded into the MEVs provided herein. The loaded heterologous cargo can be selected so that it only is expressed or produced in targeted cells. Transcription regulatory signals can be selected so that the encoded product is expressed in targeted cells. For example, for expression in the liver, the encoded product can be expressed under control of a liver-specific promoter, or the product can be targeted to a receptor or target expressed in targeted cells, such as in tumors or in the tumor microenvironment.

### 3. Isolation of MEVs

Methods for isolation are discussed in the sections above and detailed in the Examples.

### 4. Exemplary Heterologous Cargo and Exemplary Uses of the Endogenously Loaded MEVs

### a Heterologous Cargo

As described above, the MEVs are loaded with cargo that can be used for any purpose of interest, including any for which other delivery vehicles are used. These uses include delivery of mRNA, such as mRNA encoding therapeutic proteins, including enzymes, RNAi, such as siRNA, and anti-sense RNA, to silence genes, such as genes that suppress the immune system and tumor genes, such as oncogenes. The cargo also can include a therapeutic protein, enzymatic or non-enzymatic, such as a therapeutic antibody. Therapeutic antibodies, include, but are not limited to, anti-cancer antibodies, antibodies to treat autoimmune or inflammatory disease, antibodies to treat transplant rejection, antibodies to treat graft-versus-host-disease (GVHD), and antibodies to treat infectious diseases.

### 1) RNA Cargo and Nucleic Acid Cargo

### RNA Interference and Microalgae

The mechanism of RNA interference or RNAi originally was described as a process of sequence-specific silencing of gene expression in the nematode Caenorhabditis elegans (Fire et al. (1998) Nature 391 (6669): 806-11; Fire and Mello, 2006 Nobel Prize in Medicine awarded to Andrew Fire and Craig Mello). The process of small RNAs targeting (and silencing) messenger RNAs involves a particular RNAi machinery (including silencing factors, such as DICER and ARGONAUTE).

In the plant kingdom, RNAi is involved in antiviral defense mechanisms, and in defense mechanisms against phytopathogenic fungi and oomycetes. Small regulatory RNAs can be active in silencing genes inside bacterial cells, which lack the said RNAi machinery. The silencing activity of siRNA heterologous has been demonstrated to be interkingdom (see, e.g., PCT/EP2019/072169, published as International PCT publication No. WO2020/035619, which shows that siRNA against bacteria can protect plants against infection by the bacteria; PCT/EP2019/072170, published as International PCT publication No WO2020/035620; and Singla et al. (2019c) bioRxiv, doi: doi.org/10.1101/863902). International PCT publication No. WO 2022/053687 shows that Chlorella species can package bacterial-targeted siRNA. Chlorella naturally packages siRNAs into MEVs. This publication and related publications do not show that Chlorella and/or other microalgae can package larger molecules, such as mRNA, plasmids, and polypeptides, into MEVs, as shown and described herein. RNAi-mediated regulation of gene expression has been exploited for several years in the field of biotechnology to confer resistance to viruses (Baulcombe (2015) Current Opinion in Plant Biology 26:141-146). The interkingdom RNAi has been used to characterize the function of genes of eukaryotic pathogens/parasites as well as to induce protection against these organisms.

In Drosophila and Caenorhabditis, RNAi plays a role in antiviral defense by directly targeting viral RNAs via the small RNAs produced by the host in response to viruses. Plant EVs naturally loaded (loaded by the plant cells producing the EVs) with small RNAs, from human edible plants, can modify the composition of the human gut microbiota and oral microbiota by silencing the expression of specific genes in certain commensal bacteria (Teng et al. (2018) Cell Host & Microbes 24:637-652; Sundaram et al. (2019) iScience 21:308-327).

Small interfering RNAs (siRNAs) and microRNAs (miRNAs) are noncoding RNAs with important roles in gene regulation. They also serve as classes of therapeutic agents for the treatment of a wide range of disorders including cancers and infections. Clinical trials of siRNA- and miRNA-based drugs have been initiated. siRNAs and miRNAs share many similarities, both are short duplex RNA molecules that exert gene silencing effects at the post-transcriptional level by targeting messenger RNA (mRNA), yet their mechanisms of action and clinical applications are distinct. Major difference between siRNAs and miRNAs is that the former is highly specific with only one mRNA target, whereas the latter have multiple targets. The siRNAs and miRNAs have a role in gene regulation and serve as targets for drug discovery and development. Compared with conventional small therapeutic molecules, siRNAs and miRNAs offer the potential to be highly potent and able to act on “non-druggable” targets (for example, proteins which lack an enzymatic function); RNAi can be designed to target and/or affect expression of any gene of interest.

RNAi is a conserved gene regulatory mechanism present in all living cells, by which small RNA molecules are involved in sequence-specific suppression of gene expression. The core mechanism of silencing involves the recognition and processing of double-stranded RNAs (dsRNAs) by the RNAse III enzyme Dicer (or Dicer-like) leading to the production of double-stranded short interfering RNA (siRNA) of 20-25 base pairs in length. These siRNA molecules subsequently bind to a central component, the Argonaute protein, of the RNA Induced Silencing Complex (RISC), for post-transcriptional silencing of the complementary transcripts. Dicer-like (DCL) enzyme and Argonaute (AGO) protein are key elements of the RNAi machinery. The former is responsible for processing long double-stranded RNAs (dsRNAs) into mature small RNAs.

In higher plants, DCLs form a gene family being composed of two, four, or five members; for example, four Dicer-like proteins (DCL1-DCL4) with different roles occur in Arabidopsis thaliana (see, e.g., Liu et al. (2009). Dicer-like (DCL) proteins in plants. Functional & integrative genomics 9:277-286). AGO subfamily proteins are present in a wide range of organisms in varied gene copy numbers; in plants, the AGO family has expanded during evolution—e.g. there are 10 different AGOs in Arabidopsis thaliana (see, Fang et al. (2016). RNAi in Plants: An Argonaute-Centered View. The Plant cell 28:272-285). The RNAi machinery in Chlorella, is relatively simple, as the Chlorella genome contains single DCL and AGO proteins (Cerutti et al. (2011). RNA-mediated silencing in Algae: biological roles and tools for analysis of gene function. Eukaryotic cell, 10 (9), 1164-1172).

EP 3967746A1, also published as International PCT Publication No. WO 2020/035620, and its family of applications (entitled “Chlorella-Based Production of Extracellular Vesicle-Embedded Small RNAs for Prophylactic or Therapeutic Applications”), describes methods exploiting this pathway in Chlorella for producing anti-infectives, for plants, based on antimicrobial small RNAs encoded in and expressed in Chlorella cells. Chlorella cells naturally produce and release Extracellular Vesicle (EV)-embedded antimicrobial small RNAs, and the resulting EVs. These EVs are endogenously loaded with siRNA and can be exploited, for example for in EV-based anti-infective products. To exploit the RNA interference pathway, this family of applications describes transforming Chlorella cells with a siRNA or miRNA precursor, which is either long double-stranded RNAs (long dsRNAs) or long single-stranded RNAs (long ssRNAs), The long RNA is enzymatically processed into shorter RNA molecules in Chlorella cells. The precursor length is about 80 to 3000 base pairs or bases. Long dsRNA can be directly processed by the DCL enzyme encoded in the Chlorella genome. The long ssRNA must be converted into long dsRNA molecule, which is subsequently processed by the Chlorella DCL enzyme. The products of DCL enzyme are RNA duplexes produced from their respective long dsRNA precursors. The RNA duplex is a double-stranded structure, a first (sense) and a second (antisense) strand of at least 15 base pairs, where the antisense strand comprises a region of at least 15 contiguous nucleotides that are complementary to a transcript of the targeted gene. The RNA duplex undergoes maturation into a single-stranded molecule in the RISC complex of Chlorella cell and is loaded in the AGO protein and/or associated with other RNA-binding proteins. Thereby, the Chlorella cells can produce functional small interfering RNAs such as siRNAs or miRNAs. These small RNAs have a short size, which is generally between 15 and 30 base pairs or bases and are released into the extracellular medium or naturally embedded into extracellular vesicles at the surface of the Chlorella cells. It does not describe that larger molecules can be packaged into MEVs; it shows use of the Chlorella machinery for producing and packaging small RNA molecules.

The above-family of applications is directed to EV-embedded antimicrobial small RNAs; they do not describe using microalgae to produce EVs loaded with cargo other than small RNA molecules, which generally are no larger than about 50 base pairs or bases. In contrast, it is shown and described herein, that it is possible to exploit Chlorella EVs and load mRNA, particularly for translation in a host cell, proteins, peptides, editing complexes, and other endogenously produced molecules. As exemplified in the PCT publication, Chlorella cells are engineered by transforming them with an inverted repeat (coding for either long double-stranded RNAs (long dsRNAs) or long single-stranded RNAs (long ssRNAs), bearing sequence homology with key virulence factors from pathogenic bacteria.

mRNA and larger RNA and larger products can be packaged into MEVs

In contrast the disclosures discussed above in which siRNA relying on endogenous machinery in the microalgae, it is described and shown herein that the MEVs can be endogenously loaded with mRNA, which is much larger than RNAi. mRNA generally is larger, typically more than 300 bases, and mRNA encodes proteins. In nature, mRNA is for translation into proteins; mRNA is not, itself, the therapeutic product. For delivery herein, the mRNA can be the therapeutic product that is delivered upon administration, such as delivery of mRNA as a vaccine where the mRNA is translated by the host to whom the vaccine is administered. For delivery of mRNA, the microalgae can be transformed, such as with an episomal plasmid encoding the mRNA, or alternatively, genome-modified, where the encoded mRNA contains regulatory signals. mRNA and RNAi fundamentally differ in the mechanism of action used to elicit the appearance or disappearance of specific protein products: while the immediate, direct product of a mRNA is the appearance of a protein, the immediate direct product of an RNAi is the disappearance, via inhibition, of an mRNA.

In has been reported that the amount of mRNA inside mammalian EVs largely reflects the cellular abundance of mRNAs or lncRNAs. The mRNA contents of mammalian EVs is not a mere reflection of the cellular transcriptome. mRNA contained in EVs tends to be richer in GC and are also enriched in motifs that can bind certain RBP (RNA binding proteins) that contribute to the sorting of mRNA. The nucleic acid, such as plasmid encoding the mRNA can be designed so that the mRNA is produced in abundance.

mRNA packaging in MEVs can be accomplished by operatively linking the nucleic acid encoding the mRNA of interest to a eukaryotic promoter, generally a strong promoter, including plant promoters, algae and microalgae promoters, or virus promoters, and optionally other regulatory sequences, such enhancers, in a plasmid that is introduced into the microalgae cells, such as by methods exemplified herein or any other methods known to those of skill in the art. The mRNA then is expressed at high levels and is packaged in the MEVs produced by the microalgae cells for delivery by the MEVs to host cells upon administration.

Exemplary Promoters for expression of encoded nucleic acid

Exemplary promoters for expression of encoded mRNA and proteins in microalgae, include plant promoters. Sequence of plant promoters are well known (see, e.g., Shahmuradov et al. (2003) PlantProm: a database of plant promoter sequences Nucleic Acids Res. 31:114-117, URL: softberry.com/plantprom2016/). The following table includes a list of exemplary plant promoters; sequences of exemplary promoters from the table are set forth in SEQ ID NOs: 86-206 and include any having at least 95%, 96%, 97%, 98%, 99% or more sequence identity therewith and retaining the ability to interact with a RNA polymerase II or III to initiate transcription.

| Promoter name | Origin | Reference |
|---|---|---|
| SVFLt | Peanut chlorotic streak | Maiti I B and Shepherd R J (1998) Isolation and |
| | caulimovirus (PC1SV) | expression analysis of peanut chlorotic streak |
| | | caulimovirus (PCISV) full-length transcript |
| | | (FLt) promoter in transgenic plants. Biochem. |
| | | Biophys. Res. Commun. 244(2): 440-444; see |
| | | GenBank: KM407007.1 |
| Enhanced 35S (E35S) | Cauliflower mosaic | Li Z, Jayasankar S and Gray D J 2001 |
| | virus 35S | Expression of a bifunctional green fluorescent |
| | | protein (GFP) fusion marker under the control of |
| | | three constitutive promoters and enhanced |
| | | derivatives in transgenic grape (Vitis vinifera). |
| | | Plant Sci. 160(5): 877-887; see also Kay et al., |
| | | Science 236(4806): 1299-1302 |
| Sgt | Figwort mosaic virus | Bhattacharyya S, Dey N and Maiti I B 2002 |
| | (FMV) | Analysis of cis-sequence of subgenomic |
| | | transcript promoter from the Figwort mosaic |
| | | virus and comparison of promoter activity with |
| | | the cauliflower mosaic virus promoters in |
| | | monocot and dicot cells. Virus Res. 90(1-2): 47- |
| | | 62; see also GenBank: X06166.1 |
| CsVMV | Cassava vein mosaic | Seemanpillai M, Dry I, Randles J and Rezaian A |
| | virus (CsVMV) | 2003 Transcriptional silencing of geminiviral |
| | | promoter-driven transgenes following |
| | | homologous virus infection. Mol. Plant |
| | | Microbe. Interact. 16(5): 429-438; see also |
| | | GenBank: U59751.1; see also Verdaguer et al., |
| | | (1996) Isolation and expression in transgenic |
| | | tobacco and rice plants, of the cassava vein |
| | | mosaic virus (CVMV) promoter. Plant Mol. |
| | | Biol. 31: 1129-1139 |
| sgRNA β 1, β 2 and γ | Barley stripe mosaic | Johnson J A, Bragg J N, Lawrence D M and |
| | virus (BSMV) | Jackson A O 2003 Sequence elements |
| | | controlling expression of Barley stripe mosaic |
| | | virus subgenomic RNAs in vivo. Virology 313 |
| | | 66-80; see also β 1, β 2: GenBank Accn. No.: |
| | | KY615802.1; see also γ: GenBank Accn. No.: |
| | | M16576.1 |
| SV (antisense) | Peanut chlorotic streak | Bhattacharyya S, Pattanaik S and Maiti I B 2003 |
| | caulimovirus (PC1SV) | Intron-mediated enhancement of gene |
| | | expression in transgenic plants using chimeric |
| | | constructs composed of the Peanut chlorotic |
| | | streak virus (PCISV) promoter-leader and the |
| | | antisense orientation of PC1SV ORF VII (p7R). |
| | | Planta 218 115-24; see also GenBank: U13988.1; |
| 35S | Cauliflower mosaic | Yang I C, Iommarini J P, Becker D K, Hafner G J, |
| | virus 35S | Dale J L and Harding R M 2003 A promoter |
| | | derived from taro bacilliform badnavirus drives |
| | | strong expression in transgenic banana and |
| | | tobacco plants. Plant Cell Rep. 21 1199-1206; |
| | | see also GenBank: V00141.1; see also Fang et |
| | | al., (1989) Multiple cis regulatory elements for |
| | | maximal expression of the cauliflower mosaic |
| | | virus 35S promoter in transgenic plants. The |
| | | Plant Cell 1(1): 141-150 |
| CLCuBuV | Cotton leaf curl | Obertello M, Santi C, Sy M O, Laplaze L, Auguy |
| Rep/CLCuBuV CP | Burewala virus | F, Bogusz D and Franche C (2005) Comparison |
| | (CLCuBuV) | of four constitutive promoters for the expression |
| | | of transgenes in the tropical nitrogen-fixing tree |
| | | Allocasuarina verticillata. Plant Cell Rep. |
| | | 24(9): 540-548; see also U.S. Pat. No. |
| | | US06610907; see also Khan et al., (2015) |
| | | Functional Characterization of a Strong Bi- |
| | | directional Constitutive Plant Promoter Isolated |
| | | from Cotton Leaf Curl Burewala Virus. PLOS |
| | | ONE. 10(3): e0121656 |
| FMV | Figwort mosaic virus | Govindarajulu M, Elmore J M, Fester T and |
| | (FMV) | Taylor C G 2008 Evaluation of constitutive viral |
| | | promoters in transgenic soybean roots and |
| | | nodules. Mol. Plant Microbe Interact. |
| | | 21(8): 1027-1035; see also GenBank: X06166.1; |
| | | see also US patent pub. No. 2008/0227966 |
| Pptcal | Cytomegalovirus | Sakaue K, Harada H and Matsuda Y 2008 |
| | (CMV) | Development of gene expression system in a |
| | | marine diatom using viral promoters of a wide |
| | | variety of origin. Physiol. Plant. 133(1): 59-67; |
| | | see also GenBank: OU594942.1 |
| e35S-4ocs | Cauliflower mosaic | Govindarajulu M, Elmore J M, Fester T and |
| | virus 35S | Taylor C G 2008 Evaluation of constitutive viral |
| | | promoters in transgenic soybean roots and |
| | | nodules. Mol. Plant Microbe Interact. |
| | | 21(8): 1027-1035; see also U.S. Patent Pub No. |
| | | 2006/0031966 |
| AV3 | Ageratum yellow vein | Wang P, Du Y, Zhao X, Miao Y and Song C P |
| | virus (AYVV) | 2013 The MPK6-ERF6-ROS-responsive cis- |
| | | acting Element7/GCC box complex modulates |
| | | oxidative gene transcription and the oxidative |
| | | response in Arabidopsis. Plant Physiol. |
| | | 161: 1392-1408; see also U.S. Patent Pub No. |
| | | US 2011/0053258 |
| TLCV | Tomato leaf curl virus | Khan Z A, Abdin M Z and Khan J A 2015 |
| | | Functional characterization of a strong bi- |
| | | directional constitutive plant promoter isolated |
| | | from cotton leaf curl Burewala virus. PLoS One |
| | | 10(3): e0121656; see also U.S. Patent Pub No. |
| | | 2014/0227230 |
| AMT-1 | Chlorella virus NC-1A | U.S. Pat. No. US6673537B1 |
| AMT-2 | Chlorella virus XZ-6E | U.S. Pat. No. US6673537B1 |
| CMT-1 | Chlorella virus IL-3A | U.S. Pat. No. US6673537B1 |
| UBQ1/UBQ2 | Oryza sativa | Wang J and Oard J H 2003 Rice ubiquitin |
| (RUBQ1/2) | | promoters: deletion analysis and potential |
| | | usefulness in plant transformation systems: |
| | | Genetic transformation and hybridization. Plant |
| | | Cell Rep. 22(2): 129-34; see also Wang, Jianlin, |
| | | Junda Jiang, and James H. Oard 2000, |
| | | “Structure, expression and promoter activity of two |
| | | polyubiquitin genes from rice (Oryza sativa L.).” |
| | | Plant Science 156(2): 201-211; see also |
| | | Accession Nos. AF184280.1 |
| UBQ1 | Arabidopsis | Obertello M, Santi C, Sy M O, Laplaze L, Auguy |
| | | F, Bogusz D and Franche C 2005 Comparison of |
| | | four constitutive promoters for the expression of |
| | | transgenes in the tropical nitrogen-fixing tree |
| | | Allocasuarina verticillata. Plant Cell Rep. |
| | | 24(9): 540-548; see also NCBI Reference |
| | | Sequence: NC_003074.8 |
| VR-ACS1 | Vigna radiata | Cazzonelli C I, McCallum E J, Lee R and Botella |
| | | J R 2005 Characterization of a strong constitutive |
| | | mung bean (Vigna radiata L) promoter with a |
| | | complex mode of regulation in planta. |
| | | Transgenic Res. 14: 941-967; see also NCBI |
| | | Reference Sequence: NC_028357 |
| Ubi | Glycine max | Hernandez-Garcia C M, Martinelli A P, Bouchard |
| | | R A and Finer J J 2009 A soybean (Glycine max) |
| | | polyubiquitin promoter gives strong constitutive |
| | | expression in transgenic soybean. Plant Cell |
| | | Rep. 28(5): 837-849; see also Chiera, Joseph M., |
| | | et al. “Isolation of two highly active soybean |
| | | (Glycine max (L.) Merr.) promoters and their |
| | | characterization using a new automated image |
| | | collection and analysis system.” Plant Cell |
| | | Reports 26(9): 1501-1509; see also NCBI ref. |
| | | seq. NC 038256.2 |
| Ubi1 | Zea mays | Bhattacharyya J, Chowdhury A H, Ray S, Jha |
| | | J K, Das S, Gayen S, Chakraborty A, Mitra J, |
| | | Maiti M K, Basu A and Sen S K 2011 Native |
| | | polyubiquitin promoter of rice provides |
| | | increased constitutive expression in stable |
| | | transgenic rice plants Plant Cell Rep. 31(2): |
| | | 271-279 |
| Ubi1 | Oryza sativa | Bhattacharyya J, Chowdhury A H, Ray S, Jha |
| | | J K, Das S, Gayen S, Chakraborty A, Mitra J, |
| | | Maiti M K, Basu A and Sen S K 2011 Native |
| | | polyubiquitin promoter of rice provides |
| | | increased constitutive expression in stable |
| | | transgenic rice plants Plant Cell Rep. 31(2): 271- |
| | | 279; see also NCBI Accession No. AY785814 |
| GAI | Oryza sativa | Bhattacharyya J, Chowdhury A H, Ray S, Jha |
| | | J K, Das S, Gayen S, Chakraborty A, Mitra J, |
| | | Maiti M K, Basu A and Sen S K 2011 Native |
| | | polyubiquitin promoter of rice provides |
| | | increased constitutive expression in stable |
| | | transgenic rice plants Plant Cell Rep. 31(2): 271- |
| | | 279; see also U.S. Pat. No. 11,220,694 |
| Ubi1/Ubi2 | Panicum virgatum | Mann D G, King Z R, Liu W, Joyce B L, |
| | | Percifield R J, Hawkins J S, LaFayette P R, Artelt |
| | | B J, Burris J N, Mazarei M, Bennetzen J L, Parrott |
| | | W A and Stewart C N 2011 Switchgrass |
| | | (Panicum virgatum L) polyubiquitin gene |
| | | (PvUbi1 and PvUbi2) promoters for use in plant |
| | | transformation. BMC Biotechnol. 11(1): 74; see |
| | | also GenBank No.: HM209467.1; see also U.S. |
| | | Patent pub. No 2011/0023183 |
| uceApro2 | Gossypium | Viana A A, Fragoso R R, Guimaraes L M, Pontes |
| | | N, Oliveira-Neto O B, Artico S et al 2011 |
| | | Isolation and functional characterization of a |
| | | cotton ubiquitination-related promoter and |
| | | 5′UTR that drives high levels of expression in |
| | | root and flower tissues. BMC Biotechnol. |
| | | 11: 115; see also US Patent Pub. No. |
| | | 2017/0029843 |
| SUI1 | Ananas comosus | Koia J, Moyle R, Hendry C, Lim L and Botella |
| | | JR 2013 Pineapple translation factor SUI1 and |
| | | ribosomal protein L36 promoters drive |
| | | constitutive transgene expression patterns in |
| | | Arabidopsis thaliana. Plant Mol. Biol. 81: 327- |
| | | 336 |
| L36 | Ananas comosus | Koia J, Moyle R, Hendry C, Lim L and Botella |
| | | JR 2013 Pineapple translation factor SUI1 and |
| | | ribosomal protein L36 promoters drive |
| | | constitutive transgene expression patterns in |
| | | Arabidopsis thaliana. Plant Mol. Biol. 81: 327- |
| | | 336 |
| EF1α | Marchantia | Althoff F, Kopischke S, Zobell O, Ide K, |
| | polymorpha | Ishizaki K, Kohchi T and Zachgo S 2014 |
| | | Comparison of the MpEF1alpha and CaMV35 |
| | | promoters for application in Marchantia |
| | | polymorpha overexpression studies. Transgenic |
| | | Res. 23(2): 235-244; see also Aida et a. 2005, |
| | | Efficient Transgene Expression in |
| | | Chrysanthemum, Chrysanthemum morifolium |
| | | Ramat., with the Promoter of a Gene for |
| | | Tobacco Elongation Factor 1 α Protein; see also |
| | | GenBank: AB630337.1 |
| EF1α/UBI10/GLU1 | Brachypodium | Coussens G, Aesaert S, Verelst W, |
| | distachyon | Demeulenaere M, De Buck S, Njuguna E, Inze |
| | | D and Van Lijsebettens M 2012 Brachypodium |
| | | distachyon promoters as efficient building |
| | | blocks for transgenic research in maize. J. Exp. |
| | | Bot. 63(11): 4263-4273 |
| Con1 | Oryza sativa | Li J, Xu R F, Qin R Y, Ma H, Li H, Zhang Y P, Li |
| | | L, Wei P C and Yang J B 2014 Isolation and |
| | | functional characterization of a novel rice |
| | | constitutive promoter. Plant Cell Rep. |
| | | 33(10): 1651-60 |
| TCTP | Arabidopsis | Han Y J, Kim Y M, Hwang O J and Kim J I 2015 |
| | | Characterization of a small constitutive |
| | | promoter from Arabidopsis translationally |
| | | controlled tumor protein (AtTCTP) gene for |
| | | plant transformation. Plant Cell Rep. 34(2): 265- |
| | | 275 |
| RD29A, RD29B | Arabidopsis | Bihmidine S, Lin J, Stone J M, Awada T, Specht |
| | | J E and Clemente T E 2013 Activity of the |
| | | Arabidopsis RD29A and RD29B promoter |
| | | elements in soybean under water stress. Planta |
| | | 237(1): 55-64; see also GenBank accession |
| | | number D13044.1 |
| UEP | Jatropha curcas | Tao Y B, He L L, Niu L J and Xu Z F 2015 |
| | | Isolation and characterization of an ubiquitin |
| | | extension protein gene (JcUEP) promoter from |
| | | Jatropha curcas. Planta 241(4): 823-836; see |
| | | also GenBank: KJ202645 |
| KST1 | Solanum tuberosum | Kelly G, Lugassi N, Belausov E, Wolf D, |
| | | Khamaisi B, Brandsma D, Kottapalli J, Fidel L, |
| | | Ben-Zvi B, Egbaria A, Acheampong A K, Zheng |
| | | C, Or E, Distelfeld A, David-Schwartz R, Carmi |
| | | N and Granot D 2017 The Solanum tuberosum |
| | | KST1 partial promoter as a tool for guard cell |
| | | expression in multiple plant species. J. Exp. Bot. |
| | | 68(11): 2885-2897; see also NCBI: AJ242852.1 |
| CsCYP, CsGAPC2, | Citrus sinensis | Erpen L, Tavano E C R, Harakava R, Dutt M, |
| CsEF1 | | Grosser J W, Piedade S M S and Moura o Filho |
| | | F A A 2018 Isolation, characterization and |
| | | evaluation of three Citrus sinensis-derived |
| | | constitutive gene promoters. Plant Cell Rep. |
| | | 37(8): 1113-1125; see also GenBank: |
| | | MN097548.1 (Citrus sinensis isolate CsMDx8 |
| | | EF1 gene, promoter region) |
| AtSCPL30 | Arabidopsis | Jiang P, Zhang K, Ding Z, He Q, Li W, Zhu S |
| | | and Li K 2018 Characterization of a strong and |
| | | constitutive promoter from the Arabidopsis |
| | | serine carboxypeptidase-like gene AtSCPL30 as |
| | | a potential tool for crop transgenic breeding. |
| | | BMC Biotech. 18: 59 |
| CsGAPC2, CsEF1 | Citrus sinensis | Erpen-Dalla Corte L, Mendes B M J, Mourao |
| | | Filho F A A, Grosser J W and Dutt M 2020 |
| | | Functional characterization of full-length and 50 |
| | | deletion fragments of Citrus sinensis-derived |
| | | constitutive promoters in Nicotiana |
| | | benthamiana. In Vitro Cell. Dev. Biol. Plant 56 |
| | | 280-289: see also GenBank: MN097541.1, |
| | | MN097542.1, MN097543.1 |
| 16R | Solanum tuberosum | Sztafrowski, D., Aksamit-Stachurska, A., |
| | | Kostyn, K., Mackiewicz, P., & Łukaszewicz, M. |
| | | (2017). Electromagnetic Field Seems to Not |
| | | Influence Transcription via CTCT Motif in |
| | | Three Plant Promoters. Frontiers in Plant |
| | | Science, 8, 178. |
| | | doi.org/10.3389/fpls.2017.00178; see also |
| | | GenBank Accn. No. AY070220.1 |
| 20R | Solanum tuberosum | Sztafrowski, D., Aksamit-Stachurska, A., |
| | | Kostyn, K., Mackiewicz, P., & Łukaszewicz, M. |
| | | (2017). Electromagnetic Field Seems to Not |
| | | Influence Transcription via CTCT Motif in |
| | | Three Plant Promoters. Frontiers in Plant |
| | | Science, 8, 178. |
| | | doi.org/10.3389/fpls.2017.00178; GenBank acc. |
| | | no. AY518222 |
| 5UGT | Solanum tuberosum | Sztafrowski, D., Aksamit-Stachurska, A., |
| | | Kostyn, K., Mackiewicz, P., & Łukaszewicz, M. |
| | | (2017). Electromagnetic Field Seems to Not |
| | | Influence Transcription via CTCT Motif in |
| | | Three Plant Promoters. Frontiers in Plant |
| | | Science, 8, 178. |
| | | doi.org/10.3389/fpls.2017.00178; see also |
| | | GenBank acc. no. AY033489 (mRNA |
| | | sequence); see also GenBank acc. no. |
| | | AY177376.1 (includes gene, promoter region |
| | | and partial CDS). |
| HSP90 promoter | Nannochloropsis | Ramarajan M, Fabris M, Abbriano R M, Pernice |
| (Nga00934) | gaditana | M, Ralph P J (2019) Novel endogenous |
| | | promoters for genetic engineering of the marine |
| | | microalga Nannochloropsis gaditana CCMP526, |
| | | Algal Research 44, 101708; See also NCBI No. |
| | | AZIL01000126.1 |
| EPPSII promoter | Nannochloropsis | Ramarajan M, Fabris M, Abbriano R M, Pernice |
| (Nga02101) | gaditana | M, Ralph P J (2019) Novel endogenous |
| | | promoters for genetic engineering of the marine |
| | | microalga Nannochloropsis gaditana CCMP526, |
| | | Algal Research 44, 101708 |
| ATPase promoter | Nannochloropsis | Ramarajan M, Fabris M, Abbriano RM, Pernice |
| (Nga06354.1) | gaditana | M, Ralph PJ (2019) Novel endogenous |
| | | promoters for genetic engineering of the marine |
| | | microalga Nannochloropsis gaditana CCMP526, |
| | | Algal Research 44, 101708 |
| TCT; RPL24; 4AIII; | Nannochloropsis | Ajjawi, I., Verruto, J., Aqui, M. et al. (2017) |
| EIF3 | gaditana | Lipid production in Nannochloropsis gaditana is |
| | | doubled by decreasing expression of a single |
| | | transcriptional regulator. Nat Biotechnol 35, |
| | | 647-652 |
| EF; TUB; NR | Nannochloropsis | de Grahl, I., Rout, S. S., Maple-Grødem, J., & |
| | oceanica | Reumann, S. (2020). Development of a |
| | | constitutive and an auto-inducible high-yield |
| | | expression system for recombinant protein |
| | | production in the microalga Nannochloropsis |
| | | oceanica. Applied microbiology and |
| | | biotechnology, 104(20), 8747-8760. |
| | | doi.org/10.1007/s00253-020-10789-4 |
| AcRbcS | Ankistrodesmus | Thanh, T., Chi, V. T. Q., Omar, H., Abdullah, |
| | convolutus | M. P., and Napis, S. (2012). Sequence analysis |
| | | and potentials of the native RbcS promoter in |
| | | the development of an alternative eukaryotic |
| | | expression system using green microalga |
| | | Ankistrodesmus convolutus. Int. J. Mol. Sci. 13, |
| | | 2676-2691 |
| HSP70A | Chlamydomonas | Schroda, M., Blöcker, D., & Beck, C. F. (2000). |
| | reinhardtii | The HSP70A promoter as a tool for the |
| | | improved expression of transgenes in |
| | | Chlamydomonas. Plant J. 21(2), 121-131 |
| ARG7 | Chlamydomonas | Specht, E. A., Nour-eldin, H. H., Hoang, K. T. |
| | reinhardtii | D., and Mayfield, S. P. (2015). An improved |
| | | ARS2-derived nuclear reporter enhances the |
| | | efficiency and ease of genetic engineering in |
| | | Chlamydomonas. Biotechnol. J. 10, 473-479 |
| AR; METE; NIT1; A*R; | Chlamydomonas | Crozet, P., Navarro, F. J., Willmund, F., |
| PSAD; β-TUB2; VIPP2 | reinhardtii | Mehrshahi, P., Bakowski, K., Jonathan, K., et al. |
| | | (2018). Birth of a photosynthetic chassis: a |
| | | MoClo toolkit enabling synthetic biology in the |
| | | microalga Chlamydomonas reinhardtii. ACS |
| | | Synth. Biol. 7, 2074-2086 |
| GPDH3 | Chlamydomonas | Beltran-Aguilar, A. G., Peraza-Echeverria, S., |
| | reinhardtii | López-Ochoa, L. A., Borges-Argáez, I. C., & |
| | | Herrera-Valencia, V. A. (2019). A novel salt- |
| | | inducible CrGPDH3 promoter of the microalga |
| | | Chlamydomonas reinhardtii for transgene |
| | | overexpression. Applied microbiology and |
| | | biotechnology, 103(8), 3487-3499 |
| CvNDI1 and CvNDI2 | Chlorella vulgaris | Shin, J. H., Choi, J., Jeon, J., Kumar, M., Lee, J., |
| | | Jeong, W. J., & Kim, S. R. (2020). The |
| | | establishment of new protein expression system |
| | | using N starvation inducible promoters in |
| | | Chlorella. Scientific reports, 10(1), 12713 |
| psaD | Chlorella vulgaris | Kim, J., Liu, L., Hu, Z., and Jin, E. (2018). |
| | | Identification and functional analysis of the |
| | | psaD promoter of Chlorella vulgaris using |
| | | heterologous model strains. Int. J. Mol. Sci. |
| | | 19: 1969 |
| α-tubulin | Chlorella vulgaris | Fernández-Rodríguez R, Garro-Monge G, |
| | | Guerrero-Barrantes M, Gómez-Espinoza O. |
| | | (2020) Alpha-tubulin promoter from Chlorella |
| | | vulgaris allows genetic transformation of green |
| | | coccoid microalga. Tecnología en Marcha. 33- |
| | | 2: 27-36 |
| Rol A/B/C | Agrobacterium | Schmulling T, Schell J and Spena A 1989 |
| | rhizogens | Promoters of the rolA B and C genes of |
| | | Agrobacterium rhizogenes are differentially |
| | | regulated in transgenic plants. Plant Cell 1: 665- |
| | | 670; see also GenBank No.: X64255.1 |
| Ocs | Agrobacterium | Zhou J, Yang Y, Wang X, Yu F, Yu C, Chen J, |
| | rhizogens | Cheng Y, Yan C and Chen J 2013 Enhanced |
| | | transgene expression in rice following selection |
| | | controlled by weak promoters. BMC Biotechnol. |
| | | 27 13-29; see also Bouchez et al 1989, The ocs- |
| | | element is a component of the promoters of |
| | | several T-DNA and plant viral genes |
| Mas | Agrobacterium | Zhou J, Yang Y, Wang X, Yu F, Yu C, Chen J, |
| | rhizogens | Cheng Y, Yan C and Chen J 2013 Enhanced |
| | | transgene expression in rice following selection |
| | | controlled by weak promoters. BMC Biotechnol. |
| | | 27 13-29 |
| Nos | Agrobacterium | Zhou J, Yang Y, Wang X, Yu F, Yu C, Chen J, |
| | rhizogens | Cheng Y, Yan C and Chen J 2013 Enhanced |
| | | transgene expression in rice following selection |
| | | controlled by weak promoters. BMC Biotechnol. |
| | | 27 13-29 |
| pCL | synthetic | Li F, Han Y, Feng Y, Xing S, Zhao M, Chen Y |
| | | and Wang W 2013 Expression of wheat |
| | | expansin driven by the RD29 promoter in |
| | | tobacco confers water-stress tolerance without |
| | | impacting growth and development. J. |
| | | Biotechnol. 163 281-291 |
| Sab/sba | synthetic | Li F, Han Y, Feng Y, Xing S, Zhao M, Chen Y |
| | | and Wang W 2013 Expression of wheat |
| | | expansin driven by the RD29 promoter in |
| | | tobacco confers water-stress tolerance without |
| | | impacting growth and development. J. |
| | | Biotechnol. 163 281-291 |
| 4 x CCTC | synthetic | Lota F, Wegmu\ddot{ } ller S, Buer B, Sato S, |
| | | Bra\ddot{ }utigam A, Hanf B and Bucher M 2013 The |
| | | cis-acting CTTC-P1BS module is indicative for |
| | | gene function of LjVTI12 a Qb-SNARE protein |
| | | gene that is required for arbuscule formation in |
| | | Lotus japonicas. Plant J. 74 280-293 |
| 4xRE/B4REA | synthetic | Liu W, Mazarei M, Peng Y, Fethe M H, Rudis |
| | | M R, Lin J, Millwood R J, Arelli P R and Stewart |
| | | C N 2014 Computational discovery of soybean |
| | | promoter cis-regulatory elements for the |
| | | construction of soybean cyst nematode-inducible |
| | | synthetic promoters. Plant Biotechnol. J. 12 |
| | | 1015-1026 |
| FsFfCBD | synthetic | Patro S, Maiti I B and Dey N 2013 Development |
| | | of an efficient bi-directional promoter with |
| | | tripartite enhancer employing three viral |
| | | promoters. J. Biotechnol. 163 311-317 |
| 4 × GCC | synthetic | Van der Does D, Leon-Reyes A, Koornneef A, |
| | | Van Verk M C, Rodenburg N, Pauwels L, |
| | | Goossens A, Korbes A P, Memelink J, Ritsema |
| | | T, Van Wees S C and Pieterse C M 2013 |
| | | Salicylic acid suppresses jasmonic acid |
| | | signaling downstream of SCFCOI1-JAZ by |
| | | targeting GCC promoter motifs via transcription |
| | | factor ORA59. Plant Cell 25 744-761 |
| 4 x ROSE1~7 | synthetic | Wang P, Du Y, Zhao X, Miao Y and Song C P |
| | | 2013 The MPK6-ERF6-ROS-responsive cis- |
| | | acting Element7/GCC box complex modulates |
| | | oxidative gene transcription and the oxidative |
| | | response in Arabidopsis. Plant Physiol. 161 |
| | | 1392-1408 |
| FSgt-PFlt; MSgt-PFlt; | synthetic | Acharya S, Ranjan R, Pattanaik S, Maiti I B and |
| PFlt-UAS-2X | | Dey N 2014 Efficient chimeric plant promoters |
| | | derived from plant infecting viral promoter |
| | | sequences. Planta 239 381-396 |
| 4 x RSRE | synthetic | Benn G, Wang C Q, Hicks D R, Stein J, Guthrie |
| | | C and Dehesh K 2014 A key general stress |
| | | response motif is regulated non-uniformly by |
| | | CAMTA transcription factors. Plant J. 80 82-92 |
| p35S-PCHS-Ω; p35S-Ω; | synthetic | Du L, Lou Q, Zhang X, Jiao S, Liu Y and Wang |
| LCHS-Ω; pOCS-PCHS- | | Y 2014 Construction of flower-specific chimeric |
| Ω; pOCS-LCHS-Ω | | promoters and analysis of their activities in |
| | | transgenic Torenia. Plant Mol. Biol. Rep. 32 |
| | | 234-245 |
| CL | synthetic | Li M, Xie C, Song B, Ou Y, Lin Y, Liu X, |
| | | Zhang H and Liu J 2015 Construction of |
| | | efficient tuber-specific and cold-inducible |
| | | promoters in potato. Plant Sci. 235: 14-24 |
| MAMP-responsive | synthetic | Kanofsky K, Lehmeyer M, Schulze J and Hehl |
| synthetic promoter | | R 2016 Analysis of microbe-associated |
| | | molecular pattern-responsive synthetic |
| | | promoters with the parsley protoplast system. |
| | | Methods Mol. Biol. 1482 163-174 |
| P_DRE::35S | synthetic | Gerasymenko I M and Sheludko Y V 2017 |
| | | Synthetic cold-inducible promoter enhances |
| | | recombinant protein accumulation during |
| | | Agrobacterium-mediated transient expression in |
| | | Nicotiana excelsior at chilling temperatures |
| | | Biotechnol. Lett. 39 1059-1067 |
| SynP16 | synthetic | Jameel, A., Noman, M., Liu, W., Ahmad, N., |
| | | Wang, F., Li, X., & Li, H. (2020). Tinkering Cis |
| | | Motifs Jigsaw Puzzle Led to Root-Specific |
| | | Drought-Inducible Novel Synthetic Promoters. |
| | | International Journal of Molecular Sciences, |
| | | 21(4), 1357 |
| AβSAP(i) | synthetic | Einhaus, A., Baier, T., Rosenstengel, M., |
| | | Freudenberg, R. A., & Kruse, O. (2021). |
| | | Rational Promoter Engineering Enables Robust |
| | | Terpene Production in Microalgae. ACS |
| | | synthetic biology, 10(4), 847-856. |
| | | doi.org/10.1021/acssynbio.0c00632 |

Additional exemplary promoters include, but are not limited to:

| EF2 (elongation factor 2) | Phaeordactium tricornutum |
|---|---|
| Histone 4 | Penicillium funiculosum |
| Lhcf.1 (Light-harvesting complex protein) | Phaeodactium tricornutum |
| Ubi.U4 | Nicotiana tabacum |
| MAS Mannopine Synthase | Agrobacterium tumefaciens |
| CAM 35S | Cauliflower mosaic virus |
| PAR (Pseudoautosomal region) | Arabidopsis thaliana |
| Cv PAS D (Photosystem protein D) | Chlorella vulgaris |
| CMV | Cytomegalovirus |
| CMV + enhancer | Cytomegalovirus |
| SV40 | Simian virus 40 |

Regulatory sequences and binding sites for control of translation in genetically-modified microalgae

Regulatory signals and binding sites for controlling translation are well known. The following is an overview and description of signals and sites in mRNA for controlling translation, including sites that can be modified or deleted so that the mRNA is not translated in the microalgae, but is delivered by MEVs (for a review, see, Fátima Gebauer et al. (2004) Nature Reviews Molecular Cell Biology 5:827-835).

Structural features and regulatory sequences within the mRNA include: the canonical end modifications of mRNA molecules—the cap structure and the poly(a) tail—which are required for translation initiation; internal ribosome-entry sequences (IRESs), which mediate cap-independent translation initiation; upstream open reading frames (uORFs and sORFs), which normally reduce translation from the main ORF; secondary or tertiary RNA structures, such as hairpins and pseudoknots, which generally block initiation, but can also be part of IRES elements and therefore promote cap-independent translation; and, specific binding sites for regulatory complexes. Most of the regulatory mechanisms that are inhibitory; absent any change, mRNAs are translated. For endogenously packaging mRNA in MEVs, the mRNA that is encoded by DNA introduced into the microalgae cells can be modified, such as by deletion of the IRES, or modifying or interfering with ribosome binding proteins, or other such method known to those of skill in the art.

In some embodiments, the microalgae cells are transformed with a plasmid that is then integrated into the genome, and mRNA is transcribed (produced). Alternatively, a plasmid that remains episomal can be introduced. The mRNA can be translated by the microalgae ribosomes. In other embodiments, the mRNA can contain modifications so that it is optimized or designed for translation in an animal, such as a human, subject. For example, the mRNA can contain optimized codons for expression in a subject, such as human, for translation so that it is not efficiently or not translated by the microalgae ribosomes, but is translated by higher order species, such as animal, such as a human. The mRNA can be “optimized for codons” (“codon optimization”) that translate well in the cell type where the mRNA is intended to be translated. The encoding plasmid sequence can be “optimized for codons” such that the mRNA will not be translated, or translated inefficiently by microalgae ribosomes, but is translated by mammalian ribosomes, or will in such a way that the mRNA transcribed out of that plasmid will not translate (or will do it very inefficiently) in the microalgae cell but will efficiently translate in the cells of those to whom the MEVs are administered. For example, the IRES and/or Kozak sequences encoded in the mRNA can be optimized or designed for expression or efficient or high expression in a mammal, not microalgae. Other regulatory elements can be optimized or designed for translation in cells of the target host, such as mammalian host, or a particular cell type.

mRNA generally includes the m⁷GpppN cap structure at the 5′ end of the mRNA, and the poly(A) tail at the 3′ end, which are motifs that promote translation initiation. Secondary structures, such as hairpins, block translation. Internal ribosome entry sequences (IRESs) mediate cap-independent translation. Upstream open reading frames (uORFs) normally function as negative regulators by reducing translation from the main ORF. Also included are binding sites for proteins and/or RNA regulators, which usually inhibit, but also promote, translation. These sequences can be optimized for translation in the intended host, such as mammalian cell, and/or selected so that they are not or not efficiently translated by the microalgae ribosomes, but are translated by mammalian, such as human, ribosomes. For example, the mRNA can include a Kozak sequence that is optimized for mammalian translation.

### 2) Protein Cargo

Protein cargo includes therapeutic proteins. These can be encoded by DNA introduced into the microalgae cell by any method known to the skilled person, such as those discussed above. The DNA can include regulatory sequences, such as strong promoters, to ensure production of a relatively large amount of the protein, that is then packaged in the MEVs. The protein cargo is encoded by DNA constructs that include regulatory sequences, as well as codon optimization, for an efficient transcription and, subsequently, translation in the microalgae. The nucleic acid will include appropriate sequences for translation into proteins. In general, the constructs will include strong promoters, such as strong plant promoters, and eukaryotic viral promoters, as well as enhancers to ensure that high levels of proteins are produced in the microalgae cells and packaged in the MEVs.

Exemplary of heterologous protein cargo are antibodies, antigens, and anticancer therapeutic proteins, such as, but not limited to, anti-cancer antibodies, such as those that target tumor antigens, and checkpoint inhibitors. Examples of anti-cancer antibodies and other antibodies, include, but are not limited to, anti-17-IA cell surface antigen antibodies such as the antibody sold or provided under the trademark Panorex™ (edrecolomab); anti-4-1BB antibodies; anti-4Dc antibodies; anti-A33 antibodies such as A33 and CDP-833; anti-al integrin antibodies such as natalizumab; anti-a437 integrin antibodies such as LDP-02; anti-aVB1 integrin antibodies such as F-200, M-200, and SJ-749; anti-aVB3 integrin antibodies such as abciximab, CNTO-95, Mab-17E6, and humanized monoclonal antibody against the vitronectin receptor sold under the trademark Vitaxin™; anti-complement factor 5 (C5) antibodies such as 5G1.1; anti-CA125 antibodies such as sold or provided under the trademark OvaRex™ (oregovomab); anti-CD3 antibodies such as those sold or provided under the trademark Nuvion™ (visilizumab), and Rexomab; anti-CD4 antibodies such as IDEC-151, MDX-CD4, OKT4A; anti-CD6 antibodies such as Oncolysin B and Oncolysin CD6; anti-CD7 antibodies such as HB2; anti-CD19 antibodies such as B43, MT-103, and Oncolysin B; anti-CD20 antibodies such as 2H7, 2H7.v16, 2H7.v114, 2H7.v115, the product sold or provided under the trademark Bexxar™ (tositumomab), the antibody sold or provided under the trademark Rituxan® (rituximab), and the antibody sold or provided under the trademark Zevalin® (Ibritumomab tiuxetan); anti-CD22 antibodies such as the those sold or provided under the following generic names, tradenames, or trademarks: Lymphocide™ (epratuzumab); anti-CD23 antibodies, such as IDEC-152; anti-CD25 antibodies such as basiliximab and Zenapax™ (daclizumab); anti-CD30 antibodies such as AC10, MDX-060, and SGN-30; anti-CD33 antibodies, such as Mylotarg® (gemtuzumab ozogamicin), Oncolysin M, and Smart M 195; anti-CD38 antibodies; anti-CD40 antibodies, such as SGN-40 and toralizumab; anti-CD40L antibodies, such as 5c8, Ruplizumab (tradename Antova), and IDEC-131; anti-CD44 antibodies, such as bivatuzumab; anti-CD46 antibodies; anti-CD52 antibodies such as Campath® (alemtuzumab); anti-CD55 antibodies such as SC-1; anti-CD56 antibodies such as huN901-DM1; anti-CD64 antibodies such as MDX-33; anti-CD66e antibodies such as XR-303; anti-CD74 antibodies such as IMMU-1 10; anti-CD80 antibodies such as galiximab and IDEC-1 14; anti-CD89 antibodies such as MDX-214; anti-CD123 antibodies; anti-CD138 antibodies such as B-B4-DM1; anti-CD146 antibodies such as AA-98; anti-CD148 antibodies; anti-CEA antibodies such as cT84.66, labetuzumab, the bispecific Fab MN14-734 (Pentacea™, IBC Pharmaceuticals); anti-CTLA-4 antibodies such as MDX-101; anti-CXCR4 antibodies; anti-EGFR antibodies such as ABX-EGF, Erbitux® (cetuximab), IMC-C225, and Merck Mab 425; anti-EpCAM antibodies such as Crucell's anti-EpCAM, ING-1, and IS-IL-2; anti-ephrin B2/EphB4 antibodies; anti-Her2 antibodies such as Herceptin®, MDX-210; anti-FAP (fibroblast activation protein) antibodies such as sibrotuzumab; anti-ferritin antibodies such as NXT-211; anti-FGF-1 antibodies; anti-FGF-3 antibodies; anti-FGF-8 antibodies; anti-FGFR antibodies, anti-fibrin antibodies; anti-G250 antibodies, such as WX-G250, and Girentuximab (sold as Rencarex®); anti-GD2 ganglioside antibodies such as EMD-273063 and TriGem; anti-GD3 ganglioside antibodies such as BEC2, KW-2871, and mitumomab; anti-gpIIb/IIIa antibodies such as ReoPro®; anti-heparinase antibodies; anti-Her2/ErbB2 antibodies such as Herceptin® (trastuzumab), MDX-210, and pertuzumab; anti-HLA antibodies such as MA5-41779 (sold as Oncolym™), Smart 1D10; anti-HM1.24 antibodies; anti-ICAM antibodies such as ICM3; anti-IgA receptor antibodies; anti-IGF-1 antibodies such as CP-751871 and EM-164; anti-IGF-1R antibodies such as IMC-A12; anti-IL-6 antibodies such as CNTO-328 and elsilimomab; anti-IL-15 antibodies such as the antibody sold as HuMax®-IL15; anti-KDR antibodies; anti-laminin 5 antibodies; anti-Lewis Y antigen antibodies such as Hu3S193 and IGN-311; anti-MCAM antibodies; anti-Muc1 antibodies, such as BravaRex and TriAb; anti-NCAM antibodies, such as ERIC-1 and ICRT; anti-PEM antigen antibodies such as Theragyn and Therex; anti-PSA antibodies; anti-PSCA antibodies, such as IG8; anti-Ptk antibodies; anti-PTN antibodies; anti-RANKL antibodies such as AMG-162; anti-RLIP76 antibodies; anti-SK-1 antigen antibodies such as Monopharm C; anti-STEAP antibodies; anti-TAG72 antibodies such as CC49-SCA and MDX-220; anti-TGF-β antibodies such as CAT-152; anti-TNF-α antibodies such as CDP571, CDP870, D2E7, Humira® (adalimumab), and Remicade® (infliximab); anti-TRAIL-R1 and TRAIL-R2 antibodies; anti-VE-cadherin-2 antibodies; and anti-VLA-4 antibodies (Antegren™). Furthermore, anti-idiotype antibodies including but not limited to the GD3 epitope antibody BEC2 and the gp72 epitope antibody 105AD7, can be used. In addition, bispecific antibodies including but not limited to the anti-CD3/CD20 antibody Bi20 can be used.

Other heterologous cargo include, for example, immunomodulatory agents that increase or decrease production of one or more cytokines, up- or down-regulate self-antigen presentation, mask MHC antigens, or promote the proliferation, differentiation, migration, or activation state of one or more types of immune cells. Examples of immunomodulatory agents include but are not limited to cytokines such as TGFβ, IFNα, IFNβ, IFNγ, IL-2, IL4, IL-10; cytokine, chemokine, or receptor antagonists including antibodies, soluble receptors, and receptor-Fc fusions, such as those against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D), CTLA4, eotaxin, Fas, ICAM, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as anti-idiotypic antibodies for MHC binding peptides and MHC fragments.

Other heterologous cargo include cytokines which include, but are not limited to lymphokines, monokines, and traditional polypeptide hormones, and also chemokines, which can be classified under the umbrella of cytokines. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; Müllerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha,-beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

Other exemplary heterologous cargo include cytokines and other agents that stimulate cells of the immune system and enhance desired effector function. For example, agents that stimulate NK cells include IL-2; agents that stimulate macrophages include but are not limited to C5a, formyl peptides such as N-formyl-methionyl-leucyl-phenylalanine. Heterologous cargo include agents that stimulate neutrophils, such as, for example, G-CSF and GM-CSF. Additional agents include, but are not limited to, interferon gamma, IL-3 and IL-7.

Additional exemplary heterologous cargo include uses and treatments that can be effected with cargo-loaded MEVs are described, by way of example, as follows.

### b. Diseases and Methods of Treatment

As described above, the MEVs can be loaded with any desired heterologous cargo, including, but not limited to, nucleic acid molecules, proteins, detectable marker proteins and tags, gene editing systems, and others, and combinations thereof for delivering therapeutic molecules, serving as vaccines, and for use in human health, cosmetic, dermatological and diagnostic applications, industrial uses, and other uses. The MEVs can deliver regulators of gene pathways to produce a beneficial product, gene editing systems, such as CRISPR/cas to effect gene editing, and gene therapy products.

MEVs can carry cargo, for example, for treating a disease characterized by a genetic defect that results in a deficiency of a functional protein, or for treating a disease characterized by overexpression of a polypeptide. Non-limiting examples of diseases that can be treated by silencing of a target gene, for example using siRNA or microRNA (see, e.g., International Pub. No. WO 2013/048734) include cancer (e.g., lung cancer, leukemia and lymphoma, pancreatic cancer, colon cancer, prostate cancer, glioblastoma, ovarian cancer, breast cancer, head and neck cancer, liver cancer, skin cancer, and uterine cancer), cardiovascular diseases, ocular diseases (e.g., age-related macular degeneration, herpetic stromal keratitis, Glaucoma, dry eye syndrome, diabetic retinopathy, and conditions associated with ocular angiogenesis and ocular hypertension), neurological diseases (e.g., amyotrophic lateral sclerosis, Alzheimer's disease, myasthenic disorders, Huntington's disease, Spinocerebellar ataxia, frontotemporal dementia, Parkinson's disease, prion diseases, and Lafora disease, and those arising from ischemic or hypoxic conditions), kidney disorders, inflammatory or autoimmune diseases (e.g., ischemia or reperfusion injury, restenosis, Rheumatoid arthritis, inflammatory Bowel Disease, e.g., Crohn's Disease or Ulcerative Colitis, lupus, multiple sclerosis, diabetes, e.g., type II diabetes, and diabetic conditions, arthritis, e.g., rheumatoid or psoriatic), respiratory diseases (e.g., asthma, Chronic obstructive pulmonary diseases (COPD), cystic fibrosis, acute respiratory distress syndrome (ARDS), emphysema, and acute lung injury), hearing disorders, epilepsy, spinal cord injuries, oral mucositis, male infertility, uterine disorders, endometrial disorders or conditions, as well as conditions relating to metabolism (e.g., obesity), ischemia, stroke, alcohol metabolism and liver function (see, e.g., International Pub. Nos. WO 2006/029161, WO 2007/022470, WO 2007/130604, WO 2008/021157, WO 2009/104051, WO 2009/142822, WO 2019/217459, WO 2020/123083; European Pub. No. EP 2504435; and U.S. Patent Pub. Nos. U.S. 2011/0223665, U.S. 2012/0116360, U.S. 2012/0071540, U.S. 2016/0257956, U.S. 2015/0196648, and U.S. 2017/0304459). The RNAi molecule may target a gene that encodes, for example, an oncogene, a transcription factor, a receptor, an enzyme, a structural protein, a cytokine, a cytokine receptor, a lectin, a selectin, an immunoglobulin, a kinase and a phosphatase.

Other heterologous cargo and uses are contemplated. For example, MEVs can carry heterologous cargo, for example, for treating conditions resulting from trauma, such as wounds, burns, skin cuts, broken bones, hair loss, dermis exposure, mucosal exposure, fibrosis, lacerations, and ulcerations. MEVs can carry heterologous cargo, for example, for treating conditions resulting from natural or induced aging, in particular on the skin, or of the vision.

MEVs can be used to deliver heterologous cargo to treat, e.g., with gene silencing, or prevent, e.g., through vaccination, infectious diseases. For example, MEVs derived from antigen-pulsed macrophages or dendritic cells were shown to elicit an immune response when introduced into naïve animals (György et al. (2015) Annu. Rev. Pharmacol. Toxicol. 55:439-464). Gene silencing also can be used to target a pathogen-associated protein, such as a viral protein involved in immunosuppression of the host, replication of the pathogen, transmission of the pathogen, or maintenance of the infection; or a host protein which facilitates entry of the pathogen into the host, drug metabolism by the pathogen or host, replication or integration of the pathogen's genome, establishment or spread of infection in the host, or assembly of the next generation of pathogen. Pathogens can include, for example, RNA and DNA viruses such as arenaviruses, coronaviruses, influenza viruses, paramyxoviruses, flaviviruses (e.g., West Nile virus), picornaviruses (e.g., Coxsakievirus, Poliovirus, and Rhinovirus), rhabdoviruses, filoviruses, retroviruses (e.g., lentiviruses, and Rous sarcoma virus), adenoviruses, poxviruses, herpes viruses, human papilloma viruses, cytomegaloviruses, hepadnaviruses (e.g., Hepatitis B and C), rotaviruses, respiratory syncytial viruses, polyomaviruses, and others; bacteria; fungi; helminths; schistosomes; trypanosomes; parasites including plasmodiums (e.g., Plasmodium malariae and others); and mammalian transposable elements (see, e.g., International Pub. Nos. WO 2010/141724, WO 2011/071860, WO 2011/072292, WO 2013/126803, WO 2020/035620, and WO 2020/097540; Australian Pub. Nos. AU 2004257373 A1, AU 2013203219 B2, and AU 2016225873 A1; European Pub. Nos. EP 2395012, and EP 2888240; U.S. Patent Pub. Nos. U.S. 2011/0223665, U.S. 2014/0256785, and U.S. 2019/0032051; Japanese Pub. No. JP 2018-197239A; and Taiwanese Pub No. TW 201204351A).

MEVs also can be used to deliver mRNA molecules that encode therapeutically useful polypeptides. For example, in cases where subjects lack a specific gene product, the gene can be encoded in a nucleic acid molecule, such as an RNA molecule. The nucleic acid molecule encoding the gene product can be loaded into a MEV and delivered to a subject lacking the gene product. For example, diseases that occur due to the absence or deficiency of a gene product, include, but are not limited to, lysosomal storage disorders, metabolic disorders of the urea cycle, SMN1-related spinal muscular atrophy (SMA); amyotrophic lateral sclerosis (ALS); GALT-related galactosemia; cystic fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia; Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1-, EIF2B2-, EIF2B3-, EIF2B4- and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); Notch-3 related cerebral autosomal dominant arteriopathy with subcortical infarcts and leukoencephalopathy (CADASIL); SCNIA- and SCN1B-related seizure disorders; the Polymerase G-related disorders, including Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; and Wilson's disease (see, e.g., International Pub. Nos. WO 2011/068810, WO 2019/243574, WO 2019/092287, and WO 2020/099682).

The MEVs can be loaded with a gene editing system such as clustered regularly interspaced short palindromic repeats (CRISPR)-CRISPR-associated protein 9 (Cas9), and also other technologies that are used to edit genomes. These include, for example: transcription activator-like effector nucleases (TALENs), zinc-finger nucleases (ZFNs), and homing endonucleases or meganucleases. Nucleic acid encoding the editing system is introduced into the microalgae or expression and packaging in the MEVs.

CRISPR technology allows for the modification of the genome in a living organism, and is based on the bacterial CRISPR/Cas9 antiviral defense system (see e.g., SEQ ID NOs: 70 and 71 for an exemplary Cas9 coding and encoded protein sequences). The system allows for DNA cleavage at a target site. The type II CRISPR system incorporates sequences from invading foreign nucleic acids, such as DNA from viruses or plasmids, between CRISPR repeat sequences encoded within the host genome. Transcripts from the CRISPR repeat sequences are processed into CRISPR RNAs (crRNAs). Each crRNAs harbors a variable sequence transcribed from the foreign DNA and a part of the CRISPR repeat. Each crRNA hybridizes with a second transactivating CRISPR RNA (tracrRNA) and these two RNAs complex with and direct the Cas9 nuclease to cleave the target DNA sequence. By delivering a Cas nuclease complexed with a synthetic guide RNA (gRNA), which contains a fusion of a crRNA and a tracrRNA, into a cell, the cell's genome can be cut at a desired location, allowing existing genes to be removed and/or new ones added in vivo (Sander et al. (2014) Nat. Biotechnol. 32 (4): 347-355). The CRISPR technology can be used with the Cas polypeptide or the single RNA guided endonuclease Cpf1 to effect genome modification, and can be delivered in lipid nanoparticles, EVs and other vesicles (see, e.g., International PCT publication Nos. WO 2017/161010, WO 2019/238626, and WO 2020/097540).

MEVs also can be used to treat diseases, including but not limited to those listed above, by introduction of an endogenous cargo in form of a therapeutic protein, or polypeptide, to a target cell. Non-limiting examples of such therapeutic can be a biologic therapeutic agent selected from an allergen, adjuvant, antigen, or immunogen, antibody (e.g., whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv) 2, Fab, Fab′, and F (ab′) 2, F (ab) 2, Fv, dAb, and Fd fragments, diabodies, and antibody-related polypeptides), cytokine, hormone, factor, cofactor, cell component protein, metabolic enzyme, immunoregulatory enzyme, interferon, interleukin, gastrointestinal enzyme, an enzyme or factor implicated in hemostasis, growth regulatory enzyme, vaccine, antithrombotic, toxin, antitoxin, or diagnostic or imaging biologic agent (see, e.g., International Pub. Nos. WO 2017/203260, WO 2018/102397, WO 2019/081474, WO 2019/155060, WO 2020/041720; Australian Pub. No. AU 2018365299A1; Singapore Pub. No. SG 11201811149TA; and U.S. Patent Pub. No. U.S. 2019/0202892). For example, MEV therapies can be used to treat Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barre syndrome, acute myocardial infarction, acute respiratory distress syndrome (ARDS), sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy and other muscular dystrophies, lysosomal storage diseases, neurodegenerative diseases, cancer-induced cachexia, anorexia, diabetes mellitus type 2, and cancers (e.g., acute lymphoblastic leukemia, ALL), acute myeloid leukemia, adrenocortical carcinoma, AIDS-related cancers, AIDS-related lymphoma, anal cancer, appendix cancer, astrocytoma, cerebellar or cerebral, basal-cell carcinoma, bile duct cancer, bladder cancer, bone tumor, brainstem glioma, brain cancer, brain tumor (cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumor (childhood, gastrointestinal), carcinoma of unknown primary, central nervous system lymphoma, cerebellar astrocytoma/malignant glioma, cervical cancer, chronic lymphocytic leukemia, chronic myelogenous leukemia, chronic myeloproliferative disorders, colon Cancer, cutaneous T-cell lymphoma, desmoplastic small round cell tumor, endometrial cancer, ependymoma, esophageal cancer, extracranial germ cell tumor, extragonadal germ cell tumor, extrahepatic bile duct cancer, eye cancer (intraocular melanoma, retinoblastoma), gallbladder cancer, gastric cancer, gastrointestinal carcinoid tumor, gastrointestinal stromal tumor (GIST), germ cell tumor (extracranial, extragonadal, or ovarian), gestational trophoblastic tumor, glioma (glioma of the brain stem, cerebral astrocytoma, visual pathway and hypothalamic glioma), gastric carcinoid, hairy cell leukemia, head and neck cancer, heart cancer, hepatocellular (liver) cancer, Hodgkin lymphoma, hypopharyngeal cancer, intraocular melanoma, islet cell carcinoma (endocrine pancreas), Kaposi sarcoma, kidney cancer (renal cell cancer), laryngeal cancer, leukemias (acute lymphoblastic, acute myeloid, chronic lymphocytic, chronic myelogenous, hairy cell leukemia), lip and oral cancer, cavity cancer, liposarcoma, liver cancer (primary), lung cancer (non-small cell, small cell), lymphomas, AIDS-related lymphoma, Burkitt lymphoma, cutaneous T-cell lymphoma, Hodgkin lymphoma, non-Hodgkin, medulloblastoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic/myeloproliferative diseases, myelogenous leukemia, chronic myeloid leukemia, myeloma, nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic islet cell cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, pleuropulmonary blastoma, prostate cancer, rectal cancer, renal cell carcinoma (kidney cancer), retinoblastoma, rhabdomyosarcoma, salivary gland cancer, sarcoma (Ewing family of tumors sarcoma, Kaposi sarcoma, soft tissue sarcoma, uterine sarcoma), Sézary syndrome, skin cancer (nonmelanoma, melanoma), small intestine cancer, squamous cell, squamous neck cancer, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine cancer, uterine sarcoma, vaginal cancer, vulvar cancer, Waldenstrom's macroglobulinemia, and/or Wilms' tumor) (see, e.g., International Pub. Nos. WO 2017/203260 and WO 2019/155060A1; and U.S. Patent Pub. No. U.S. 2019/0388347).

### c. Cosmetic and Dermatological Applications

MEVs carrying cargo of pharmacological agents also can be used for cosmetic and dermatological applications. For example, skin care products such as creams, lotions, gels, emulsions, ointments, pastes, powders, liniments, sunscreens, and shampoos comprising EVs, particularly from stem cells, can be used to improve and/or alleviate symptoms and problems such as dry skin, elasticity, wrinkles, folds, ridges, and/or skin creases (see, e.g., Singapore Pub. No. SG 11201811149TA). Stem cell EVs, which inherently carry cytokines, growth and transcription factors among their cargo, also have been shown to control inflammation, accelerate skin cell migration and proliferation, control wound scarring, improve angiogenesis, and ameliorate signs of skin aging. Although the exact mechanisms are being elucidated, the effect of stem cell EVs on wound healing may rely in the vertical transfer on microRNAs or proteins to skill cells. Angiogenesis, a part of wound healing, can be induced by stem cell EVs. Stem cell EVs also have beneficial effects for cellular matrix maintenance and collagen production, and have been shown to play a role in rejuvenating skin cells (da Fonseca Ferreira, A. and Gomes, D. (2019) Bioengineering (Basel) 6 (1): 4). MEVs loaded with a desired cargo can thus be used for cosmetic and dermatological applications.

### E. Pharmaceutical Compositions, Formulations, Kits, Articles of Manufacture, and Combinations, and Drug Delivery Systems

### 1. Pharmaceutical Compositions and Formulations

The compositions containing the MEVs and loaded MEVs provided herein can be formulated as pharmaceutical compositions provided for administration by a desired route, such as oral, mucosal, intravenous, intranasal, and others. Pharmaceutically acceptable compositions are prepared in view of approvals for a regulatory agency or other agency prepared in accordance with generally recognized pharmacopeia for use in human applications. Typically, compounds are formulated into pharmaceutical compositions using techniques and procedures well-known in the art (see e.g., Ansel Introduction to Pharmaceutical Dosage Forms, Fourth Edition, 1985, 126).

The pharmaceutical composition can be used for therapeutic, prophylactic, vaccinal, cosmetic, and/or diagnostic applications. The cargo-loaded MEVs provided herein can be formulated with a pharmaceutical acceptable carrier or diluent. Generally, such pharmaceutical compositions include components that do not significantly impair the biological properties or other properties of the cargo. Each component is pharmaceutically and physiologically acceptable so that it is compatible with the other ingredients and not injurious to the subject to whom it is to be administered. The formulations can be provided in unit dosage form and can be prepared by methods well-known in the art of pharmacy, including but not limited to, tablets, pills, powders, liquid solutions or suspensions (e.g., including injectable, ingestible and topical formulations (e.g., eye drops, gels, pastes, creams, or ointments)), aerosols (e.g., nasal sprays, and inhalers), liposomes, suppositories, pessaries, injectable and infusible solution and sustained release forms. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY. When administered systemically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well-known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

Pharmaceutical compositions provided herein can be in various forms, e.g., in solid, semi-solid, liquid, powder, aqueous, and lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, and powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20th Edition. Baltimore, MD: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

Pharmaceutical compositions can include a carrier, such as a diluent, adjuvant, excipient, or vehicle. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

Compositions can contain, along with an active ingredient, a diluent, such as lactose, sucrose, dicalcium phosphate, and carboxymethylcellulose; a lubricant, such as magnesium stearate, calcium stearate and talc; and a binder, such as starch, natural gums, such as gum acacia, gelatin, glucose, molasses, polyvinylpyrrolidone, celluloses and derivatives thereof, povidone, crospovidone, and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. A composition, if desired, also can contain suitable amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

The pharmaceutical compositions provided herein can contain other additives, including, for example, antioxidants, preservatives, antimicrobial agents, analgesic agents, binders, disintegrants, colorings, diluents, excipients, extenders, glidants, solubilizers, stabilizers, tonicity agents, vehicles, viscosity agents, flavoring agents, emulsions, such as oil-in-water or water-in-oil emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20th Edition. Baltimore, MD: Lippincott Williams & Wilkins). Such carriers and/or additives can be formulated by conventional methods and can be administered to the subject at a suitable dose. Stabilizing agents, such as lipids, nuclease inhibitors, polymers, and chelating agents, can preserve the compositions from degradation within the body.

The formulation should suit the mode of administration. For example, the active compound can be formulated for parenteral administration by injection (e.g., by bolus injection, or continuous infusion). The injectable compositions can take such forms as suspensions, solutions or emulsions in oily or aqueous vehicles. The sterile injectable preparation also can be a sterile injectable solution, or a suspension in a non-toxic parenterally-acceptable diluent or solvent, for example, as a solution in 1,4-butanediol. Sterile, fixed oils are conventionally employed as a solvent or suspending medium. For this purpose, any bland fixed oil can be employed, including, but not limited to, synthetic mono- or diglycerides, fatty acids (including oleic acid), naturally occurring vegetable oils, such as sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation.

The compositions can take the form of solutions, suspensions, emulsions, tablets, pills, capsules, powders, granules, and sustained release formulations. Capsules and cartridges of, e.g., gelatin, for use in an inhaler or insufflator, can be formulated containing a powder mix of a therapeutic compound and a suitable powder base, such as lactose or starch. A composition can be formulated as a suppository, with traditional binders and carriers, such as triglycerides.

For oral administration, for example, the MEVs can be formulated as a liquid, including as an emulsion, such as a nanoemulsion or microemulsion, or can be provided in capsule form, such as in a soft gel, in which the liquid is introduced, or as soft gel or other such capsule in which the liquid is introduced. The MEVs can be lyophilized to produce a powder, which can be introduced into capsules or formed into tablets, such as compressed powder tablets or layered tablets, in any desired geometry. For oral administration the MEVs can be provided as a liquid pharmaceutical composition, as discussed above.

For inhalation and also for intranasal administration, the MEVs can be formulated for administration as an aerosol. The MEVs can be formulated as an emulsion for use as an aerosol for inhalation into the lungs or for intranasal administration.

Oral formulations can include standard carriers, such as pharmaceutical grades of mannitol, lactose, starch, magnesium stearate, sodium saccharine, cellulose, magnesium carbonate, and other such agents. Preparations for oral administration also can be suitably formulated with protease inhibitors, such as a Bowman-Birk inhibitor, a conjugated Bowman-Birk inhibitor, aprotinin and camostat. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier, so as to provide the compound in a form for proper administration to a subject or patient.

In some examples, the MEVs can be dried, such as by lyophilization, to form a dry powder, which can be stored. The dry powder can be reconstituted by adding a pharmaceutically acceptable carrier, to produce a composition for administration as a liquid, such as orally or intravenously or other suitable route. The dry powder can be introduced into a capsule, such as soft gel capsule, or pressed into a tablet or mixed with other carriers to form a layered tablet.

Tablets and other forms typically contain other excipients. These excipients include, for example, tablet disintegrants, such as corn starch, glidants, such as silicon dioxide, and lubricants such as magnesium stearate. The compositions contain minor amounts by weight of glidants and lubricants, e.g., each two percent (2%) or less by weight. Tablet disintegrants are optionally present, and, if present, are included in sufficient amounts to assure that the tablet disintegrates upon ingestion. For example, disintegrants, such as corn starch, can be employed at concentrations of from about zero to about 30 percent by weight of the composition.

Free flowing powders also can be used to administer the active agent by inhalation using a dry powder inhaler. Such dry powder inhalers typically administer the active agent as a free-flowing powder that is dispersed in a patient's air-stream during inspiration. In order to achieve a free flowing powder, the active agent is typically formulated with a suitable excipient such as lactose or starch. For example, such a dry powder formulation can be made, for example, by combining the lactose with the active agent and then dry blending the components. Alternatively, if desired, the active agent can be formulated without an excipient. The pharmaceutical composition is then typically loaded into a dry powder dispenser, or into inhalation cartridges or capsules for use with a dry powder delivery device. Examples of dry powder inhaler delivery devices include Diskhaler® (GlaxoSmithKline, Research Triangle Park, NC) (see, e.g., U.S. Pat. No. 5,035,237); Diskus (GlaxoSmithKline) (see, e.g., U.S. Pat. No. 6,378,519; Turbuhaler™ (AstraZeneca, Wilmington, Del.) (see, e.g., U.S. Pat. No. 4,524,769); Rotahaler (GlaxoSmithKline) (see, e.g., U.S. Pat. No. 4,353,365) and HandiHaler® (Boehringer Ingelheim). Further examples of suitable DPI devices are described in U.S. Pat. Nos. 5,415,162, 5,239,993, and 5,715,810 and references cited therein.

The route of administration is in accord with known methods. These include, but are not limited to, intramuscular injection or other injection, subcutaneous administration, infusion by intravenous, intranasal, intraperitoneal, intracerebral, intramuscular, subcutaneous, intraocular, intraarterial, intrathecal, inhalation or intralesional routes, topical, rectal, mucosal, and by sustained release systems. The MEVs or cargo-loaded MEVs can be administered continuously by infusion or by bolus injection.

One can administer the MEVs or cargo-loaded MEVs in a local or systemic manner, such as by oral administration. Oral formulations can be solids or liquids or combinations thereof. Solid formulations for oral administration include, for example, liquids, tablets, pills, powders, granules, capsules, films, wafers, and other such forms. Liquids for oral administration include, for example, elixirs, suspensions, and emulsions.

Solid formulations are prepared by mixing lyophilized MEVs or a composition containing the MEVs with one or more suitable excipients such as starch, calcium carbonate, sucrose, lactose, and/or gelatin, as discussed above. If liquid MEVs are mixed, the resulting compositions can be lyophilized to produce a powder, which can be formed into an oral dosage form such as a tablet or film, or loaded into gel capsules. Liquid formulations for oral administrations include suspensions, solutions, and emulsions. Formulations for parenteral administration are sterilized aqueous solutions, water-insoluble excipients, suspensions, emulsions, lyophilized preparations and suppositories.

As shown herein, biodistribution of the MEVs is a function of the route of administration. For example, as shown oral administration of the MEVs enters the gastro-intestinal tract and subsequently are found in the spleen, including the white spleen. Following intranasal administration, MEVs are found in the brain.

The MEVs or cargo-loaded MEVs can be prepared in a mixture with a pharmaceutically acceptable carrier. Techniques for formulation and administration of the compounds are known to one of skill in the art (see e.g., “Remington's Pharmaceutical Sciences,” Mack Publishing Co., Easton, Pa.). This therapeutic composition can be administered intravenously or through the nose or lung, such as a liquid or powder aerosol (lyophilized). The composition also can be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

Pharmaceutical compositions suitable for use include compositions wherein the MEVs or cargo-loaded MEVs are contained in an amount effective to achieve their intended purpose. Determination of a therapeutically effective amount is well within the capability of those skilled in the art. Therapeutically effective dosages can be determined by using in vitro and in vivo methods, and/or by a skilled person.

Therapeutic formulations can be administered in many conventional dosage formulations. Dosage formulations of Cargo-loaded MEVs provided herein are prepared for storage or administration by mixing the compound having the desired degree of purity with physiologically acceptable carriers, excipients, or stabilizers. Such materials are non-toxic to the recipients at the dosages and concentrations employed, and can include buffers such as Tris HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN (polysorbates), Pluronic, polyethylene glycol, and others.

In particular examples herein, provided are pharmaceutical compositions that contain a stabilizing agent. The stabilizing agent can be an amino acid, amino acid derivative, amine, sugar, polyol, salt or surfactant. In some examples, the stable co-formulations contain a single stabilizing agent. In other examples, the stable co-formulations contain 2, 3, 4, 5 or 6 different stabilizing agents. For example, the stabilizing agent can be a sugar or polyol, such as a glycerol, sorbitol, mannitol, inositol, sucrose or trehalose. In particular examples, the stabilizing agent is sucrose. In other examples, the stabilizing agent is trehalose. The concentration of the sugar or polyol is from or from about 100 mM to 500 mM, 100 mM to 400 mM, 100 mM to 300 mM, 100 mM to 200 mM, 200 mM to 500 mM, 200 mM to 400 mM, 200 mM to 300 mM, 250 mM to 500 mM, 250 mM to 400 mM, 250 mM to 300 mM, 300 mM to 500 mM, 300 mM to 400 mM, or 400 mM to 500 mM, each inclusive.

In examples, the stabilizing agent can be a surfactant that is a polypropylene glycol, polyethylene glycol, glycerin, sorbitol, poloxamer and polysorbate. For example, the surfactant can be a polypropylene glycol, polyethylene glycol, glycerin, sorbitol, poloxamer and polysorbate, such as a poloxamer 188, polysorbate 20 and polysorbate 80. In particular examples, the stabilizing agent is polysorbate 80. The concentration of surfactant, as a % of mass concentration (w/v) in the formulation, is between or about between 0.005% to 1.0%, 0.01% to 0.5%, 0.01% to 0.1%, 0.01% to 0.05%, or 0.01% to 0.02%, each inclusive.

When used for in vivo administration, the formulation should be sterile and can be formulated according to conventional pharmaceutical practice. This is readily accomplished by filtration through sterile filtration membranes, prior to or following lyophilization and reconstitution. The MEVs or cargo-loaded MEVs can be stored in lyophilized form or in solution; they can be frozen or refrigerated. Other vehicles such as naturally occurring vegetable oil like sesame, peanut, or cottonseed oil or a synthetic fatty vehicle like ethyl oleate can be included. Buffers, preservatives, and antioxidants can be incorporated according to accepted pharmaceutical practice.

The MEVs or cargo-loaded MEVs provided herein, can be provided at a concentration in the composition of from or from about 0.1 to 10 mg/mL or higher or lower amounts, depending upon the application and the subject, such as, for example a concentration that is at least or at least about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 mg/mL or more. The volume of the solution can be at or about 1 to 100 mL, such as, for example, at least or about at least or 0.5, 1, 2, 3, 4, 5, 10, 15, 20, 25, 30, 35, 40, 45, 50, 55, 60, 65, 70, 75, 80, 85, 90, 95, 100 mL or more. In some examples, the MEVs or cargo-loaded MEVs are supplied in phosphate buffered saline.

The MEVs or cargo-loaded MEVs provided herein can be provided as a controlled release or sustained release composition. Polymeric materials are known in the art for the formulation of pills and capsules which can achieve controlled or sustained release of the Cargo-loaded MEVs provided herein (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Langer et al. (1983) J. Macromol. Sci. 23:61; see also Levy et al. (1985) Science 228:190; During et al. (1989) Ann. Neurol. 25:351; Howard et al. (1989) J. Neurosurg. 71:105; U.S. Pat. Nos. 5,679,377, 5,916,597, 5,912,015, 5,989,463, 5,128,326; and PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulation can be used to produce a sustained release formulation containing the MEVs or cargo-loaded MEVs provided herein.

In some examples, the pharmaceutical composition contains the MEVs or cargo-loaded MEVs provided herein and one or more additional agents, such as an antibody or other therapeutic, for combination therapy.

### 2. Articles of Manufacture/Kits and Combinations

Pharmaceutical compositions of the MEVs or cargo-loaded MEVs can be packaged as articles of manufacture containing packaging material, a pharmaceutical composition which is effective for treating a disease or condition that can be treated by administration of the particular MEVs or cargo-loaded MEVs, such as the diseases and conditions described herein or known in the art, and a label that indicates that the cargo, such as an antibody or nucleic acid molecule, is to be used for treating the infection, disease or disorder. The pharmaceutical compositions can be packaged in unit dosage forms containing an amount of the pharmaceutical composition for a single dose or multiple doses. The packaged compositions can contain a lyophilized powder of the pharmaceutical compositions containing the cargo-loaded MEVs which can be reconstituted (e.g., with water or saline) prior to administration.

The articles of manufacture provided herein contain packaging materials. Packaging materials for use in packaging pharmaceutical products are well-known to those of skill in the art (see, e.g., U.S. Pat. Nos. 5,323,907, 5,052,558 and 5,033,252). Examples of pharmaceutical packaging materials include, but are not limited to, blister packs, bottles, tubes, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment.

The MEVs or cargo-loaded MEVs can be provided as combinations and as kits. Kits optionally can include one or more components such as instructions for use, devices and additional reagents (e.g., sterilized water or saline solutions for dilution of the compositions and/or reconstitution of lyophilized protein), and components, such as tubes, containers and syringes for practice of the methods. Exemplary kits can include the MEVs or cargo-loaded MEVs provided herein, and can optionally include instructions for use, a device for administering the MEVs or cargo-loaded MEVs to a subject, a device for detecting MEVs or cargo-loaded MEVs in samples obtained from a subject, and a device for administering an additional therapeutic agent to a subject.

The kit can, optionally, include instructions. Instructions typically include a tangible expression describing the MEVs or cargo-loaded MEVs, and, optionally, other components included in the kit, and methods for administration, including methods for determining the proper state of the subject, the proper dosage amount, dosing regimens, and the proper administration method for administering the MEVs or cargo-loaded MEVs. Instructions also can include guidance for monitoring the subject over the duration of the treatment time.

Kits also can include a pharmaceutical composition described herein and an item for diagnosis. For example, such kits can include an item for measuring the concentration, amount or activity of the Cargo-loaded MEVs, in a subject.

In some examples, the MEVs or cargo-loaded MEVs are provided in a diagnostic kit for the detection of the MEVs or cargo-loaded MEVs or cargo in an isolated biological sample (e.g., tumor cells, such as circulating tumor cells obtained from a subject or tumor cells excised from a subject).

Kits provided herein also can include a device for administering the MEVs to a subject. Any of a variety of devices known in the art for administering medications to a subject can be included in the kits provided herein. Exemplary devices include, but are not limited to, a hypodermic needle, an intravenous needle, a catheter, a nebulizer, and an inhaler. Typically, the device for administering the compositions is compatible with the desired method of administration of the composition.

### 3. Administration of Endogenously Loaded MEVs and Routes of Administration

The cargo-loaded MEVs provided herein can be administered to a subject by any method or route known in the art for the administration of pharmaceuticals, including biologics. The cargo-loaded MEVs can be administered by routes. Routes of administration, include, but are not limited to, systemic, topical, and local administration. Routes of administration as discussed, include, oral administration, enteral administration, parenteral, which includes intradermal, intramuscular, intraperitoneal, intravenous, subcutaneous, and intracavity administration, topical, epidural, mucosal, which includes topical, intranasal, vaginal, vulvovaginal, esophageal, oroesophageal, bronchial, rectal, and pulmonary.

The cargo-loaded MEVs can be administered externally to a subject, at the site of the disease for exertion of local or transdermal action. Compositions containing the cargo-loaded MEVs can be administered, as discussed above by any route depending upon the target tissue or organ for the disease, disorder, or condition, treated. For targeting the spleen, for example, oral administration is employed. The MEVs for such administration are formulated as a liquid or solid as discussed above. As shown in the Examples, following oral administration, the MEVs pass through the stomach and into the intestines, and, as shown then are found in the spleen. This is a distinction and advantage of the MEVs compared to mammalian MEVs, which cannot be administered orally because they cannot survive the harsh conditions in the stomach. Administration can be by infusion, inhalation, by bolus injection, by absorption through epithelial or mucocutaneous linings (e.g., topical, oral, vaginal, rectal and intestinal mucosa).

Compositions containing the cargo-loaded MEVs can be administered alone and/or together with or sequentially with other biologically active agents. For example, the cargo-loaded MEVs are administered by infusion delivery, such as by infusion pump or syringe pump, and can be administered in combination with another therapeutic agent or as a monotherapy.

The method and/or route of administration can be altered to alleviate adverse side effects associated with administration provided herein. For example, if a patient experiences a mild or moderate (i.e., Grade 1 or 2) infusion reaction, the infusion rate can be reduced (e.g., reduced by 10%, 20%, 30%, 40%, 50%, 60%, 70%, 80%, 90% or more). If the patient experiences severe (i.e., Grade 3 or 4) infusion reactions, the infusion can be temporarily or permanently discontinued.

In some examples, if the subject experiences an adverse side effect, such as severe skin toxicity, for example severe acneiform rash, treatment adjustments can be made. For example, after the occurrence of an adverse side effect, administration can be delayed, such as for 1 to 2 weeks or until the adverse side effect improves. In some examples, after additional occurrences of an adverse side effect, the dosage can be reduced. A particular regimen and treatment protocol can be established by the skilled physician or other skilled practitioner in the art.

Appropriate methods for delivery, can be selected by one of skill in the art based on the properties of the dosage amount of the cargo-loaded MEVs or the pharmaceutical composition containing the cargo-loaded MEVs. Such properties include, but are not limited to, solubility, hygroscopicity, crystallization properties, melting point, density, viscosity, flow, stability, and degradation profile. As detailed in the Examples, the route of administration of the MEVs determines their biodistribution. As shown, following administration, the MEVs distribute in tissues and organs that differ from mammalian and other EVs.

### 4. Drug Delivery Systems

As discussed above, and demonstrated in the Examples, the MEVs and compositions containing the MEVs can be considered drug delivery systems in which the MEVs are formulated for a particular route of administration for targeting to an organ or tissue involved in a disease, disorder, or condition, that can be treated by the selected cargo. Drug delivery systems can optionally include additional components, such as another therapeutic agent for combination therapy and/or devices for administration.

A composition or drug delivery system, for example, contains extracellular vesicles (MEVs) formulated for oral delivery, intravenous delivery, intramuscular delivery, intranasal delivery, subcutaneous delivery, topical delivery, mucosal delivery, intraperitoneal delivery, intratumoral delivery, or inhalation delivery. The MEVs are isolated from the cell culture, cell culture medium, or genetically-modified microalgae cell, or produced by the methods as described herein. The MEVs are formulated for a route of delivery, whereby the endogenous cargo is delivered to a target organ or tissue. Target organs or tissues include lungs, liver, spleen, intestine, brain, spinal cord, peripheral nerves, lymphoid tissues, eyes, mucosal tissue, skin, hematopoietic tissues, pancreas, muscle, bones, heart, endocrine tissues and kidneys. The target organ or tissue is mucosal tissue can be is nasobuccal, ocular, urogenital, vaginal, or rectal. The drug delivery system can be formulated as a suspension or emulsion, which can be a nanoemulsion or is a microemulsion. The drug delivery system can be formulated as a tablet, capsules, gel capsule, powder, troche, granules, liquid for oral administration, oil, or is a suspension or emulsion for nasal administration or oral administration, or inhalation, or nebulization, or intratracheal administration.

As described above, exemplary of MEVs are Chlorella extracellular vesicles that contain heterologous (to the Chlorella microalgae) cargo that produce the MEVs with endogenously loaded bioactive molecule cargo by the genetically-modified microalgae cells that produced the MEVs. Chlorella species include, but are not limited to, Chlorella ellipsoidea, Chlorella pyrenoidosa, Chlorella sorokiniana, Chlorella vulgaris, and Chlorella variabilis.

The endogenous cargo can be a therapeutic for treating or preventing a disease, disorder, or condition, or treating or preventing a symptom thereof, such as, for example, the endogenous cargo comprises or encodes a protein a therapeutic that is a prophylactic for preventing or reducing the risk of getting a disease, disorder, or condition, or reducing the severity of a disease, disorder, or condition. The endogenous cargo comprises a nucleic acid, a protein, a small peptide, a peptide, and/or a polypeptide, with the provisos recited throughout the disclosure.

The cargo includes any described herein for endo-loading into the MEVs, and/or known to those of skill in the art. For example, the endogenous cargo for the drug delivery systems, as well as the compositions and MEVs described in the sections above and below, can comprise nucleic acid encoding an immunomodulatory agent to increase or decrease production of one or more cytokines; up- or down-regulate self-antigen presentation; mask MHC antigens; or promote the proliferation, differentiation, migration, or activation state of one or more types of immune cells. The endogenous cargo can comprise or encode a hormone or a cytokine or a chemokine; or comprises nucleic acid encoding a hormone, or a cytokine, or a chemokine. Exemplary thereof is one or more of a hormone or cytokine or growth factor selected from among human growth hormone; N-methionyl human growth hormone; bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factors; fibroblast growth factors; prolactin; placental lactogen; tumor necrosis factor-alpha and -beta; Müllerian-inhibiting substance; gonadotropin-associated peptide; inhibin; activin; vascular endothelial growth factors; integrin; thrombopoietin (TPO); nerve growth factors, transforming growth factors (TGFs); insulin-like growth factor-I and -II; erythropoietin (EPO); osteoinductive factors; interferons such as interferon-alpha,-beta, and -gamma; colony stimulating factors (CSFs) such as macrophage-CSF (M-CSF); granulocyte-macrophage-CSF (GM-CSF); and granulocyte-CSF (G-CSF); or an interleukin (IL). The cargo can comprise for mRNA-mediated gene therapy, gene silencing, gene substitution, gene overexpression, and/or gene editing, can comprise one or more of peptides or proteins for gene regulation, gene substitution, gene overexpression, gene editing, regulation of cell metabolism, cell functions, and protein therapy. The mRNA-mediated gene therapy or protein therapy can be treating an inborn error of metabolism. The cargo can comprise a vaccine, such as a protein vaccine or mRNA vaccine. Vaccines, as described herein can be immunoprotective and/or prophylactic, and/or can treat a disease, disorder, or condition.

The MEV cargo can comprise a nucleic acid or protein or a nucleic acid encoding a protein that is a therapeutic product for treatment of cancer, or an infectious disease, or metabolic diseases, or a neurodegenerative disease or other CNS disorder, or aging, or an aging associated disease, or genetic diseases, or ophthalmic disorders, or immunological disorders, or involving internal organs urogenital organs, the cardiovascular system and associated organs and tissues, hematopoietic or lymphoid tissues, sensory organs and tissues, urogenital organs and tissues, muscle tissues, bones, and/or endocrine tissues. Internal organs include, for example, liver, or the pancreas, or spleen, or brain. The compositions and drug delivery systems that contain the MEVs can be formulated for oral administration, parenteral administration, topical administration, local administration, intratumoral administration, systemic administration, mucosal administration, intravenous administration, subcutaneous administration, intramuscular administration, intraperitoneal administration, transdermal administration, intranasal administration, inhalation, intratracheal administration. They can be formulated for oral delivery, or as an aerosol for intranasal, inhalation, or nebulization. The drug delivery systems and compositions provided herein can be used or delivering endogenous cargo in an MEV to an organ or tissue, wherein the mode of administration is selected to target the organ or tissue.

It is shown herein that the fate of administered MEVs is a function of the route of administration. MEVs have unique trafficking patterns, and provide an opportunity for targeted delivery of the loaded cargo. For example, MEVs can be delivered orally and, upon oral administration, they traffic through the GALT. Sections F and G detail biodistribution and delivery of cargo-loaded MEVs.

### 5. Combination Therapies

The cargo-loaded MEVs provided herein can be administered before, after, or concomitantly with one or more other therapeutic regimens or agents. The skilled medical practitioner can determine empirically, or by considering the pharmacokinetics and modes of action of the agents, the appropriate dose or doses of each therapeutic regimen or agent, as well as the appropriate timings and methods of administration. The additional therapeutic regimens or agents can improve the efficacy or safety or other properties of the cargo-loaded MEVs. In some examples, the additional therapeutic regimens or agents can treat the same disease or a comorbidity. In some examples, the additional therapeutic regimens or agents can ameliorate, reduce or eliminate one or more side effects known in the art or described herein that are associated with administration of the cargo-loaded MEVs or the cargo.

For example, the cargo-loaded MEVs described herein can be administered with chemotherapy, radiation therapy, or both chemotherapy and radiation therapy, or for anti-viral or anti-bacterial or other pathogen therapy, the cargo-loaded MEVs can be administered with other anti-pathogen therapeutics and treatments. The cargo-loaded MEVs can be administered in combination with one or more other prophylactic or therapeutic agents, including but not limited to antibodies, cytotoxic agents, chemotherapeutic agents, cytokines, growth inhibitory agents, anti-hormonal agents, kinase inhibitors, anti-angiogenic agents, cardio-protectants, immunostimulatory agents, immunosuppressive agents, agents that promote proliferation of hematological cells, angiogenesis inhibitors, protein tyrosine kinase (PTK) inhibitors, FcγRIIb or other Fc receptor inhibitors, or other therapeutic agents.

The one or more additional agents can be administered simultaneously, sequentially or intermittently with the cargo-loaded MEVs. The agents can be co-administered, for example, as part of the same pharmaceutical composition or same method of delivery. In some examples, the agents can be co-administered at the same time as the cargo-loaded MEVs, but by a different means of delivery. The agents also can be administered at a different time than administration of the cargo-loaded MEVs, but close enough in time to have a combined prophylactic or therapeutic effect. In some examples, the one or more additional agents are administered subsequent to or prior to the administration of the cargo-loaded MEVs separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the cargo-loaded MEVs provided herein are administered multiple times.

### F. Biodistribution of Mevs Following Administration Via Various Routes

### 1. Biodistribution of Mammalian EVs

Pharmacokinetics and biodistribution in organs and tissues of mammalian EVs have been extensively studied for their pharmacokinetics and distribution in organs and tissues (Vader et al. (2016) Advanced drug delivery reviews 106 (Pt A): 148-156, doi.org/10.1016/j.addr.2016.02.006; Morishita et al. (2017) Journal of pharmaceutical sciences 106 (9): 2265-2269, hdoi.org/10.1016/j.xphs.2017.02.030). Treatments with mammalian cell-derived EVs are generally based on intravenous or intraperitoneal routes of administration. Primary target organs upon systemic administration of mammalian EVs are the liver, spleen and lungs. A comprehensive study (see, Wiklander et al. (2015) J. Extracellular Vesicles 4:26316) of the tissue distribution of fluorescently-labelled mammalian EVs from various cell sources demonstrated that 24 hours after intravenous (i.v.) injection in mice, the highest fluorescence signal was in the liver, followed by spleen, gastrointestinal tract and lungs. Furthermore, cell source, EV dose, and route of administration was shown to affect EV distribution; for example, injection of higher EV doses resulted in relatively lower liver accumulation compared to lower doses, possibly caused by saturation of the mononuclear phagocyte system (MPS). Comparison between intraperitoneal (i.p.), subcutaneous (s.c.) and i.v. administrations showed that intraperitoneal and subcutaneous doses resulted in reduced EV accumulation in liver and spleen and enhanced pancreas and gastrointestinal tract accumulation compared to i.v. injections. Systemically administered EVs are reported to be rapidly taken up by the mononuclear phagocyte system (MPS), particularly in the liver and spleen. The mechanism of clearance resembles that described for synthetic nanoparticles, such as liposomes (Van der Meel et al. (2014) J. Control. Release 195:72-8). The majority of splenic accumulation is caused by EV storage in the spleen rather than uptake by the spleen (Lai C. P. et al. (2014) ACS Nano 8:483-494). Biodistribution of mammalian EVs following other routes of administration also has been investigated. For targeting of the central nervous system, intranasal administration of curcumin-loaded mammalian EVs resulted in EV localization in the brain. Drug levels peaked at 1 hour after administration, and a significant amount detected after 12 hours with no toxic effects observed (Zhuang et al. (2011) Mol. Ther. 19:1769-1779).

In general, mammalian EVs are not employed for oral delivery because of their low stability at various pH and temperatures, rapid degradation of biomolecules in the digestive tract, and the limitations of industrial scale production for oral dosing (Cheng et al. (2019) Protein Cell 10:295-299). The only exception so far are bovine milk-derived EVs, which upon oral delivery to mice have shown a pattern of distribution that, analyzed with whole-body in vivo imaging system (IVIS), included rapid accumulation in the intestine, where the EVs were detectable after 2 and 6 hours, followed by fluorescence signal observed in liver, spleen, lungs, kidney, heart, and the gastrointestinal tract at 24 hours. After 48 hours, the fluorescence signal subsided within most of the organs indicating the clearance of nanovesicles from the system (Samuel et al. (2021) Nat Commun 12:3950, doi.org/10.1038/s41467-021-24273-8). Thus, mammalian EVs (derived from sources other than milk) cannot be absorbed by the intestinal tract and from the intestines to become bioavailable in target organs (Zhong et al. (2021) Biomaterials. 277:121126. doi: 10.1016/j.biomaterials.2021.121126).

Treatments with mammalian cell-derived EVs generally employ intravenous or intraperitoneal routes of administration for systemic administration where the target organs are the liver, spleen and lungs. As noted, most mammalian EVs have not been employed for oral delivery due to their low stability at various pH and temperatures, rapid degradation of biomolecules in the digestive tract, and the limitations of industrial scale production for oral dosing (Cheng et al. (2019) Protein Cell 10 (4): 295-299). The only exception are bovine milk-derived EVs, which upon oral delivery to mice have shown a pattern of distribution that, analyzed with whole-body in vivo imaging system (IVIS), include rapid accumulation in the intestine, where the EVs were detectable after 2 and 6 hours, followed by fluorescence signal observed in liver, spleen, lungs, kidney, heart, and the gastrointestinal tract at 24-hour time point. After 48 hours, the fluorescence signal subsided within most of the organs indicating the clearance of nanovesicles from the system.

As shown herein in the Examples, and discussed below, MEVs have different properties from mammalian EVs. For example, they are stable in the harsh environment of the gastrointestinal tract compared to mammalian cell-derived EVs. Thus, the microalgae EVs, as described herein, are particularly suitable for oral administration and drug delivery, as well as other routes of delivery as described herein.

### 2. Microalgae EVs Biodistribution

It is shown herein that MEVs, including those provided herein from Chlorella, have properties that are distinct from mammalian EVs, including bovine milk EVs. For example, a striking difference, discussed below, is that the MEVs can be administered orally, and that the primary target is the spleen, likely the white pulp of the spleen (white spleen).

The MEVs provided herein can deliver a variety of bioactive molecules, such as RNAs, such as mRNA, siRNA, and miRNA; proteins; peptides; and small molecules, which can be exogenously or endogenously loaded. These include products such as tissue-specific products and/or disease specific products. As discussed below, each route can be used to target particular organs and treat particular diseases. The MEVs can be formulated for administration by each route. Thus, provided are compositions containing MEVs that are for treating particular disease and for particular routes of administration.

It is shown herein, the route of administration determines the fate of the MEVs, and that the ultimate location of the MEVs is a function of the route of administration. Targets and endpoints of the MEVs include, but are not limited to, the liver, spleen, lungs, the intestines, and brain. Routes of administration include, but are not limited to, respiratory (nose, lungs), oral (digestive), intravenous, central nervous system (CNS), and topical. The selection of route depends upon the ultimate target and the payload. It is shown herein that intranasal administration goes to the lungs, intratracheal via a spray goes to the lung(s), intravenous accumulates in the spleen and liver, oral (per Os) goes to the digestive tract and spleen. In contrast, mammalian EVs cannot be taken orally.

MEVs are readily internalized by human cells. For example, in vitro, when administered to cells in culture, such as A549 cells, at a ratio of MEV/cell of 1000/1, 93% of the cells internalized the MEVs, and this occurred within 24 to 48 hours after contacting the cells with the MEVs.

DIR-labeled MEVs were administered to mice via four routes: intranasal (IN), intratracheal (IT), intravenous (IV), and oral, and, by full-body imaging as a function of time, the fate of the MEVs was visualized for 3 days, followed by sacrificing the mice to harvest organs for study. As shown in the examples, intravenous administration targets the liver at about 4-12 hours following administration, and the spleen, appearing to be in the red pulp of the spleen (red spleen), at 10-30 hours. Oral administration targets the intestine and spleen. It is shown herein that the MEVs are orally available; they resist passage through the stomach, and reach the intestine at 0.5 hour to 4 hours, and then the spleen at 0.5 hour to 10 hours. Of interest is the route to the spleen; there are two possible routes to the spleen, via the blood (to red spleen), and via lymphocytes (to white spleen), which has implications for targeting and delivering cargo to the immune system, accumulating from 4 hours to 28 hours. This can be effected by internalization by lymphocytes that are activated and end up in the spleen where they multiply, and/or by lymphocytes that phagocytose the MEVs, which are not activated, and go to the white pulp of the spleen (white spleen) from where they are disseminated through the immune system.

### a. Oral Administration

Thus, orally ingested MEVs go into the intestine, then, as shown, end up in the spleen, likely the white spleen. The spleen is responsible for initiating immune reactions to blood-borne antigens, and for filtering foreign material and old or damaged red blood cells from the blood. These functions are performed by two different compartments in the spleen: the white spleen, and red spleen. The two compartments are vastly different in structure, vascular organization, and cellular composition (see, e.g., Cesta (2006) Toxicologic Pathology 34:455-465 for a review of the structure, function and histology of the spleen).

White blood cells, which are plentiful in the intestine, migrate to the white spleen. When ingested orally the MEVs can be internalized by intestinal cells and, as discussed below, including by intestinal lymphocytes, which carry the MEVs to the spleen. This is in contrast to mammalian vesicles, which cannot be administered orally. Thus, MEVs provide a delivery vehicle for agents for which the immune system is a target, such as for immune modulating cargo. As discussed above, the pathway to the white spleen can occur, for example, via activated lymphocytes and/or phagocytic lymphocytes. Lymphocytes can phagocytose the MEVs, and are homed to the spleen. The MEVs, unlike mammalian EVs, provide a way to orally deliver small molecule drugs and proteins and other therapeutics, such as nucleic acid therapeutics, that cannot be administered orally. In particular, orally administered MEVs provide a route for treatment of diseases, such as cancers and inflammatory diseases, in which the immune system is involved or in which the treatment can be effected by targeting the immune system. Such diseases include, but are not limited to, infectious disease, autoimmune diseases, cancers, prevention of organ transplant rejection. These diseases are treated by suppressing or augmenting the activity of immune cells.

### 1) Components of the Lymphatic System

The lymphatic system includes lymph, lymphatic vessels and lymphatic organs (see, discussion in Zgair et al., (2016) Targeting Immunomodulatory Agents to the Gut-Associated Lymphoid Tissue. In: Constantinescu C., Arsenescu R., Arsenescu V. (eds) Neuro-Immuno-Gastroenterology. Springer, Cham. (doi.org/10.1007/978-3-319-28609-9_14) and summarized below).

### Lymph

Lymph is a generally clear and colorless fluid that drains from the interstitium, and contains recovered fluids and plasma proteins, and also can contain lipids, immune cells, hormones, bacteria, viruses, cellular debris, and cancer cells.

### Lymphatic Vessels

The lymphatic system is the body's second circulatory system. The lymphatic system is a unidirectional, blind-ended and thin-walled system of capillary vessels where lymph is driven. Lymphatic capillaries drain in the afferent collecting vessels, which then pass through one or more gatherings of lymph nodes. Lymph fluid then passes through the efferent collecting vessels, larger trunks and then the lymphatic duct, which drain lymph to the systemic circulation. Primary lymphatic organs include the thymus gland and bone marrow, which produce mature lymphocytes, which identify and respond to antigens; secondary lymphatic organs include lymph nodes, spleen and mucosa-associated lymph tissues (MALT). Within the secondary lymphatic organs, lymphocytes initiate immune responses. MALTs are distributed throughout mucous membranes and provide a defensive mechanism against a wide variety of inhaled or ingested antigens. MALTs are categorized according to their anatomical location as: bronchus-associated lymphoid tissue (BALT), nasal-associated lymphoid tissue (NALT), salivary gland duct-associated lymphoid tissue (DALT), conjunctiva-associated lymphoid tissue (CALT), lacrimal duct-associated lymphoid tissue (LDALT) and gut-associated lymphoid tissue (GALT).

### Gut-Associated Lymphoid Tissue (GALT)

GALT is composed of effector and immune induction sites. Effector sites include lymphocytes distributed throughout the lamina propria (LP) and intestinal epithelium; induction sites involve tissues, such as mesenteric lymph nodes (MLN), PP and smaller isolated lymphoid follicles (ILF). Mesenteric lymph nodes (MLN), which occur in the base of the mesentery, are the largest gatherings of lymph nodes in the body. The structure of MLN is divided into two regions: the medulla and cortex. The cortex primarily is composed of T-cell areas and B-cell follicles. Within the T-cell area, circulating lymphocytes enter the lymph node, and dendritic cells (DC) present antigens to T-cells. Lymph (containing cells, antigens and chylomicrons) is collected from the intestinal mucosa and reaches the MLN via the afferent lymphatics. Lymph fluid subsequently leaves the MLN through efferent lymphatics to reach the thoracic duct that drains to the blood.

Peyer's patches (PP) are a collection of lymphoid nodules distributed in the mucosa and submucosa of the intestine. They contain a sub-epithelial dome area and B-cell follicles dispersed in a T-cell area. A single layer of epithelial cells, called follicle-associated epithelium (FAE), separates lymphoid areas of PP from the intestinal lumen. FAE is permeated by specialized enterocytes called microfold (M) cells. These cells are a gate for the transport of luminal antigens to PP.

Isolated lymphoid follicles (ILF) are a combination of lymphoid cells in the intestinal LP. ILF are composed of germinal centers covered by FAE containing M-cells. ILF is a complementary system to PP for the induction of intestinal immunity.

GALT is the largest lymphatic organ in the human body and contains more than half of the body's lymphocytes. GALT is exposed to more antigens in the form of commensal bacteria and alimentary antigens, in addition to those from invasive pathogens, than any other part of the body. Intestinal lymphatic transport avoids hepatic first-pass metabolic loss by diverting the absorption of lipophilic drugs towards intestinal lymphatics rather than the portal vein. The intestinal immune system must distinguish antigens that require a protective immune response and develop a state of immune hypo-responsiveness (oral tolerance) for harmless antigens. This is effected by sampling of luminal antigens in the intestinal epithelium by DC. Antigens can cross the epithelium through M-cells, which are specialized epithelial cells of the follicle-associated epithelium of the GI tract. The antigens interact with DC in the underlying sub-epithelial dome region. Antigens are presented to local T-cells in PP by DC.

DC also migrate to the draining MLN where they present antigens to local lymphocytes. Alternative pathways for antigen transport across the intestinal epithelial cells involve receptor-mediated transport, and direct sampling from the lumen by DC projections. Antigen-loaded DC then migrate to the MLN through afferent lymphatics where they present antigens to T-cells. Subsequently, differentiated lymphocytes migrate from MLN through the thoracic duct and blood stream and eventually accumulate in the mucosa for an appropriate immune response.

### 2) Targeting GALT

Orally administered MEVs can target gut-associated lymphoid tissue (GALT). Thus, GALT is a target (effective compartment) and/or a route through which MEVs and their therapeutic agent cargo can be used to deliver cargo to organs, tissues, and/or systemic circulation. GALT is an advantageous target for various pharmacological agents such as, for example, immunomodulators, chemotherapeutic agents, anti-infective agents. The lymphatic system is a main pathway for intestinal and other tumor metastases; therefore, targeting cytotoxic drugs to the intestinal lymphatics can be used to treat tumor metastases. GALT is a delivery target for antiviral agents, as some viruses, such as, for example, human immunodeficiency virus (HIV), morbillivirus, canine distemper virus, severe acute respiratory syndrome (SARS)-associated coronaviruses, hepatitis B and hepatitis C, spread and develop within the lymphatic system.

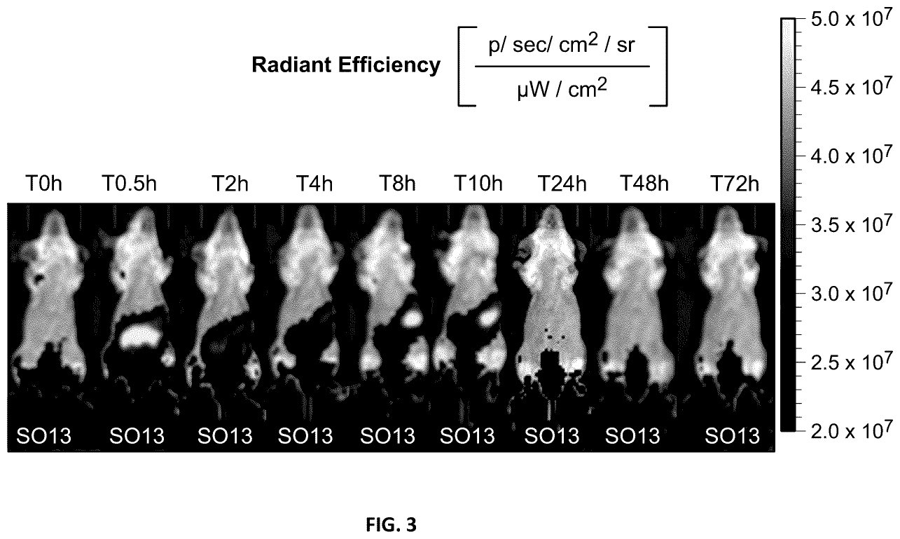

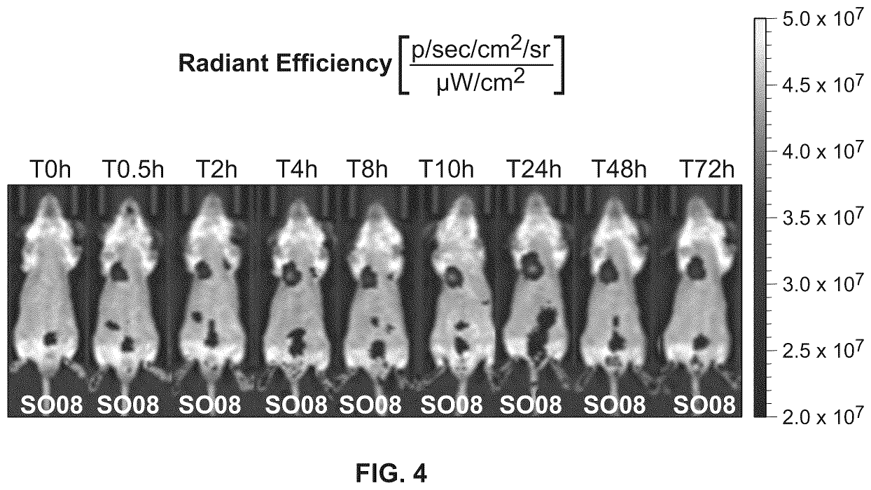

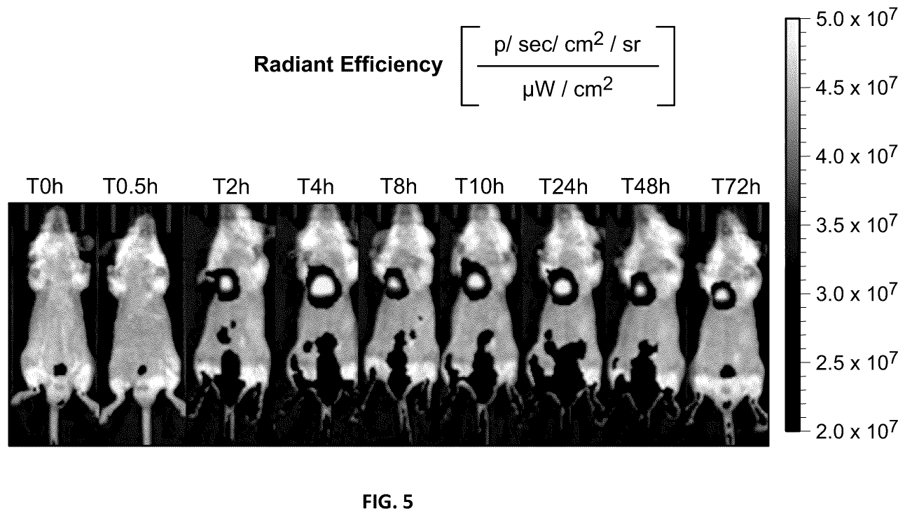

Thus, MEVs, including the Chlorella MEVs exemplified herein, can be used to target immune cells upon oral delivery. As described above, the microalgae MEVs show a distinct pattern of biodistribution when administered orally. This pattern includes initial intestine accumulation followed by targeting the spleen, where they are detectable up to 24 hours (see, e.g., FIG. 3).

Since the microalgae MEVs are delivered to the spleen, the mechanism of this delivery can be based on cells of the immune system. Immune cells are abundant in the single-cell layer of intestinal epithelium and underlying lamina propria of the gut-associated lymphoid tissue (GALT). The immune cells include, T cells, plasma cells, mast cells, dendritic cells, and macrophages (Luongo et al. (2009) Current perspectives. International Reviews of Immunology 28 (6): 446-464, doi.org/10.3109/08830180903236486). Macrophages, dendritic cells, neutrophils, and also B cells perform phagocytosis. The immune cells in the gut, thus, can phagocytose the MEVs to deliver them to the spleen. After phagocytosis, the fate of the MEV cargo can depend upon the type of cargo. For example, macrophage and dendritic cells participate in antigen presentation, and present proteins delivered in the MEVS, or the products in the MEVs can be secreted, or the products, such RNA, can be translated.

Immune cells present in the intestinal epithelium and lamina propria of the intestine migrate to the spleen and back to the intestine. This homing to the spleen can be involved in MEV transfer from the gut to secondary lymphatic organs, especially to the spleen. T cells exhibit a specific lymphocyte recirculation pathway (Mackay et al. (1990) J Exp Med 171:801-17) that can be part of MEV trafficking to the spleen upon oral delivery. Therefore, cells of the immune system are targeted by orally-administered MEVs, and this phenomenon contributes to MEV localization in the spleen within hours post-administration.

As shown herein, upon oral administration the MEVs go to the intestine and then migrate to the spleen. The route to the spleen can be via absorption into the blood and/or by internalization by immune cells in the intestine. The blood route is an unlikely route, because the MEVs then would appear in the liver as shown for intravenous administration. When MEVs are administered intravenously they primarily reach the liver (massively) and to a much lesser extent the spleen. It is shown herein that clearance of the MEVs from the spleen follows different kinetics depending upon their origin (oral or IV). The migration to the spleen following oral administration therefore uses a different pathway from the MEVs administered intravenously. When MEVs are administered by mouth, they reach the spleen after having passed through the intestine. These results indicate that the MEVs are located in “different compartments” inside the spleen, depending on the route of arrival: either from the intestine or from the blood. As discussed, upon oral administration, the likely route is that the MEVs in the intestine are internalized by lymphocytes present in the GALT, and that the subsequent migration of the MEVs from the intestine/GALT to the spleen occurs because the MEVs are transported by the lymphocytes. Coming from the intestine/GALT, the MEVs end up in the white spleen compartment. Thus, the MEVs provide a way to deliver cargo to different organs from mammalian EVs, which cannot be administered orally.

### 3. Diseases and Conditions Treated by MEVs

Based upon the targeted organs, a variety of diseases and disorders can be treated by MEVs. The MEVs can be loaded or produced to contain therapeutic agents for treating these diseases and conditions. The appropriate route of administration for the targeted organ and disease is selected. For example, for targeting the spleen and intestines, oral administration is selected; and for targeting the lungs, inhalation or nasal administration is selected. Based on the biodistribution and pharmacokinetic data the following organs can be targeted to treat diseases exemplified as follows. liver: cancer, cancer metastases, metabolic syndrome, genetic disorders (delivery of gene therapy), alpha-anti-trypsin (AAT) deficiency and other inborn errors of metabolism, hemophilia, hypercholesterolemia, liver inflammation, steatohepatitis, and other diseases and disorders that can be treated by delivery of a therapeutic to the liver;

spleen: diseases treated by immune modulation, including cancers, and immune cell disorders, and cancer, and other diseases that can be treated by administration to the spleen, particularly by immune cells that occur in or traffic to the white spleen;

intestine: diseases and disorders treated or prevented by vaccines, intestinal infections, microbiota modulation, Crohn's disease, cancer, ulcers, diseases treated by orally administered drugs, such as small molecules and proteins, and other such diseases, disorders, and conditions; and

lungs: infectious diseases, particularly respiratory diseases, chronic obstructive pulmonary disease (COPD), pulmonary hypertension, asthma, other inflammatory lung diseases, cystic fibrosis, ATT-deficiency, lung disease, cancer, cancer metastases, and other such diseases and disorders.

### G. Formulations, Routes of Administration, and Disease and Disorders

Provided are compositions containing the MEVs in an amount suitable for effecting treatment for a particular disease or disorder. The amount can depend upon the therapeutic cargo, the disease, or disorder, and the subject treated. It is within the level of skill in the art to ascertain a particular dosage of MEVs. Formulations include any known to those of skill and include, for example:
injectables for intravenous administration, to reach the liver and the spleen;
oral, such as, for example tablets, capsules, films, and troches;
drops for per os administration, to reach the intestine, such as a vaccine, the immune system (immune cells), and the spleen;
compositions, such as emulsions (microemulsions and nanoemulsions) for inhalation, such for intratracheal, intrapulmonary administration; to reach the lungs;
drops for intranasal administration; and
formulations, such as creams, oils, gels, lotions, ointments for the skin and the mucosa.

Provided are pharmaceutical compositions containing, in a pharmaceutically acceptable vehicle microalgae extracellular vesicles (MEVs). The MEVs can contain an agent, generally a therapeutic or biologically active agent, such as nucleic acid, particularly an RNA, a protein, a small molecule, and other such agents. The compositions contain an amount of the MEV that can be diluted to deliver a therapeutically effective amount of the agent, or are formulated for direct administration without dilution. The particular concentration of MEVs depends upon a variety of parameters within the skill of a skilled artisan, including, for example, the treated indication; the active agent; the route of administration; the disease, disorder, or condition to be treated; and the regimen. Routes of administration include systemic and local routes, oral, rectal, intravenous, intramuscular, subcutaneous, mucosal, inhalation, nasal, eye, peritoneal, intratracheal, intravitreal, vaginal, and any suitable route known to the skilled person.

Pharmaceutical carriers or vehicles suitable for administration of the compounds provided herein include any such carriers known to those skilled in the art to be suitable for the particular mode of administration.

### Exemplary Formulations

Pharmaceutical compositions containing the MEVS can be formulated in any conventional manner, by mixing a selected amount of the active compound with one or more physiologically acceptable carriers or excipients. Selection of the carrier or excipient is within the skill of the administering professional, and can depend upon a number of parameters. These include, for example, the mode of administration (i.e., systemic, oral, nasal, pulmonary, local, topical, or any other mode), and the disorder treated. The formulations also can be co-formulations with other active agents for combination therapy.

A selected amount of MEVs are formulated in a suitable vehicle for administration by a selected route. The pharmaceutical compositions can be formulated in any conventional manner, by mixing a selected amount of MEVs with one or more physiologically acceptable carriers or excipients or vehicles. The pharmaceutical composition can be used for therapeutic, prophylactic, cosmetic and/or diagnostic applications. The concentration of the MEVs in a composition, depends on a variety of factors, including those noted above, as well as the absorption, inactivation, and excretion rates of the active agent cargo, the release of the cargo, the mechanism of release, the dosage schedule, and the amount administered, the age and size of the subject, as well as other factors known to those of skill in the art, and related to the properties of the MEVs.

The pharmaceutical compositions provided herein can be in various forms, such as, but not limited to, in solid, semi-solid, liquid, emulsions, powder, aqueous, and lyophilized forms. The pharmaceutical compositions provided herein can be formulated for single dosage (direct) administration, or for dilution, or other regimen. The concentrations of the compounds in the formulations are effective, either following dilution or mixing with another composition, or for direct administration, for delivery of an amount, upon administration, that is effective for the intended treatment. The compositions can be formulated in an amount for single or multiple dosage direct administration. The form of composition depends on a variety of factors, including the intended mode of administration. The resulting mixtures are solutions, suspensions, emulsions and other such mixtures, and can be formulated as creams, gels, ointments, emulsions, solutions, lotions, suspensions, tinctures, pastes, foams, aerosols, irrigations, and sprays. For oral administration, the MEVs can be formulated as tablets, capsules, lozenges, liquids, and others.

For local internal administration, such as intramuscular, parenteral or intra-articular administration, the MEVs can be formulated in isotonically buffered saline. The effective concentration of the MEVs is sufficient to provide a sufficient amount of the cargo agent for the intended purpose, and can be empirically determined.

Generally, pharmaceutically acceptable compositions are prepared in view of approvals for a regulatory agency, or other agency, and/or are prepared in accordance with generally recognized pharmacopeia for use in animals and in humans. Pharmaceutical compositions can include a carrier, such as a diluent, adjuvant, excipient, or vehicle, with which a polypeptide is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain, along with an active ingredient, a diluent, such as lactose, sucrose, dicalcium phosphate, and carboxymethylcellulose; a lubricant, such as magnesium stearate, calcium stearate and talc; and a binder, such as starch, natural gums, such as gum acacia, gelatin, glucose, molasses, polyvinylpyrrolidone, celluloses and derivatives thereof, povidone, crospovidone, and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents. These compositions can take the form of solutions, suspensions, emulsions, tablets, pills, capsules, powders, granules, and sustained release formulations. Capsules and cartridges of, e.g., gelatin, for use in an inhaler or insufflator, can be formulated containing a powder mix of a therapeutic compound and a suitable powder base, such as lactose or starch. A composition can be formulated as a suppository, with traditional binders and carriers, such as triglycerides. Oral formulations can include standard carriers, such as pharmaceutical grades of mannitol, lactose, starch, magnesium stearate, sodium saccharine, cellulose, magnesium carbonate, and other such agents. Preparations for oral administration also can be suitably formulated with protease inhibitors, such as a Bowman-Birk inhibitor, a conjugated Bowman-Birk inhibitor, aprotinin and camostat. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier, so as to provide the compound in a form for proper administration to a subject or patient.

The pharmaceutical compositions provided herein can contain other additives, including, for example, antioxidants, preservatives, antimicrobial agents, analgesic agents, binders, disintegrants, colorings, diluents, excipients, extenders, glidants, solubilizers, stabilizers, tonicity agents, vehicles, viscosity agents, flavoring agents, emulsions, such as oil-in-water or water-in-oil emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20th Edition. Baltimore, MD: Lippincott Williams & Wilkins). Such carriers and/or additives can be formulated by conventional methods and can be administered to the subject at a suitable dose. Stabilizing agents, such as lipids, nuclease inhibitors, polymers, and chelating agents, can preserve the compositions from degradation within the body.

The formulation should suit the mode of administration. For example, the MEVs can be formulated for parenteral administration by injection (e.g., by bolus injection, or continuous infusion). The injectable compositions can take such forms as suspensions, solutions or emulsions in oily or aqueous vehicles. The sterile injectable preparation also can be a sterile injectable solution, or a suspension in a non-toxic parenterally-acceptable diluent or solvent, for example, as a solution in 1,4-butanediol. Sterile, fixed oils are conventionally employed as a solvent or suspending medium. For this purpose, any bland fixed oil can be employed, including, but not limited to, synthetic mono- or diglycerides, fatty acids (including oleic acid), naturally occurring vegetable oils, such as sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation. The MEVs provided herein, can be formulated as the sole pharmaceutically active ingredient in the composition, or can be combined with other active ingredients. Suspension of the MEVs can be suitable for administration. These can be prepared according to methods known to those skilled in the art.

Suitable compositions for intranasal administration include but are not limited to, powders, sprays, liquids, suspensions, emulsions, and any other form that can be administered directly to the nose and that can contain the MEVs. The concentration of MEVs can be empirically determined, and depends upon the cargo, the indication treated, or intended use.

The therapeutical concentration of the MEVs can be determined empirically by testing the compounds in known in vitro and in vivo systems. Determination of a therapeutically effective amount is well within the capability of those skilled in the art.

### H. Biodistribution and Delivery of Mevs to the Brain Via Intranasal (in) Administration for Treating Diseases, Disorders, and Conditions of the Brain and Cns

As discussed throughout the disclosure herein, MEVs provide numerous advantages for delivery of bioactive molecules, including therapeutic and diagnostic or detectable molecules, compared to other vehicles, including EVs from other sources, including plant sources (see discussion in the section below, and throughout the disclosure). In particular, as described and shown herein, the MEVs can be administered intranasally, and traffic via unique pathways to areas of interest in the brain. The MEVs can be loaded with cargo that includes nucleic acids, such as plasmids, anti-sense oligonucleotides (ASO), mRNA, lncRNA, siRNA, miRNA, and other RNAs, peptides including proteins/peptides/polypeptides, small molecules, drugs, diagnostic agents, and other molecules. MEVs can serve as vectors to the brain of pharmacologically active compounds with poor stability in gastrointestinal fluids, poor intestinal absorption and/or extensive hepatic first-pass elimination, such as polar drugs.

Administration of MEVs by IN administration provides for transporting drugs that act in the brain, such as, for example, anti-depressants, antipsychotics, anxiolytics, memory enhancers, agents for treatment of dementia, and agents for treatment of cancers, to target cells where their effects are manifested, without general distribution in the body that can occur by systemic, such as intravenous, or by oral administration. MEVs provide a non-invasive solution for delivering drugs targeted for CNS and brain diseases, disorders, and conditions. MEVs can deliver molecules that are unable to cross the blood-brain barrier (BBB). For hydrophilic compounds, access to the brain is restricted by the BBB which does not allow the transfer from the vascular compartment to the brain tissue. IN administration also can deliver molecules that are not able to reach the brain after first-pass metabolism.

### 1. Brain Structure

In view of the findings described and exemplified herein regarding biodistribution of MEVs in the brain (see, e.g., the working examples and accompanying figures) the following brain areas/nuclei are of interest in view of their recognized involvement in brain diseases, disorders, and conditions, including psychiatric and neurologic disorders, and major functions of the central nervous system.

### a. Anterior Olfactory Nucleus

The anterior olfactory nucleus refers to the most rostral group of nerve cells that receive input from the olfactory bulb. In the human and the macaque they form small groups scattered within the olfactory tract from the olfactory bulb through the olfactory peduncle to a much larger group on the dorsal surface of the tract on the underside of the orbital gyri. It is composed of several subgroups, which are defined by topology.

The anterior olfactory nucleus is located posterior to the olfactory bulb in the olfactory peduncle. It is one of the major olfactory processing centers; the olfactory bulb is its major afferent input and also is the principal target of its axons. The anterior olfactory nucleus (AON) is the initial recipient of odor information from the olfactory bulb, and the target of dense innervation conveying spatiotemporal cues from the hippocampus. Episodic and contextually-relevant odor engrams are stored within the AON; its activity is necessary and sufficient for the behavioral expression of odor memory.

### b. Tenia Tecta

The tenia tecta refers to a continuation ventrally of the supracallosal gyrus beyond the rostrum of the corpus callosum (see, e.g.,

URL:/braininfo.rprc.washington.edu/centraldirectory.aspx?ID=1870). In the human and the macaque it lies on the rostral surface of the lamina terminalis and is considered identical to or part of the paraterminal gyrus. In the rat and the mouse it is located similarly in relation to the supracallosal gyrus; it is a more prominent layered structure that extends rostrally on the medial surface overlying the anterior olfactory nucleus. It is considered part of the olfactory areas (rodent) of the cerebral cortex. In rodents it consists of two parts, the dorsal tenia tecta and the ventral tenia tecta. The ventral tenia tecta (vTT) is a component of the olfactory cortex and receives both bottom-up odor signals and top-down signals. Tenia tecta (vTT), an area of the olfactory cortex located in the ventromedial aspect of the olfactory peduncle, transforms the perception of odor signals into reward-directed behaviors.

### c. Olfactory Tubercle

The olfactory tubercle refers to a predominantly cellular structure defined on the basis of a Nissl stain. It is located on the ventral surface of the endbrain caudal to the anterior olfactory nucleus, medial to the olfactory tract, rostral to the piriform area and ventral to the nucleus accumbens and substantia innominata. It contains some of the islands of Calleja.

In humans the olfactory tubercle is not very developed; it is barely distinguishable from the overlying nucleus accumbens. In the macaque it is somewhat more prominent and bounded medially by the tenia tecta. In primates it does not protrude from surrounding areas and is penetrated by numerous small blood vessels. These give it the appearance on dissection that accounts for the name ‘anterior perforated substance’ in human neuroanatomy. The location in the rat and the mouse is the same as in the macaque. It protrudes on the rostroventral surface of the endbrain where it is more clearly stratified and much larger in proportion to the size of the brain than in primates. It is involved in the proper sense of smell.

### d. Piriform Cortex

Primary olfactory cortex or Piriform Cortex is located in the temporal lobe. The piriform cortex (PC) is a key brain area involved in processing and coding of olfactory information. It is implicated in various brain disorders, such as epilepsy, Alzheimer's disease, and autism. The PC consists of the anterior (APC) and posterior (PPC) parts, which are different anatomically and functionally.

The piriform cortex (PC) is located in the ventrolateral region of the forebrain and extends broadly along the anterior to posterior (AP) axis in mammals. As one of the primary olfactory cortex, the PC is involved in encoding odor identification (Gottfried et al. (2006) Neuron 49:467-479; Howard et al. (2009) Nature Neuroscience 12:932-938; Wilson et al. (2011) Neuron 72:506-519; Bekkers et al. (2013) Trends in Neuroscience 36:429-438; Courtiol et al. (2017) Perception 46 (3-4): 320-332, doi.org/10.1177/0301006616663216), odor-associated values or contexts (Gottfried et al. (2003) Neuron 39:375-386; Calu et al. (2007) In: Cerebral cortex (New York, N.Y.: 1991) 17:1342-1349, Roesch et al. (2007) In: Cerebral cortex (New York, N.Y.: 1991) 17:643-652), and odor memory (Zelano et al. (2011) Neuron 72:178-187; Strauch et al. (2018) Cerebral Cortex 28:764-776).

The PC also is implicated in various neurological disorders, such as epilepsy (Loscher et al. (1996) Progress in Neurobiology 50:427-481; Vismer et al. (May 2015) Front. Neural Circuits, 29, doi.org/10.3389/fncir.2015.00027; Young et al. (2019) Experimental Neurology 320:113013), Alzheimer's disease (Samudralwar et al. (1995) Journal of the Neurological Sciences 130:139-145; Saiz-Sanchez et al. (2015) Brain Struct Funct 220:2011-2025.,doi.org/10.1007/s00429-014-0771-3), autism spectrum disorder (Menassa et al. (2018) Neuroscience Letters 665:86-91; Koehler et al. (2018) Chemical Senses 43:627-634), and Parkinson's disease (Wu et al. (2011) Human Brain Mapping 32:1443-1457).

### e. Amygdala

Social behaviors are disrupted in several psychiatric disorders. The amygdala is a key brain region involved in social behaviors, and amygdala pathology has been implicated in disease states ranging from social anxiety disorder to autism. Frequently implicated in psychotic spectrum disorders, the amygdala serves as a hub for elucidating the convergent and divergent neural substrates in schizophrenia and bipolar disorder, the two most studied groups of psychotic spectrum conditions.

### f. Entorhinal Cortex

Entorhinal cortex (EC) relays object-related and spatial information from the perirhinal and parahippocampal cortices (PRC, PHC) to the hippocampus (HC). The entorhinal cortex projects weakly to the basal nucleus. Efferent fibers from the entorhinal cortex pass through the lateral nucleus, but it is not clear if the fibers form synapses or terminal plexuses within the nucleus. The projection from the amygdala to the entorhinal cortex arises primarily from the lateral nucleus and is most robust passing to anterior portions of the cortex. Unlike its projections to other areas of cortex, the basal nucleus contributes only a weak projection to entorhinal cortex. Two-thirds of all cortical projections to the hippocampus are relayed through the entorhinal cortex. It is not known if the entorhinal cortex provides the same information to the amygdala as it does to the hippocampus.

The entorhinal cortex is located in the mesial temporal lobe and acts as the interface between the hippocampus and the neocortex. It has been considered part of the hippocampal formation. It occupies the middle portion of the medial temporal region and includes part of the parahippocampal gyrus and gyrus ambiens 2. It is increasingly defined by its connectivity to the hippocampus.

### g. Frontal Cortex

The frontal cortex (FC) is the cerebral cortex covering the front part of the frontal lobe. This brain region is implicated in planning complex cognitive behavior, personality expression, decision making, and moderating social behavior. The basic activity of this brain region is orchestration of thoughts and actions in accordance with internal goals. Functions carried out by the frontal cortex area are referred to as executive functions.

### h. Striatum: Caudate Nucleus and Putamen

Two subcortical nuclei within the basal ganglia, the bilateral caudate nucleus and bilateral putamen, form the striatum. The caudate nucleus primarily is involved with emotion regulation, reward processing, decision making and executive functioning, while the putamen is primarily associated with the planning and production and purification, from the regulatory perspective, independent dossiers implementation of motor functions. Because of the strategic location and connectivity of the caudate nuclei and the putamen within frontostriatal circuits, morphological changes to these nuclei have been linked to the clinical functioning of patients with Parkinson's disease

### i. Nucleus Accumbens

Nucleus accumbens is considered as a neural interface between motivation and action, having a key-role in food intake, sexual behavior, reward-motivated behavior, stress-related behavior and substance-dependence. It is involved in several cognitive, emotional and psychomotor functions, altered in some psychopathology. Moreover it is involved in some of the most common and most severe psychiatric disorders, such as depression, schizophrenia, obsessive-compulsive disorder and other anxiety disorders, as well as in addiction, including drug abuse, alcoholism and smoking. Nucleus accumbens has also a role in other psychiatric disorders such as bipolar disorder, attention deficit/hyperactivity disorder and post-traumatic stress disorder. Nucleus accumbens deep brain stimulation has been also associated with antidepressant and anxiolytic effect, as well as quality of life improvement in patients suffering from severe resistant depression. Finally, nucleus accumbens deep brain stimulation has been proved beneficial for all phenotypic components of the Tourette syndrome, with remarkable reduction of the syndrome's motor manifestations, including tics.

### j. Thalamus

The thalamus is concerned in the higher nervous functions such as language, cognition, memory and intelligence. Severe nerve cell loss with proliferation of hypertrophic astroglia is observed in the association nuclei and sensory relay nuclei in the thalami of patients suffering from Creutzfeldt-Jakob disease. In a brain imaging study, volume reduction of the thalamus, especially of dorsomedial nuclei, and degradation of glucose metabolism were observed in the thalami of patients with schizophrenia. Schizophrenia has been considered to be a subcortical neurotransmitter imbalance syndrome. Schizophrenia has been described as a misconnection syndrome or cognitive dysmetria induced by dysfunction of the cortico-cerebellar-thalamic-cortical circuit (CCTCC).

### k. Hypothalamus

The hypothalamus is responsible for the control of important and vital functions by the release of several hormones such as CRH (corticotropin-releasing hormone), TRH (thyrotropin-releasing hormone), GnRH (gonadotropin-releasing hormone or luteinizing-releasing hormone, oxytocin, vasopressin, somatostatin (growth hormone-inhibiting hormone, GHIH), and GHRH (growth hormone-releasing hormone), which are responsible among others for the control of body temperature regulation, maintaining daily physiological cycles, controlling appetite, managing sexual behavior and regulating emotional responses.

### I. Substantia Nigra Pars Compacta

Pathologically, Parkinson's disease is characterized by the loss of dopaminergic neurons in the pars compacta of the substantia nigra.

### m. Hippocampus

The hippocampus has a pivotal role in learning and in the formation and consolidation of memory and is critically involved in the regulation of emotion, fear, anxiety, and stress. Studies of the hippocampus have been central to the study of memory in humans. The hippocampus is a model for the study of neuroplasticity as many examples of synaptic plasticity such as long-term potentiation and depression have been identified and demonstrated in hippocampal circuits.

### n. Colliculus

The extensive connections of the superior colliculus make it a major center for initiating eye movements and coordinating them with movements of the head and neck. The superficial layers of the superior colliculus contain a retinotopic map of the environment and the deeper layers contain premotor neurons with connections to networks that generate saccades and head movements. The auditory, somatosensory and visual signals that converge on the superior colliculus move the eyes, head and body to direct the line of sight towards objects of interest for orienting behavior.

### o. Pontine Raphe Nuclei

Raphe nuclei are characterized by high content in serotonin (5HT). They are responsible for the release of 5HT to other parts of the brain. Selective serotonin reuptake inhibitor (SSRI) drugs, for example, are thought to act on the raphe nucleus for achieving their antidepressant action.

### 2. The Blood-Brain Barrier

Drug development for central nervous system (CNS) diseases and psychiatric disorders is challenging due to the side effects of drugs, the complexity of the brain, and notably, the lack of efficient strategies to deliver drugs across the blood-brain barrier (BBB). The blood-brain barrier (BBB) restricts drug access to the brain, limiting the lipophilic drugs. In this context, development of molecules for delivery to the brain is challenging because of: (1) the design of active molecules, according to structure-activity rules discovered from 3D models, crystal structure of the target, in agreement with Lipinski's rule of five (Lipinski (2004) Drug Discovery Today: Technologies 1 (4): 337-34), which describes molecular properties important for a drug's pharmacokinetics in the human body; and (2) the specific constraints imposed by the BBB, which requires optimization of the permeation of molecules across the BBB, which in turn depends on molecular weight, lipophilicity, H bond donors and acceptors, charge, and polar surface area.

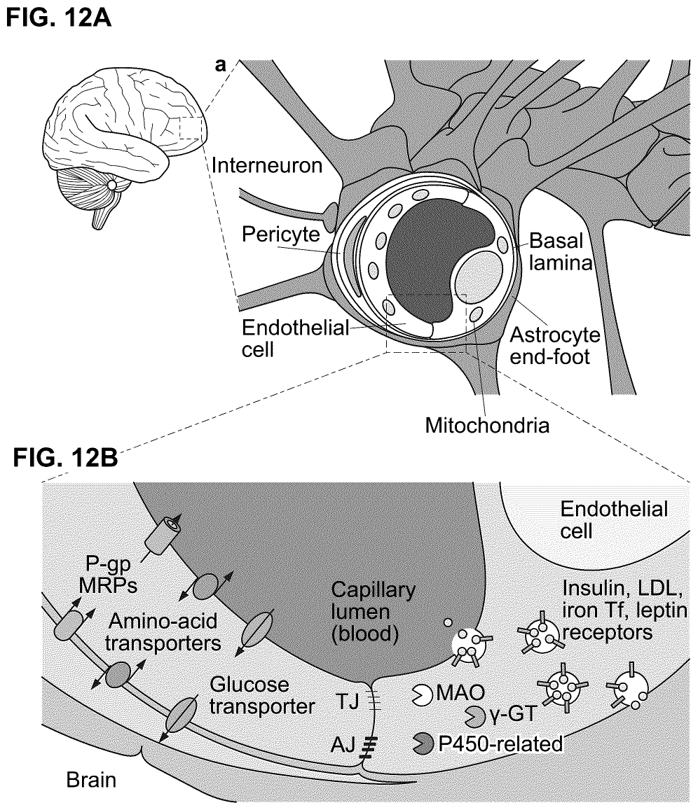

These problems can be solved by IN delivery of the MEVs, which bypass or are not restricted by the BBB. The blood-brain barrier (BBB) is formed by endothelial cells at the level of the cerebral capillaries. These endothelial cells interact with perivascular elements such as basal lamina and closely associated astrocytic end-feet processes, perivascular neurons (represented by an interneuron in FIG. 12A) and pericytes to form a functional BBB. Cerebral endothelial cells are unique in that they form complex tight junctions (TJ) produced by the interaction of several transmembrane proteins that effectively seal the paracellular pathway (FIG. 12B). These complex molecular junctions make the brain practically inaccessible for polar molecules, unless they are transferred by transport pathways of the BBB that regulate the microenvironment of the brain. There also are adherens junctions (AJ), which stabilize cell-cell interactions in the junctional zone. In addition, the presence of intracellular and extracellular enzymes such as monoamine oxidase (MAO), Y-glutamyl transpeptidase (Y-GT), alkaline phosphatase, peptidases, nucleotidases and several cytochrome P450 enzymes endow this dynamic interface with metabolic activity. Large molecules such as antibodies, lipoproteins, proteins and peptides can be transferred to the central compartment by receptor-mediated transcytosis or non-specific adsorptive-mediated transcytosis. Included are proteins, are receptors for insulin, low-density lipoprotein (LDL), iron transferrin (Tf), and leptin, that are involved in transcytosis. Others include, for example the multidrug resistance-associated protein family, such as P-glycoprotein.

Soluble molecules can cross the BBB via different mechanisms. Several lipid-soluble molecules can enter the brain by passive diffusion. In this mechanism, the molecule lipophilicity generally defines the penetration rate and extent into the brain. Many of these molecules are usually pumped back to the circulatory system by some efflux pumps expressed in the BBB. Small polar molecules, such as amino acids, glucose, nucleosides, and organic anions and cations, are transported by carrier-mediated transport. Another mechanism is receptor-mediated transcytosis, which transports large molecules, such as iron Tf, insulin, and leptin. Similar to Lipinski's rule of five, the permeation of a molecule across the BBB depends on its molecular weight, lipophilicity, H bond donors and acceptors, charge, and polar surface area. Thus, only a small number of hydrophobic and low molecular weight molecules can cross the BBB, whereas others are restricted by the barrier characteristics of the BBB, which makes it difficult to develop drugs that target the brain.

### 3. Brain and Target Cells

The central nervous system (which includes the brain and spinal cord) is composed primarily of two cell types: neurons, and glial cells. Glial cells come in several types, and perform a number of critical functions, including structural support, metabolic support, insulation, and guidance of development. Both glial cells and neurons can be a target for MEVs, as delivery of a therapeutic cargo to either type of cells is of clinical relevance. Dysfunction in glial cells associates with a variety of brain diseases such as Alzheimer's disease, Parkinson's disease, multiple sclerosis, glioblastoma, autism and psychiatric disorders. Neuronal degeneration, on the other hand, is also involved in Alzheimer's disease and Parkinson's disease, as well as ischemic stroke and a number of genetic neurodegenerative conditions, including amyotrophic lateral sclerosis and Huntington's disease. Both cell types may undergo malignant transformation, resulting in brain tumors such as astrocytoma, glioblastoma and medulloblastoma.

### 4. Differences Between Biodistribution of MEVs and Other Delivery Vehicles

MEVs are different from any other kind EV and nanoparticle. Mammalian EVs are a very heterogenous group of EVs because they arise from different cells, tissues, and organs, such as from stem cells, dendritic cells, tumor cells, and other sources. There is no single mammalian EV; each has different properties and must be separately developed. They share some common phenotypic markers, but they are structurally different, carry different payloads in vivo, and, originate from different cell types. From an industrial/pharmaceutical perspective, each kind will require a more or less adapted process and must be constituted for each kind of mammalian EV, and they are not necessarily routine or easy to produce.

MEVs, as shown herein, are uniform in composition. For exogenously-loaded MEVs, a single and common process for production and purification can result in a myriad of products using the same MEVs, which when exogenously loaded, can carry different payloads, such as small molecules, RNA products, proteins, peptides, polypeptides, and others. The MEVs are uniform in structure and contents. From a regulatory perspective, as the outside of the MEV is the same (irrespective from the payload inside) the MEVs will share the same toxicities, if any, or lack thereof. Formulations can be developed for each route of delivery independent of cargo (payload). Endogenously-loaded MEVs will be similarly uniform in structure.

It is shown and described herein, that the MEVs traverse unique pathways, depending on the route of administration. For example, as discussed elsewhere herein, MEVs provide unique routes of delivery via oral administration compared to EVs from other sources, nanoparticles, and viruses. With respect to intranasal administration, prior art has established, for example that:
1) the IN route of administration is a suitable way to access the brain and to deliver drugs and biologicals;
2) Extracellular vesicles from mammalian origin have been reported to deliver payloads to the brain (including proteins, siRNA, miRNA, mRNA) by IN and other routes of administration. See, e.g., ncbi.nlm.nih.gov/pmc/articles/PMC6202788/, ncbi.nlm.nih.gov/pmc/articles/PMC7409518/, ncbi.nlm.nih.gov/pmc/articles/PMC8363003/;
3) Synthetic nanoparticles (also referred to as nanovectors) made of synthetic molecules/lipids plus lipids extracted from grapefruit exosome-like nanoparticles, have been reported to deliver siRNA to the brain by IN administration. These synthetic nanovectors are reported to reach the olfactory bulb, the hippocampus, the thalamus, the cerebellum, the cerebral cortex, and the striatum.
4) Synthetic nanoparticles made of polycaprolactone (polycaprolactone nanoparticles (PCL NPs) and PEG-modified PCL NPs have been reported to deliver curcumin (small molecule) to the brain by IN administration. These particles are reported to enter the brain via the trigeminal nerve; they cannot get through the olfactory nerve.
5) Infectious viruses as well as endogenous intracellular vesicles have been reported to travel within the brain via axonal transportation.
6) The passage of viruses and intracellular vesicles over the synapses has been demonstrated.

The following Table provides a summary of prior art extracellular vesicles (EV) from other sources, including mammalian EVs, and other nanoparticles, and their pathways upon administration, to contrast with the results using MEVs as shown and described herein. It is apparent from the table that MEVs, when administered, behave differently from EVs from other sources, and differently from other vehicles, such as lipid nanoparticles. This is particularly apparent when administration is via intranasal administration (and as discussed elsewhere herein via oral administration). It is apparent from the table and description and results presented here that MEVs follow unique pathways upon administration. MEVs behave differently from EVs from other sources. Because of the pathways followed by MEVs they can provide for targeted delivery to brain as well as other organs.

| | | Route of | | | Pathways | |
|---|---|---|---|---|---|---|
| | Type of | administration | Entry site as | Travel | (brain regions | |
| # | nanoparticle | to the brain | reported | route | targeted/reached) | Reference |
| 1 | EVs from | intranasal | Primary | Extraneuronal | Rostral brain | Zhuang et |
| | cultured | | location in the | pathway | regions | al., Mol |
| | mammalian | | olfactory bulb | | | Ther. |
| | cells | | | | | 19(10): |
| | | | | | | 1769-1779 |
| | | | | | | (2011). |
| 2 | EVs from | Intravenous, | N/A | BBB crossing | Neurons, | Alvarez- |
| | GM | systemic | | | microglia, | Erviti et |
| | mammalian | | | | oligodendrocytes | al., ″ Nat. |
| | cells | | | | in the brain | Biotechnol. |
| | | | | | | 29(4): |
| | | | | | | 341-5 (2011). |
| 3 | grapefruit- | intranasal | Not specified | Not specified | “the majority of | Wang et |
| | derived | | | | the GNVs were | al., Nat. |
| | nanovectors | | | | located in the | Commun. |
| | (GNVs) | | | | lung and brain” | 4: 1867 |
| | | | | | | (2013). |
| 4 | enzyme- | intranasal | Transport | Route not | Predominantly | Haney et |
| | loaded | | along the | specified. | cerebral frontal | al., J. |
| | mammalian | | olfactory | Hypothesis: | cortex, central | Control |
| | EVs | | nerve cells is | tetraspanins | sulcus, and | Release |
| | | | indicated | and integrins | cerebellum | 207: 18-30 |
| | | | | on exosomes | | (2015). |
| | | | | are able to | | |
| | | | | attachment to | | |
| | | | | the plasma | | |
| | | | | membranes | | |
| | | | | of target cells | | |
| 5 | grapefruit- | intranasal | by olfactory | Route not | Primary | Zhuang et |
| | derived | | bulb | specified, | locations in the | al., Mol. |
| | nanovectors | | | Hypothesis: | olfactory bulb, | Ther. |
| | (GNVs) | | | extraneuronal | hippocampus, | 24(1): |
| | | | | pathway | thalamus, and | 96-105 |
| | | | | because rapid | cerebellum | (2016). |
| | | | | movement of | | |
| | | | | GNVs into | | |
| | | | | the brain | | |
| | | | | within 1.5 | | |
| | | | | hour of IN | | |
| | | | | administration | | |
| 6 | gold | intranasal, | by olfactory | Route not | Widespread | Betzer et |
| | nanoparticle- | intravenous | bulb | specified. | biodistribution | al., ACS |
| | labeled EVs | | | Hypothesis: | in normal brain; | Nano. |
| | from | | | extraneuronal | accumulation in | 11(11): |
| | mammalian | | | pathway; | ischemic-like | 10883-10893 |
| | cells | | | transcytosis | damage region | (2017). |
| | | | | across blood | | |
| | | | | brain barrier | | |
| | | | | also indicated | | |
| 7 | solid lipid | Intraperitoneal, | Not specified, | IN route: | IN: nasal | Esposito et |
| | nanoparticles | intranasal | Hypothesis: | SLN reaches | region, liver, | al., Eur J |
| | (SLN) | | olfactory bulb | CNS through | spleen and | Pharm |
| | | | for IN | 1) direct | kidneys; some | Biopharm. |
| | | | administration | olfactory | in the brain | 115: |
| | | | BBB after IP | transport, | | 285-296 |
| | | | administration | along the | | (2017). |
| | | | | olfactory and | | |
| | | | | trigeminal | | |
| | | | | nerves | | |
| | | | | pathway or 2) | | |
| | | | | systemic | | |
| | | | | circulation | | |
| | | | | pathway via | | |
| | | | | the transmucosal | | |
| | | | | absorption | | |
| | | | | IP route: | | |
| | | | | hepatic portal | | |
| | | | | system and BBB | | |
| 8 | modified | intravenous | N/A | BBB crossing | Ischemic lesion | Tian et al., |
| | EVs from | | | | region, entering | Biomaterials |
| | mammalian | | | | microglia, | 150: |
| | cells | | | | neurons and | 137-149 |
| | | | | | astrocytes | (2018). |
| 9 | grapefruit- | intranasal | by olfactory | Route not | Primary | US |
| | derived | | bulb | specified | locations in the | application |
| | nanovectors | | | Hypothesis: | olfactory bulb, | publication |
| | (GNVs) | | | extraneuronal | hippocampus, | 20180362974 |
| | | | | pathway | thalamus, and | |
| | | | | because the | cerebellum | |
| | | | | authors found | | |
| | | | | rapid movement | | |
| | | | | of GNVs into | | |
| | | | | the brain | | |
| | | | | within 1.5 | | |
| | | | | hour of IN | | |
| | | | | administration | | |
| 10 | EVs from | intranasal | Not specified | EVs are taken | Microglia at 4 | Hu et al., |
| | stimulated | | | up by | hrs: primarily in | Mol. Ther. |
| | mammalian | | | microglial | the lungs, brain, | Nucleic |
| | cells | | | cells | gut, liver, and | Acids. |
| | | | | | heart; at 24 hrs: | 13: |
| | | | | | in the brain and | 450-463 |
| | | | | | heart, but | (2018). |
| | | | | | absent in the | |
| | | | | | lungs and the | |
| | | | | | gut | |
| 11 | drug-loaded | intranasal | Olfactory | Nasal mucosa | High | Natsheh et |
| | artificial | | region | membrane | accumulation in | al., Drug |
| | phospholipid- | | | indicated | the cerebrum | Deliv. |
| | based | | | from in vitro | and also in the | Transl. |
| | vesicles | | | experiments. | olfactory bulb | Res. |
| | | | | Hypothesis: | (within 10 | 8(3): |
| | | | | systemic | minutes) | 806-819 |
| | | | | absorption | | (2018). |
| | | | | in vivo | | |
| 12 | gold | intranasal | Migration of | BBB crossing | in brain and | Guo et al., |
| | nanoparticle- | | EVs to the | | olfactory bulbs | ACS Nano. |
| | labeled EVs | | spinal cord is | | in healthy | 13(9): |
| | from human | | by chemotaxis, | | animals, spinal | 10015-10028 |
| | stem cells | | but entry of | | cord lesions in a | (2019). |
| | | | EVs not | | model of injury | |
| | | | specified | | | |
| 13 | gold | intranasal | Migration of | BBB crossing | brain and | WO2019186558A1 |
| | nanoparticle- | | EVs to the | | olfactory bulbs | |
| | labeled EVs | | spinal cord is | | in healthy | |
| | from human | | by chemotaxis, | | animals, spinal | |
| | stem cells | | but entry of | | cord lesions in a | |
| | | | EVs not | | model of injury | |
| | | | specified | | | |
| 14 | enzyme- | intrathecal, | Not specified | Not specified | hypothalamus, | Haney et |
| | loaded | intranasal, | | | thalamus, pons, | al., Cells. |
| | mammalian | intravenous, | | | spinal cord | 9(5): 1273 |
| | EVs | intraperitoneal | | | little, if any, | (2020). |
| | | | | | DiR signal | |
| | | | | | after IN | |
| | | | | | administration | |
| | | | | | (over 6-480 hrs) | |
| 15 | EVs from | Intra- | Not specified. | Extracellular | Various | Ioannides |
| | human stem | hippocampal | Depends on | space or | subregions of | et al. J. |
| | cells | transplantation, | route of | circulation | the brain at | Cancer |
| | | retro-orbital | administration: | | roughly | Metastasis |
| | | vein injection, | i.h.-Migrates | | equivalent | Treat |
| | | intranasal | through CA1 | | levels | 6(15): |
| | | | stratum | | | 10.20517/2394- |
| | | | radiatum, r.o.- | | | 4722 |
| | | | Trigeminal | | | (2020). |
| | | | nerve, i.n.- | | | |
| | | | Olfactory | | | |
| | | | bulb/trigeminal | | | |
| | | | nerve. | | | |
| 16 | EVs from | intranasal | Not specified, | Not detailed, | CA1 region of | Losurdo et |
| | stimulated | | likely | but rapid | medial | al. ″ Stem |
| | human stem | | olfactory and | detection in | hippocampus | Cells |
| | cells | | trigeminal | brain | (EVs robustly | Transl |
| | | | nerve | indicates | incorporated | Med. 9(9): |
| | | | pathways. | extraneuronal | into CA1 | 1068-1084 |
| | | | | pathway; | microglia and, | (2020). |
| | | | | transcytosis | to some extent, | |
| | | | | across blood | into neurons, | |
| | | | | brain barrier | but not in | |
| | | | | | astrocytes) | |
| 17 | EVs from | intranasal | Not specified, | Not specified, | EVs did not | Tsivion- |
| | human stem | | likely | likely BBB | accumulated in | Visbord et |
| | cells | | olfactory and | crossing | the brain over a | al. Transl. |
| | | | trigeminal | | 24 h in healthy | Psychiatry |
| | | | nerve | | mice; | 10(1): 305 |
| | | | pathways. | | accumulation in | doi: 10.1038 |
| | | | | | the prefrontal | (2020). |
| | | | | | cortex in a | |
| | | | | | model of | |
| | | | | | schizophrenia | |
| 18 | RNA-loaded | intranasal | Not specified. | Not detailed. | Microglia; at 4 | Chivero et |
| | EVs from | | olfactory and | The rapid | hrs labelled | al. Front |
| | cultured | | trigeminal | detection in | RNA cargo is | Cell Dev |
| | mammalian | | nerve | brain | detectable in | Bio. 8: 573 |
| | cells | | pathways. | indicates | the brain. | doi: 10.3389 |
| | | | | extraneuronal | | (2020). |
| | | | | pathway; | | |
| | | | | transcytosis | | |
| | | | | across blood | | |
| | | | | brain barrier | | |
| 19 | drug-loaded | intranasal | Not specified. | BBB crossing | Brain | Touitou et |
| | artificial | | Hypothesis: | | accumulation | al. Int. J. |
| | multilamellar | | nasal mucosa, | | confirmed, by | Pharm. |
| | vesicles | | likely | | functional | 580: 119243 |
| | | | olfactory and | | assays | (2020). |
| | | | trigeminal | | | |
| | | | pathways. | | | |
| 20 | enzyme- | intranasal | Transport | BBB crossing | Predominantly | US patent |
| | loaded | | along the | | cerebral frontal | application |
| | mammalian | | olfactory | | cortex, central | pub |
| | EVs | | nerve cells is | | sulcus, and | 20200297631 A1 |
| | | | indicated | | cerebellum | |
| 21 | EVs from | intranasal | Primary | Extraneuronal | Intranasal | U.S. Pat. No. |
| | cultured | | location in the | pathway | delivery | 10,799,457 |
| | mammalian | | olfactory bulb | indicated; | accumulates in | B2 |
| | cells | | | able to cross | microglia | |
| | | | | BBB | | |
| 22 | EVs from | intranasal | Not specified, | Not specified, | Cortical | U.S. Pat. No. |
| | human stem | | likely | likely BBB | damage region | 10,857,187 |
| | cells | | olfactory and | crossing | (traumatic brain | B2 |
| | | | trigeminal | | injury model). | |
| | | | nerve | | | |
| | | | pathways. | | | |
| 23 | EVs from | intranasal | Not specified, | BBB crossing | Cortical | Moss et al. |
| | human stem | | likely | | damage region | Neurochem |
| | cells | | olfactory and | | (traumatic brain | Int. 150: |
| | | | trigeminal | | injury model) | 105173 |
| | | | nerve | | | (2021). |
| | | | pathways. | | | |
| 24 | EVs from | intranasal | Olfactory and | mechanism | Rostral portion | Pusic et al. |
| | stimulated | | trigeminal | unclear. | of the brain and | PloS |
| | mammalian | | nerve | Hypothesis: | the cervical | one 16(8): |
| | cells | | pathways | nasal mucosa | spinal cord | e0255778 |
| | | | | through | | (2021). |
| | | | | paracellular | | |
| | | | | or transcellular | | |
| | | | | route; BBB | | |
| | | | | crossing | | |
| 25 | nanoparticle- | intravenous | Systemic | BBB crossing | Experimental | Niu et al. |
| | loaded | | circulatory | | brain tumor | Nano Lett. |
| | natural | | system | | uptake | 21(3): |
| | grapefruit | | | | | 1484-1492 |
| | EVs | | | | | (2021). |
| 26 | enzyme- | intranasal | olfactory and | BBB crossing | extensive signal | Hayes et al. |
| | loaded | | trigeminal | | in the prefrontal | Neurotox. |
| | mammalian | | tracts; blood | | cortex, corpus | Res. 39(5): |
| | EVs | | via the nasal | | callosum, and | 1418-1429 |
| | | | epithelium | | hippocampus | (2021). |
| 27 | EVs from | intranasal | Transport | BBB crossing | Inflamed brain | Zhao et al. |
| | GM | | along the | | tissues via | Cells 11(12): |
| | mammalian | | olfactory | | LFA1/ICAM1 | 1933 |
| | cells | | nerve cells is | | interactions | (2022). |
| | | | indicated | | | |
| 28 | magnetic | intranasal | Intranasal | BBB crossing | In the brain 1 | Kutchy et |
| | nanoparticle- | (multiple | perfusion | | day post- | al. Front. |
| | loaded EVs | adminis- | along | | administration | Pharmacol. |
| | from | trations) | olfactory and | | | 13, |
| | mammalian | | trigeminal | | | 819516: |
| | cells | | neural | | | 1-9 |
| | | | pathways | | | (2022). |
| 29 | drug-loaded | intranasal | Not specified | BBB crossing | Only functional | Elsheikh et |
| | artificial | (daily | | | assays showing | al. |
| | bilosomes | administrations | | | improvement in | Pharmaceutics |
| | (bile-salt- | over 21 days | | | a mouse model | vol. |
| | based nano- | | | | of Alzheimer's | 14(3): 576 |
| | vesicles) | | | | Disease | (2022). |

It is shown and described herein that MEVs have properties, including biodistribution, that are distinct from other EVs. The pathways by which the MEVs enter the brain and their ultimate destination differ from those observed for other delivery vehicles. IN administration allows the penetration of MEVs into the brain. As discussed and demonstrated herein, the primary route of penetration of the brain and biodistribution upon IN administration of MEVs is via the olfactory nerve (ON), the mitral/tufted neurons, and the lateral olfactory tract (LOT) in contrast to biodistribution of prior art EVs and nanoparticles via the trigeminal nerve. The MEVs are internalized by the olfactory sensory neurons (OSNs) and travel intracellularly from the olfactory epithelium to the olfactory bulb, through the cribriform plate in the skull. This is in contrast to routes of other delivery vehicles and prior art that indicate or from which it can be inferred that prior art vehicles traffic from the olfactory epithelium to the brain by paracellular transport or transcellular transport. As shown and described herein, intranasally administered MEVs travel from one brain region to another via axonal transport, and more specifically, along the axons of the mitral/tufted neurons throughput the LOT circuit. As shown and described herein, MEVs pass through the synaptic space, in at least at three stages: (1) synapses between the olfactory sensory neurons (OSNs) and mitral/tufted neurons inside the glomeruli in the olfactory bulb; (2) synapses between mitral/tufted axons and neurons resident in brain regions primary colonized by the lateral olfactory tract (LOT), which includes the anterior olfactory nucleus, the tenia tecta, the piriform cortex, the amygdala, the entorhinal cortex; and (3) synapses between the axons afferent from those primary regions of the olfactory tract and the neurons in other brain regions, such as the cortex, the hippocampus, and the hypothalamus connected to the olfactory tract.

As shown in the examples, the brain regions in which the MEVs are found after IN administration perfectly match: (1) the regions that are directly colonized by the mitral and tufted neurons in the LOT (the anterior olfactory nucleus, the tenia tecta, the piriform cortex, the amygdala, the entorhinal cortex); and (2) the regions in the brain that are secondarily connected with the primary target regions, such as the cortex, the hippocampus, the hypothalamus. It is these loci in the brain that can be targeted with therapeutic and diagnostic agents selected for treatment and/or detection of diseases, disorders, and conditions of the brain and the CNS, or diseases, disorders, and conditions that involve the brain and/or CNS.

### 5. Intranasal Administration

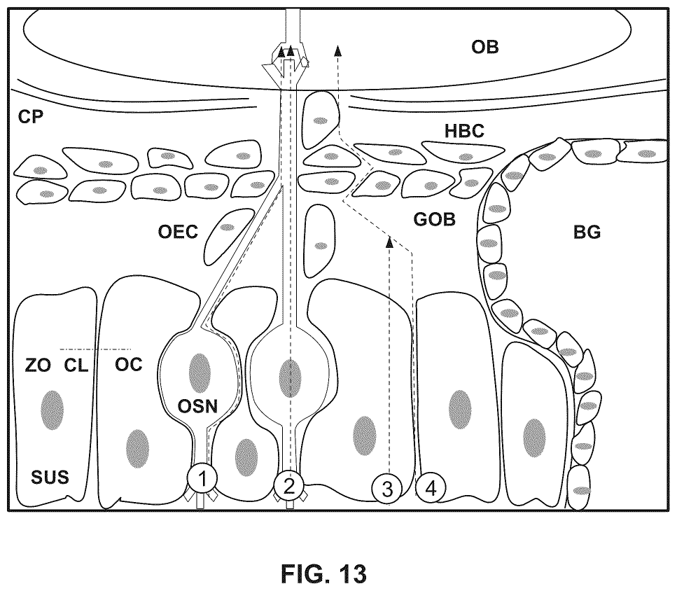

With increasing knowledge of the pathways and functions inside the brain, and as the need for new therapeutics increases, there is a demand for new and more potent CNS drugs. One area is neurodegenerative disorders, where, not only is the pathogenesis not understood, but also drug molecules are not able to reach the target tissue in the brain at an appropriate concentration level. IN administration can provide a way for delivery of therapeutically effective drug concentrations in the brain parenchyma. The absorption of the drugs from the nasal cavity and how they are transported via extracellular (intercellular) or intracellular (transcellular) pathways is under study. Intracellular transport involves a first step of endocytosis into the olfactory sensory neurons. After the neuronal uptake, the molecules move away along the axons to the synapse where they are exocytosed (FIG. 13) and transported further into the brain throughout various synapses.

However, there are limitations for IN delivery of naked drugs (unprotected, non-encapsulated, not loaded inside a nanoparticle, a vesicle or other delivery vehicle). IN delivery of naked drugs is limited to potent drugs delivered in small volumes (25-200 μL in humans), with active mucociliary clearance, short retention time, enzymatic degradation by nasal cytochrome P450/peptidases/proteases (pseudo first pass effect), low permeability for hydrophilic drugs, the need for absorption enhancers, low nasal epithelial pH, inter individual variability, low CNS delivery for proteins, and nasal secretion.

A few products have been approved for IN administration; a few more are in development; all of them are small molecules. Approved products are IMITREX® (GlaxoSmithKline) approved in 1997; MIGRANAL® (Bausch Health Companies) approved in 1997; ZOMIG® (Amneal Pharmaceuticals) approved in 2003, ONZETRA® X (Currax Pharmaceuticals) approved in 2016; and TOSYMRA® (Upsher-Smith Laboratories) approved in 2019. Although these products are administered by the IN route, they enter the blood through the highly vascularized respiratory epithelium (not the olfactory epithelium) and once in the blood they still need to go through the blood-brain barrier. The challenge of being able to bypass the blood-brain barrier by entering the olfactory epithelium has so far not been achieved, and the full potential of IN delivery is yet to be captured.

The olfactory route, trigeminal route, and vomeronasal route can provide direct access to certain regions of the brain, that will otherwise not be reachable. There is still need for optimization of this route(s) as well as full understanding of dosing and safety following nasal drug administration. IN delivery of therapeutic agents is a future perspective to treat neurological diseases.

Administration through the IN route has been recognized as a route for the administration of medicines to the brain, but it is not well-developed nor has it been effectively exploited. Most naked drugs are unstable in the nasal epithelium, and/or unable to cross the barriers from the olfactory epithelium to the olfactory bulb, and/or unable to efficiently move inside the neurons following the axonal networks to reach intimate and specific regions of the brain. Such endeavor has only been used for small chemical molecules; most (if not all) biological molecules (such as DNA, RNA (mRNA, siRNA), complex proteins (antibodies) cannot be administered via an IN route. Hence there is a need to develop vehicles to effectively exploit this route of delivery. As demonstrated herein, MEVs provide such a vehicle.

The olfactory region of the nose is the only part in the whole body where the CNS is in contact with the peripheral environment due to the presence of olfactory receptors neuronally linked to the olfactory bulb. Olfactory and trigeminal nerve pathways allow active agents to be absorbed in the olfactory region and transferred directly into the brain bypassing the BBB.

The nasal cavity is divided into the respiratory area (closer to the nostrils) and the olfactory area (situated high up in the cavity) (Sahin-Yilmaz et al. (2011) Proc. American Thoracic Society 8:31-39). The nasal epithelium is well vascularized (Sahin-Yilmaz (2011) Proc. American Thoracic Society 8:31-39), and, within the olfactory area, olfactory neurons are exposed providing the transport of naked drug compounds directly into the brain via the olfactory neurons. Absorption of naked molecules takes place at the olfactory and respiratory epithelia (Lochhead et al. (2012) Advanced Drug Delivery Reviews 64:614-628). The routes of transportation of naked molecules from the nasal olfactory area to the olfactory bulb are transcellular through either the sustentacular cells or the exposed olfactory sensory neurons. The route of transfer of such compounds from the nasal respiratory epithelium to the brain is via the trigeminal nerves (Ying (December 2007 online) Future Neurol.3 no. 1, doi.org/10.2217/14796708.3.1.1; Lochhead et al. (2012) Advanced Drug Delivery Reviews 64:614-628). Transport to other brain areas after entry to the brain (e.g., to the mid brain from the olfactory bulb or to the brain stem from the trigeminal nerve) is thought to be mainly by either extracellular convective bulk flow (Lochhead et al. (2012) Advanced Drug Delivery Reviews 64:614-628) or via perivascular routes (Lochhead et al. (2015) J. Cerebral Blood Flow Metab. 35:371-381, doi.org/10.1038/jcbfm.2014.215).

In general, direct nose-to-brain delivery obtained demonstrated for rats or mice will overestimate direct nose-to-brain transport in humans if differences in the relative surface area are not adequately accounted for. In contrast, the nasal respiratory epithelium line approximately 50% of the nasal cavity in rats and 80-90% in humans. Therefore, it is possible that access to the brain through trigeminal pathway in primates is underestimated from experiments performed in rodents.

### 6. MEVs and Delivery to the Brain Following Intranasal Administration

As discussed above and herein, the fate of MEVs upon administration via various routes, including intranasal administration, was not known, nor could it have been predicted from the prior art describing trafficking and biodistribution of administration, including IN administration of other delivery vehicles, such as nanoparticles and EVs from other sources. As discussed in more detail in the following section, it is shown and described herein that MEVs, as exemplified by IN administration of Chlorella MEVs, are delivered to the brain following IN administration. As discussed above, and shown in the Examples, MEVs are internalized by the dendrites of the olfactory sensory neurons (OSN) and subsequently are intracellularly passaged from the olfactory epithelium to the olfactory bulb, through the cribriform plate in the skull. As shown herein, the delivery to the brain of MEVs upon IN administration occurs via the olfactory nerve (ON), the mitral/tufted neurons, and/or the lateral olfactory tract (LOT) in the biodistribution and penetration of the brain by MEV. Known EVs are reported to use the trigeminal nerve route and they do not enter the olfactory nerve, and some of the prior art, noted in the table above, that describe nanovectors do not mention the olfactory nerve, the mitral/tufted or the LOT. As noted, there is no suggestion in the prior art of the involvement of axonal transport in the movement of any delivery vehicle from one region to another region, inside the brain. Moreover, in the prior art it is systematically suggested that the entry of the particles to the brain and their movement inside the brain is paracellular or extracellular. Thus, those particles would be either reaching the interstitial liquid between brain cells, or the surrounding capillaries in which case they still need to cross the BBB. As shown herein, regions traversed by the MEVs match the regions that are directly colonized by the mitral and tufted neurons (i.e., the olfactory network), and also those regions in the brain cortex that are secondarily connected with the primary target regions.

### 7. Trafficking and Biodistribution of MEVs Following Intranasal (IN) Administration

Trafficking and biodistribution studies on the fate of MEVs following IN administration were performed and the distribution pathways and patterns were mapped. In the exemplified studies, MEVs were stained with DiR (DiIC18 (7); 1,1′-dioctadecyl-3,3,3′,3′-tetramethylindotricarbocyanine iodide), which is a lipophilic, near-infrared fluorescent cyanine dye. The dye is suitable for labeling lipid membranes detected with near-infrared in vivo imaging. The two long 18-carbon chains insert into the membrane, resulting in specific and stable vesicle staining with no or minimal dye transfer between vesicles. The studies were conducted in recognized rodent models.

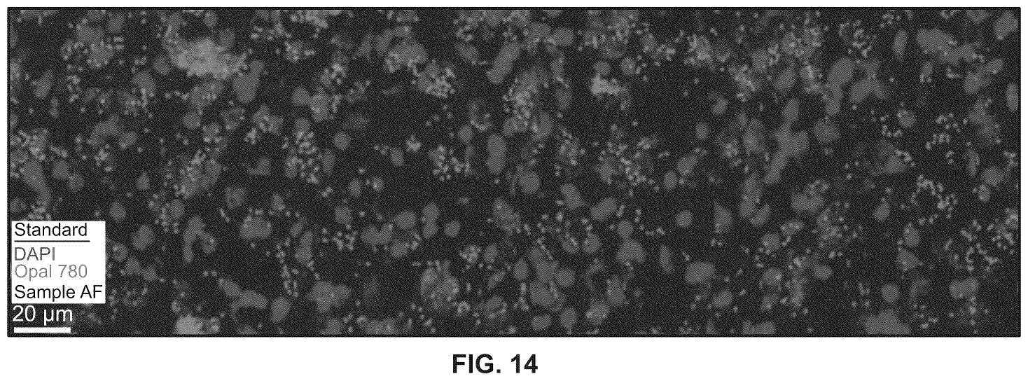

The size of neural cells in mouse brain varies, but the typical nucleus diameter is approx. 5-8 microns. The size of MEVs is about 50-200 nm, with median diameter of 125 nm or 0.125 micron, as measured by Nanoparticle Tracker Analysis (NTA). Images from the positive control slides (FIG. 14) confirm the presence and detectability of the Dir-labelled MEV and demonstrate the size-homogeneity of the MEV material.

It is shown herein that from 1 h to 8 h after IN administration to mice, MEVs can be observed in the following regions of the brain: (1) the olfactory bulb, (2) cortical regions (primary somatosensory, primary visual, primary motor, piriform, agranular insular, frontal, retrosplenial granular, temporal association, auditory, entorhinal), (3) the amygdaloid nuclei (basomedial and basolateral amygdaloid nuclei, amygdalo-hippocampal area, amygdala-piriform transition area), (4) the arcuate hypothalamic nucleus, and (5) the mammillary nucleus. Upon 16 h after IN administration, MEVs can be seen in the thalamus, the cortex, and in different levels of the hippocampus (fimbria and dentate gyrus). The olfactory bulb still contains fluorescent MEVs by 16 h after administration, suggesting that MEVs may continue coming into the olfactory bulb/brain from the nasal cavity over a significant period of time.

Thus, after intra-nasal (IN) administration in an in vivo model, MEVs are found in regions of the brain corresponding to projections of the olfactory bulb (OB) throughout the olfactory network as well as in other regions connected to the olfactory network. Such regions include the piriform cortex (which plays an important role in focal epileptogenesis, forms the major part of the primary olfactory cortex and has extensive connections with other parts of the olfactory network), as well as other cortical regions, such as the temporal association cortex (responsible for identification and integration of complex stimuli), the ectorhinal cortex (under control of OB during working memory performance), and the auditory cortex where auditory and olfactory codes are subjected to cross-modal modulation.

The highest density of vesicles was found in (1) the arcuate nucleus of the hypothalamus which has a central role in homeostasis, (2) the amygdala at the center of behaviors related to panic, anxiety, stress, addiction, and (3) the mammillary bodies which are very important in episodic memory processing within the Papez circuit. In accord with the trafficking data, following IN administration, MEVs are transported following the olfactory circuitry up to various brain regions that are connected to brain nuclei strongly involved in behavior, memory, emotions, and perception of the environment. Consequently, MEVs can be used for delivering directly to the brain cargos that are therapeutic tools for a large diversity of brain and CNS diseases, disorders, and conditions, including, for example, those related to related to epilepsy, food intake, appetite, sexual behavior, stress (PTSD), anxiety, depression, addiction, memory, and others. They also can be used for carrying and delivering detectable cargos for diagnostics and/or theranostics.

The data show that the IN administration of the MEVs leads to a progressive biodistribution of the vesicles in different but specific brain areas. This biodistribution is time-dependent and observed from the rostral to the caudal regions of the brain. One hour after MEV administration, MEVs are only detected in the olfactory nerve layer but not in other more caudal regions. At 2 hours post administration, MEVs reach cortical regions such as the primary motor cortex, the piriform cortex, the frontal cortex, the agranular insular cortex, the primary somatosensory cortex, the auditory cortex, the retrosplenial granular cortex and the temporal association cortex. MEVs also were found in the basolateral amygdaloid nucleus, and the arcuate hypothalamic nucleus. No fluorescence was observed in other regions at the same bregma; bregma is a unit that measures the distance between a location in the brain and the point of junction between the coronal and the sagittal sutures of the skull.

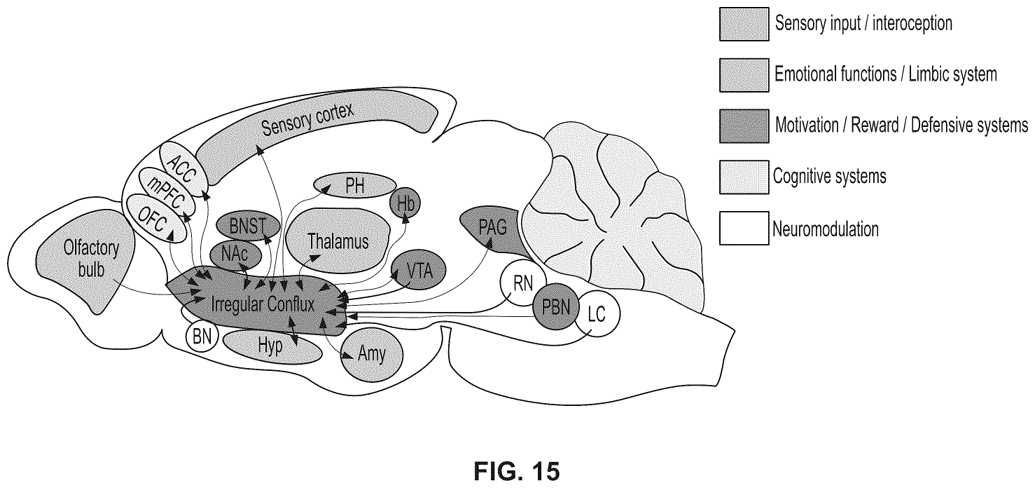

The insula, one of the regions of the brain that is reached rapidly by the MEVs upon IN administration, is a core region that is affected by or involved in many psychiatric and neurological disorders. Many of the anatomical and functional features of the insula are shared across rodents and men, so that rodent models are useful to demonstrate effects and uses for humans. The insula is a hub linking large-scale brain systems. The insular cortex is a true anatomical integration hub with heavy connectivity to an extensive network of cortical and subcortical brain regions serving sensory, emotional, motivational, and cognitive functions (see FIG. 15). The insula receives heavy sensory inputs from all modalities. Direct thalamic and horizontal cortical afferents carry information to the insula from outside the body (auditory, somatosensory, olfactory, gustatory, and visual information) and from inside the body (interoceptive information). Several of these inputs project to topographically organized insular sensory regions, giving rise to the ‘visceral insular cortex’, the ‘gustatory cortex’ (the primary taste cortex), and the insular auditory and somatosensory fields. None of these sensory regions processes only its major sensory input; all regions of the insula receive heavy cross-modal afferents and can be considered to be multimodal integration sites. In addition to its sensory afferents, the insula makes reciprocal connections with the limbic system. For instance, the lateral and basolateral amygdala heavily project to the granular and dysgranular regions of the insula, which in turn send dense efferent signals to the basolateral, lateral and central amygdala nuclei. The insula also connects to the lateral part of the bed nucleus of the stria terminalis, the mediodorsal nucleus of the thalamus, the lateral hypothalamus, and parahippocampal regions, including the perirhinal and the lateral entorhinal cortices. The insula reciprocally connects with frontal brain regions such as the anterior cingulate, the orbitofrontal, and the medial prefrontal cortices, which are implicated in cognitive, emotional and executive functions, and projects to parts of the brain implicated in motivation and reward, such as the nucleus accumbens and the caudate putamen. The insular cortex receives strong neuromodulatory input from cholinergic, dopaminergic, serotonergic, and noradrenergic afferents.

At 4 hours post administration, MEVs migrate up to more caudal brain regions reaching the main body of the amygdala, the left and right auditory cortex, the temporal association cortex and the ectorhinal cortex.

Within 8 h after administration, MEVs are not observed in the caudate putamen, the thalamus, the hippocampus, or the substantia nigra, nor in the most caudal sections examined corresponding to structures like the trigeminal nucleus, the inferior colliculus, the tegmental nucleus and the parabrachial nucleus, indicating that the MEVs do not reach the most caudal part of the brain within the 8 h time window.

It is known that direct nose-to-brain transport of molecules or other structures may occur either via the olfactory nerve or via the trigeminal nerve. Projections of the olfactory nerve originating in the olfactory bulb (OB) penetrate the cribriform plate and terminate at the apical surface of the olfactory neuroepithelium; located at the roof of the nasal cavity. Filaments of the olfactory nerves are present both in the anterior and posterior parts at the middle turbinate. On the other side, the respiratory mucosa (located on the walls of the nasal cavity) is densely innervated by sensory and parasympathetic trigeminal nerves and is even more extensive than the olfactory nerve. Sensory maxillary branches innervate the deepest nasal segments, including the olfactory cleft. Unlike olfactory sensory neurons, the trigeminal nerve endings do not penetrate the mucosal surface. Access of molecules to the dense network of trigeminal nerve endings is thus limited by their ability to cross the mucosal layer.

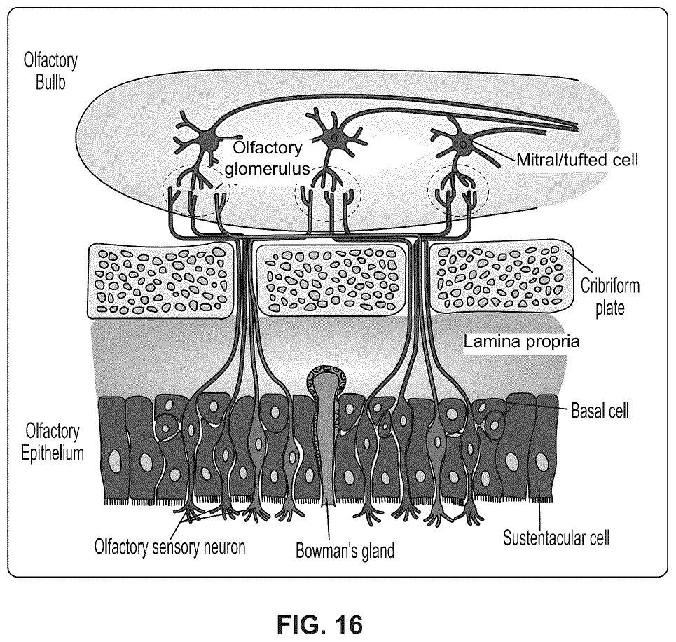

The mechanisms involved in the transport of naked molecules from the nasal cavity to the brain are well known; they have been investigated by using [¹²⁵I]-proteins. Transport across the ‘barriers’ presented by the olfactory and/or respiratory epithelia can occur either by intracellular or extracellular pathways. Intracellular pathways across the olfactory epithelium include (i) endocytosis into olfactory sensory neurons (OSN) and subsequent intraneuronal transport, including intraneuronal axonal transport and also transport between neurons across synapses, to the olfactory bulb and (ii) transcytosis (i.e., transcellular transport) across sustentacular cells to the lamina propria as shown in FIGS. 13 and 16.

FIG. 13 depicts routes of passage through the olfactory epithelium. Four different routes have been described for nose-to-brain drug delivery: (1) OSN extracellular transport: the compound enters directly to the CNS along the OSN (or trigeminal nerve which is not shown in the figure) via bulk flow processes through the tissular liquid surrounding the cells, (2) OSN intracellular transport: the compound is endocytosed and then shuttled to the CNS by a well-organized pathway of intracellular structures inside the OSN), (3) Epithelial or supporting cells intracellular transport: the compound is endocytosed by the supporting epithelial cells and then travel through the intracellular space, (4) Epithelial or supporting cells extracellular pathway: the compound has to pass through the tight junctions like zonula occludens (ZO), claudin (CL), and occludin (OC). Abbreviations: supporting cells (SUS); olfactory sensory neurons (OSN); olfactory ensheathing cells (OEC); globose basal cells (GOB); horizontal basal cells (HBC); Bowman's gland (BG); cribriform plate (CP); olfactory bulb (OB).

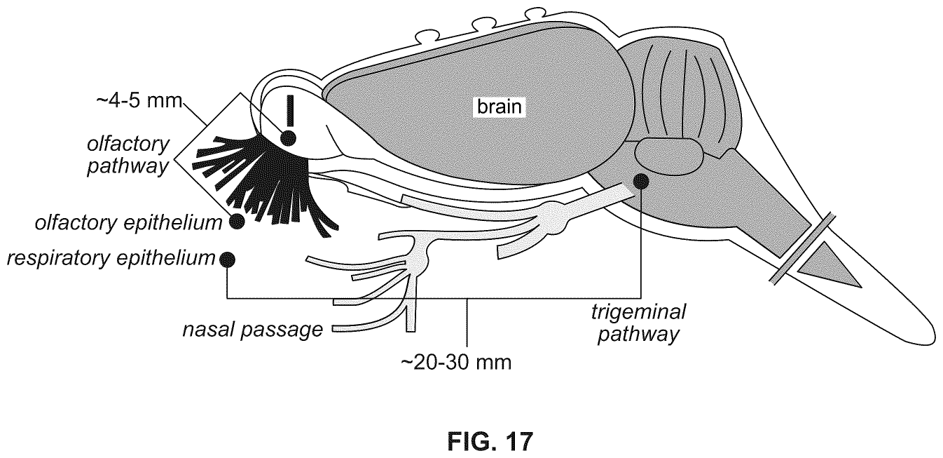

FIG. 17 is a schematic showing the pathways and approximate average distances from the olfactory and respiratory epithelium to CNS targets. Figure reference: Perivascular and Perineural Pathways Involved in Brain Delivery and Distribution of Drugs after Intranasal Administration, Jeffrey J. Lochhead and Thomas P. Davis, Department of Pharmacology, University of Arizona. Pharmaceutics 2019, 11 (11): 598; doi.org/10.3390/pharmaceutics11110598

OSN have the ability to endocytose certain viruses, such as herpes, poliomyelitis, rhabdoviruses, coronaviruses, and also large molecules, such as horseradish peroxidase (HRP), wheat germ agglutinin-horseradish peroxidase (WGA-HRP), and albumin, from the nasal passages and then transport them intracellularly along the axon in the anterograde direction towards the olfactory bulb. HRP is taken up by OSN to a limited extent via fluid-phase endocytosis whereas WGA-HRP is internalized by OSN more avidly via adsorptive endocytosis. (Gänger et al. (2018) Pharmaceutics 10:116, doi: 103390/10030116).

Olfactory sensory neurons (OSN) have several unique attributes: they are the only first order neurons possessing cell bodies located in a distal epithelium and the tips of their dendritic processes, which end as enlarged knobs with several non-motile cilia, extend far into the overlying mucus layer that is directly exposed to the external environment. (FIG. 16; Son et al. (2021) BMB Reports 54 (6): 295-304).

Alternatively, intracellular pathways across the respiratory epithelium can include endocytosis into peripheral trigeminal nerve neurons located near the epithelial surface and subsequent intracellular transport to the brainstem or transcytosis across other cells of the respiratory epithelium to the lamina propria (see FIG. 17). As observed in the OSN, intranasal WGA-HRP is internalized and transported intra-neuronally within the trigeminal nerve to the brainstem. Viruses and bacteria also can be transmitted to the CNS along trigeminal nerve components within the nasal passages (Lochhead et al. (2012) Advanced Drug Delivery Reviews 64:614-628).

Based on data shown and described herein, MEVs are transported through the olfactory nerve and the olfactory tract into the olfactory networks in the brain. While not observed because of the duration of the studies, the data do not exclude transportation of the MEVs through the trigeminal nerve and trigeminal network, as the transport through the trigeminal nerves would take substantially longer before the entry point in the posterior part of the brain (the pons) is reached. The observed pathways, however, are unique to MEVs.

The respective distances from olfactory or respiratory epithelium to CNS have allowed the calculation of transportation time for proteins. Using published data (Buchner et al. (1987) Neuroscience 22:697-707) for fast (130 mm/day) and slow (36 mm/day) axonal transport of exogenous proteins in pike olfactory nerves (corrected to 37° C.), indicate that intracellular transport (via axonal transport) within olfactory neurons to the olfactory bulb for MEVs should take 0.74 h-2.7 h, and 3.7 h-13 h for intracellular transport (via axonal transport) within trigeminal ganglion cells to the brainstem, assuming similar transport rates in OSN and trigeminal cells. As discussed below, based on data herein, there is no evidence indicating involvement of the trigeminal nerve in the brain penetration of MEVs at least through the duration of study post-administration.

The data indicate that MEVs are transported through the axons projecting from mitral/tufted cells in the olfactory bulb. Axons of mitral/tufted cells are fasciculated and form the two lateral olfactory tracts (LOT), one on each side of the brain. They extend multiple collaterals that project to various areas of the olfactory cortex, including the anterior olfactory nucleus, the olfactory tubercle, the piriform cortex, the lateral entorhinal cortex, the cortical amygdala, among other (see, FIGS. 13, 16, and 18-20).

FIG. 16 presents a schematic diagram of the brain neuronal pathway from the olfactory sensory neurons (OSN) through the olfactory bulb (OB) to the mitral and tufted neurons, to the olfactory tract (OT).

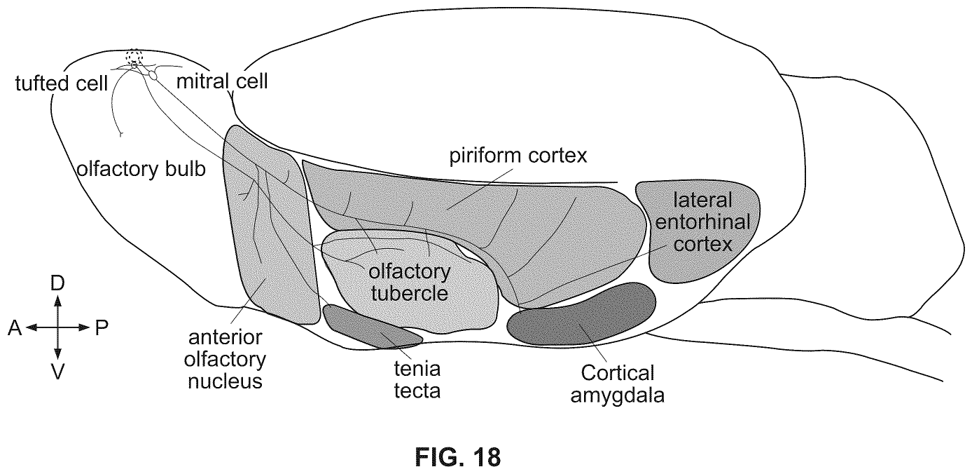

FIG. 18 presents a cortical projection of mitral and tufted cells. Ventrolateral view of the brain is schematically shown (reproduced from Construction of functional neuronal circuitry in the olfactory bulb, November 2014, Seminars in Cell and Developmental Biology 35, DOI: 10.1016/j.semcdb.2014.07.012, Takeshi Imai, Kyushu University).

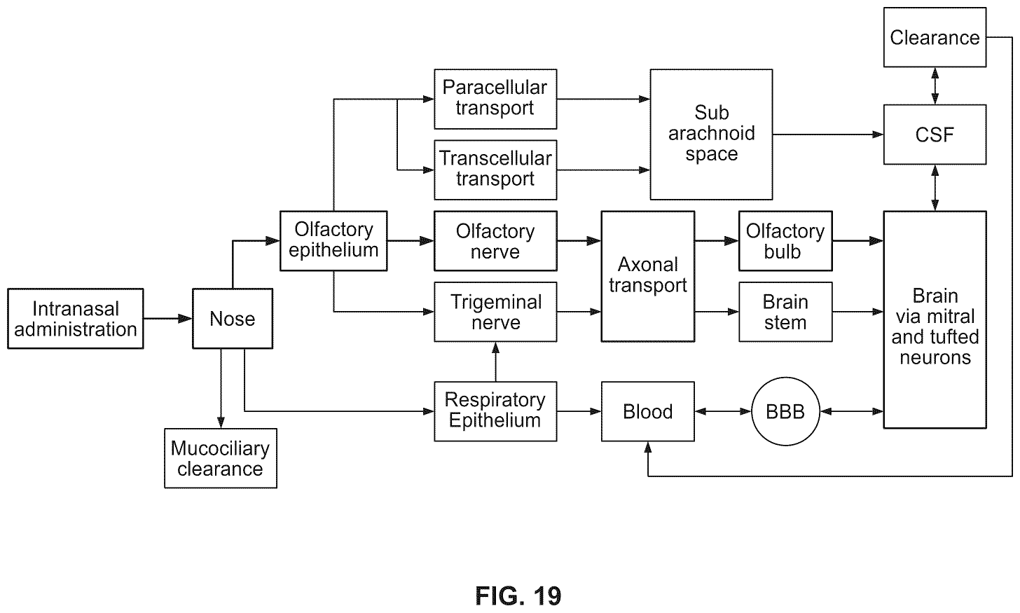

FIG. 19 shows alternative possible pathways following IN administration. Based on data herein, for the MEVs, after IN administration, the pathway is from the nose to the olfactory epithelium and then to the olfactory neurons, then the MEVs are transported by axonal transport to the olfactory bulb, then by mitral and tufted neurons to the primary olfactory regions that process the olfactory signal (Figure reproduced from Selvaraj et al. (2018) Artificial Cells, Nanomedicine, and Biotechnology An International Journal 46:2088-2095, doi.org/10.1080/21691401.2017.1420073).

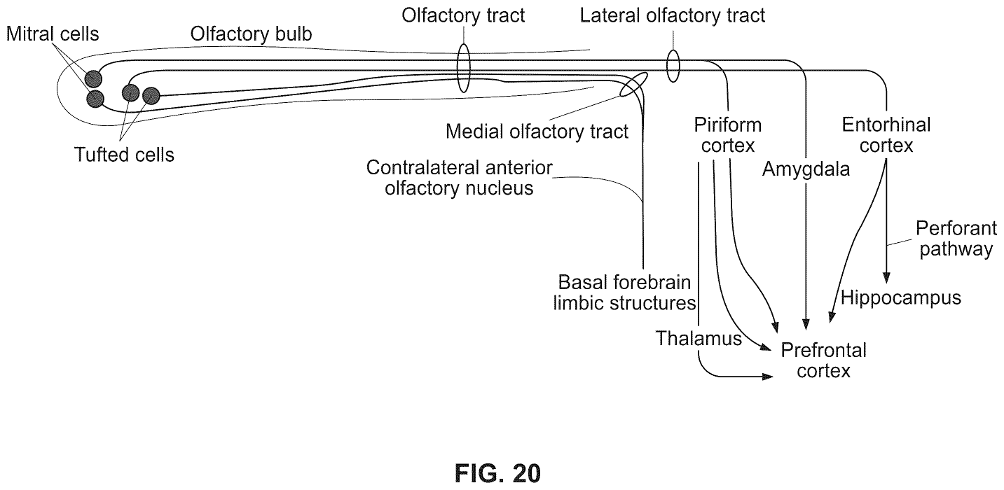

FIG. 20 (reproduced from “What-when-how in Depth tutorials and information, Olfaction and Taste, Sensory system, part 1” (what-when-how.com)) shows schematics of the pathways following IN administration after reaching the OB as described for the Figure. The olfactive pathway used by MEVs after IN administration is as follows. The olfactory bulb (OB) is the first region of the CNS where sensory signals from olfactory sensory neurons (OSNs) are processed. Axons of the OSN travel in olfactory nerves and spread over the surface OB, forming an olfactory nerve layer. Located near the surface of the OB is the glomerular layer. Each glomerulus contains clusters of nerve terminals from OSN, dendrites of the tufted cells, mitral cells, and y-aminobutyric acid (GABA)-ergic interneurons, called the periglomerular cells. The terminals of first order OSN form synapses with the dendrites of the tufted, mitral, and peri-glomerular neurons. The projections of the axons of the mitral and tufted cells are shown schematically in the Figure. Olfactory tracts (LOT), located on the ventral (inferior) surface of the frontal lobe, arise from the OB. The largest bundle of axons from the mitral and tufted cells exits from the OB in the LOT, and they project to the primary olfactory cortex, piriform cortex, amygdala, and entorhinal cortex. The entorhinal and piriform cortices, hippocampus, and amygdala are in the temporal lobe; the hippocampus lies in the medial temporal lobe. The neurons in the piriform cortex, amygdala, and entorhinal cortex project to the prefrontal cortex. Note that the olfactory projection system differs from other sensory systems in that the projection pathway can reach the prefrontal cortex without having to make a synapse in the thalamus first, which is typical of other sensory systems. Neurons in the entorhinal cortex project to the hippocampus (a major limbic structure) via a fiber bundle called the perforant fiber pathway. Therefore, olfactory inputs play an important role in modulating hippocampal functions in a manner like that for the amygdala. Although olfactory projections can reach the prefrontal cortex without making a synapse in the thalamus, there are direct tertiary inputs from the piriform cortex to the mediodorsal thalamic nucleus, which projects to wide areas of the frontal lobe, including the prefrontal cortex. Some fibers from the mitral and tufted cells exit the LOT via the medial olfactory tract. These axons project ipsilaterally to basal limbic forebrain structures, such as the substantia innominata, medial septal nucleus, and bed nucleus of the stria terminalis. Other fibers in the medial olfactory stria arise from the contralateral anterior olfactory nucleus. This nucleus, located in the posterior part of each OB, receives sensory signals from mitral and tufted cells and relays them to the contralateral OB via the anterior commissure.

MEVs trafficking and regions of the brain that can be targeted or affected by delivery of MEV cargo.

The amygdala has been implicated in aspects of emotional processing. The direct connection of the olfactory bulb with some subregions of the amygdala indicates a particular role in olfactory processing; the amygdala is involved in the generation of rapid responses to olfactory stimuli (including fight/flight) particularly in approach/avoid contexts, in olfactory-related reward processing, including learning and memory of approach/avoid responses (Noto et al. (2021) Front Syst Neurosci 15:752320, doi: 10.3389/fnsys.2021.752320). The amygdala is one of the regions targeted by psychedelics, including psilocin, to elicit the biological response. The amygdala also plays a role in posttraumatic stress disorder (PTSD) (Badura-Brack et al. (2018) Psychiatry Research: Neuroimaging 271:135-141; Badura-Brack et al. (2018) Biological Psychology 132:228-232). These findings indicate that MEVs can be used to transport drugs and other agents for treatment of diseases, disorders, and conditions involving such responses.

Mammillary bodies have unique connectivity to the anterior olfactory nucleus (Zhou et al. (2019) eLife 8: e47177, doi.org/10.7554/eLife.47177). Mammillary bodies have a central role in the Papez circuit involving amygdala and thalamus and play an active role in how recognitional memory is processed.

The presence of the MEVs in the arcuate hypothalamic nuclei is consistent with the fact that ghrelin containing neurons in the olfactory bulb send collateralized projections to this brain area (Russo et al. (2018) Algorithms 11:134, doi.org/10.3390/a11090134). Ghrelin is involved in eating behaviors and the arcuate nucleus is a major integration center for peripheral satiety signals and feeding behavior.

The data show that by 16 h after IN administration of the MEVs, they are in the corpus callosum, the dorsal fornix, the dorsal hippocampal commissure and the fimbria of the hippocampus which is connected to the fornix. All these regions correspond to bundle of nerves forming white matter.

The olfactory bulb projects directly to a number of primary cortical brain structures, projections from each of these structures to the rest of the brain constitute a widespread olfactory network. Each of such primary brain structures of the olfactory network, which includes the anterior olfactory nucleus, the olfactory tubercle, and the frontal and temporal piriform cortices, subsequently form and connect to dissociable whole-brain networks. Such networks are characterized by unique functional connectivity profiles for each subregion, leading to higher profile, large-scale processing pathways of the olfactory system (Zhou et al. (2019) Nucleic Acids Research 47 (Issue W1): W234-W241, doi.org/10.1093/nar/gkz240).

The data herein show effective IN delivery of MEVs, via the olfactory nerve and throughout the lateral olfactory tracts (LOT), to a number of brain regions. Upon IN administration, MEVs enter the brain via the olfactory nerve. MEVs are internalized by the dendrites of the olfactory sensory neurons (OSN) in the olfactory epithelium (at the roof of the nasal cavity) and then be transported to the olfactory bulb (OB), intracellularly, through the body of the OSN. The olfactory nerve starts at the nasal olfactory epithelium and ends at the olfactory bulb (OB). As noted, based on the data herein, there is no evidence indicating involvement of the trigeminal nerve in the brain penetration of MEVs, in the times post-administration that have been evaluated.

Inside the olfactory bulb (OB) are the glomeruli where the incoming axons from the OSN synapse with dendrites of mitral neurons and of tufted neurons. The mitral/tufted neurons are the principal neurons in the OB. LOTs are composed of the long axons of mitral and tufted neurons that travel from the OB to the various anterior-posterior brain regions directly involved in the olfactory system, which is composed of the: anterior olfactory nucleus, olfactory tubercle, tenia tecta, piriform cortex, amygdala, and entorhinal cortex. Lateral ramifications of the main long axons of the mitral/tufted neurons enter and colonize each of the brain regions (anterior olfactory nucleus, olfactory tubercle, tenia tecta, piriform cortex, amygdala, and entorhinal cortex). Inside those regions, the mitral/tufted axons are connected (via synapses) with neurons from other regions (having a more secondary olfactory role), including the frontal cortex, the hypothalamus, and the hippocampus.

Data show that MEVs administered by IN reach all and each of the brain regions reached by the olfactory nerve and the lateral olfactory tracts (LOT) throughout the brain. MEVs arriving to the glomeruli from the OSN axons enter the mitral neurons and tufted neurons and travel following a clear pathway with a clear kinetics throughout the lateral olfactory tract (LOT) in both hemispheres: ventral, lateral and dorsal regions; external and internal regions; along the antero-posterior axis. The brain regions reached by the MEVs within 1 and 16 hours after IN administration are the anterior olfactory nucleus, the olfactory tubercle, the tenia tecta, the piriform cortex, the amygdala, the entorhinal cortex, the primary motor cortex, the frontal cortex, the agranular insular cortex, the primary somatosensory cortex, the auditory cortex, the retrosplenial granular cortex, the temporal association cortex, the basolateral amygdaloid nucleus, the arcuated hypothalamic, the corpus callosum, the internal capsule, the thalamus, the hippocampus (fimbria, dentata gyrus). The biodistribution pattern of the MEVs, thus, perfectly matches the pathways and connections of the olfactory nerve, the mitral/tufted axons, and the LOT (lateral olfactory tract) throughout the entire brain.

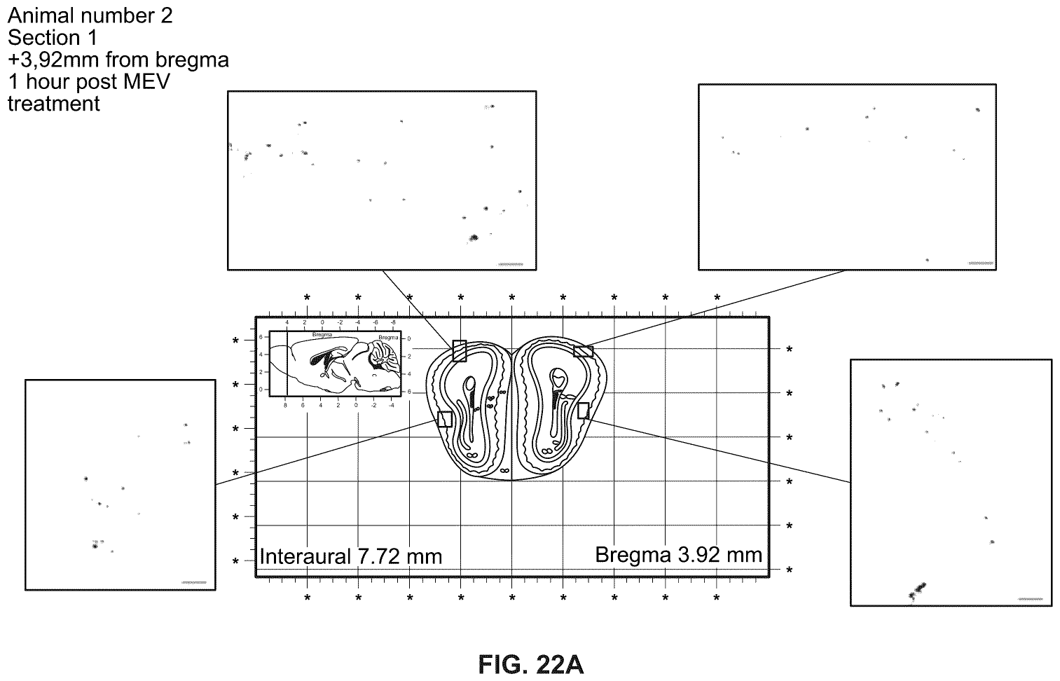

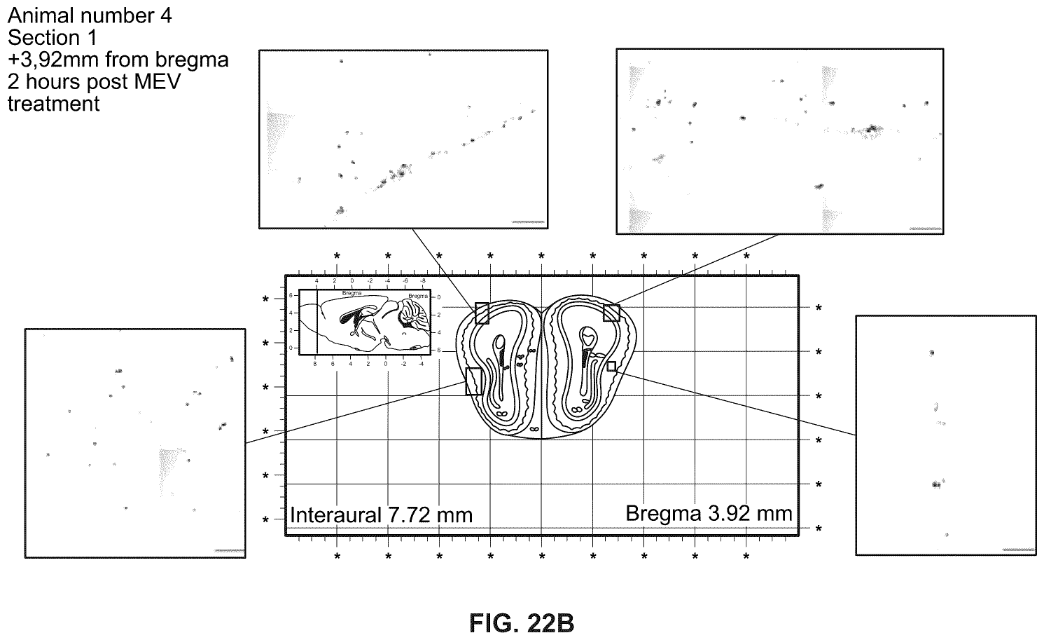

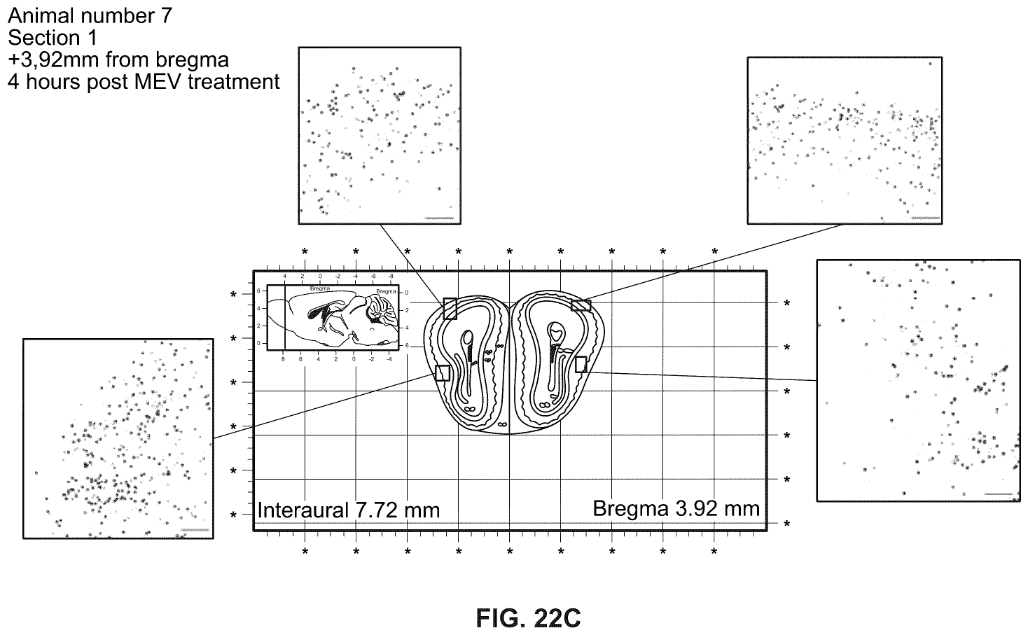

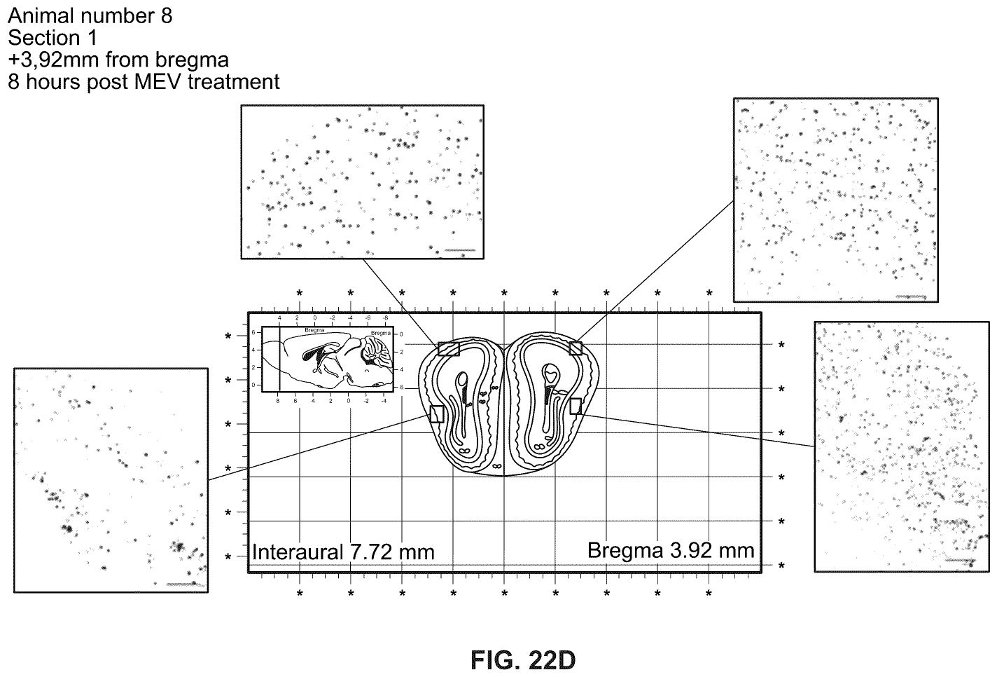

The images in the figures (see, e.g., FIGS. 22A-D, 24A-D, 26A-D, 28A-D) show that, in the different regions they reach, MEVs are disposed in clusters rather than as individual spots, indicating that they are released in the extracellular space surrounding the axon terminals that transported them to those regions. Most of the brain areas where MEVs have been localized correspond to the olfactory bulb circuitry (see, e.g., Imai (2014) Seminars in Cell & Developmental Biology 35:180-188; Igarashi et al. (2012) Journal of Neuroscience 32:7970-7985; doi.org/10.1523/JNEUROSCI.0154-12.2012; and Nagayama et al. (23 Sep. 2010) Front. Neural Circuits, 23; doi.org/10.3389/fncir.2010.00120).

Most brain regions reached by the MEVs are consistent with the conclusion of direct axonal connections from the olfactory bulb, such as (1) projections of ghrelin neurons from mitral cells into the arcuate hypothalamic nucleus (Russo et al. (2018) Experimental Brain Research 236:2223-2229); (2) the direct modulation of entorhinal cortex activity by the olfactory bulb (Salimi et al. (2021) The Journal of Physiological Sciences 71: Article number 21); and (3) the reciprocal connections of the agranular insular cortex with the olfactory bulb (Gehrlach et al. (2020) eLife 9: e55585). The presence of the MEVs in other cortical regions is not due to direct axonal projections from the olfactory bulb but rather indirect projections from areas like the amygdala or the mammillary bodies (direct connections between amygdala and motor cortex (Grezes et al., 2014), connections between the retrosplenial granular cortex and the RGC (Buckwalter et al. (2008) Experimental Brain Research 186:47-57), connections to the somatosensory cortex (Macdonald et al. (1998) J. Neurophysiol. 79:474-477), direct connections between mammillary bodies and RGC (Groen et al. (2003) J. Comparative Neurology 463:249-263, doi.org/10.1002/cne.10757). On the other side, the presence of MEV in the auditory cortex and in the primary visual cortex appears to be due to diffusion from proximal entorhinal cortex or RGC, respectively, as no direct neuronal connections have been reported from any of the areas of distribution.

### 8. Primary and Secondary Circuitry of the Olfactory System and Regions Reached by the MEVs Upon IN Administration

In this section, brain regions reached by the MEVs upon IN administration are indicated in bold and with an asterisk*. The olfactory sensory neurons*(OSN) are primary sensory neurons located within the olfactory epithelium in the upper nasal cavity. Axons of the OSN leave the olfactory epithelium and synapse in the olfactory bulb*(OB). These axons enter the anterior cranial fossa through the cribriform plate of the ethmoid bone. Neurons in the OB called mitral cells are secondary sensory neurons of the olfactory system. Their axons leave the OB and enter the olfactory tract*(two lateral olfactory tracts, or LOTs), which is not a peripheral nerve but part of the central nervous system.

MEVs reach cortical regions such as the primary motor cortex*, the piriform cortex*, the frontal cortex*, the agranular insular cortex*, the primary somatosensory cortex*, the auditory cortex*, the retrosplenial granular cortex*, the temporal association cortex*, the basolateral amygdaloid nucleus*, and the arcuate hypothalamic nucleus*.

Some fibers of the LOT turn laterally and project themselves to the olfactory cortex*(all gray matter areas that receive output from the OB). The main areas of the olfactory cortex are the piriform cortex*(the most posterior part of the orbitofrontal cortex), the cortical amygdala*(this is the superficial part of the amygdala). The olfactory cortex projects to the hypothalamus*. The hypothalamus is described as using olfactory information to affect feeding, reproductive activity, and autonomic reflexes triggered by olfactory signals.

The olfactory cortex (and the gustatory cortex) project to the orbital prefrontal cortex*(the inferior surface of the frontal lobe), where information from both sensory modalities can be combined for the sensation of flavor. The limbic system contains the limbic lobe and other cortical regions that have connections with the limbic lobe. This group of structures is associated with learning, memory, emotion, and motivation. The limbic lobe is composed by the cingulate gyrus, the parahippocampal gyrus, and the hippocampus.

The cingulate gyrus*is the cortex adjacent to the corpus callosum*. The anterior half of the parahippocampal gyrus is called entorhinal cortex*, which is one of the four components of the hippocampus. The entorhinal cortex is part of both the parahippocampal gyrus and the hippocampus*.

There are four components of the hippocampus: the entorhinal cortex and the three components of the rolled-in part of the hippocampus—the dentate gyrus, the Ammon's horn, and the subiculum. The entorhinal axons synapse on the granule cells in the dentate gyrus, the granule cell axons synapse on the pyramidal cells of Ammon's horn, which synapse on the pyramidal cells of the subiculum. The hippocampus is necessary for storing recent memories of facts and events. It receives processed information from all sensory cortices which projects to the inferior temporal cortex, which in turn sends axons to the entorhinal cortex. Through these connections, the hippocampus is informed about sensory processing in all cortical areas.

A circle of connections from the hippocampus to the mammillary body*, to anterior thalamus*, to cingulate cortex, and back to the hippocampus through the cingulum and parahippocampal gyrus is known as the Papez circuit. Axons of cells in the hippocampus form a layer of white matter known as the fimbria*. The fornix is the major projection from the hippocampus*, it dives into the hypothalamus*and terminates in the mammillary body*. The mammillary body*projects to the anterior nucleus of the thalamus*, which at its turn sends axons to the cingulate gyrus. Axons from the cingulate gyrus go to the parahippocampal gyrus and the entorhinal cortex*, completing the circuit back to hippocampus*.

The observed nose-to-brain passage of the MEVs via the olfactory neurons in the olfactory epithelium (as opposed to via the trigeminal nerve) is advantageous for various reasons that include, for example: the turnover of the mucus in the epithelium of the upper nose cavity (where the olfactory epithelium is located) occurs in days, which allows and supports consistent drug absorption over time; while in the lower nose cavity (where the respiratory epithelium containing the terminals of the trigeminal nerve is located) turnover occurs in minutes.

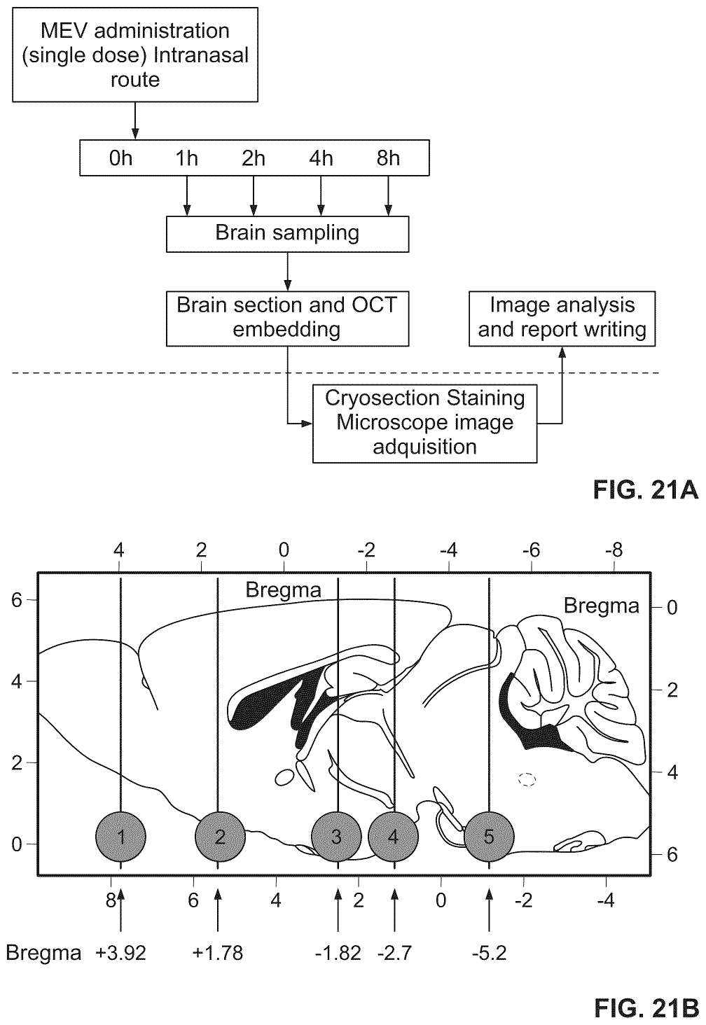

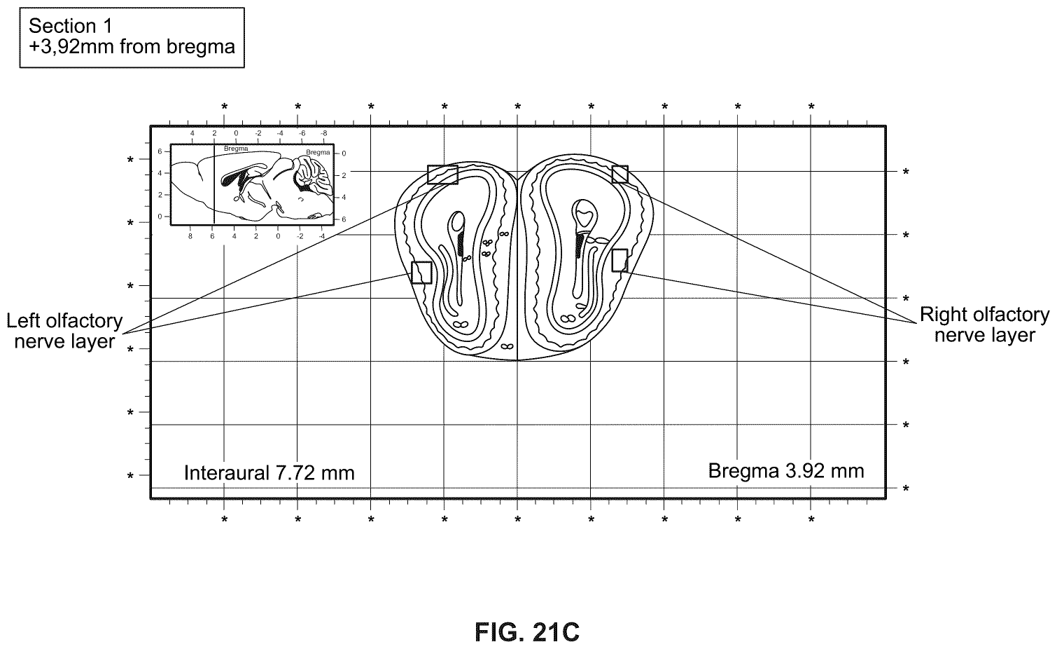

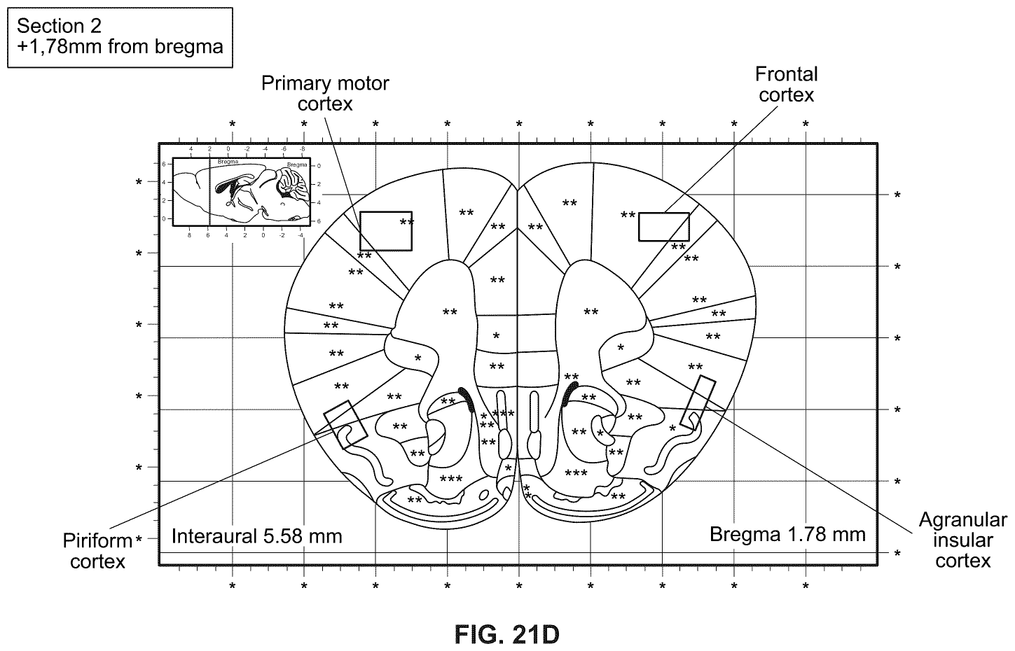

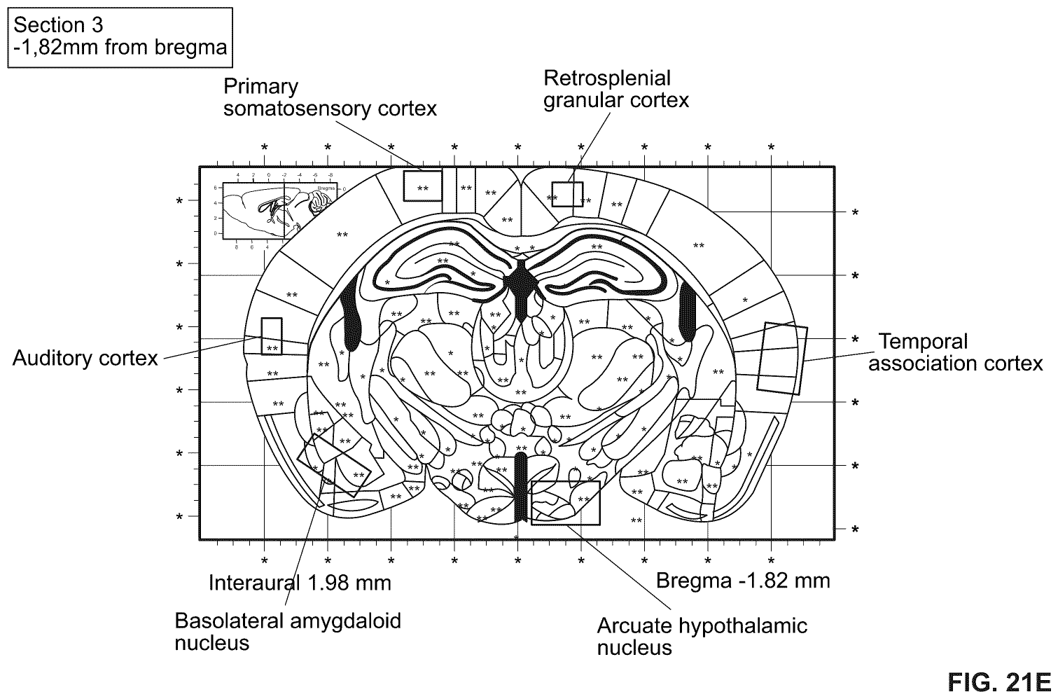

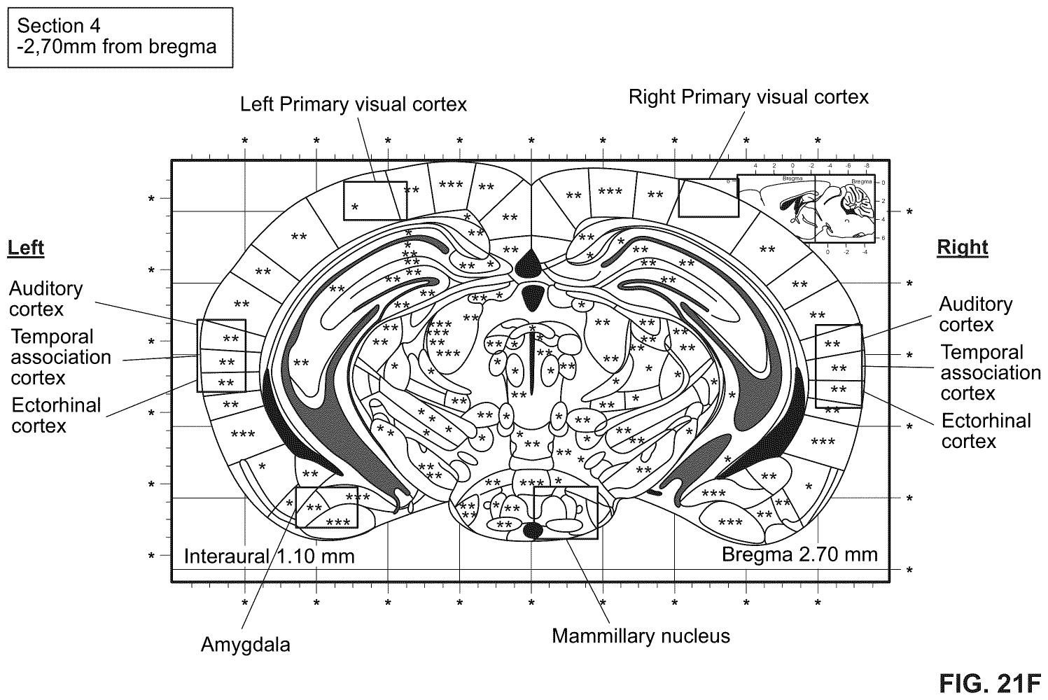

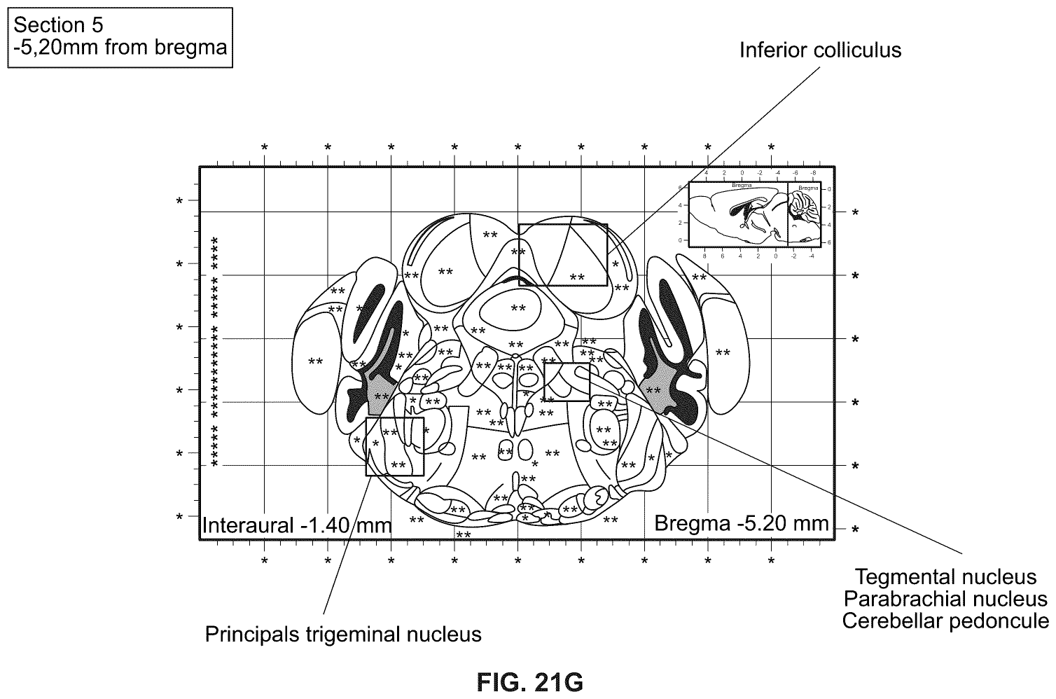

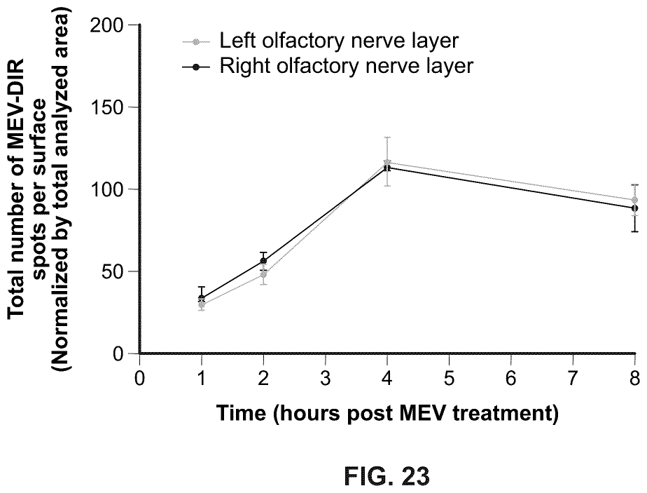

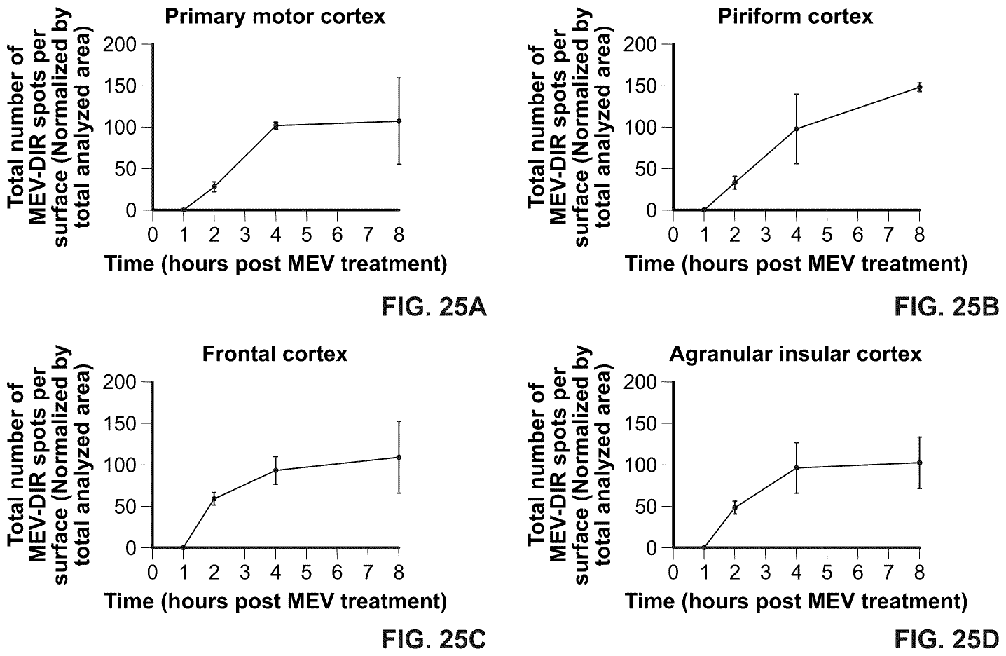

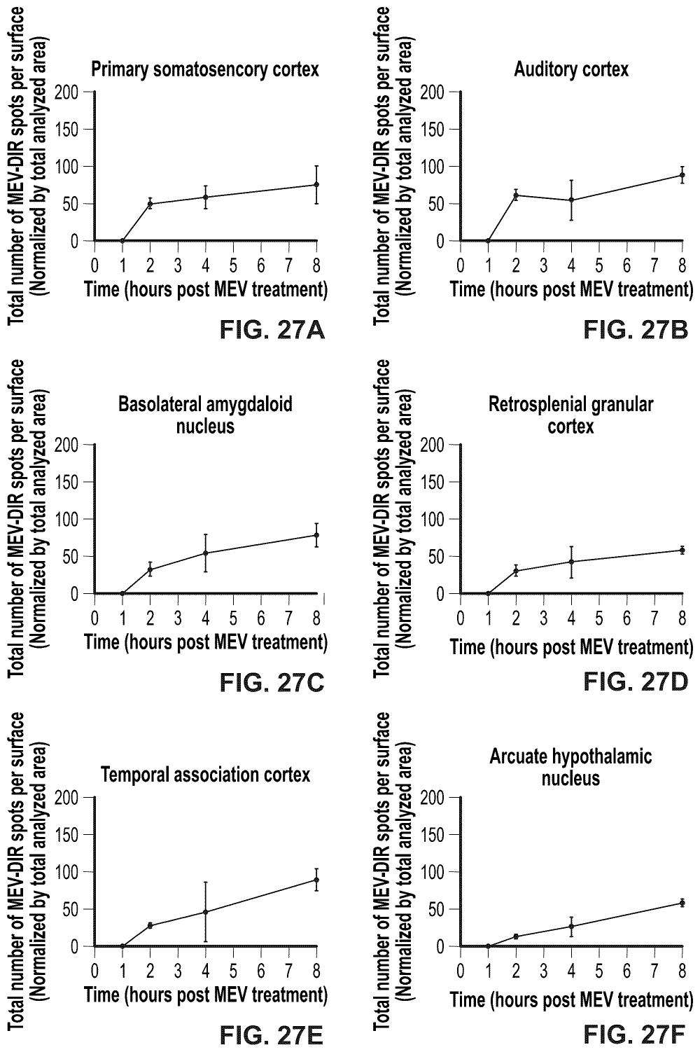

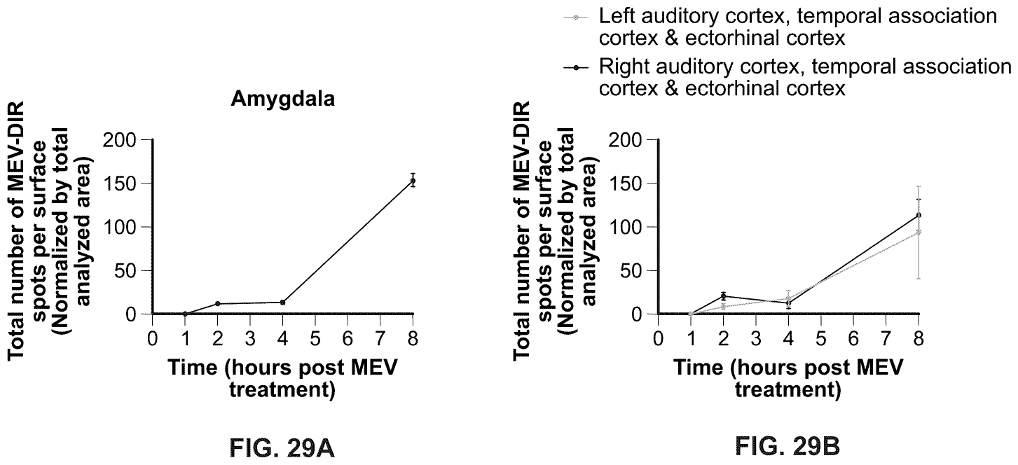

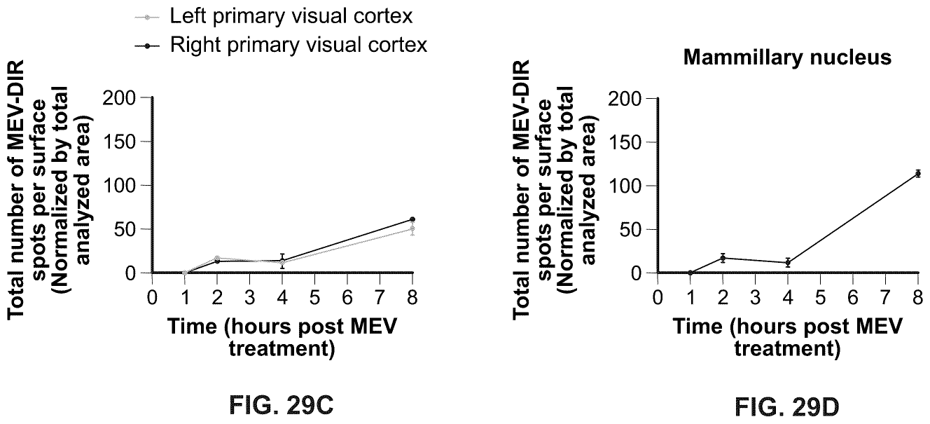

The kinetic analysis of distribution from 1 h to 8 h, shows an increasing time-dependent density of MEV in all the regions analyzed from rostral to caudal parts of the brain (FIGS. 21A-31B). Kinetic studies show a strong directionality in the migration of the MEV inside the brain. There is clear movement of the MEVs from the site of entrance in the anterior or frontal part of the brain (the olfactory bulb) to the more posterior or distal parts, all along the axons of the mitral/tufted neurons, the regions they colonize and the secondary regions in their neural network.

It is known that neuron organelles and mitochondria, as well as endogenous intracellular vesicles (intracellular to the neurons) travel via a well-developed and structured mechanism of axonal transport. The mechanism and underlying structures for axonal transport also are used by viruses, such as, for example, herpes viruses, SARS-COV-2, and others, when they infect the nerves, such as, for example, the olfactory nerve, to move to internal brain structures and expand the infection. Infecting viruses and intracellular neuronal vesicles travelling via axon transport can pass over the synaptic junctions and move from one neuron to another neuron.

As shown and described herein, MEVs travel intracellularly, via axonal transport, and can cross-over synapses, at least over (i) the synapses between the OSN and the mitral/tufted neurons, (ii) the synapses between the mitral/tufted neurons and the local neurons in the various brain regions colonized by the LOT, and (iii) the synapses between the neurons in the brain regions colonized by the LOT and neurons from the frontal cortex, the hippocampus, and the hypothalamus. Alternative ways of passage from the olfactory epithelium to the brain could have been paracellular transport or transcellular transport; but these kinds of transport are not compatible with the observed data.

In summary, upon IN administration, MEVs are internalized by the dendrites of the olfactory sensory neurons (OSN) and move intracellularly (inside the olfactory sensory neurons (OSN)) from the olfactory epithelium to the olfactory bulb, through the cribriform plate in the skull. The olfactory nerve (ON), the mitral/tufted neurons, and the lateral olfactory tract (LOT) are the main actors involved in the biodistribution and penetration of the brain by the MEVs. MEVs travel from one brain region to another via axonal transport, and more specifically, along the axons of the mitral/tufted neurons throughput the LOT circuit.

There is a strong correlation between the brain regions primarily reached by the MEVs upon IN administration, and the regions that are the target sites for some psychoactive drugs, such as psychedelics, such as psilocybin and/or psilocybin derivatives, and other drugs for treating the CNS. MEVs, as described herein, can be used for delivery of cargos, such as psilocybin derivatives, to the target sites in the brain where biological activity as an antidepressant or other activity is elicited.

### 9. Delivery of MEVs Via IN Administration to the Brain-Exemplary Bioactive Cargo and Uses Thereof

The cargo-loaded MEVs can be used and administered intranasally for treatment of brain disorders, psychiatric disorders, dementia, brain cancers, brain trauma, brain injury, and for treatment of any disease, disorder, or condition in which the brain is involved. The MEVs also can be used for diagnosis to detect brain disorders, such as cancers, and to monitor treatments. In general the MEVs are used for treatment of humans, but they can be used for treatment of animals, as they often suffer from similar or the same brain-associated disorders as humans. For brain delivery, cargo generally comprises molecules (small molecule drugs, proteins, nucleic acids, and others) that affect or treat or detect diseases, disorders, and conditions of the brain and/or CNS.

Diseases, disorders, and conditions involving the brain, include, for example, human psychiatric disorders, and also animal disorders, including anxiety disorders (panic disorders, social anxiety, phobia-related disorders, and generalized anxiety disorders); attention deficit hyperactivity disorders (inattentive type, hyperactive-impulsive type, combination type); autism spectrum disorders (Asperger's syndrome, Childhood Disintegrative Disorder (CDD), Kanner's syndrome, Pervasive Developmental Disorder (PDD-NOS)); bipolar disorders (Bipolar I disorder, Bipolar II disorder, bipolar with mixed features, bipolar with seasonal pattern major depression, Cyclothymia, rapid cycling bipolar); eating disorders (anorexia nervosa, bulimia nervosa, muscle dysmorphia, binge eating disorder, other specified eating or feeding disorder (OSFED), compulsive over eating, Prader Willi syndrome, Diabulimia, orthorexia nervosa, selective eating, drunkorexia, pregorexia); personality disorders (antisocial personality disorder, borderline personality disorder, histrionic personality disorder, narcissistic personality disorder, avoidant personality disorder, dependent personality disorder, obsessive-compulsive disorder (OCD)); post-traumatic stress disorders (PTSD) (acute stress disorder, uncomplicated PTSD, complex PTSD, comorbid PTSD); schizophrenia (catatonic schizophrenia, disorganized schizophrenia, paranoid schizophrenia, residual schizophrenia, and undifferentiated schizophrenia); other such psychiatric and brain-related conditions.

The MEVs provide for the delivery, via intranasal administration, and the other routes, as described herein, and expression of active small molecules, proteins, anti-sense oligonucleotide (ASOs), and RNA inside the brain. The MEV cargo for delivery to the brain includes psychoactive agents, enzymes, growth factors, detectable products, such as psilocybin derivatives, harmine, temozolomide, rivastigmine rhodamine (small molecules); catalase, GFP, nerve growth factors (NGFs), TrkA (tropomyosin kinase A), neurotrophic factors (NT-3, NT-4, BDNF (brain derived neurotrophic factor, CNTF (ciliary neurotrophic factor), EPO, IGF-1, bFGF (basic fibroblast growth factor), hGH, or luciferase (proteins); GABAB1A receptor, eGFP or Luciferase (mRNA); GABABIA receptor (siRNA), PTEN (siRNA; SEQ ID NOs: 136-138); miR-17 (miRNA; SEQ ID NOs: 139-141); or MALAT1 (SEQ ID NO:142; lncRNA) into the brain by the IN route. MALATI (SEQ ID NO: 142), a metastasis associated lung adenocarcinoma transcript 1, is a long non-coding RNA (Inc RNA). In the brain, over-expression of long noncoding RNA MALATI ameliorates traumatic brain injury induced brain edema by inhibiting AQP4 and the NF-κB/IL-6 pathway (see, e.g., Zhang et al., first published 19 Jun. 2019, J. Cellular Biochemistry 120 (10): 17584-17592, doi.org/10.1002/jcb.29025).

Cargo for delivery in the MEVs can comprise any of the following single molecules, or in combination, for treatment of any of the above conditions, for example: 5-hydroxytryptamine-1A (5-HT1A) and 5-hydroxytryptamine-3 (5-HT3) receptor agonists, for example, Azapirones, Methylphenidate, Dexmethylphenidate, Ondansetron (Zofran®, GlaxoSmithKline); Acetylcholinesterase inhibitors, for example, Donepezil, Galantamine, Rivastigmine; Alpha-1-receptor antagonists, for example, Prazosin; Anticonvulsants, for example, Gabapentin, Pregabalin, Topiramate (sold under the trademark Topamax®, Ortho-McNeil Pharmaceutical), Carbamazepine, Eslicarbazepine, Levetiracetam, Licarbazepine, Oxcarbazepine, Valproic acid and derivatives, Lamotrigine; Anti-inflammatory drugs and supplements of various mechanisms, including non-steroidal anti-inflammatory drugs (NSAIDs) and selective and non-selective COX-2 inhibitors, for example, celecoxib, acetylsalicylic acid (Aspirin), ibuprofen, and naproxen, Infliximab, Omega-3 polyunsaturated fatty acids, Pioglitazone, TNF-alpha inhibitors, N-Acetyl-Cysteine, Dexamethasone, Minocycline; Antipsychotics, for example, Aripiprazole, Asenapine, Cariprazine, Chlorpromazine, Clozapine, Haloperidol, Lumateperone tosylate (Caplyta®, Intra-Cellular Therapies), Olanzapine, Paliperidone, Quetiapine, Risperidone, Ziprasidone; Beta blockers, for example, Azapirones, Propranolol. Drugs that modulate the cholinergic system, for example, Biperiden, scopolamine; Corticotropin Releasing Factor (CRF) antagonists; Drugs that modulate the GABAergic system, for example, Benzodiazepine, Brexanolone, Sage-217; Glucocorticoid receptor agonists, for example, Hydrocortisone; Drugs involved in glutamatergic modulation, for example, AGN-241751, AV-101, AVP-786, AVP-923, AXS-05, D-cycloserine, Dextromethorphan, Rapastinel; Glycine, and glycine reuptake inhibitors, for example, Sarcosine; Drugs that modulate the hypothalamic-pituitary-adrenal (HPA) axis, for example, Fludrocortisone, Metyrapone, Mifepristone, and probiotics; Drugs that modulate the Kynurenine Pathway (KP); Drugs that modulate the limbic and paralimbic brain areas, for example, Cannabidiol (CBD); Drugs that modulate the melatonergic system, for example, Agomelatine; Fatty acids, peptides, nucleic acids and other precursor molecules, for example, alpha-omega fatty acids, Coenzyme Q10, Myo-inositol, Methylfolate, S-adenosylmethionine, Cysteamine, and Oxytocin; Monoamine oxidase inhibitors (MAOIs), for example, Isocarboxazid (Marplan®, Validus Pharmaceuticals, Inc.), Phenelzine (Nardil®, Warner-Lambert Pharmaceutical Company), Selegiline (Emsam®, Somerset Pharmaceuticals, Inc.), and Tranylcypromine (Parnate®, GlaxoSmithKline); Mood stabilizers, for example, lithium salts, Valproate, Ebselen, and Divalproex; Multimodal antidepressants, for example Vilazodone and Vortioxetine; N-Nitrosodimethylamine (NDMA)-receptor antagonists, for example, Amantadine, Arketamine, Ketamine, Memantine, Riluzole, Esketamine; Neurokinin-1 (NK1) receptor antagonists; Neuropeptide Y (NPY) receptor agonists; Drugs with Neurotrophic effects, Cilostazol, Sildenafil, and Vildagliptin; Norepinephrine-dopamine reuptake inhibitors (NDRIs), Bupropion (Wellbutrin®, GlaxoSmithKline; Zyban®, Glaxo Group Ltd.; Aplenzin®, Biovail Laboratories International); Drugs that act on the opiate system, for example, ALKS-5461, AZD2327, BTRX-246040 (LY2940094), Buprenorphine, JNJ-67953964, Nalmefene, and Naltrexone; Protein Kinase C inhibitors or anti-estrogen drugs, for example, Endoxifen, Tamoxifen, and Verapamil; Psychedelic drugs, for example, 3,4-methylenedioxymethamphetamine (MDMA), Ayahuasca, Lysergic acid diethylamide (LSD), psilocybin and/or derivatives thereof; Selective serotonin reuptake inhibitors (SSRIs), for example, Citalopram (Celexa®, Forest Laboratories Inc.), Escitalopram (Lexapro®, Forest Laboratories Inc.), Fluvoxamine, Paroxetine (Paxil; GlaxoSmithKline; Pexeva®, Synthon Pharmaceuticals Inc.), and Sertraline (Zoloft®, Pfizer); Selective norepinephrine transporter inhibitors, for example, Atomoxetine; Serotonin-norepinephrine reuptake inhibitors (SNRIs), for example, Desvenlafaxine (Pristiq®, Wyeth Corporation), Duloxetine (Cymbalta®, Eli Lily and Company), Levomilnacipran (Fetzima®, Forest Laboratories Inc.), and Venlafaxine; Stimulants, including adenosine receptor antagonists, and alpha-2-adrenergic receptor agonists, for example, Caffeine, Clonidine, Guanfacine, Extended Release amphetamines XR-OS, Dextroamphetamine sulfate, Lisdexamfetamine, Methamphetamine, Mixed amphetamine salts, Racemic amphetamine sulfate, and Triple-bead mixed amphetamine salts; Substance P-antagonists, for example, MK-0869; Tricyclic serotonin-norepinephrine reuptake inhibitors, for example, Amitriptyline (Elavil®, Merck and Co.), Amoxapine, Buspirone (Buspar, Bristol Meyers Squibb), Clomipramine, Desipramine (Norpramin®, Lakeside Laboratories Inc.; Aventis, Aventis Corporation), Doxepin, Imipramine (Tofranil®, Mallinckrodt LLC; Ciba Geigy, Maria Sistro), Maprotiline, Nortriptyline (Pamelor, American Home Products of Delaware), Protriptyline, and Trimipramine; and Vasopressin 1B (V1B) receptor antagonists, for example, SSR149415.

Thus, the MEVs, via IN administration can deliver therapeutic and diagnostic molecules that include peptides, small peptides, proteins, polypeptides, nucleic acids, and small molecules. Such cargo includes, but is not limited to, anti-cancer therapeutics and diagnostics, psychoactive molecules, such as psilocybin/psilocin, other molecules including harmine, temozolomide, rivastigmine, rhodamine (small molecules); catalase, GFP, nerve growth factors (NGFs), TrkA (tropomyosin kinase A), neurotrophic factors (NT-3, NT-4, BDNF (brain derived neurotrophic factor, CNTF (ciliary neurotrophic factor), EPO, IGF-1, bFGF (basic fibroblast growth factor), hGH, and luciferase (proteins), to the brain by the IN route using any kind of EVs. The expression, in the brain, of an active mRNA, siRNA, or miRNA for GABABIA receptor, eGFP and Luciferase (mRNA); nerve growth factors (NGFs), TrkA (tropomyosin kinase A), neurotrophic factors (NT-3, NT-4, BDNF (brain derived neurotrophic factor, CNTF (ciliary neurotrophic factor), EPO, IGF-1, bFGF (basic fibroblast growth factor), hGH, GABABIA receptor (siRNA), PTEN (siRNA); miR-17 (miRNA); and MALAT1 (lncRNA) carried by the EVs as a payload. MALATI can be used to treat brain injury and trauma; it also is over-expressed in some conditions, such as cancer, and can be a target for inhibition.

Of interest is delivery of psychoactive drugs for treatment of psychiatric and other such disorders. Exemplary bioactive molecules and drugs that can be loaded in MEVs for such treatments are set forth in the following table.

| | Drug Classification or Mechanism | |
|---|---|---|
| Bioactive molecule or Drug | of Action | Condition(s) |
| 3,4-methylenedioxymeth- | Psychedelic; induces short-term | Anxiety, Bipolar |
| amphetamine (MDMA; “ecstasy”) | release of serotonin, dopamine, and/or | Disorder, |
| | norepinephrine | Schizophrenia |
| 5-hydroxytryptamine (5-HT₁) | Agonizes 5-hydroxytryptamine | Schizophrenia |
| receptor agonists | receptors or serotonin receptors | |
| AGN-241751 | Glutamatergic modulation | Depression |
| Agomelatine | Modulates melatonergic system | Depression |
| ALKS-5461 | Opiate system | Depression |
| Amantadine | NDMA-receptor antagonist | Autism |
| Amitriptyline (Elavil ®, Merck | Tricyclic: serotonin-norepinephrine | Anxiety, |
| and Co.) | reuptake inhibitors | Depression, Eating |
| | | Disorders |
| Amoxapine | Tricyclic: serotonin-norepinephrine | Anxiety, Depression |
| | reuptake inhibitors | |
| Amphetamines: | Stimulant; Increases dopamine and | ADHD |
| (Extended Release amphetamines | norepinephrine; inhibits monoamine | |
| XR-OS, EROS; | oxidase; interacts with ACH, 5-HT, | |
| Dextroamphetamine sulfate; | opioid, and glutamate pathways | |
| Lisdexamfetamine; | | |
| Methamphetamine; Mixed | | |
| amphetamine salts; Racemic | | |
| amphetamine sulfate; Triple-bead | | |
| mixed amphetamine salts) | | |
| Aripiprazole | Anti-psychotic | Autism, Bipolar |
| | | Disorder, Borderline |
| | | Personality Disorder, |
| | | Schizophrenia |
| Arketamine | NDMA-receptor antagonist; | Bipolar Disorder |
| | Glutamatergic modulation | |
| Asenapine | Anti-psychotic | Bipolar Disorder, |
| | | Schizophrenia |
| Aspirin | Anti-inflammatory; COX-1 and COX- | Bipolar Disorder, |
| | 2 inhibitor | Depression |
| Atomoxetine | Selectively inhibits NE transporter; | ADHD |
| | increases extracellular synaptic levels | |
| | of NE and dopamine in prefrontal | |
| | cortex | |
| AV-101 | Glutamatergic modulation | Depression |
| AVP-786 | Glutamatergic modulation | Depression |
| AVP-923 | Glutamatergic modulation | Depression |
| AXS-05 | Glutamatergic modulation | Depression |
| Ayahuasca | Psychedelic effects-Multiple | Depression |
| | mechanisms | |
| Azapirones | 5-HT_{1A} agonists | Anxiety |
| AZD2327 | Opiate system | Depression |
| Benzodiazepines | GABAergic system | Anxiety, Bipolar |
| | | Disorder |
| Beta blockers/Azapirones | Beta blockers | Anxiety |
| Biperiden | Cholinergic system | Depression |
| Brexanolone | GABAergic system | Depression |
| BTRX-246040 (former | Opiate system | Depression |
| LY2940094) | | |
| Buprenorphine | Opiate system | Depression |
| Bupropion (Wellbutrin ®, | norepinephrine-dopamine reuptake | Anxiety, Depression |
| Zyban ®, Aplenzin ®) | inhibitors (NDRIs) | |
| Buspirone (Buspar ™, Bristol | Tricyclic: serotonin-norepinephrine | Depression, Eating |
| Myers Squibb) | reuptake inhibitors | disorders |
| Caffeine | Stimulant, antagonism of adenosine | OCD |
| | receptors | |
| Cannabidiol (CBD) | Acts directly on limbic and paralimbic | Anxiety |
| | brain areas | |
| Carbamazepine | Anticonvulsants | Bipolar Disorder |
| Cariprazine | Anti-psychotic | Bipolar Disorder, |
| | | Schizophrenia |
| Celecoxib | Anti-inflammatory; COX-2 inhibitor | Bipolar Disorder |
| Chlorpromazine | Anti-psychotic | Bipolar Disorder, |
| | | Schizophrenia |
| Cilostazol | Neurotrophic effects | Depression |
| Citalopram (Celexa ®) | Selective Serotonin Reuptake | Anxiety, |
| | Inhibitor (SSRI) | Depression, OCD, |
| | | PTSD |
| Clomipramine | Tricyclic: serotonin-norepinephrine | OCD, Autism |
| | reuptake inhibitors | |
| Clonidine | Stimulant; α-2 receptor agonists | Autism |
| Clonidine-XR | Stimulates post-synaptic α-2- | ADHD |
| | adrenergic receptors | |
| Clozapine | Anti-psychotic | Bipolar Disorder, |
| | | Schizophrenia, |
| | | Autism |
| Coenzyme Q10 | Miscellaneous; Anti-oxidant, aides in | Bipolar Disorder |
| | metabolism | |
| Corticotropin Releasing Factor | Corticotropin Releasing Factor (CRF) | Anxiety |
| (CRF) antagonists | antagonists | |
| Cysteamine | Miscellaneous; Precursor Molecule; | Depression |
| | Neurotrophic effects | |
| D-cycloserine | Glutamatergic modulation | Depression |
| Desipramine (Norpramin ®) | Tricyclic: serotonin-norepinephrine | Anxiety, Depression |
| | reuptake inhibitors | |
| Desipramine (Norpramin ®, | Tricyclic: serotonin-norepinephrine | Eating disorders |
| Aventis) | reuptake inhibitors | |
| Desvenlafaxine (Pristiq ®) | serotonin-norepinephrine reuptake | Anxiety, Depression |
| | inhibitors (SNRIs) | |
| Dexamethasone | anti-inflammatory, | PTSD |
| | immunosuppressive; antiemetic | |
| Dextromethorphan | Glutamatergic modulation | Depression |
| Divalproex | Mood stabilizers | Borderline |
| | | Personality Disorder |
| Donepezil | Acetylcholinesterase inhibitors | Autism |
| Doxepin | Tricyclic: serotonin-norepinephrine | Anxiety, Depression |
| | reuptake inhibitors | |
| Duloxetine (Cymbalta ®) | serotonin-norepinephrine reuptake | Anxiety, |
| | inhibitors (SNRIs) | Depression, |
| | | Borderline |
| | | Personality Disorder |
| Ebselen | Mood stabilizer; Lithium mimetic | Bipolar Disorder |
| Endoxifen | Protein Kinase C inhibitor, anti- | Bipolar Disorder |
| | estrogen | |
| Escitalopram (Lexapro ®) | Selective Serotonin Reuptake | Anxiety, |
| | Inhibitor (SSRI) | Depression, OCD, |
| | | PTSD, Autism |
| Esketamine | NDMA-receptor antagonist; | Bipolar Disorder, |
| | Glutamatergic modulation; mu-opioid | Depression |
| Eslicarbazepine | Anticonvulsants | Bipolar Disorder |
| Fludrocortisone | Hypothalamic-Pituitary-Adrenal | Depression |
| | (HPA) axis | |
| Fluoxetine (Prozac ®) | Selective serotonin | Anxiety, |
| | | Depression, |
| | | Borderline |
| | | Personality |
| | | Disorder, OCD, |
| | | PTSD, Autism, |
| | | Eating Disorders |
| Fluvoxamine | Selective Serotonin Reuptake | Depression, OCD, |
| | Inhibitor (SSRI) | Autism |
| Gabapentin | Alpha-delta calcium channel | Anxiety, PTSD |
| | anticonvulsants | |
| Galantamine | Acetylcholinesterase inhibitors | Autism |
| Glycine | Miscellaneous; Amino acid, precursor | OCD |
| | molecule | |
| Guanfacine-XR | Stimulates post-synaptic alpha-2A- | ADHD |
| | adrenergic receptors | |
| Guanfacine | Stimulant; Alpha-2 receptor agonists | Autism |
| Haloperidol | Anti-psychotic | Bipolar Disorder, |
| | | Borderline |
| | | Personality Disorder |
| Hydrocortisone | Glucocorticoid receptor agonists | PTSD |
| ibuprofen | Anti-inflammatory; COX-1 and COX- | Bipolar Disorder, |
| | 2 inhibitor | Depression |
| Imipramine (Tofranil ®, Ciba | Tricyclic: serotonin-norepinephrine | Anxiety, |
| Geigy) | reuptake inhibitors | Depression, Eating |
| | | disorders |
| Infliximab | Anti-inflammatory; TNF alpha | Depression |
| | inhibitor; Immune/inflammatory | |
| | system | |
| Isocarboxazid (Marplan ®) | monoamine oxidase inhibitors | Anxiety, Depression |
| | (MAOIs) | |
| JNJ-67953964 | Opiate system | Depression |
| Ketamine | NDMA-receptor antagonist; | Anxiety, |
| | Glutamatergic modulation | Depression, OCD, |
| | | PTSD, Bipolar |
| | | Disorder |
| Lamotrigine | Anticonvulsants; modulates neuronal | Bipolar Disorder, |
| | glutamate levels | OCD, Autism |
| Levetiracetam | Anticonvulsants | Autism |
| Levomilnacipran (Fetzima ®) | serotonin-norepinephrine reuptake | Anxiety, Depression |
| | inhibitors (SNRIs) | |
| Licarbazepine | Anticonvulsants | Bipolar Disorder |
| Lithium salts | Mood stabilizer | Bipolar Disorder |
| lumateperone tosylate (Caplyta ®) | Anti-psychotic | Bipolar Disorder, |
| | | Schizophrenia |
| Lysergic acid diethylamide (LSD) | Psychedelic effects-Multiple | Anxiety, Depression |
| | mechanisms | |
| Memantine | NDMA-receptor antagonist; | Bipolar Disorder, |
| | Glutamatergic modulation | Autism, OCD |
| Methylfolate supplementation | Miscellaneous; Precursor molecule, | Depression |
| | nutritional supplement | |
| Methylphenidate: | 5-HT1a receptor agonist; psycho- | ADHD, Autism |
| Dexmethylphenidate ® | stimulant | |
| Metyrapone | Hypothalamic-Pituitary-Adrenal | Depression |
| | (HPA) axis | |
| Mifepristone | Hypothalamic-Pituitary-Adrenal | Depression |
| | (HPA) axis | |
| Minocycline | Anti-inflammatory; Immune | Bipolar Disorder, |
| | Modulator; Alpha-2 receptor | Depression, OCD, |
| | antagonist; Activation of immune | Anxiety |
| | system | |
| MK-0869 | Substance P antagonism | Depression |
| Myo-inositol | Miscellaneous; Precursor molecule, | OCD |
| | can stimulate glucose uptake and | |
| | decrease blood sugar levels. | |
| N-Acetyl-Cysteine | Anti-inflammatory, anti-oxidant with | Bipolar Disorder, |
| | glutamate-modulating properties | OCD |
| Nalmefene | Opiate system | Borderline |
| | | Personality Disorder |
| Naltrexone | Opiate system | Depression, Autism, |
| | | Borderline |
| | | Personality Disorder |
| Naproxen | Anti-inflammatory; COX-1 and COX- | Bipolar Disorder, |
| | 2 inhibitor | Depression |
| Neurokinin-1 (NK₁) antagonists | Neurokinin-1 NK(sub. 1) antagonists | Anxiety, Depression |
| Neuropeptide-Y (NPY) receptor | Neuropeptide Y (NPY) receptor | Anxiety, Bipolar |
| agonists | agonists | Disorder, |
| | | Depression, PTSD |
| Nortriptyline (Pamelor ™) | Tricyclic: serotonin-norepinephrine | Anxiety, Autism, |
| | reuptake inhibitors | Depression |
| Olanzapine | Anti-psychotic | Bipolar Disorder, |
| | | Borderline |
| | | Personality |
| | | Disorder, PTSD, |
| | | Schizophrenia |
| Omega-3 Polyunsaturated Fatty | Anti-inflammatory | Borderline |
| Acids; Fish oil | | Personality |
| | | Disorder, |
| | | Depression, PTSD |
| Ondansetron (Zofran ®) | 5-HT3 receptor agonist; Anti-emetic | OCD, Eating |
| | | Disorders |
| Oxcarbazepine | Anticonvulsants | Bipolar Disorder |
| Oxytocin | Miscellaneous; Prosocial | Anxiety, Autism, |
| | Neuropeptide | Depression, PTSD, |
| | | Schizophrenia |
| Paliperidone | Anti-psychotic | Bipolar Disorder, |
| | | Borderline |
| | | Personality |
| | | Disorder, |
| | | Schizophrenia |
| Paroxetine (Paxil ®, Pexeva ®) | Selective Serotonin Reuptake | Anxiety, Autism, |
| | Inhibitor (SSRI) | Depression, OCD, |
| | | PTSD |
| Phenelzine (Nardil ®) | monoamine oxidase inhibitors | Anxiety, Depression |
| | (MAOIs) | |
| Pioglitazone | Anti-inflammatory | Bipolar Disorder |
| Prazosin | Alpha1-Adrenergic receptor | PTSD |
| | antagonist | |
| Pregabalin | Anticonvulsant | Anxiety |
| Probiotics | Hypothalamic-Pituitary-Adrenal | Anxiety, Bipolar |
| | (HPA) axis | Disorder, |
| | | Depression |
| Propanolol | Blocks beta 1&2 adrenergic receptors | PTSD |
| Protriptyline | Tricyclic: serotonin-norepinephrine | Anxiety, Depression |
| | reuptake inhibitors | |
| Psilocybin | Psychedelic effects-Multiple | Anxiety, Depression |
| | mechanisms | |
| Quetiapine | Anti-psychotic | Anxiety, Bipolar |
| | | Disorder, Borderline |
| | | Personality Disorder |
| Rapastinel | Glutamatergic modulation | Depression |
| Riluzole | NDMA-receptor antagonist; | Anxiety, OCD |
| | Glutamatergic modulation | |
| Risperidone | Anti-psychotic | Anxiety, Autism, |
| | | Bipolar Disorder, |
| | | OCD, PTSD, |
| | | Schizophrenia |
| Rivastigmine | Acetylcholinesterase inhibitors | Autism |
| S-adenosylmethionine (SAMe) | Miscellaneous; Precursor molecule | Depression |
| SAGE-217 | GABAergic system | Depression |
| Sarcosine | Glycine reuptake inhibitor | OCD |
| Scopolamine | Cholinergic system | Depression |
| Selegiline (Emsam ®) | monoamine oxidase inhibitors | Anxiety, Depression |
| | (MAOIs) | |
| Sertraline (Zoloft ®) | Selective Serotonin Reuptake | Anxiety, Autism, |
| | Inhibitor (SSRI); Atypical Anti- | Borderline |
| | psychotic | Personality |
| | | Disorder, OCD, |
| | | PTSD |
| Sildenafil | Neurotrophic effects | Depression |
| SSR149415 | Vasopressin 1b receptor antagonist | Depression |
| Tamoxifen | Protein Kinase C inhibitor, anti- | Bipolar Disorder |
| | estrogen | |
| TNF-α inhibitor | Anti-inflammatory | Bipolar Disorder |
| Topiramate (Topamax ®) | Anticonvulsant; modulates neuronal | Bipolar disorder, |
| | glutamate levels | Eating Disorders, |
| | | OCD, PTSD |
| Tranylcypromine (Parnate ®) | monoamine oxidase inhibitors | Anxiety, Depression |
| | (MAOIs) | |
| Trimipramine. Tetracyclic: | Tricyclic: serotonin-norepinephrine | Anxiety |
| Maprotiline | reuptake inhibitors | |
| Valproate | Mood stabilizer | Bipolar Disorder |
| Valproic acid and derivatives | Anticonvulsants | Autism, Bipolar |
| | | Disorder, Borderline |
| | | Personality Disorder |
| Vasopressin (V1B) antagonists | Vasopressin (V1B) antagonists | Anxiety, Depression |
| Venlafaxine | serotonin-norepinephrine reuptake | Anxiety, Autism, |
| | inhibitors (SNRIs) | Depression, OCD, |
| | | PTSD |
| Verapamil | Protein Kinase C inhibitor, anti- | Bipolar Disorder |
| | estrogen | |
| Vilazodone | Multimodal antidepressant effect | Depression |
| Vildagliptin | Neurotrophic effects | Depression |
| Vortioxetine | Multimodal antidepressant effect | Depression |
| Ziprasidone | Anti-psychotic | Bipolar Disorder, |
| | | Schizophrenia |

Thus provided herein are methods for using MEVs for the delivery of drugs, via intranasal administration, for the treatment of psychiatric conditions, brain diseases, disorders, and conditions of the CNS. As an example, the table below summarizes: (1) the brain regions reported to be sites of action for the biological activity of psilocybin (or derivatives) and other psychedelics; and (2) regions that are also targeted by MEVs when administered by the IN route.

TABLE
Neuroanatomy of brain areas targeted by hallucinogens and MEVs

| | MEV | | |
|---|---|---|---|
| Brain area | density | Psychedelic drug target | References |
| Olfactory bulb | +++ | Existence of binding sites with | (Grailhe et al. (1999) Neuron |
| | | high affinity for [125I]LSD | 22(3): 581-591; Increased |
| | | that correspond to 5-HT5A | Exploratory Activity and Altered |
| | | receptors and that are | Response to LSD in Mice Lacking |
| | | concentrated in the olfactory | the 5-HT5A Receptor) |
| | | bulb, neocortex, and medial | |
| | | habenula | |
| Primary motor | + | A single dose of psilocybin | (Shao, Liao et al. (2021) Neuron |
| cortex | | triggers dendritic remodeling, | 109(16): 2535-2544; Psilocybin |
| | | enhances excitatory | induces rapid and persistent growth |
| | | neurotransmission in the | of dendritic spines in frontal cortex |
| | | medial prefrontal cortex | in vivo) |
| Piriform cortex | ++ | SD and DOI act as highly | (Marek and Aghajanian (1996) Eur. |
| | | potent partial agonists at | J. Pharmacol. 305(1-3): 95-100; |
| | | cortical 5HT21 receptors in the | Alpha 1B-adrenoceptor-mediated |
| | | piriform cortical cortex | excitation of piriform cortical |
| | | | interneurons) |
| Frontal cortex | + | We found that a single dose of | (Shao et al. (2021) Neuron |
| | | psilocybin led to | 109(16): 2535-2544; Psilocybin |
| | | approximately 10% increases | induces rapid and persistent growth |
| | | in spine size and density, | of dendritic spines in frontal cortex |
| | | driven by an elevated spine | in vivo) |
| | | formation rate. The structural | |
| | | remodeling occurred quickly | |
| | | within 24 h and was persistent | |
| | | 1 month later. | |
| | | Studies show that a single | (de Vos et al. (2021) Frontiers in |
| | | administration of a psychedelic | Psychiatry 12: 724606; Psychedelics |
| | | produces rapid changes in | and Neuroplasticity: A Systematic |
| | | plasticity mechanisms on a | Review Unraveling the Biological |
| | | molecular, neuronal, synaptic, | Underpinnings of Psychedelics) |
| | | and dendritic level. | |
| Agranular | + | Psilocybin and ketamine | (Davoudian, Shao et al. (2022) |
| insular cortex | | produced acutely comparable | bioRxviv, doi: |
| | | elevations in c-Fos expression | doi.org/10.1101/2022.03.18.484437; |
| | | in numerous brain regions, | Shared and distinct brain regions |
| | | including anterior cingulate | targeted for immediate early gene |
| | | cortex, locus coeruleus, | expression by ketamine and |
| | | primary visual cortex, central | psilocybin) |
| | | and basolateral amygdala, and | |
| | | claustrum | |
| Primary | + | The region containing the | (Lopez-Gimenez and Gonzalez- |
| somatosensory | | highest density of 5- | Maeso (2018) Curr. Top Behav. |
| cortex | | HT_{2A} binding sites was the | Neurosci. 36: 45-73; Hallucinogens |
| | | neocortex, where the | and Serotonin 5-HT2A Receptor- |
| | | autoradiographic signal | Mediated Signaling Pathways) |
| | | displayed a banded pattern | |
| | | corresponding predominantly | |
| | | to pyramidal neurons | |
| | | distributed according to the | |
| | | cytoarchitectural organization | |
| | | of different cortical areas | |
| Auditory cortex | + | Psychedelic drugs also have | (Barrett et al. (2018) Intl. Rev. |
| | | notable effects on the | Psychiatry 30(4): 350-362; |
| | | perception of music. This is | Psychedelics and music: |
| | | not surprising, as | neuroscience and therapeutic |
| | | 5HT_{2A} signaling has been | implications) |
| | | shown to alter neuronal | |
| | | responses to auditory stimuli | |
| | | from the cochlear nucleus | |
| | | along the precortical primary | |
| | | auditory sensory pathway | |
| | | through to primary auditory | |
| | | cortex | |
| | | LSD enhances the emotional | (Kaelen et al. (2015) |
| | | response to music. | Psychopharmacology (Berl) |
| | | | 232(19): 3607-14; LSD enhances the |
| | | | emotional response to music) |
| | | Single oral administration of | (Preller et al. (2017) Curr. Biol. |
| | | LSD (100 μg) increased blood | 27(3): 451-457; The Fabric of |
| | | oxygen-level in the prefrontal | Meaning and Subjective Effects in |
| | | cortex in response to music, | LSD-Induced States Depend on |
| | | suggesting an exaggerated | Serotonin 2A Receptor Activation) |
| | | activity in brain regions related | |
| | | to personal relevance | |
| | | processing | |
| Retrosplenial | + | Correlation of psilocybin's | (Davoudian et al. (2022) bioRxviv, |
| granular cortex | | effects with gene expression | doi.org/10.1101/2022.03.18.484437; |
| | | highlighted potential roles of | Shared and distinct brain regions |
| | | 5-HT2A and GluN2B subunit | targeted for immediate early gene |
| | | of NMDA receptors | expression by ketamine and |
| | | | psilocybin) |
| Temporal | + | | |
| association | | | |
| cortex | | | |
| Ectorhinal | + | | |
| cortex | | | |
| Amygdaloid | +++ | Acute treatment with | (Krachenmann et al. (2015) Biol. |
| nucleus | | psilocybin decreased amygdala | Psychiatry 78(8): 572-581; |
| | | reactivity during emotion | Psilocybin-Induced Decrease in |
| | | processing and that this was | Amygdala Reactivity Correlates |
| | | associated with an increase of | with Enhanced Positive Mood in |
| | | positive mood in healthy | Healthy Volunteers) |
| | | volunteers | |
| | | The administration of LSD | (Mueller et al. (2017) Transl. |
| | | reduced reactivity of the left | Psychiatry 7(4): e1084; Acute effects |
| | | amygdala and the right medial | of LSD on amygdala activity during |
| | | prefrontal cortex relative to | processing of fearful stimuli in |
| | | placebo during the | healthy subjects) |
| | | presentation of fearful faces | |
| | | LSD administration produced | (Gresch et al. (2002) Neuroscience |
| | | a five- to eight-fold increase in | 114(3): 707-713; Lysergic acid |
| | | Fos-like immunoreactivity in | diethylamide-induced |
| | | medial prefrontal cortex, | Fos expression in rat brain: role of |
| | | anterior cingulate cortex, and | serotonin-2A Receptors) |
| | | central nucleus of amygdala. | |
| | | Thus, we show psilocybin's | (Grimm et al. (2018) Eur. Neuro- |
| | | effect as a modulator of major | psychopharmacol. 28(6): 691-700; |
| | | connectivity hubs of the | Psilocybin modulates functional |
| | | amygdala. | connectivity of the amygdala during |
| | | | emotional face discrimination) |
| | | Local infusion of the 5-HT2A | (Pedzich et al. (2022) |
| | | receptor antagonist M100907 | Neuropsychopharmacology |
| | | into the amygdala reversed the | 47: 1304-1314; Effects of a |
| | | effect of systemic | psychedelic 5-HT2A receptor |
| | | administration of DOI on fear | agonist on anxiety-related behavior |
| | | expression while local | and fear processing in mice) |
| | | administration of DOI into the | |
| | | amygdala was sufficient to | |
| | | suppress fear expression. | |
| | | The correlation of fear | (Dolder et al. (2016) Neuropsycho- |
| | | perception and amygdala | pharmacology 41(11): 2638-2646; |
| | | reactivity may be useful during | LSD Acutely Impairs Fear |
| | | psychotherapy. | Recognition and Enhances |
| | | | Emotional Empathy and Sociality) |
| Arcuate | +++ | | |
| hypothalamic | | | |
| nucleus | | | |
| Mammillary | +++ | Rat brain exhibited a high | (Watts et al. (1994) Neurochem. Intl. |
| nucleus | | level of specific binding of | 24(6): 565-574; Autoradiographic |
| | | [125I]LSD in the tenia tecta | comparison of [125I]LSD-labeled 5- |
| | | and mammillary nuclei. | HT2A receptor distribution in rat |
| | | | and guinea pig brain) |
| | | High concentrations of | (Nakada et al. (1984) Neuroscience |
| | | [¹²⁵I]LSD sites were observed | Letters 49(1-2): 13-18; Localization |
| | | in layer IV of the cerebral | and characterization by quantitative |
| | | cortex, caudate-putamen, | autoradiography of [125I]LSD |
| | | claustrum, olfactory tubercle, | binding sites in rat brain) |
| | | nucleus accumbens, | |
| | | ependyma, mammillary | |
| | | nucleus and inferior olive. | |

Because the target regions for psychedelics and the target regions for MEVs upon IN administration coincide, it is described and shown herein that this can be exploited for treatment of psychiatric and other diseases, disorders, and conditions that are or can be treated by administration of a psychedelic. Disorders responsive to treatment by psychedelics include, but are not limited to, PTSD and depression, among others. Advantages that are associated with the administration of therapeutical psychedelics by means of MEV-mediated nose-to-brain delivery, include, but are not limited to, the following:
1) Direct entry to the brain avoids passage through the blood stream, which leads to systemic exposure of the entire body, degradation, metabolism, and clearance by filtering organs (liver, kidneys).
2) Protection of psilocybin and/or derivatives thereof against oxidative inactivation in the body (kidney, intestinal mucosa).
3) Avoidance of undesirable effects in the gastrointestinal tract (the gastrointestinal tract is among the tissues with the highest level of expression of the 5-HT2 receptor) and other organs (liver, kidneys, and cardiovascular system) expressing levels of the receptor that are significantly higher than those in the brain.
4) Better control of the timing involved in the process (from the administration of the drug to the end of the treatment), and lower variability from patient to patient due to the fluctuations introduced by the oral administration and the passage through the gastrointestinal tract, and blood.

As shown herein, MEV-mediated delivery provides entry to the brain via the olfactory nerve, resulting in the distribution of a psychoactive drug, such as psilocybin and/or derivatives thereof, via the mitral/tufted axons throughout the olfactory tract, and, accordingly, directly to the relevant brain regions. Thus provided herein are methods for using MEVs for the delivery of psychiatric drugs such as psychedelics and others, via intranasal administration, for the treatment of psychiatric conditions.

### I. Mev-Mediated Intracellular Signaling

Other in vivo therapeutic targets, in general, not limited to the brain, include receptors that are involved in a disease, disorder, or condition. These include cell surface receptors, and internalized receptors, in which the MEVs deliver agonists, antagonists, ligands, or other modulators of activity. Targets of interest include, for example, modulation of toll-like receptors (TLRs) and other receptors, including receptors, such as the TLRs, that are internalized. Such receptors are internalized into vesicles and can interact with ligands delivered by MEVs. This is exemplified in the Examples in which the ligand flagellin is delivered into cells via MEVs; flagellin activates TLR-5, which in turn activations inflammatory cytokines.

Flagellin is a conserved protein, a component of the bacterial flagellum in mobile bacteria. The immune systems in plants and in animals that assure the defense against bacterial infections are set to recognize flagellin, or subrogate peptides thereof, and to react against the invading bacteria. Thus, the presence of bacteria, their flagella, or of fragments thereof triggers a signaling pathway, that leads to a reaction of the host to the putative infection agent. In plants, flagellin is recognized by the FLS2 receptor (Flagellin Sensing 2 receptor). For instance, the presence of flagellin, or of surrogate peptides of it, is detected by the leaf epithelium that surrounds the stomata (the respiratory pores on the surface of the leaves). Open stomata are entry doors into the leaf parenchyma for infective agents such as flagellin-bearing bacteria. When the presence of flagellin is detected in the leaf epithelium, the plant triggers an immune response, which includes the immediate closing of the stomata in order to physically prevent the entry of bacteria thereby. The signaling pathway, that starts by the detection of flagellin (or of subrogate peptides) and ends with the closing of the stomata, is triggered by the binding of flagellin (or of subrogate peptides) to the FLS2 receptor. The transmembrane protein receptor, FLS2, is the very first component in the signaling pathway. In Arabidopsis thaliana, a species commonly used as a plant model, the FLS2 receptor is found in the plasma membrane and in the membranes of endosomal vesicles inside the plant cell (see, Beck et al., (2012) The Plant cell 24 (10): 4205-4219 and Otegui et al. (2008) Traffic (Copenhagen, Denmark) 9 (10): 1589-1598). The flagellin binding domains are oriented either to the “extracellular space” (for the FLS2 molecules located in the plasma membrane); or to the “intra-endosomal space” (for the FLS2 molecules located in intracellular endosomal vesicles). The FLS2 domains in charge of the triggering of the signaling pathway are in both cases oriented towards the cytoplasmic side of the membranes. Thus, it can be expected that a signaling pathway and the subsequent biological immune response triggered by the binding of flagellin (or of a subrogate peptide) to FLS2 are indistinguishable whether the triggering FLS2 is located in the plasma membrane and detects flagellin in the cell surface or located in an endosomal vesicle and detects flagellin from within the same endosomal vesicle. Although, it is straightforward for FLS2 located in the plasma membrane to identify and bind to flagellin (or to subrogate peptides), it is less likely to expect that FLS2 will find flagellin (or a subrogate peptide) inside the endosomal vesicle where the intracellular FLS2 form is located. Experiments shown in the working Examples herein, demonstrate that MEVs that have been loaded with flp22, a subrogate peptide of flagellin, can trigger the expected biological immune reaction, i.e. the immediate closing of the stomata, presumably by the delivery of the bioactive peptide straight into the endosomal vesicles where FLS2 is located. As flp22-loaded MEVs are treated with proteases to destroy any trace of external flp22 that may remain, if exo-loaded, from the loading reaction; the immune reaction cannot be explained by free flp22 binding to the FLS2 in the plasma membrane. There is no evidence that MEVs (or other EVs) spontaneously release their cargo in the extracellular or culture medium that might trigger a signaling pathway from the FLS2 in the plant's membrane. Thus, the observed data indicated that flp22-loaded MEVs are endocytosed by the epithelial plant cells, and that once inside the cells the endocytic vesicles carrying the loaded-MEVs find and fuse with endosomal vesicles that carry the FLS2 protein.

The flg22 peptide, is a 22-amino acid synthetic peptide, which mimics a conserved N-terminal region of bacterial flagellin. It has been shown that flp22 binds to plasma membrane FLS2 triggering a defense response of the cell against bacteria. However, it has never been shown in the prior art that free flp22 can be delivered directly to endosomal vesicles or that from there it can trigger the same biological response that it triggers from the cell surface. Effective delivery of the peptide inside the endosomal vesicles, mediated by peptide loaded-MEVs, allows such phenomenon to take place and to be observed.

Multiple clathrin-independent mechanisms of endocytosis have been described and characterized; some of which play a role in membrane bulk flow and cell membrane turnover. It is also known that FLS2 can be internalized into vesicles, not only upon ligand binding, but also in a ligand-independent manner for constitutive recycling (see, Beck et al., (2012) The Plant cell 24 (10): 4205-4219). FLS2 signaling also occurs from endosomes after internalization (see, Otegui et al. (2008) Traffic (Copenhagen, Denmark) 9 (10): 1589-1598). Plant endosomes are highly dynamic organelles; hence a rendezvous of ligand-carrying MEVs and receptor-carrying vesicles is possible inside the cell. This intracellular interaction results in fusion of MEVs and endosomes, providing ligand-mediated activation of FLS2 and production of the effector signaling. Similar mechanisms of endosome turnover are present in yeast and mammals, where endosomes are also known to recycle vacuolar cargo receptors back to the trans Golgi network and sort membrane proteins for degradation in the vacuole/lysosome.

These results indicate that when a MEV loaded with a ligand (agonist or antagonist) for either an internalized receptor, or for an endosomal intracellular receptor, such as the FLS2 receptor, the TLRs or alike, are internalized, as in the Examples, they may happen to reach and deliver their payload inside the vesicles where the target receptors are located and thus elicit a biological response from “within the cell”. Thus, MEVs can deliver agonists/antagonists or ligands to internalized or to intracellular receptors. No other alternative technologies have shown to be able of such an endeavor.

A significant relevance of the above observations comes from the fact that the plant FLS2 protein is a member of the large family of the so-called TLRs (or Toll-like Receptors), which play a pivotal role in innate immunity and in the modulation and triggering of immune responses in humans, mammals, and other animals.

There are similarities between how plants and animals perceive pathogens. Plants have highly sensitive perception systems that stimulate defense responses, and innate immunity in animals is based on the recognition of similar pathogen-associated molecular patterns. FLS2 in Arabidopsis, which is essential for flagellin perception, shares homology with the Toll-like receptor (TLR) family, which is a first line of defense against infectious diseases in animals (see, Gómez-Gómez et al. (2002) Trends in plant science 7 (6): 251-256).

TLR5 is responsible for flagellin perception in mammals, but other receptors from this family, including TLR3, TLR7, TLR8, TLR9, TLR13, (naturally located in cellular endocytic vesicles) also can be targets for intracellular MEV-mediated ligand delivery. This phenomenon paves the way for treatment of a number of therapeutic indications using specific ligand-loaded MEVs to trigger the immunomodulatory signaling pathways related to the different TLR family members, such as for infections (like sepsis and other), for inflammation (like rheumatoid arthritis and other), as vaccine adjuvants (including cancer vaccines) as well as for allergy treatment.

The following tables summarizes TLR members, their ligands and agonists/antagonists, and downstream effects:

| TLR | | |
|---|---|---|
| Member | Ligand(s)/Agonists | Actions/Downstream effects |
| TLR1 | Triacyl lipopeptides | Myeloid differentiation primary response |
| | (Pam3CSK4) | (MyD88)* and MAL/TIRAP** -dependent |
| | | activation of inflammatory cytokines, NF-KB and |
| | | AP-1; Induces phagocytosis of ligand, or bound |
| | | molecules |
| TLR2 | Zymosan, Porin, Modulin, | MyD88 and MAL/TIRAP-dependent activation of |
| | Lipoproteins, Lipotechoic acid, | inflammatory cytokines, NF-KB and AP-1; |
| | Diacyl lipopeptides, Atypical | Induces phagocytosis of ligand, or bound |
| | LPS, Peptidoglycan, Triacyl | molecules |
| | lipopeptides | |
| TLR3 | dsRNA | Trif dependent induction of Inflammatory |
| | | cytokines and Type I IFN; Initiates adaptive |
| | | immunity; Activates local cytokine burst and local |
| | | inflammatory response; Production of TNF-alpha, |
| | | IL-12, MCP1. |
| TLR4 | Mannans, Taxol, LPS | Trif and TRAM dependent induction of |
| | | inflammatory cytokines and Type -I IFN, TIRAP |
| | | and MyD88 induction of inflammatory cytokines; |
| | | Promotes addiction; Neuroinflammatory response |
| TLR5 | bacterial flagellin, profilin, | MyD88-dependent induction of NF-kB and |
| | HMGB1, Small molecule | inflammatory cytokines, activation of innate and |
| | agonists (CBLB502) | adaptive immunity |
| TLR6 | Zymosan, Porin, Modulin, | MyD88 and MAL/TIRAP-dependent activation of |
| | Lipoproteins, Lipoteichoic acid, | inflammatory cytokines, NF-KB and AP-1; |
| | Diacyl lipopeptides | Induces phagocytosis of ligand, or bound |
| | (Pam2CSK4), Atypical LPS, | molecules |
| | Peptidoglycan | |
| TLR7 | imidazoquinolinone, loxoribine, | MyD88 dependent induction of inflammatory |
| | ssRNA, bropirimine, resiquimod | cytokines and Type I IFN |
| TLR8 | ssRNA, small synthetic | MyD88 dependent induction of inflammatory |
| | compounds | cytokines and Type I IFN |
| TLR9 | CpG DNA | MyD88 dependent induction of inflammatory |
| | | cytokines and Type I IFN |
| TLR10 | Diacyl and Triacyl lipopeptides | TRIF-dependent activation of IFN-B; TRADD |
| | | dependent and/or MyD88 and TIRAP-dependent |
| | | activation of pro-inflammatory cytokines; |
| | | PI3K/Akt dependent activation of IL-1Ra; Direct |
| | | inhibition of MyD88 or MAPK--Anti- |
| | | inflammatory; |
| TLR11 | Profilin-like protein, non- | MyD88 dependent induction of inflammatory |
| | pathogenic bacteria | cytokines |

*TIR-containing adaptor protein (Tirap), also called MyD88 adaptor-like protein (MAL)
**toll/Interleukin-1 receptor domain containing adaptor protein

| TLR | | |
|---|---|---|
| Member | Ligand(s)/Antagonists | Action/Downstream Effects |
| TLR1 | Small molecule antagonists | Competitive inhibition of signaling cascade; |
| | (CU-T12-9, MMG-11) | Inhibition heterodimerization of TLR1/2; |
| | | Induce M1 macrophages to M2 phenotype; |
| | | Inhibition of immune response; |
| | | Inhibition of inflammatory response |
| TLR2 | Small Molecule | Competitive inhibition of signaling cascade; |
| | Antagonists(AT1-AT8, CU- | Inhibition of heterodimerization of TLR1/2; |
| | CPT22, CU-T12-9, MMG-11, | Induction of M1 macrophages to M2 phenotype; |
| | NPT1220-312), Phloretin, | Reduction of immune response; |
| | Sulfoglycolipids | Reduction of inflammatory Response; |
| | | Reduction of TNF-alpha production, reduced nitric |
| | | oxide (NO) formation; Reduced TLR2-induced |
| | | NF-KB transcriptional activity; |
| | | Reduction of NLRP3 including IL-1B, and 1L-18; |
| TLR3 | Small Molecule Antagonists | Decreased production of IL-6, IL-8, MIP1-alpha, |
| | (CU-CPT4a), Monoclonal | and TNF-alpha |
| | antibodies (CNTO4685, | |
| | CNTO5429) | |
| TLR4 | Small Molecule Antagonists | Decreased NF-kB activation and cytokine |
| | (Norbinaltorphimine, T4ICs, | production, reduction of nitric oxide (NO) |
| | T5342126, Simvastatin | formation; Decreased addiction; |
| TLR5 | Small Molecule Antagonist | Decreased NF-kB, AP-1 signaling and decreased |
| | (TH1020) | induction of proinflammatory cytokines; Altered |
| | | gut microbiome, tumor growth, metabolic |
| | | syndrome, liver fibrosis; |
| TLR6 | Simvastatin | Reduction in cytokine production; Prevention of |
| | | macrophage activation; |
| TLR7 | Chloroquine, | Reduction of IFN-1 and cytokine signaling, |
| | hydroxychloroquine, quinacrine | reduction of inflammation |
| TLR8 | Small Molecule Antagonist (CU- | Reduced induction of TNF, IL-6, and IL-10 |
| | CPT8m, CU-CPT9a) | |
| TLR9 | Small Molecule | Blocks release of inflammatory cytokines in |
| | Antagonists(NPT1220-312), | monocyte/macrophage cells; decreased |
| | chloroquine, | immunostimulatory response; |
| | hydroxychloroquine, quinacrine; | |
| | Suppressive or inhibitory | |
| | oligonucleotides | |
| TLR10 | — | — |

Thus, therapeutic targets for MEV-mediated delivery and expression, include receptors involved in diseases, disorders, or conditions, such that said receptors are cell surface receptors, or, most important, internalized receptors or internal (intracellular endosomal) receptors, like TLRs and others. MEVs may deliver agonists, antagonists, ligands, or other modulators of activity of such receptors. Targets of interest include, for example, modulation of toll-like receptors (TLRs). Such receptors are internalized into endosomal vesicles and can interact with ligands delivered by MEVs. This is exemplified in the Examples in which flagellin, surrogate thereof, and other known ligands for TLRs is delivered into cells via MEVs; activation of TLRs in turn activates inflammatory cytokines.

### J. EXAMPLES

The following examples are included for illustrative purposes only and are not intended to limit the scope of the invention(s).

### Example 1A

Production of Chlorella cells and isolation of Microalgae Extracellular Vesicles (MEVs)

### A. Batch Production of the Inoculum

Chlorella vulgaris of UTEX B 265 strain or UTEX 395 or UTEX 26, or UTEX 30 or UTEX 259 or UTEX 2219 or UTEX 2714 or UTEX B 1811 (available from the UTEX Culture Collection) or the strain designated CCAP 211/19, GEPEA, University of Nantes, France, or any other suitable strain. The selected strain is genetically modified to encode and/or express a heterologous product, such as, but not limited to, one or more of RNA (coding or non-coding), small peptides, peptides, polypeptides and protein, complexes, such as editing complexes). The strains are used to produce the algal cell material. Chlorella is stored on nutrient agar slopes until flask/photobioreactor (PBR) inoculation. For different experiments, different scales of production, between 400 mL (flasks) to 170 L (several PBRs with different total volume) cultures, are used. This description relates to the highest volume of PBR used (170 L, HECTOR PBR [“Hector” photobioreactor designed by the Laboratory of Process Engineering-Environment-Agri-food (GEPEA)/CNRS for the culture of microalgae. Reference: 20160067_0017. Year of production: 2016. Maximum size: 56.43×37.66 cm/170 L/300 dpi]).

A 5-Liter PBR is filled with 4 L of sterile BG-11 medium (see, e.g., utex.org/products/bg-11-medium for a description of its preparation and see table below (Table 1) for autotrophic growth), and inoculated directly from the stock algal slope on nutrient agar. Then, the Chlorella strain is grown as a batch culture in a bubble column using the following culture parameters: temperature of 23° C.; medium pH 7.5-8.0; light intensity: 100 μmol·m⁻²·s⁻¹; light cycle: continuous. Biomass concentration, specific growth rate and biomass productivity of Chlorella are estimated daily. Typically, after 6 days of continuous growth the cultures reached biomass concentrations of approx. 1.2 g/L. The total crop volume of 20 L is collected for subsequent production scale-up to the HECTOR PBR.

TABLE 1
Composition of the BG-11 medium

| | Conc | MI |
|---|---|---|
| Salt Mix | 100x | 10 |
| Iron* | 1000x | 1 |
| Nitrate* | 100x | 10 |
| Trace metals | 1000x | 1 |
| agar | | 14 |
| H₂O | | up to 1 L |
| pH 7 | | |

*In case of precipitation, add after sterilization.

Stock solutions for BG-11 medium:

| | g/L | MW | μM |
|---|---|---|---|
| Nitrate 100x | | | |
| NaNO₃ | 150 | 85 | 1764.71 |
| Iron 1000x | | | |
| Fe-EDTA | 3 | 344.06 | 8.72 |
| Salt Mix 100x | | | |
| K₂PO₄ | 4 | 173.17 | 23.10 |
| MgSO₄•7H₂O | 7.5 | 246.48 | 30.43 |
| CaCl₂•2H₂O | 3.6 | 147.01 | 24.49 |
| Na₂CO₃ | 2 | 105.99 | 18.87 |

add HCl if needed

| | | | |
|---|---|---|---|
| Trace Metals 1000x | | | |
| H₃BO₃ | 2.9 | 61.84 | 46.25 |
| MnCl₂•4H₂O | 1.8 | 197.91 | 9.15 |
| ZnSO₄•7H₂O | 0.2 | 287.56 | 0.77 |
| Na₂MoO₄•2H₂O | 0.4 | 241.95 | 1.61 |
| CuSO₄•5H₂O | 0.1 | 249.68 | 0.32 |
| CoCl₂•6H₂O | 0.3 | 237.93 | 0.14 |

### B. Production Scale-Up in a Semi-Industrial Photobioreactor (PBR)

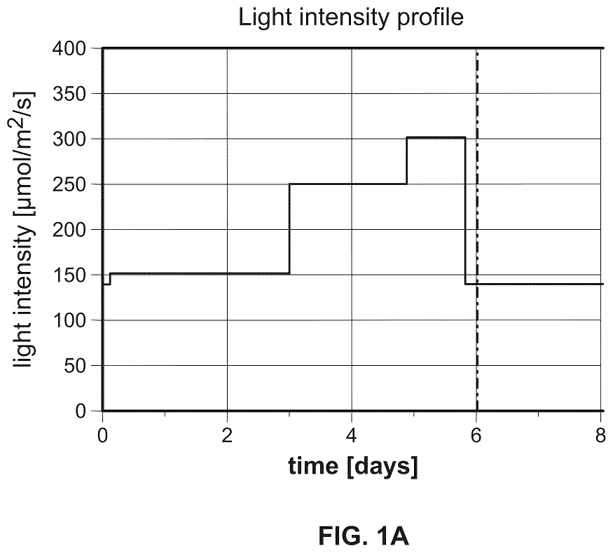

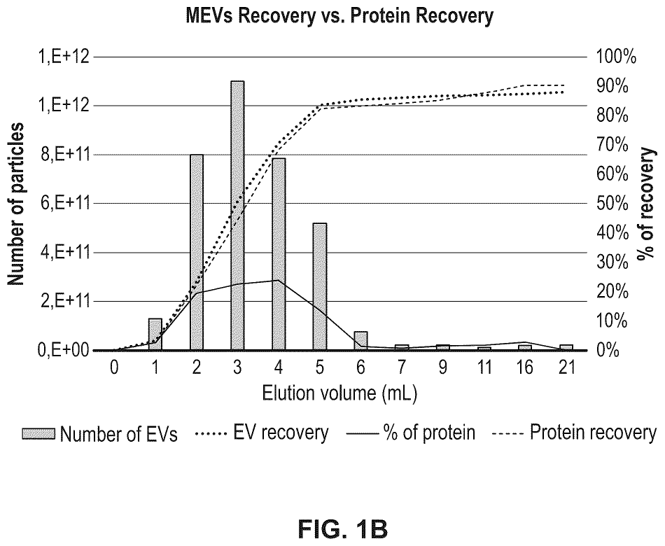

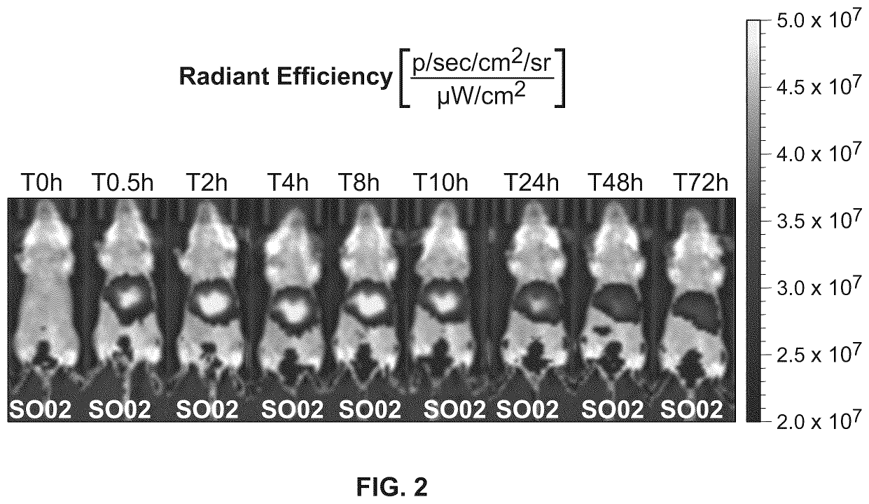

The Chlorella cells are cultured further in a 170-Liter photobioreactor system (HECTOR). The inoculum is added to sterile BG-11 medium (see Table 1) to the total volume of 150 L and the cells are grown autotrophically as a semi-batch culture with bubble column mixing. The following culture parameters are used: temperature of 18±4° C.; medium pH 8.0±0.05; light intensity: 150-300 μmol·m⁻²·s⁻¹; between 150 μmol·m⁻²·s⁻¹ the three first days of each batch, 250 μmol·m⁻²·s⁻¹ days four and five and 300 μmol·m⁻²·s⁻¹ days six and seven before the harvesting as light cycle: continuous, with gradual increase in light intensity (FIG. 1A). Biomass concentration, growth rate and biomass productivity of Chlorella are estimated daily. On the 6th day of the cultivation, at the biomass concentration of approx. 1.5 g/L Chlorella production harvesting is performed. FIG. 1A shows a profile of light intensity used in HECTOR PBR cultures.

### C. Production of 3 Consecutive Batches of Chlorella

The Chlorella production is performed in 3 semi-batches of 130 L, from which about 80% of the culture volume was aseptically removed for downstream treatment and supplemented with sterile BG-11 medium. Following the harvest, the light intensity is lowered to 150 μmol·m⁻²·s⁻¹ to avoid excessive photon intake. A seeding line is set up to go from 100 mL of culture to 150 L of culture. Three consecutive batches lasting 6-7 days are carried out with the aim of extracting a vesicle concentrate devoid of microalgae.

### Culture Parameters Monitoring

### 1. Determination of the Protein Content

The protein content of cultures is determined by elemental analysis, resorting to Vario EL III (Vario EL, Elementar Analyser systeme, GmbH, Hanau, Germany), according to the procedure provided by the manufacturer. The final protein content is calculated by multiplying the percentage of nitrogen given by the elemental analysis by 6.25.

### 2. Estimation of Chlorophyll Content

Culture samples are centrifuged at 2547 g for 15 min using a Hermle centrifuge (HERMLE Labortechnik GmbH, Wehingen, Germany). Pigments are extracted from the resulting pellet by bead milling in acetone. The full absorbance spectrum of the extract is obtained with a Genesys™ 10S UV-VIS spectrophotometer (Thermo Scientific, Massachusetts, USA) and iteratively decomposed to the standard pigment spectra to obtain the total chlorophyll content.

### 3. Growth Estimation

Dry weight is obtained by filtration of culture samples using pre-weighed 0.7 μm GF/C 698 filters (VWR, Pennsylvania, USA) and dried at 120° C. until constant mass is obtained using a DBS 60-30 electronic moisture analyzer (KERN & SOHN GmbH, Balingen, Germany). All dry weight samples are washed with demineralized water to remove growth medium salts.

### D. Isolation of Microalgae Extracellular Vesicles: Production of Concentrated MEV Preparation (Down-Stream Processing: Clarification and Concentration Step)

The culture harvested from the photobioreactor is centrifuged at 2,700 g for 5 minutes at room temperature for cell removal. The supernatant is transferred into fresh bottles and centrifuged again at 2,700 g for 5 minutes at room temperature. The clear MEV-containing solution is then subjected to membrane filtering using a 1.2 μm cut-off cartridge filter. The filtrate is concentrated with the use of a 100 kDa MWCO tangential filtration system. At each isolation step the material is analyzed spectrophotometrically for chlorophyll and particulate matter. Dry weight of the final product is <0.01 g/L and the concentration factor relative to the initial volume of the processed culture is approx. 20. Thus, obtained MEV solution is stored at −50° C. in 1 L pockets for further purification.

### E. Detailed Protocol for Purification of Chlorella Microalgae Extracellular Vesicles

The MEV preparation, clarified and concentrated and stored (1.0-1.2 L) as described in section D, is thawed in a cold room at 4° C. overnight. When the preparation is thawed, harvest the biomass by centrifugation (set the temperature to 4° C.): 2×10000 g for 10′ at 4° C. The MEV supernatant is collected and filtered by vacuum filter onto 0.65 μm filters to get rid of the remaining cells. The MEVs are concentrated and purified by tangential flow filtration (TFF) using Sartorius VivaFlow® systems. The membrane is washed by running water at ˜100 ml/minute, as described by the manufacturer. After that, the circuit is washed with cell-free medium (BG-11 medium) at ˜200 ml/minute (pressure reading at 2/2,5 bars).

The MEV preparation (supernatant) is run in the circuit at ˜200 ml/minute (pressure reading at 2/2,5 bars). When the residual volume in the circuit plus the reservoir is about 200 mL, the TFF is used to diafiltrate and change the medium from BG-11 to PBS using 1 L of PBS. When the residual volume is about 200 mL in PBS medium the flow is slowed down to ˜100 ml/minute (20 minutes, 1 bar). After TFF MEV are recovered in a volume of 30 to 60 mL.

MEVs are then filtered using 0.45 μm filters and purified by ultracentrifugation. The filtered MEVs are loaded on the ultracentrifuge tubes and centrifuged for 1 h at 4° C., at 100000 g (27400 rpm) (acceleration and deceleration at max), for example in a SorVall® WX ultra 80 TST 28.38. Pellets containing the MEVs are resuspended in 1-2 ml of PBS buffer and sterilized by filtration using a 0.2 μm filter and analyzed by nanoparticle tracking analysis (NTA; dilute up to 1:1000 before the NTA analysis).

### F. Protocol for Purification of Chlorella Microalgae Extracellular Vesicles by Size Exclusion Chromatography (SEC)

When higher purity of preparations is needed, a last step of purification is added. The MEVs previously concentrated by TFF and purified by ultracentrifugation and formulated in PBS at concentration of 10Exp11 to 10Exp13 per mL are seeded in a pre-packed column qEV1 (IZON, Lyon France). The MEVs are eluted using PBS solution. The elution fractions of 0.5 mL are collected. MEVs are recovered in the first fractions as shown in FIG. 1B. MEV concentrations in the initial sample and in the fractions collected throughout the elution were evaluated with the Zeta View®(Nanoparticle Tracking Analyzer from Particle Metrix) as the quantity of proteins by Bradford assay. The most concentrated fractions (4-5) were pooled and stored at 4° C. before use.

### Example 1B

Small scale production of Chlorella vulgaris for (1) screening and selection of producer cell lines and (2) MEVs endo-loading

### A. Conservation of Chlorella Producer Cell Lines

After the screening of cell clones, the positive clones (cell lines) are resuspended and grown, repeating the process every 3-4 days in order to maintain the clones in an exponential phase of growth. The conditions of growing are the following: BG-11 medium with a light/night periodicity of 12 h/12 h at 20° C.-25° C. and maximum of light intensity of 3200 lux. The clones are also maintained in BG-11 agar plate and are frozen using the method describe above.

### B. Amplification of Chlorella Producer Cell Lines

Amplification phase (three weeks): For the first amplification from 96 wells plate to T25 flasks, 50 μL of cell culture are added to 5 mL of BG-11 medium and cultured for one week in the conditions described above. The second amplification round is performed by taking 4 mL from the T25 cell culture and diluted in 26 mL of BG-11 medium in a T75 flask. Then, the producer cell lines are cultured for one week. The last amplification before characterization is performed by taking 15 mL from the second amplification (T75 flasks) and diluting it with 75 mL of fresh BG-11 medium in a T225 flask, for 1 week. The end of that week is the end of the amplification phase.

Harvesting phase (three weeks): Starting from the end of the amplification phase, every 7 days, 50 mL of culture are harvested for semi-purification and testing (see below), and 50 mL of fresh BG-11 medium are added, during the 3 weeks. The samples taken every 7 days are semi-purified to isolate the extracellular vesicles (MEVs) and then MEVs are tested for protein content (GFP or Luciferase) or mRNA content (GFP or Luciferase).

During the amplification phase, Chlorella cells are exposed to antibiotics (Hygromycin or Kanamycin) to select for transformed clones. The antibiotics are absent during the harvesting phase.

### C. Semi-Purification of Endo-Loaded MEVs from Small Scale Production of Chlorella Producer Cell Lines

To isolate the MEVs in the samples, the harvested culture medium (cells+MEVs) is treated as follows. The cell-free medium is obtained through a series of low-speed centrifugations: first at 300 g, second at 1000 g, third at 3000 g, and finally at 10000 g. Then, the medium is filtered under vacuum with 0.65 μm filters. After that, a Tangential Flow Filtration is performed for each sample using VivaFlow 50R-300K system (Sartorius®) to concentrate the MEVs. Samples are further filtered on 0.45 μm filters. The concentration and size distribution of semi-purified MEVs then is analyzed by Nanoparticle Tracking Analysis (NTA; ZetaView® (Nanoparticle Tracking Analyzer from Particle Metrix) as described in Example 2 and 4.

### Example 2

MEV characterization

### A. Nanoparticle Tracking Analysis (NTA)

MEVs are analyzed for size and dispersity (size distribution) using a Zetaview® Classic Z laser 488 nm (PARTICLEMETRIX). The instrument is equipped with a 488 nm laser, a high sensitivity sCMOS camera and a syringe pump. The MEV samples are diluted in particle-free PBS (0.22 μm filter) to obtain a concentration within the recommended measurement range (1-10×10⁸ particles/mL), corresponding to dilutions of from 1/1000 to 1/10000 depending on the initial sample concentration.

For each sample, 5 experiment videos of 60 seconds duration are analyzed using NTA 3.4 Build 3.4.003 (camera level 15-16) with syringe pump speed 30. A total of 1500 frames are routinely examined per sample, captured, and analyzed by applying instrument-optimized settings using a suitable detection threshold so that the observed particles are marked with a red cross and that no more than 5 blue crosses are seen. Further settings are set to “automatic” and viscosity to “water”.

### B. Transmission Electron Microscopy (TEM)

To verify the presence and morphology of intact MEVs, the preparations are analyzed using transmission electron microscopy (TEM). MEV samples are allowed to attach to Formvar/carbon-coated grids for 15-20 min. The excess of sample is removed and the grides, fixed with 2% formaldehyde, 1% glutaraldehyde in PBS (0.1M, pH 7.4) for 20 min at room temperature. After that, washed again with PBS followed by distilled water and finally stained with 0.4% uranyl acetate/1.8% methyl cellulose for 10 min, and then dried. The preparations are observed using a Tecnai Spirit electron microscope (Thermo Fisher) equipped with a 4k CCD camera.

### C. DiR Fluorescent Labelling

For further characterization studies, MEVs are labelled with DiR, a lipophilic carbocyanine derivative (1,1′-Dioctadecyl-3,3,3′,3′-Tetramethylindotricarbocyanine Iodide; ThermoFisher Scientific) that has low fluorescence in water, but becomes highly fluorescent upon membrane incorporation, and diffuses laterally within the plasma membrane. Fresh samples of MEVs (prepared as above) are re-suspended in 1 ml of BG-11 culture medium. 5 μl of 1 mg/ml DiR solution are added to the samples, following incubation at 37° C. for 1 hour. Samples are then ultra-centrifuged at 100,000 g for 30 min using a Kontron TST 55.5 rotor at 28,100 rpm. The supernatant is removed, while the pellets are washed twice with 1 ml of PBS and centrifugation at 100,000 g for 30 min. Finally, the pellet is re-suspended in 1 ml of PBS. The DiR-labelled MEVs are stored at 4° C. and used promptly to ensure the highest possible fluorescent intensity. DiR fluorescence of the labelled MEVs is measured using a SpectraMax® fluorescence microplate reader (Molecular Devices, USA) with excitation at 750 nm and emission at 780 nm.

### D. PKH26 Fluorescent Labelling

For uptake and internalization studies, MEVs are labelled with PKH26 (Sigma-Aldrich), a fluorochrome in the red spectrum with peak excitation (551 nm) and emission (567 nm) that may also be excited by a 488 nm laser. Fresh samples of MEV (prepared as above) are re-suspended in 1 ml of Diluent C from the PKH26 kit. 6 μl of PKH26 dye is added to the samples, followed by continuous mixing for 30 seconds by gentle pipetting. After 5-minute incubation at room temperature, the samples are quenched by adding 2 ml of 10% BSA in 1×PBS. The volume is brought up to 8.5 ml in media and 1.5 ml of 0.971 M sucrose solution is added by pipetting slowly and carefully into the bottom of the tube, making sure not to create turbulence. The PKH26-labelled MEVs remains on top of a sucrose cushion. Then, the samples are ultra-centrifuged at 100,000 g for 2 hours at 2-8° C. using a Kontron TST 55.5 rotor. The supernatant is removed, while the pellets are washed with 1×PBS by gentle pipetting and centrifuged again at 100,000 g for 30 min. Finally, the pellet is re-suspended in 1 ml of 1×PBS. The PKH26-labelled MEVs are stored at 4° C. and filtered with 0.22 μm filter before adding to cells.

### E. Flow Cytometry Experiments

The analyses are conducted using LSRII flow cytometer with CellQuest® Pro software (BD Biosciences). Latex beads (Biocitex-Megamix-Plus SSC) of 0.1 and 0.9 μm diameters and fluorescent at 488 nm wavelength, are prepared and used according to the manufacturer's recommendation to define the MEV gate. Since latex beads typically have higher refractive index and thus lower limits of size detection by flow cytometry than MEVs, the thresholds for forward and side scatter are adjusted to avoid background noise during acquisition. A predefined MEV gate is applied to all samples during analysis.

### Example 3

Chlorella vulgaris engineering to generate producer cell lines carrying exogenous coding regions and endogenous loading of biomolecule cargo into the Microalgae Extracellular Vesicles (MEVs)

### A. Transformation Using a Ti Plasmid, and Agrobacterium tumefaciens to Generate Stable Chlorella Producer Cell Lines for Endogenous Loading of Biomolecules, Such as Proteins, Peptides, siRNA, mRNA, Complexes) into the MEVs

### 1. Chlorella Culture Conditions

Chlorella cells (Chlorella vulgaris UTEX 265, UTEX 395, and CCAP 211/19 from GEPEA, University of Nantes, France) are maintained in BG-11, 1% agar plates and grown in BG-11 liquid medium pH 7, in autotrophic conditions in growth chamber under the following conditions: i) temperature: 25° C.; ii) photoperiod: 14 h/10 h; iii) light intensity: 100 μmol·m⁻²·s⁻¹

Agrobacterium strain and vectors

The plasmid vectors used for Agrobacterium transformation are generated using the green gate assembly strategy (see, Lamproulos et al. (2013) J. PLOS One 8: e83043; PMID24376629). The gene specific or chimeric constructs are cloned in modules “B” or “D” and/or “B and D” according to the cloning strategy (coding or non-coding RNA) and assembled in expression plasmid constructs under the control of Cauliflower Mosaic Virus (CaMV) 35S promoter or other promoters known to those of skill in the art for expression in microalgae, including any described herein, such as those listed in the table in the detailed description, and a specific construct encoding a product of interest, and a resistance cassette (Hygromycin resistance gene) or NPTII gene (neomycin phosphotransferase II). All chimeric constructs are obtained by simultaneous ligations of the different fragments into the “B” or “D” or/and “B and D” module. All plasmids are verified by restriction analysis, and Sanger sequencing.

The binary vector pCAMBIA1304 (cambia.org) encoding a gfp: gusA fusion reporter and a selectable marker for hygromycin B resistance driven by the CaMV 35S promoter is used for some transformations.

The Agrobacterium tumefaciens used for Chlorella transformations is a disarmed strain C58C1. Plasmids are introduced into A. tumefaciens by electroporation.

### 2. Transformation of Chlorella Cells with Agrobacterium tumefaciens

Chlorella cells (10⁸ cells) from an exponentially growing culture are plated on BG-11 agar plates and kept under normal light for 5 days. For genetic transformation, A. tumefaciens carrying the appropriate plasmid vector is pre-inoculated the day before the transformation. The day of the transformation 5 mL of A. tumefaciens pre-inoculum is seeded in 50 mL LB medium and grown up to OD600=around 1. At the defined optical density A. tumefaciens pre-inoculum is washed and resuspended in 200 μL induction medium (BG-11 medium at pH 5.6 plus acetosyringone 100 μM). Chlorella cells are gently harvested from the plates and resuspended in the 200 μL of induction medium plus the A. tumefaciens and co-cultivated for 2 days in induction medium in dark. After the co-cultivation, the cells are harvested and put in BG-11 medium pH 7 supplemented with cefotaxime and kept in dark for 2 days at 25° C. Finally, cells are harvested and plated onto BG-11 agar plates supplemented with the relevant antibiotic according to the plasmid vector used for the transformation. Plates are kept in dark for 2 more days and then exposed to light. Around 2-3 weeks later, colonies are replicated on fresh BG-11 agar plate containing relevant antibiotics. Resistant colonies are propagated on non-selective media and used for PCR analysis. Detection of contaminating Agrobacterium is performed by growing cells on LB agar plates for at least 7 days at 25° C. in the dark. The expression of the gusA reporter gene is confirmed by GUS histochemical assay, while visualization of gfp expression is performed using a fluorescent microscope (Leica DM Ire2, Wetzlar, Germany).

### B. Transformation of Chlorella Cells by Electroporation to Generate Stable Producer Cell Lines that Endogenously Load Biomolecules (e.g., Proteins, Peptides, RNAi, mRNA, Complexes) into the MEVs; Gfp or RNAi Against Luciferase Coding Regions are Exemplary Biomolecules

### a. Chlorella Culture Conditions

Chlorella cells (Chlorella vulgaris UTEX 265, UTEX 395 and CCAP 211/19 from GEPEA, University of Nantes, France) are maintained in BG-11, 1% agar plates and grown in BG-11 liquid medium pH 7, in autotrophic conditions in growth chamber under the following conditions: i) temperature: 25° C.; ii) photoperiod: 14 h/10 h; iii) light intensity: 100 μmol·m⁻²·s⁻¹.

### b. Enzyme Digestion for Chlorella Cells to Prepare Protoplasts

To prepare protoplasts, Chlorella cells are treated with an enzyme mixture containing 0.6 M sorbitol, 0.1% MES, 50 mM CaCl₂)₂H₂O, 1.0 mg/mL lysozyme, 0.25 mg/mL chitinase, and 1.0 mg/mL sulfatase in 10 mL of sterile water. A total of 1×10⁷ cells 100 μL at early exponential growth phase are used for preparing protoplasts in 10 mL of the mixture solution. Cells are incubated at room temperature in the dark up to 24 h with gentle rotation at 25 rpm. Cells are harvested by centrifugation at 1350×g for 10 min. The viability of protoplasts after enzymatic treatment is about 7%.

### c. Electroporation Conditions

Chlorella cells and Chlorella protoplasts, at a concentration of 10⁶ in 100 μL, are transformed at different conditions between 600V to 1500V pulse voltage with 3 to 5 ms pulse width and using 60 ng plasmid using a Bio-Rad Gene Pulser X cell electroporation system. Electroporation is slightly modified from previously described methods (Bai et al. (2013) PLOS one 8: e54966, doi: 10.1371/journal.pone.0054966; Run et al. (2016) Algal Res 17:196-201, doi: 10.10.106/j.algal 2016.05.002; and Kumar et al. (2018) Journal of Applied Phycology 30:1735-1745, doi.org/10.1007/s10811-018-1396-3) as follows: 45 μL of pre-cooled osmotic buffer (0.2 M mannitol and 0.2 M sorbitol) is added for the suspension of the harvested cells (1350×g for 10 min) and then incubated for 40 min on ice. Again, 45 μL of pre-cooled electroporation buffer (0.2 M mannitol, 0.2 M sorbitol, 0.08 M KCl, 0.005 M CaCl₂, and 0.01 M HEPES; pH 7.2) is added in the suspension solution. 60 ng of plasmids of pCAMBIA1302 or pIT69 (in 10 μL) is added to the suspension cells immediately along with 1.5 μg of sonicated salmon sperm DNA.

### d. Screening and Clone Selection

After electroporation, cells are kept on ice for 60 min, transferred to a 12-well plate containing 1.5 mL of BG-11 medium, and cultured in the dark at 25° C. for 24 h. The cultured cells are harvested by centrifugation, suspended in 200 μL of BG-11 medium, plated onto BG-11 agar plates containing 20 μg/mL hygromycin, and incubated in continuous fluorescent light with 60 μmol photons m⁻¹ s⁻¹ at 25° C.

Two weeks after transformation and plating, clones are picked and cultured in 96-well plate for 1 week with BG-11 medium and cultured in the dark at 25° C. and 20 μg/mL hygromycin. Chlorella cells are then divided into two groups: 1) for PCR analysis and 2) to test ability to in growing concentrations of hygromycin between 0 and 100 μg/mL.

Hygromycin resistance test of selected clones indicates that clones are obtained after transformation of Chlorella cells under all of the electroporation conditions tested (see the Tables A and B below, where+indicates clone viability for the hygromycin concentration tested after 11 days of incubation).

Strains and clones description: 265 (A1) is Chlorella vulgaris UTEX 265 strain, Ath1 to Ath7 are transformed clones obtained from Chlorella vulgaris UTEX 265 strain by electroporation, 395 (K1) is Chlorella vulgaris UTEX 395 strain, Kth1 and Kth2 are transformed clones obtained from Chlorella vulgaris UTEX 395 strain by electroporation and Hr5 is a clone transformed from Chlorella vulgaris UTEX 265 strain obtained by Agrobacterium tumefaciens.

TABLE A

| | | | | | | | |
|---|---|---|---|---|---|---|---|
| Conc. | Clone | 265(A1) | Ath1 | Ath2 | Ath3 | Ath4 | Ath5 |

| | | | | | | |
|---|---|---|---|---|---|---|
| 0 μg/mL | + | + | + | + | + | + |
| 5 μg/mL | + | + | + | + | + | + |
| 10 μg/mL | + | + | + | + | + | + |
| 20 μg/mL | | + | + | + | + | + |
| 30 μg/mL | | + | + | + | + | + |
| 50 μg/mL | | + | + | + | + | + |
| 70 μg/mL | | | | | | + |
| 100 μg/mL | | | | | | |

TABLE B

| | | | | | | | |
|---|---|---|---|---|---|---|---|
| Conc. | Clone | Ath6 | Ath7 | 395(K1) | Kth1 | Kth2 | Hr5 |

| | | | | | | |
|---|---|---|---|---|---|---|
| 0 μg/mL | + | + | + | + | + | + |
| 5 μg/mL | + | + | + | + | + | + |
| 10 μg/mL | + | + | + | + | + | + |
| 20 μg/mL | + | + | + | + | + | + |
| 30 μg/mL | + | + | | + | + | + |
| 50 μg/mL | + | | | + | + | + |
| 70 μg/mL | | | | + | + | + |
| 100 μg/mL | | | | +/− | | + |

PCR analysis, of each of 20 clones obtained by electroporation from Chlorella vulgaris UTEX 265 strain, and 10 clones obtained by electroporation from Chlorella vulgaris UTEX 395 strain were tested and confirmed the integration of hygromycin phosphotransferase gene (hpt) at the molecular level. The efficiency of transformation by electroporation of both strains of Chlorella vulgaris tested was between 30-50%.

### C. Particle Gun Transformation to Generate Stable Producer Cell Lines for Endogenous Loading of Biomolecules (e.g., Proteins, Peptides, RNAi, mRNA, Complexes) into the MEVs Using Gfp or RNAi Against Luciferase Coding Regions as Exemplary Biomolecules

### 1. Transformation of Chlorella vulgaris Cells Using Gun Microparticles Method

Chlorella vulgaris cells (1×10⁸) are collected from exponentially growing liquid cultures in BG-11 medium and spread on 10 cm 1% BG-11 agar plates. Two hours later, transformations are carried out using the microparticle bombardment method (Biolistic PDS-1000/Particle Delivery System (BioRad)) adapted from Daboussi et al. (2014, Genome engineering empowers the diatom Phaeodactylum tricornutum for biotechnology. Nature Communications 5:3831) with minor modifications. In brief, gold particles (particle diameter of 0.6 μm, BioRad) are coated with DNA using 1.25 M CaCl₂) and 20 mM spermidine. Agar plates Chlorella cells to be transformed are positioned at 7.5 cm from the stopping screen within the bombardment chamber. A burst pressure of 1,550 psi and a vacuum of 25 Hg are used. For the experiments, 5 μg of each plasmid encoding hygromycin resistance gene (pIT69) or not (p16604) are used as negative control. Five bombardments are performed using each DNA.

### 2. Cloning and Selection of Transformed Clones

Two days post-transformation, bombarded cells are spread with 50 μg/ml or 100 μg/ml of hygromycin on the agar plates and placed in the incubator under a 12 h light: 12 h dark cycle for at least 3 weeks. Chlorella colonies appear about 2 weeks after transformation by bombardment. After 2 weeks, colonies are visible on the plate with cells that contain and express the hygromycin resistance gene, and not visible on the plates containing the negative control. Colonies are observed on plates on which the cells that express the hygromycin resistance gene (pIT69) are cultured. Negative control (p16604) clones do not grow in the presence of hygromycin.

For subcloning, colonies from transformations are re-suspended in BG-11 culture medium and plated at a low density (600 cells on a 10 cm agar plate containing hygromycin antibiotic), providing for the isolation of subclones 2-3 weeks later.

### 3. Chlorella Genomic DNA Extraction

Genomic DNA is extracted from exponentially growing cultures using a NucleoSpin DNA protocol. Genomic DNA (gDNA) concentration is measured using a QuBit® fluorometer.

### 4. PCR Analysis of Transformants

Direct PCR colony analysis on gDNA is performed by collecting a little bit of the colony to be analyzed (use a pipette tip or an inoculation tool or a toothpick) and resuspending it in 20 μl of HS5 buffer (125 mM NaOH, 1 mM EDTA, 0.1% Tween 20). After 20 s of vortexing at max speed, the samples are incubated for 10-15 minutes at RT and boiled at 95° C. for 10-15 s. Next, 100 μl of H₂O is added, mixed well and briefly centrifuged to spin down the debris. 1-5 μl of supernatant are used as a template for PCR reaction. The plasmid used for the transformation is the positive control; wild type gDNA served as the negative control.

PCR analysis is performed in a 25 μL reaction containing 150 ng DNA, 1.25 mM dNTP, 2 mM MgCl₂, 1.25 μM of each primer and 1 U OneTaq DNA polymerase. The primers used to amplify a 650 bp fragment of the hygromycin resistance gene are Hygro 1: 5′-AGCGTCTCCGACCTGATG-3′ (SEQ ID NO:66) and Hygro 2: 5′-CGACGGACGACTGACGG-3′; (SEQ ID NO:67). Amplification is carried out in a thermal cycler (Eppendorf). The amplification of hygromycin resistance gene shows the expected 650 bp fragment in all positive clones (i.e., growing in BG-11 plus 50 mg/mL hygromycin).

### D. Transformation by Multisequence Pulses Electroporation to Generate Stable Producer Cell Lines that Endogenously Load Biomolecules (e.g., Proteins, Peptides, RNAi, mRNA, Complexes) into the MEVs; eGFP (Modified Codon or not) or fLUC (Modified Codon or not) or RNAi Against Luciferase Coding Regions are Exemplary Biomolecules

A highly efficient multisequence pulses method was optimized to transform Chlorella vulgaris cells; with the objective of generating stable producer cell lines and to obtain MEVs endo-loaded (with tailored mRNA, proteins, or siRNAs) by the microalgae. The multisequence pulses method generates, first, pores in the wall and membranes and, second, pulses the offered DNA into the cells.

### 1. Generation of Plasmid Vectors

For the generation of Chlorella vulgaris producer cell lines a series of 57 plasmid constructs (Table 3 listing plasmids and components thereof; see, also, SEQ Nos.: 238-294) were obtained using the Green-Gate system combining: 12 different promoters-enhancers, and 2 marker proteins eGFP and firefly luciferase, the cDNA encoding each of the two proteins was cloned in plasmid vectors using mammalian codons or Chlorella codons (different GC %), and 2 antibiotic resistance genes in order to obtain Chlorella producer cell lines and RNAi against firefly luciferase (fLUC). The constructs are verified by sequencing the complete region of interest, from the promoter of the transgene to the poly-A of the antibiotic resistance gene. Sequencing of all cloned fragments is performed using the primers set forth in Table 2.

TABLE 2
List of primers used to verify plasmid sequences

| | | | SEQ ID |
|---|---|---|---|
| Primer Name# | Primer Sequence | target | NO. |
| BSAICMVF1 | AAAAGGTCTCAACCTGTGATGCG | CMV promoter | 207 |
| | GTTTTGGCAGTA | | |
| BSAICMVR1 | AAAAGGTCTCATGTTAGCTCTGC | CMV promoter | 208 |
| | TTATATAGACCTCCCAC | | |
| BSAICMV + ENHF1 | AAAAGGTCTCAACCTCGTTACAT | CMV promoter + | 209 |
| | AACTTACGGTAAATGGC | enhancer | |
| BSAICMV + ENHR1 | AAAAGGTCTCATGTTAGCTCTGC | CMV promoter + | 210 |
| | TTATATAGACCTCCCAC | enhancer | |
| BSAISV40F1 | AAAAGGTCTCAACCTCTGAGGCG | SV40 promoter | 211 |
| | GAAAGAACCAGC | | |
| BSAISV40R1 | AAAAGGTCTCATGTTTTTGCAAA | SV40 promoter | 212 |
| | AGCCTAGGCCTCCA | | |
| BSAISV40 + AMPRF1 | AAAGGTCTCAACCTCGCGGAACC | SV40 promoter + | 213 |
| | CCTATTTGTTTATTTTTC | enhancer | |
| BSAISV40 + AMPRR1 | AAAAGGTCTCATGTTTTTGCAAA | SV40 promoter + | 214 |
| | AGCCTAGGCCTCCAAAAAAGCC | enhancer | |
| PGGA-L2151 | TTTATGCTTCCGGCTCGTAT | Other promoters used | 215 |
| PGGA-R3394-2327 | AGGGTTTTCCCAGTCACGA | Other promoters used | 216 |
| BSAIEGFPF1 | AAAAGGTCTCaGGCTATGGTGAG | CDS eGFP | 217 |
| | CAAGGGCGAGGA | | |
| BSAIEGFPR1 | AAAAGGTCTCaCTGACTTGTACA | CDS eGFP | 218 |
| | GCTCGTCCATGCC | | |
| KANR3 | GGCCATTTTCCACCATGATA | ALL antibiotic | 219 |
| | | resistance genes | |
| PGGA-L2151 | TTTATGCTTCCGGCTCGTAT | ALL antibiotic | 220 |
| | | resistance genes | |
| PGGA-R3394-2327 | AGGGTTTTCCCAGTCACGA | ALL antibiotic | 221 |
| | | resistance genes | |
| DTR-64 | CGATTTTTGTGATGCTCGTC | ALL insert CDS 5′ | 222 |
| | | region and 3′ region | |
| DTR-63COR | ACCTCTCGGGCTTCTGGAT | ALL insert CDS 5′ | 223 |
| | | region and 3′ region | |
| fLUCR1 | AGCACGGGCATGAAGAAC | CDS fLuc | 224 |
| fLUCR2 | AAGCGGTACATCAGCACCA | CDS fLuc | 225 |
| fLUCR3 | CGTCCCAGTAGGCGATGT | CDS fLuc | 226 |
| E001R1 | TTGAGAATGAACAAAAGGACCA | CDS fLuc | 227 |
| F005R1 | TGATATCTAACGATCAAACCCAGA | CDS fLuc | 228 |
| F005R3 | AGCAATCGCGCATATGAAA | CDS fLuc | 229 |
| F005R5 | TTTTAGGTTTGACCGGTTCTG | CDS fLuc | 230 |
| A432F1 | ACACCTACCGATGCGTGAA | CDS fLuc | 231 |
| E001R1 | TTGAGAATGAACAAAAGGACCA | CDS eGFP in | 232 |
| | | kanamycin resistance | |
| | | vectors | |
| KanR1 | GCCCAGTCATAGCCGAATAG | CDS eGFP in | 233 |
| | | kanamycin resistance | |
| | | vectors | |
| KanR3 | GGCCATTTTCCACCATGATA | CDS eGFP in | 234 |
| | | kanamycin resistance | |
| | | vectors | |
| A432F1 | ACACCTACCGATGCGTGAA | CDS eGFP in | 235 |
| | | kanamycin resistance | |
| | | vectors | |
| eGFPR1 | CTGGGTGCTCAGGTAGTGGT | CDS eGFP in | 236 |
| | | kanamycin resistance | |
| | | vectors | |
| eGFPR2 | TCGTCCTTGAAGAAGATGGT | CDS eGFP in | 237 |
| | | kanamycin resistance | |
| | | vectors | |
| A601F1 | GCGTGGATAGCGGTTTGA | CMV promoter | 367 |
| A601R1 | GGCGGAGTTGTTACGACATT | CMV promoter | 368 |
| A601F2 | ATGCGGTTTTGGCAGTACA | CMV promoter | 369 |
| A601R2 | CTTATAATAGACCTCCCACCGTA | CMV promoter | 370 |
| | CAC | | |
| A602F1 | TACCATGGTGATGCGGTTT | CMV promoter + | 371 |
| | | enhancer | |
| A602R1 | AAGTCCCGTTGATTTTGGTG | CMV promoter + | 372 |
| | | enhancer | |
| A602F2 | TCAATAATGACGTATGTTCCCAT | CMV promoter + | 373 |
| | AG | enhancer | |
| A602R2 | CTTARATAGACCTCCCACCGTAC | CMV promoter + | 374 |
| | AC | enhancer | |
| A603F1 | CCATAGTCCCGCCCCTAA | SV40 promoter | 375 |
| A603R1 | TCGGCCTCTGCATAAATAAAA | SV40 promoter | 376 |
| A603F2 | CAGCAACCAGGTGTGGGAAA | SV40 promoter | 377 |
| A603R2 | AAAGCCTAGGCCTCCAAAAA | SV40 promoter | 378 |
| A604F1 | GCGGAACCCCTATTTGTTTATT | SV40 promoter + | 379 |
| | | enhancer | |
| A604R1 | TTCCACAGCTGGTTCTTTCC | SV40 promoter + | 380 |
| | | enhancer | |
| A604F2 | TCCGCTCATGAGACAATAACC | SV40 promoter + | 381 |
| | | enhancer | |
| A604R2 | AGTTAGGGGGGGGACTATG | SV40 promoter + | 382 |
| | | enhancer | |

Table 3 provides a list of exemplary constructs. The constructs were used by multiple sequence pulses electroporation for the transformation of Chlorella vulgaris to generate cells line producers of MEVs endo-loaded with tailored mRNAs, proteins, and/or RNAi.

TABLE 3
List of plasmids

| Plasmid | | | | | Resistance | Seq |
|---|---|---|---|---|---|---|
| name # | Promoter | Type | CDS | Terminator | Gene | ID # |
| pAGS-0001 | EF2 | constitutive | fLUC modified | RBCS | Hygromycin | 238 |
| | (Phaeodactylum | | codon to Chlorella | | | |
| | tricornutum) | | GC content | | | |
| pAGS-0002 | Histone 4 | constitutive | fLUC modified | RBCS | Hygromycin | 239 |
| | (Penicillium | | codon to Chlorella | | | |
| | funiculosum) | | GC content | | | |
| pAGS-0003 | Lhcf.1 | inducible | fLUC modified | RBCS | Hygromycin | 240 |
| | (Phaeodactylum | (light) | codon to Chlorella | | | |
| | tricornutum) | | GC content | | | |
| pAGS-0004 | Ubi.U4 | constitutive | fLUC modified | RBCS | Hygromycin | 241 |
| | (Nicotiana | | codon to Chlorella | | | |
| | tabacum) | | GC content | | | |
| pAGS-0005 | CAM 35S | constitutive | fLUC modified | RBCS | Hygromycin | 242 |
| | (Cauliflower | | codon to Chlorella | | | |
| | mosaic | | GC content | | | |
| | virus) | | | | | |
| pAGS-0006 | MAS | constitutive | fLUC modified | RBCS | Hygromycin | 243 |
| | (Agrobacterium | | codon to Chlorella | | | |
| | tumefaciens) | | GC content | | | |
| pAGS-0007 | PAR | inducible | fLUC modified | RBCS | Hygromycin | 244 |
| | (Arabidopsis | (light) | codon to Chlorella | | | |
| | thaliana) | | GC content | | | |
| pAGS-0008 | Cv PAS D | inducible | fLUC modified | RBCS | Hygromycin | 245 |
| | (Chlorella | (light and | codon to Chlorella | | | |
| | vulgaris) | stress) | GC content | | | |
| pAGS-0009 | CMV | Constitutive | fLUC modified | RBCS | Hygromycin | 246 |
| | | | codon to Chlorella | | | |
| | | | GC content | | | |
| pAGS-0010 | CMV + | Constitutive | fLUC modified | RBCS | Hygromycin | 247 |
| | enhancer | | codon to Chlorella | | | |
| | | | GC content | | | |
| pAGS-0011 | SV40 | Constitutive | fLUC modified | RBCS | Hygromycin | 248 |
| | | | codon to Chlorella | | | |
| | | | GC content | | | |
| pAGS-0012 | SV40 + | Constitutive | fLUC modified | RBCS | Hygromycin | 249 |
| | enhancer | | codon to Chlorella | | | |
| | | | GC content | | | |
| pAGS-0013 | EF2 | Constitutive | eGFP modified | RBCS | Hygromycin | 250 |
| | (Phaeodactylum | | codon to Chlorella | | | |
| | tricornutum) | | GC content | | | |
| pAGS-0014 | Histone 4 | Constitutive | eGFP modified | RBCS | Hygromycin | 251 |
| | (Penicillium | | codon to Chlorella | | | |
| | funiculosum) | | GC content | | | |
| pAGS-0015 | Lhcf.1 | inducible | eGFP modified | RBCS | Hygromycin | 252 |
| | (Phaeordactium | (light) | codon to Chlorella | | | |
| | tricornutum) | | GC content | | | |
| pAGS-0016 | Ubi.U4 | constitutive | eGFP modified | RBCS | Hygromycin | 253 |
| | (Nicotiana | | codon to Chlorella | | | |
| | tabacum) | | GC content | | | |
| pAGS-0017 | CAM 35S | constitutive | eGFP modified | RBCS | Hygromycin | 254 |
| | (Cauliflower | | codon to Chlorella | | | |
| | mosaic | | GC content | | | |
| | virus) | | | | | |
| pAGS-0018 | MAS | constitutive | eGFP modified | RBCS | Hygromycin | 255 |
| | (Agrobacterium | | codon to Chlorella | | | |
| | tumefaciens) | | GC content | | | |
| pAGS-0019 | PAR | inducible | eGFP modified | RBCS | Hygromycin | 256 |
| | (Arabidopsis | (light) | codon to Chlorella | | | |
| | thaliana) | | GC content | | | |
| pAGS-0020 | Cv PAS D | inducible | eGFP modified | RBCS | Hygromycin | 257 |
| | (Chlorella | (light and | codon to Chlorella | | | |
| | vulgaris) | stress) | GC content | | | |
| pAGS-0021 | CMV | constitutive | eGFP modified | RBCS | Hygromycin | 258 |
| | | | codon to Chlorella | | | |
| | | | GC content | | | |
| pAGS-0022 | CMV + | constitutive | eGFP modified | RBCS | Hygromycin | 259 |
| | enhancer | | codon to Chlorella | | | |
| | | | GC content | | | |
| pAGS-0023 | SV40 | constitutive | eGFP modified | RBCS | Hygromycin | 260 |
| | | | codon to Chlorella | | | |
| | | | GC content | | | |
| pAGS-0024 | SV40 + | constitutive | eGFP modified | RBCS | Hygromycin | 261 |
| | enhancer | | codon to Chlorella | | | |
| | | | GC content | | | |
| pAGS-0025 | EF2 | constitutive | eGFP non | RBCS | Hygromycin | 262 |
| | (Phaeodactylum | | modified | | | |
| | tricornutum) | | sequence | | | |
| pAGS-0026 | Histone 4 | constitutive | eGFP non | RBCS | Hygromycin | 263 |
| | (Penicillium | | modified | | | |
| | foniculosum) | | sequence | | | |
| pAGS-0027 | Lhcf.1 | inducible | eGFP non | RBCS | Hygromycin | 264 |
| | (Phaeordactium | (light) | modified | | | |
| | tricornutum) | | sequence | | | |
| pAGS-0028 | Ubi.U4 | constitutive | eGFP non | RBCS | Hygromycin | 265 |
| | (Nicotiana | | modified | | | |
| | tabacum) | | sequence | | | |
| pAGS-0029 | CAM 35S | constitutive | eGFP non | RBCS | Hygromycin | 266 |
| | (Cauliflower | | modified | | | |
| | mosaic | | sequence | | | |
| | virus) | | | | | |
| pAGS-0030 | MAS | constitutive | eGFP non | RBCS | Hygromycin | 267 |
| | (Agrobacterium | | modified | | | |
| | tumefaciens) | | sequence | | | |
| pAGS-0031 | PAR | inducible | eGFP non | RBCS | Hygromycin | 268 |
| | (Arabidopsis | (light) | modified | | | |
| | thaliana) | | sequence | | | |
| pAGS-0032 | Cv PAS D | inducible | eGFP non | RBCS | Hygromycin | 269 |
| | (Chlorella | (light and | modified | | | |
| | vulgaris) | stress) | sequence | | | |
| pAGS-0033 | CMV | constitutive | eGFP non | RBCS | Hygromycin | 270 |
| | | | modified | | | |
| | | | sequence | | | |
| pAGS-0034 | CMV + | constitutive | eGFP non | RBCS | Hygromycin | 271 |
| | enhancer | | modified | | | |
| | | | sequence | | | |
| pAGS-0035 | SV40 | constitutive | eGFP non | RBCS | Hygromycin | 272 |
| | | | modified | | | |
| | | | sequence | | | |
| pAGS-0036 | SV40 + | constitutive | eGFP non | RBCS | Hygromycin | 273 |
| | enhancer | | modified | | | |
| | | | sequence | | | |
| pAGS-0037 | EF2 | constitutive | eGFP non | RBCS | Hygromycin | 274 |
| | (Phaeodactylum | | modified | | | |
| | tricornutum) | | sequence | | | |
| pAGS-0038 | Histone 4 | constitutive | fLUC non | RBCS | Hygromycin | 275 |
| | (Penicillium | | modified | | | |
| | funiculosum) | | sequence | | | |
| pAGS-0039 | Lhcf.1 | inducible | fLUC non | RBCS | Hygromycin | 276 |
| | (Phaeodactylum | (light) | modified | | | |
| | tricornutum) | | sequence | | | |
| pAGS-0040 | Ubi.U4 | constitutive | fLUC non | RBCS | Hygromycin | 277 |
| | (Nicotiana | | modified | | | |
| | tabacum) | | sequence | | | |
| pAGS-0041 | CAM 35S | constitutive | fLUC non | RBCS | Hygromycin | 278 |
| | (Cauliflower | | modified | | | |
| | mosaic | | sequence | | | |
| | virus) | | | | | |
| pAGS-0042 | MAS | constitutive | fLUC non | RBCS | Hygromycin | 279 |
| | (Agrobacterium | | modified | | | |
| | tumefaciens) | | sequence | | | |
| pAGS-0043 | PAR | inducible | fLUC non | RBCS | Hygromycin | 280 |
| | (Arabidopsis | (light) | modified | | | |
| | thaliana) | | sequence | | | |
| pAGS-0044 | Cv PAS D | inducible | fLUC non | RBCS | Hygromycin | 281 |
| | (Chlorella | (light and | modified | | | |
| | vulgaris) | stress) | sequence | | | |
| pAGS-0045 | CMV | constitutive | fLUC non | RBCS | Hygromycin | 282 |
| | | | modified | | | |
| | | | sequence | | | |
| pAGS-0046 | CMV + | constitutive | fLUC non | RBCS | Hygromycin | 283 |
| | enhancer | | modified | | | |
| | | | sequence | | | |
| pAGS-0047 | SV40 | constitutive | fLUC non | RBCS | Hygromycin | 284 |
| | | | modified | | | |
| | | | sequence | | | |
| pAGS-0048 | SV40 + | constitutive | fLUC non | RBCS | Hygromycin | 285 |
| | enhancer | | modified | | | |
| | | | sequence | | | |
| pAGS-0049 | EF2 | constitutive | eGFP modified | RBCS | Kanamycin | 286 |
| | (Phaeodactylum | | codon to Chlorella | | | |
| | tricornutum) | | GC content | | | |
| pAGS-0050 | Histone 4 | constitutive | eGFP modified | RBCS | Kanamycin | 287 |
| | (Penicillium | | codon to Chlorella | | | |
| | funiculosum) | | GC content | | | |
| pAGS-0051 | Lhcf.1 | inducible | eGFP modified | RBCS | Kanamycin | 288 |
| | (Phaeodactylum | (light) | codon to Chlorella | | | |
| | tricornutum) | | GC content | | | |
| pAGS-0052 | Ubi.U4 | constitutive | eGFP modified | RBCS | Kanamycin | 289 |
| | (Nicotiana | | codon to Chlorella | | | |
| | tabacum) | | GC content | | | |
| pAGS-0053 | CAM 35S | constitutive | eGFP modified | RBCS | Kanamycin | 290 |
| | (Cauliflower | | codon to Chlorella | | | |
| | mosaic | | GC content | | | |
| | virus) | | | | | |
| pAGS-0054 | MAS | constitutive | eGFP modified | RBCS | Kanamycin | 291 |
| | (Agrobacterium | | codon to Chlorella | | | |
| | tumefaciens) | | GC content | | | |
| pAGS-0055 | PAR | inducible | eGFP modified | RBCS | Kanamycin | 292 |
| | (Arabidopsis | (light) | codon to Chlorella | | | |
| | thaliana) | | GC content | | | |
| pAGS-0056 | Cv PAS D | inducible | eGFP modified | RBCS | Kanamycin | 293 |
| | (Chlorella | (light and | codon to Chlorella | | | |
| | vulgaris) | stress) | GC content | | | |
| pIT 69 | CAM 35S | constitutive | siRNA anti fLUC | RBCS | Hygromycin | 294 |
| | (Cauliflower | | | | | |
| | mosaic | | | | | |
| | virus) | | | | | |

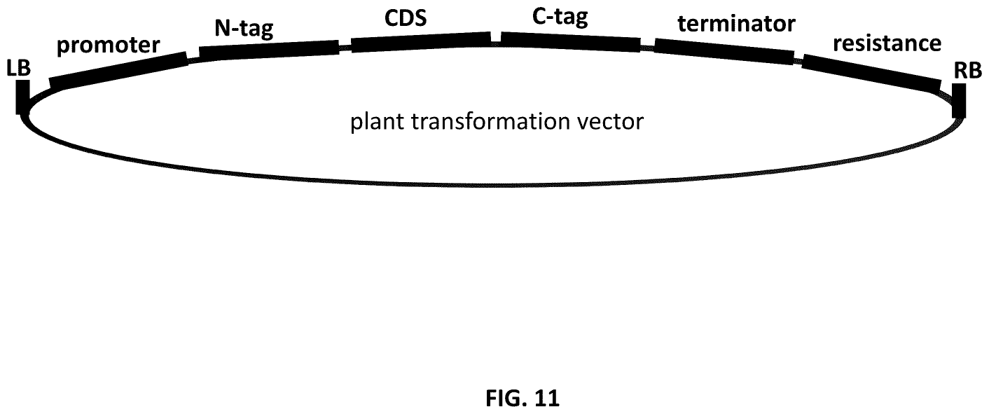

FIG. 11 depicts the structure of the T-DNA in the Ti plasmids provided herein. Plasmid sequences, as depicted, are written from LB (left border of the T-DNA (transferred DNA) element in the Ti plasmid) to RB (right border of the T-DNA element) as shown in FIG. 11.

### 2. Transformation by Multisequence Pulses

Cells of Chlorella vulgaris in the exponential growth phase are electroporated using a Nepa21 type II Electroporator (Nepa Gene), as follows: after determination of the number of cells/mL, aliquots of ten million cells per aliquot are placed in separate 1.5 mL Eppendorf tubes (one tube per transformation condition) in 100 μL of BG-11 media plus 0.7 M of mannitol; 6 μg (in 20 μL at maximum) of linearized (or not) plasmids are added to the cell suspensions. The preparations are homogenized by tapping the tubes, then placing them into electroporation chambers of 2 mm of diameter (EC002S; Sonidel) and electroporated. The conditions of electroporation are described in Tables 4.

TABLE 4A
Conditions for poring Chlorella cells
First Sequence: Poring Pulses

| | Pulse | Interval between | Pulse | Decay | |
|---|---|---|---|---|---|
| Voltage | Time (ms) | Pulses (ms) | Number | rate (%) | Polarity |
| 300 | 5 | 50 | 7 | 10 | non |

TABLE 4B
Conditions for transferring DNA into Chlorella cells
Second Sequence: Transferring Pulses

| | Pulse | Interval between | Pulse | Decay | |
|---|---|---|---|---|---|
| Voltage | Time (ms) | Pulses (ms) | Number | rate (%) | Polarity |
| 8 | 50 | 50 | 5 | 40 | non |

After transformation, Chlorella cells are diluted in 4 mL BG-11 medium+0.7M mannitol in T25 flasks. Cells are incubated in a refrigerated incubator, at 25° C. overnight, protected from light. The next day, they are resuspended using a sterile pipette, and transferred into 15 mL Falcon tubes. Cells are centrifuged 5 min at 700 g, the supernatant removed and resuspended in 200 μL of BG-11 media. The total volume (200 μL) of each cell suspension is plated on Agar/BG-11 agar plates with hygromycin (70 μg/mL) or kanamycin (100 μg/mL) according to the plasmid vector used for transformation, and incubated in the refrigerated incubator at 25° C., for 12 hours light/12 hours dark (12 L/12D). Clones are visible at day 10 and are harvested for PCR screening at day 15.

### 3. Clone Screening

For each transformation, 5 to 10 clones are picked and cultured in BG-11 medium containing an antibiotic (either 70 mg/mL of Hygromycin or 100 mg/mL of Kanamycin). After 10 days of culture clones are tested to identify positive clones, and in parallel frozen for banking.

Positive clones are identified by PCR using the corresponding primers in the following list, that match the coding regions of either the resistance gene or the transgene, or the promoter region of the vector. The sequences of primers used for the screening are listed in Table 5. Screening is performed by either multiplex or simplex PCR analysis. PCR conditions are also described in Table 5.

The number of positive clones obtained after the multiple sequence pulses is an average of 4 out of 10 clones picked.

TABLE 5
PCR conditions for the screening of Chlorella producer cell lines

| SEQ ID | Primer | | Start | Length | TM | GC | PCR band |
|---|---|---|---|---|---|---|---|
| # | Name # | 3′ sequence 5′ | (bp) | (bp) | (° C.) | (%) | length (bp) |
| 295 | A407F1 | CAAAGATACAG | 22 | 27 | 61 | 41 | 342 from |
| | | TCTCAGAAGAC | | | | | Cam35S |
| | | CAAAG | | | | | promoter |
| 296 | A407R1 | GAAGGGTCTTG | 364 | 21 | 60 | 52 | |
| | | CGAAGGATAG | | | | | |
| 297 | A407F2 | GAAGACCAAAG | 38 | 22 | 60 | 45 | 256 from |
| | | GGCTATTGAGA | | | | | Cam35S |
| 298 | A407R2 | CTTGCTTTGAA | 293 | 21 | 61 | 48 | promoter |
| | | GACGTGGTTG | | | | | |
| 299 | A401F1 | GATTCGTTCCC | 87 | 19 | 60 | 52 | 220 from EF2 |
| | | GTCACAGA | | | | | promoter |
| 300 | A401R1 | AAGATTCACCA | 306 | 20 | 60 | 50 | |
| | | CGGCTTCAC | | | | | |
| 301 | A401F2 | CCTTCCATTTTG | 9 | 22 | 61 | 41 | 324 from EF2 |
| | | ACATGTTTCC | | | | | promoter |
| 302 | A401R2 | TGTGTGGAGAG | 332 | 20 | 60 | 55 | |
| | | AACGAGCAG | | | | | |
| 303 | A403F1 | AAGTATGGCGC | 371 | 29 | 60 | 50 | 227 from H4 |
| | | AGGTATGGT | | | | | promoter |
| 304 | A403R1 | GCGAATTGTGA | 597 | 20 | 60 | 50 | |
| | | GTGCTTCCT | | | | | |
| 305 | A403F3 | AGGTCCGTGGA | 50 | 19 | 60 | 58 | 521 from H4 |
| | | CTTCTGGT | | | | | promoter |
| 306 | A403R3 | GTTCACAGGCC | 570 | 20 | 60 | 55 | |
| | | GTAGGTTTC | | | | | |
| 307 | A405F1 | GGATTATCACC | 23 | 21 | 60 | 38 | 103 from |
| | | GCAAAAATGA | | | | | Lhcf1 |
| 308 | A405R1 | TATTTGCTACCAG | 125 | 23 | 61 | 43 | promoter |
| | | AGCAATCCAC | | | | | |
| 309 | A405F2 | GGAGGATTATC | 20 | 20 | 60 | 45 | 130 from |
| | | ACCGCAAAA | | | | | Lhcf1 |
| 310 | A405R2 | ATGTTGAGCCAA | 149 | 23 | 60 | 43 | promoter |
| | | TCTAGCAGAAG | | | | | |
| 311 | A427F1 | CAAAGATACAG | 22 | 27 | 61 | 41 | 341 from |
| | | TCTCAGAAGAC | | | | | MAS |
| | | CAAAG | | | | | promoter |
| 312 | A427R1 | GAAGGGTCTTG | 364 | 21 | 60 | 52 | |
| | | CGAAGGATAG | | | | | |
| 313 | A427F2 | GAAGACCAAAG | 38 | 22 | 60 | 45 | 255 from |
| | | GGCTATTGAGA | | | | | MAS |
| 314 | A427R2 | CTTGCTTTGAA | 293 | 21 | 61 | 48 | promoter |
| | | GACGTGGTTG | | | | | |
| 315 | E001F1 | TGAAATGGAAA | 196 | 21 | 60 | 38 | 50 from |
| | | TGGATGGAGA | | | | | terminator |
| 316 | E001R1 | TTGAGAATGAA | 255 | 22 | 60 | 36 | |
| | | CAAAAGGACCA | | | | | |
| 317 | E001F2 | TCATGGATTTG | 578 | 27 | 60 | 30 | 86 from |
| | | TAGTTGAGTAT | | | | | terminator |
| | | GAAAA | | | | | |
| 318 | E001R2 | CATAGTCGATT | 664 | 22 | 59 | 45 | |
| | | GATGCATGTTG | | | | | |
| 319 | A431F1 | GCACCCATCCG | 145 | 20 | 60 | 50 | 212 from |
| | | GTATAAAAG | | | | | PAR |
| 320 | A431R1 | TAAGGGTTGCA | 356 | 20 | 60 | 45 | promoter |
| | | AGTGCTCAA | | | | | |
| 321 | A431F2 | TGAGGCTTGAC | 8 | 19 | 60 | 45 | 349 from |
| | | ATGATTGGT | | | | | PAR |
| 322 | A431R2 | TAAGGGTTGCA | 356 | 20 | 60 | 45 | promoter |
| | | AGTGCTCAA | | | | | |
| 323 | A432F1 | ACACCTACCGA | 339 | 19 | 60 | 53 | 88 from Cv- |
| | | TGCGTGAA | | | | | PSAD |
| 324 | A432R1 | CTCGGAACGAC | 426 | 20 | 60 | 55 | promoter |
| | | GACAAGAAG | | | | | |
| 325 | A432F2 | TCTCAGCATGC | 178 | 20 | 60 | 50 | 243from Cv- |
| | | CTCAACAAC | | | | | PSAD |
| 326 | A432R2 | CTCGGAACGAC | 420 | 19 | 60 | 52 | promoter |
| | | GACAAGAA | | | | | |
| 327 | fLUCF1 | CTACGGCCTGA | 218 | 19 | 59 | 58 | 72 from |
| | | ACACCAAC | | | | | fLUC MC |
| 328 | fLUCR1 | AGCACGGGCAT | 289 | 18 | 60 | 56 | |
| | | GAAGAAC | | | | | |
| 329 | fLUCF2 | CCATCTTCGGC | 685 | 18 | 61 | 56 | 130 from |
| | | AACCAGA | | | | | fLUC MC |
| 330 | fLUCR2 | AAGCGGTACAT | 814 | 19 | 60 | 53 | |
| | | CAGCACCA | | | | | |
| 331 | fLUCF3 | CTTCTTCGAGG | 1112 | 19 | 61 | 58 | 182 from |
| | | CCAAGGTG | | | | | fLUC MC |
| 332 | fLUCR3 | CGTCCCAGTAG | 1293 | 18 | 60 | 61 | |
| | | GCGATGT | | | | | |
| 333 | fLUCF4 | AAGACCATGAC | 1482 | 20 | 60 | 50 | 157 from |
| | | CGAGAAGGA | | | | | fLUC MC |
| 334 | fLUCR4 | TTGGCCTTGAT | 1639 | 20 | 60 | 50 | |
| | | CAGGATCTC | | | | | |
| 335 | eGFPF1 | CAGAAGAACGG | 472 | 20 | 60 | 50 | 144 from |
| | | CATCAAGGT | | | | | eGFP |
| 336 | eGFPR1 | CTGGGTGCTCA | 615 | 20 | 60 | 60 | |
| | | GGTAGTGGT | | | | | |
| 337 | eGFPF2 | CCCGACCACAT | 226 | 18 | 61 | 61 | 86 from |
| | | GAAGCAG | | | | | eGFP |
| 338 | eGFPR2 | TCGTCCTTGAA | 311 | 20 | 57 | 45 | |
| | | GAAGATGGT | | | | | |
| 339 | eGFPF3 | AGGAGCGCACC | 284 | 19 | 60 | 52 | 212 from |
| | | ATCTTCTT | | | | | eGFP |
| 340 | eGFPR3 | GTTCACCTTGA | 495 | 20 | 60 | 50 | |
| | | TGCCGTTCT | | | | | |
| 341 | eGFPF4 | GGCAAGCTGAC | 121 | 19 | 60 | 57 | 365 from |
| | | CCTGAAGT | | | | | eGFP |
| 342 | eGFPR4 | ATGCCGTTCTTC | 485 | 20 | 60 | 50 | |
| | | TGCTTGTC | | | | | |
| 343 | fLUC2F1 | ATTCTTTATGCC | 261 | 20 | 60 | 45 | 114 from |
| | | GGTGTTGG | | | | | fLUC |
| 344 | fLUC2R1 | ACGGTAGGCTG | 374 | 18 | 60 | 56 | |
| | | CGAAATG | | | | | |
| 345 | fLUC2F2 | TGTCGCTCTGC | 621 | 20 | 60 | 50 | 121 from |
| | | CTCATAGAA | | | | | fLUC |
| 346 | fLUC2R2 | AAAACCGTGAT | 741 | 20 | 60 | 40 | |
| | | GGAATGGAA | | | | | |
| 347 | fLUC2F3 | CGCGGTCGGTA | 1080 | 19 | 61 | 53 | 98 from |
| | | AAGTTGTT | | | | | fLUC |
| 348 | fLUC2R3 | TCACACACAGT | 1177 | 20 | 60 | 50 | |
| | | TCGCCTCTT | | | | | |
| 349 | fLUC2F4 | TGACGGAAAAA | 1478 | 20 | 59 | 45 | 50 from |
| | | GAGATCGTG | | | | | fLUC |
| 350 | fLUC2R4 | CGCGGTTGTTA | 1527 | 20 | 62 | 55 | |
| | | CTTGACTGG | | | | | |
| 351 | F005F4 | TGAAATGGAAA | 1696 | 20 | 61 | 55 | 55 from |
| | | TGGATGGAGA | | | | | Hygromycin |
| 352 | F005R4 | TTGAGAATGAA | 1751 | 20 | 60 | 50 | |
| | | CAAAAGGACCA | | | | | |
| 353 | F005F5 | TCATGGATTTG | 1994 | 20 | 59 | 40 | 81 from |
| | | TAGTTGAGTAT | | | | | Hygromycin |
| | | GAAAA | | | | | |
| 354 | F005R5 | CATAGTCGATT | 2074 | 21 | 59 | 42 | |
| | | GATGCATGTTG | | | | | |
| 355 | F005F1 | CGTTCGATCCC | 391 | 19 | 61 | 38 | 90 from |
| | | AATTTCGT | | | | | Hygromycin |
| 356 | F005R1 | TGATATCTAACG | 481 | 24 | 60 | 61 | |
| | | ATCAAACCCAGA | | | | | |
| 357 | F005F2 | AAAAGTTCGAC | 739 | 20 | 60 | 50 | 87 from |
| | | AGCGTCTCC | | | | | Hygromycin |
| 358 | F005R2 | TATCCACGCCC | 826 | 20 | 60 | 55 | |
| | | TCCTACATC | | | | | |
| 359 | F005F3 | CAAGGAATCGGT | 1104 | 24 | 60 | 42 | 51 from |
| | | CAATACACTACA | | | | | Hygromycin |
| 360 | F005R3 | AGCAATCGCGC | 1154 | 19 | 60 | 42 | |
| | | ATATGAAA | | | | | |
| 361 | KanF1 | CGAGGCATGAT | 291 | 20 | 60 | 45 | 80 from |
| | | TGAACAAGA | | | | | Kanamycin |
| 362 | KanR1 | GCCCAGTCATA | 370 | 20 | 60 | 55 | |
| | | GCCGAATAG | | | | | |
| 363 | KanF2 | GCCGTTTTACG | 60 | 20 | 59 | 45 | 112 from |
| | | TTTGGAACT | | | | | Kanamycin |
| 364 | KanR2 | AACGCGCAATA | 171 | 20 | 60 | 40 | |
| | | ATGGTTTCT | | | | | |
| 365 | KanF3 | GGATGATCTGG | 761 | 20 | 60 | 55 | 140 from |
| | | ACGAAGAGC | | | | | Kanamycin |
| 366 | KanR3 | GGCCATTTTCC | 900 | 20 | 60 | 45 | |
| | | ACCATGATA | | | | | |

After screening, 24 positive clones of Chlorella producer cell lines are produced, MEVs are collected and semi-purified as described in Example 1B. The resulting semi-purified MEVs are tested to verify the presence of the endo-loaded cargo, as described below in Example 4.

### E. Storage of Chlorella Modified Producer Cell Lines. Cryopreservation of Chlorella vulgaris Cells

Two conditions of cryopreservation and storage of isolated cells from Chlorella vulgaris in exponential growing state and in stationary state are evaluated. The first condition corresponds to the standard method recommended by UTEX collection using 10% methanol as a cryoprotective agent in BG-11 medium and the second condition using 25% glycerol in BG-11 medium as routinely used for animal cell lines.

Both cryo-storage conditions are evaluated in exponential growing phase (4-5×10⁶ cells) or stationary phase (3-5×10⁷ cells). After congelation, the cells are thawed at 37° C. Cell viability is determined in BG-11 medium for 5 days (liquid medium) or BG-11 solid medium (stria) for 15 days culture. Viability is determined after one or two cycles of freezing/thawing at 3 days and 30 days (one cycle) or 30, 60, 180 days (two cycles).

A higher viability and a better reproducibility of the results is obtained with the 25% of glycerol in BG-11 medium (freezing medium), with Chlorella cells in exponential growing phase and a dilution of 100 mL of cryomedium in 5 mL BG-11 medium liquid culture as thawing culture conditions.

### F. Determination of the Genetic Stability of Chlorella Producer Cell Lines. PCR Detection of Chlorella Transformants

Genomic DNA from transformed and wild-type (WT) Chlorella strains is isolated from exponentially growing cultures using NucleoSpin DNA protocol. Genomic DNA concentration is measured using a QuBit® fluorometer.

The genetic stability of Chlorella producer cell lines is performed by PCR analysis to detect the presence and the persistence of either the hygromycin resistance gene (using specific primers as described above (Example 3b) or firefly luciferase primers (f-LUC1-5′-CCAGGGATTTCAGTCGATGT-3′ and f-LUC2-5′-AATCTCACGCAGGCAGTTCT-3′), or GFP (SEQ ID NOs: 68 and 69) or eGFP (SEQ ID NOs: 80 and 81), or mCherry (SEQ ID NOs: 82 and 83), or GUS (SEQ ID NOs: 84 and 85) as described below (Example 4).

### Example 4

### Endo-Loaded MEV Characterization

### A. Quantification of GFP Fluorescence in Transformed Chlorella Cell Lines and in Isolated MEVs

The parental strain of Chlorella is transformed with Green Fluorescent Protein (GFP)-coding sequence using plasmids pAGS-0013 to pAGS-0024 or pAGS-0049 to pAGS-0056, as described in Example 3. The GFP fluorescence intensity is examined on a microplate reader (Molecular Device Co, Spectra Max M2). GFP samples are prepared by serial twofold dilution with phosphate buffered saline (PBS, 137 mM NaCl, 2.6 mM KCl, 10 mM Na₂HPO₄, and 1.8 mM KH₂PO₄, pH 7.4) and 50 μl of each sample was added to black-wall 96-well plates (Corning), in duplicate. The excitation and emission wavelengths are 485 and 538 nm, respectively. All measurements are performed at room temperature and the reading of non-transformed parental Chlorella cells is subtracted before graphing.

After isolation of MEVs endo-loaded with the GFP protein, the MEVs are analyzed for size and fluorescence (size distribution). The Zeta View® nanoparticle tracking analyzer (NTA) from Particle Metrix is a Nanoparticle Tracking Analysis engine. Equipment is calibrated before the experiment according to the manufacturer's recommendations with polystyrene beads. The phosphate buffered saline (PBS) used for the day's experiments is evaluated with the Zeta View® NTA (normal average number of particles on screen in PBS: 0-5). Samples are diluted with PBS to be measured by Zeta View® NTA within the manufacturer's recommended reading range (50-200 particles per frame). Dilutions are made in PBS, then the sample is vortexed and placed in a 1 ml syringe for the analysis.

The samples are analyzed with the Zeta View® NTA in scatter mode (laser 488 nm) to determine the number and size distribution of the particles. The samples are then analyzed for fluorescence with the Zeta View® NTA using a laser at 488 nm and a fluorescence filter at 500 nm at different percentages of sensitivity (95%, 90% and 88%). The analog view of the Zeta View® NTA is activated during the fluorescence analysis to visualize the background noise.

### B. Enzymatic Activity of MEVs Endo-Loaded with GUS Protein

The parental strain of Chlorella is transformed with an expression vector encoding β-Glucuronidase (GUS) protein (SEQ ID NO:79) under control of a constitutive promoter. Successful transformants are selected for antibiotic resistance as described above. The modified Chlorella is cultured and MEVs are isolated from the engineered strain to confirm their cargo. Protein content is detected in the MEVs by Western blot analysis, while the enzymatic activity is determined by hydrolysis of 5-bromo-4-chloro-3-indolyl-beta-D-glucuronic acid (X-gluc) producing DiX-indigo and measured using light microscopy. Activity is calculated as percentage of blue cells.

### C. Bioluminescence Quantification from MEVs Endo-Loaded with Luciferase Protein

The parental strain of Chlorella is transformed with firefly luciferase (luc)-coding sequence using plasmids pAGS-0001 to pAGS-0012, as described in Example 3. To measure the luciferase activity, the cell density of each transgenic cell culture is so adjusted that optical density (OD) 750 nm=0.5 at the mid-log phase. Cells are harvested from 1 mL of cell culture by centrifugation (4000×g for 3 min) and used for the luciferase activity assay. Luciferase activity is measured using the firefly-Luciferase Assay Kit (Promega, Madison, WI, USA), according to manufacturer's protocol with slight modifications. Cell pellets are resuspended in 100 μL of cell lysis buffer and mixed vigorously by vortexing for 2-3 min. The resuspension is then centrifuged at 13,000 rpm for 5 min at 4° C. After centrifugation, 90 μL of the supernatant is transferred to a new tube and 10 μL of luciferase substrate is added to it. After mixing the supernatant and substrate, the luminescence is measured immediately using Glo Max™ 20/20 (Promega, Fitchburg, WI, USA). The luciferase assay is repeated three times.

### D. Total RNA Extraction from Endo-Loaded MEVs, mRNA Detection and Quantification by RT-qPCR

### 1. Total RNA Isolation from MEV Endo-Loaded with mRNA

Total RNA from isolated and purified MEVs endo-loaded with mRNA (1,5×10¹⁰ MEVs) are obtained using RNeasy Minikit (Qiagen). The final volume after isolation is 20 μL.

### 2. Reverse Transcription (RT SuperscriptII)

For the RT, 8 μL of isolated total RNA is mixed with 1.6 μL of Mix A (Hexamer Random Primer (500 ng/μl) Ref C118A from Promega. Hexamer (250 ng/RT in 20 μl) plus DNTP mix 10 mM (each); final concentration in each tube: Oligo hexamer 0.4 μl+DNTP 0.8 μL+H₂O 0.4 μl. Each tube is centrifuged 10 s, incubated at 65° C. 5 min, chilled into Eppendorf® plate at −20° C. and centrifuged 10 s. Next, the samples are incubated at 25° C. after adding 6.4 μl MixB/tube. Mix B (5x First Strand buffer (reference 18064-014), 3.2 μl+0.1 M DTT (P/N y0014 reference 18064-014) 1.6 μl+Recombinant Rnasin (Ref N251A) ribonuclease inhibitor 0.8 μl and 0.8 μl of Superscript II (reference 100004925). Incubation goes for 12 min. Then incubation at 42° C. for 50 min and then incubation at 70° C. for 15 min and finally at 4° C.

### 3. qPCR (Light Cycler 480 Roche)

qPCR is done adding Mix 1 (cDNA/3 μl+H₂O if needed) to the Mix 2 (specific primers F+R as given below) 2 μl+Takyon No ROX SYBR 2X MasterMix blue dTTP (Eurogentec, UF-NSMT-B0701) 5 μl) final volume of 10 μl into a 384 well plate (LightCycler® 480 Multiwell plate 384 clear, Roche, 5102430001).

The plate is sealed with a transparent film and centrifuged for 3 to 5 min (1500 rpm).

The amplification run is launched with the “2-step run protocol” template:
Denaturation: 95° C. for 10 min.
45 PCR cycles: 95° C. for 10 s, then 60° C. for 40 s.

Primer sequences for detection and quantification of GFP, mCherry, which is a constitutively red fluorescent protein, and beta-glucuronidase (GUS) are as follows:

eGFP(1-1) For3Rev3 400 nM
eGFP(1-1) F3
(SEQ ID NO: 80)
CACATGAAGCAGCACGACTT
eGFP(1-1) R3
(SEQ ID NO: 81)
GCGCGGGTCTTGTAGTTG
mCherry(1-1) For1Rev1 500 nM
mCherry(1-1) F1
(SEQ ID NO: 82)
GACCACCTACAAGGCCAAGA
mCherry(1-1) R1
(SEQ ID NO: 83)
CCGCTCGTACTGCTCCAC
Gus (1-1) For1Rev1 500 nM
Gus F1
(SEQ ID NO: 84)
CGCTCACACCGATACCATCA
Gus R1
(SEQ ID NO: 85)
CGGCTGATGCAGTTTCTCCT
GFP(1-1) For1Rev1 400 nM
GFP(1-1) F1
(SEQ ID NO: 68)
GTCCAGGAGCGCACCATCTTCT
GFP(1-1) R1
(SEQ ID NO: 69)
GATGCCCTTCAGCTCGATGCGGTT

### E. Quantification of Protein Endo-Loaded in MEV (by Western Blot)

Expression of the cargo protein is verified using SDS-PAGE followed by Western blot analysis. Semi-purified MEVs, semi-purified as described in Example 1b), are lysed on ice in RIPA buffer supplemented with β-mercaptoethanol (Sigma-Aldrich). Then, 30 μg of proteins are boiled and subjected to SDS-PAGE and transferred to nitrocellulose membranes (Bio-Rad). The blots are blocked with Tris buffered saline (TBS), 0.1% Tween-20 containing 5% BSA and incubated overnight at 4° C. with the relevant antibodies specific to the cargo protein. Subsequently, the blots are incubated with the relevant HRP-conjugated secondary antibody for 1 h at room temperature. Signals are revealed by chemiluminescence (Thermo Fisher Scientific).

### F. Quantification of GFP Protein Endo-Loaded in MEV. Fluorescence Readout by Zeta View® Nanoparticle Tracking Analyzer

The Zeta View® nanoparticle tracking analyzer (NTA) from Particle Metrix is a Nanoparticle Tracking Analysis engine. Equipment is calibrated before the experiment according to the manufacturer's recommendations with polystyrene beads. The phosphate buffered saline (PBS) used for the day's experiments is evaluated with the Zeta View® NTA (normal average number of particles on screen in PBS: 0-5). Samples are diluted with PBS to be measured by Zeta View® NTA within the manufacturer's recommended reading range (50-200 particles per frame). Dilutions are made in PBS, then the sample is vortexed and placed in a 1 ml syringe for the analysis.

The samples are analyzed with the Zeta View® NTA in scatter mode (laser 488 nm) to determine the number and size distribution of the particles. The samples are then analyzed for fluorescence with the Zeta View® NTA using a laser at 488 nm and a fluorescence filter at 500 nm at different percentages of sensitivity (95%, 90% and 88%). The analog view of the ZetaView® NTA is activated during the fluorescence analysis to visualize the background noise.

### G. Quantification of Catalase in Endo-Loaded MEVs

Protein content is detected in the MEVs by Western blot analysis, while the enzymatic activity is determined by the decomposition of H₂O₂, measured spectrophotometrically by following the decrease in absorbance at 240 nm. Activity is calculated using the extinction coefficient (e=40 mM⁻¹ cm⁻¹) and a unit of CAT is defined as micromoles of decomposed H₂O₂ per gram of dry weight (DW) per minute. The specific enzyme activity for the enzyme is expressed as units per milligram of protein (1 U=1 mol/min).

H. Quantification of total proteins and preparation for Western blot analysis

Total proteins are quantified by microBCA or Bradford test. Proteins are quantified using a spectrophotometer (in cuvettes) that measures protein concentration at OD595 nm (selecting “Bradford”) on the spectrophotometer. The reagent used is Bio-Rad Protein Assay Dye Reagent concentrate Cat #5000006 (stored at 4° C., taken out of the refrigerator 10 minutes before use to bring it to room temperature). Samples are measured in 1 ml cuvettes (Fisherbrand FB55143):
For the Blank: 200 μl Bradford reagent 5x+800 μl water is prepared.
For the Samples: 200 μl Bradford reagent 5x+800 μl sample in water is prepared. After quantification, samples are diluted to 60 μg protein in a maximum volume of 40 μL, 4 μl (bromophenol blue) 2X Laemmli buffer is added to the buffer to each sample and heated at 95° C. for 5 min. The tubes then are centrifuged to recover condensate.

### Example 5

Pharmacokinetics and biodistribution of MEVs following intranasal application

MEVs produced, purified, characterized, and labelled with DiR as described in in Examples 1 and 2, above (see, also copending International PCT application No. PCT/EP2022/070371, published Jan. 26, 2023, as International PCT publication No. WO2023/001894, which describes exogenous loading, and copending U.S. provisional application Ser. No. 63/349,006, which describes endogenous loading) were administered to C57BL/6 mice by intranasal administration. Mice were euthanatized at several time points (1 h, 2 h, 4 h and 8 h) after single intranasal administration (20 mL in each nostril) per animal; the brains were carefully isolated; brains were sectioned, embedded in OCT at max. 30 min post sampling and sliced at different distance of the bregma and kinetics thereof (see, FIGS. 21A-31B). FIG. 14 shows a positive control DiR-MEV on DAPI-stained brain slice in which a drop of MEV suspension was deposited on top of a brain tissue slide; the puncta are DiR-labeled MEV.

### A. Intranasal Administration

Mice were housed in Makrolon® polycarbonate cages with filter hoods, in a room where the air is continuously filtered to avoid contamination. During experiments, paired animals were caged at a constant temperature with a day/night cycle of 12/12 hours.

Animals received water (control tap water) and nutrition ad libitum. Animal health was examined every day to ensure that only animals in good health enter to the testing procedures and follow up to the study. At the beginning of the study, animals were identified with a number on the tail and separated in 5 groups:
Group 1: PBS-treated animals and sampling at 1 h
Group 2: MEV treated animals and sampling at 1 h
Group 3: MEV treated animals and sampling at 2 h
Group 4: MEV treated animals and sampling at 4 h
Group 5: MEV treated animals and sampling at 8 h

Intranasal compound administration was made using a dominant hand, the micropipette was loaded with 20 μl of MEV (for the first nostril). The tip of the filled pipette was placed near the mouse's left nostril, usually at a 45-degree angle. The droplet was placed close enough to the mouse's nostril so that the mouse could inhale the droplet. This procedure was reproduced for the second nostril. A total of 40 μl of MEV per animal was administrated. After full administration, the mouse was held in this position for 15 seconds.

### B. Organ Sampling

Mice were sacrificed by cervical dislocation. The temporal bone was opened, and the brain was sampled. Each sample rapidly was segmented into five parts (see, FIGS. 21A-G), placed in single cryoblocks, and embedded with O.C.T. compound using isopentane and stored at −80° C. Samples were cryosectioned, and labeled with DAPI fluorescent staining and/or Cresyl violet staining.

### C. Imaging and Data Analysis

The slide images were analyzed to determine the presence of MEV-labelled with DiR in DAPI and cresyl violet staining using an Akoya Phenochart™ whole slide viewer. Descriptive analysis by groups was expressed as mean±SD for continuous variables. Each brain was analyzed independently. The cresyl violet staining was used to reference-estimate the DiR analyzed brain areas for each section.

Quantification of MEV-DiR in the brain section 1 (+3.92 mm from the bregma). For the section 1, the MEV-DiR was detected in left and right olfactory nerve layer from 1-hour post administration reaching a plateau at 4 hours post administration (see FIGS. 22A-D and 23).

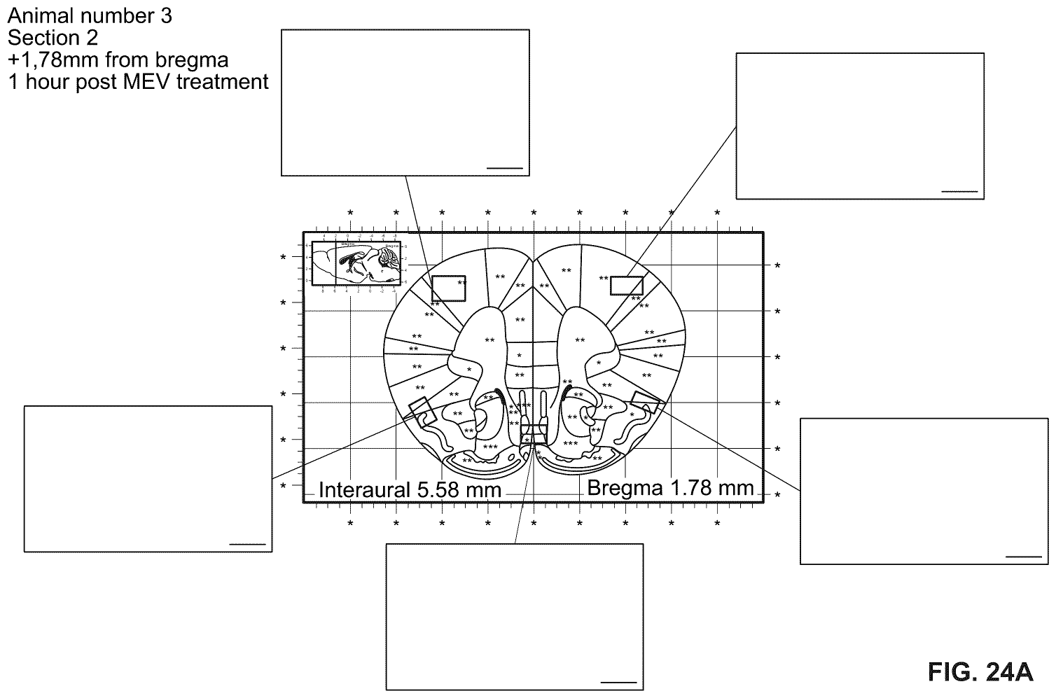

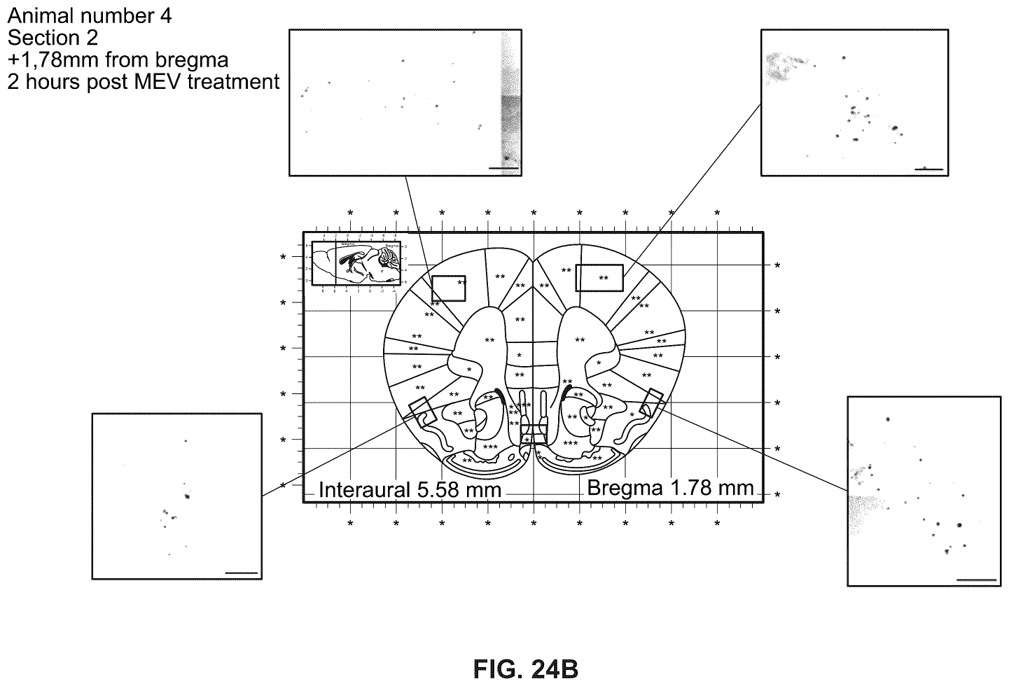

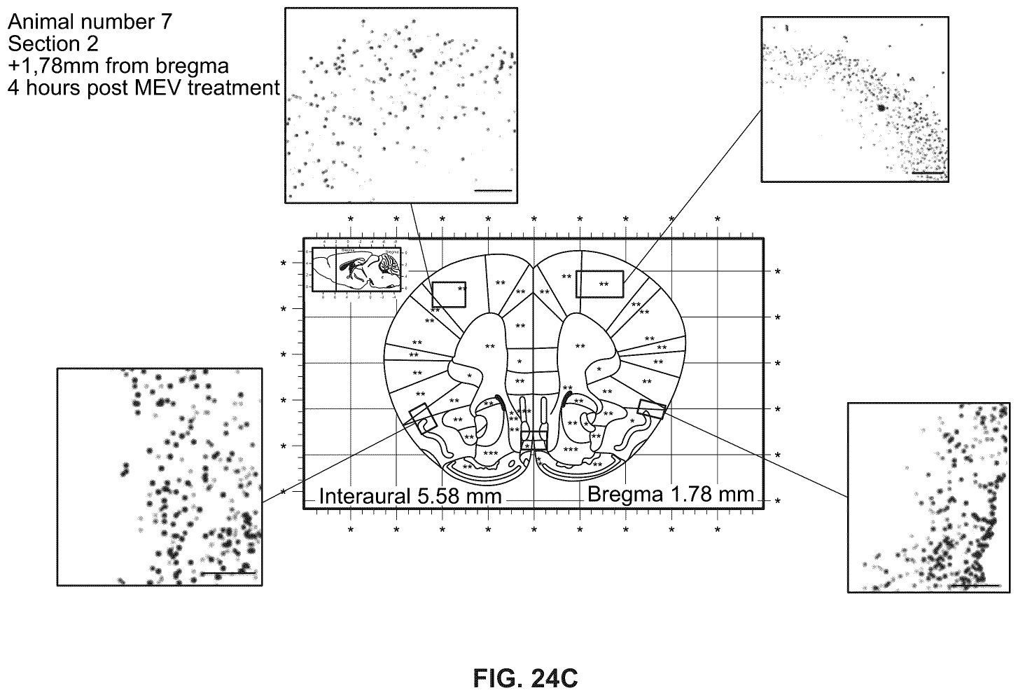

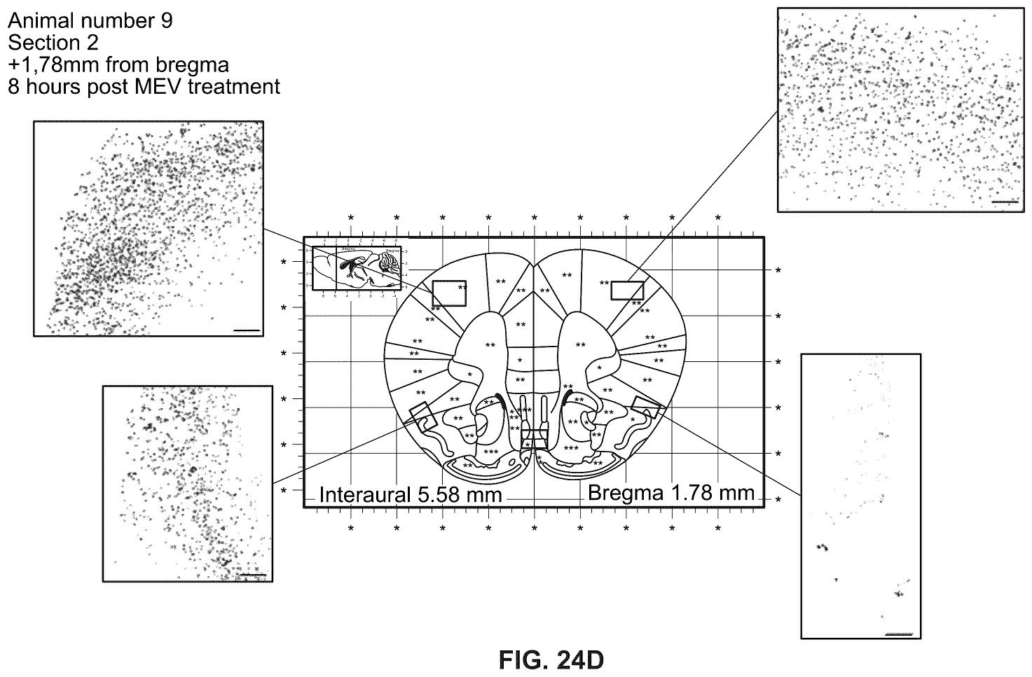

Quantification MEV-DiR in the brain section 2 (+1.78 mm from bregma). A progressive increase of MEV-DiR normalized intensity and number was observed in the primary motor cortex, piriform cortex, frontal cortex and agranular insular cortex from 2-hours post administration. Even if the number of animals per time-point was too small to profile a precise PK curve, the graphical representations suggest the MEVs reached a plateau in the primary motor cortex, frontal cortex and agranular insular cortex at 8 hours post administration (FIGS. 24A-D and 25A-D). Data indicate a progressive diffusion of the MEVs through primary motor cortex, piriform cortex, frontal cortex and agranular insular cortex when administrated by the IN route in mice.

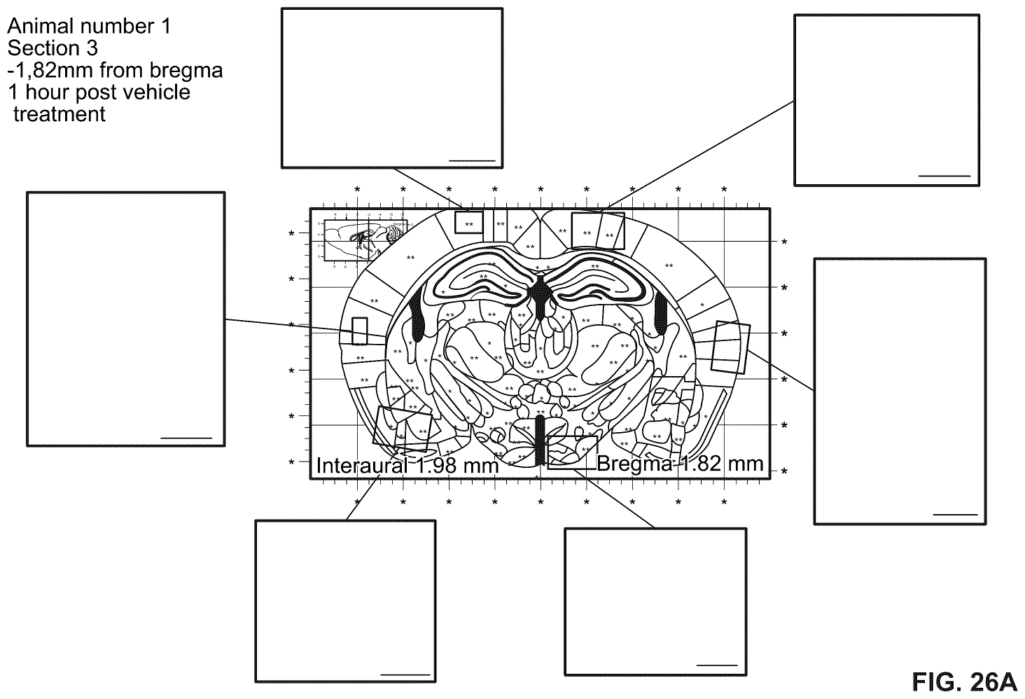

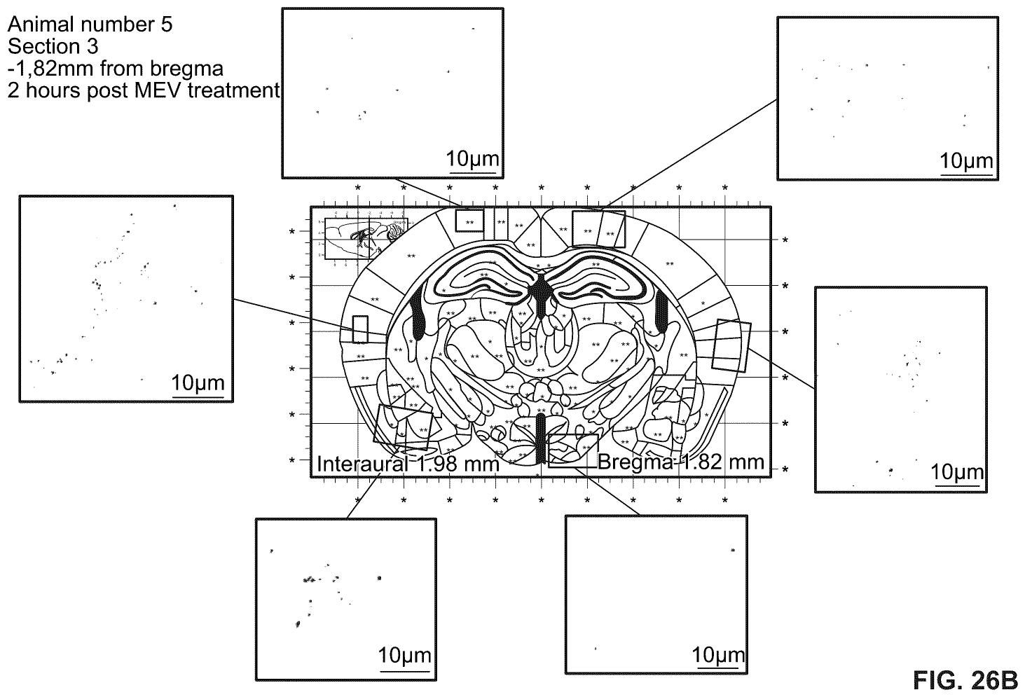

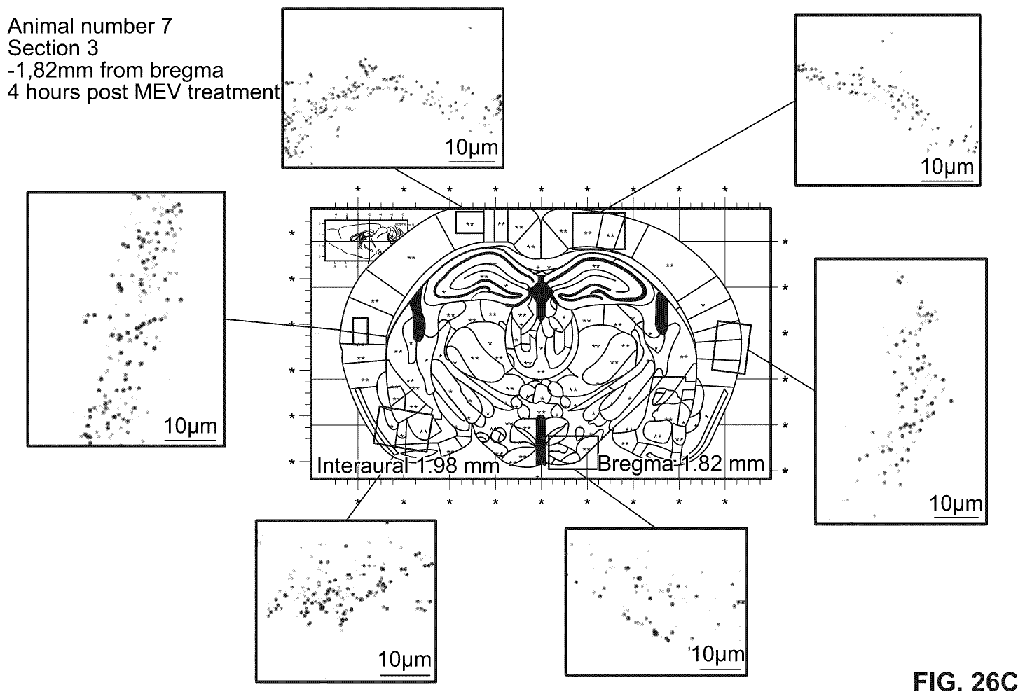

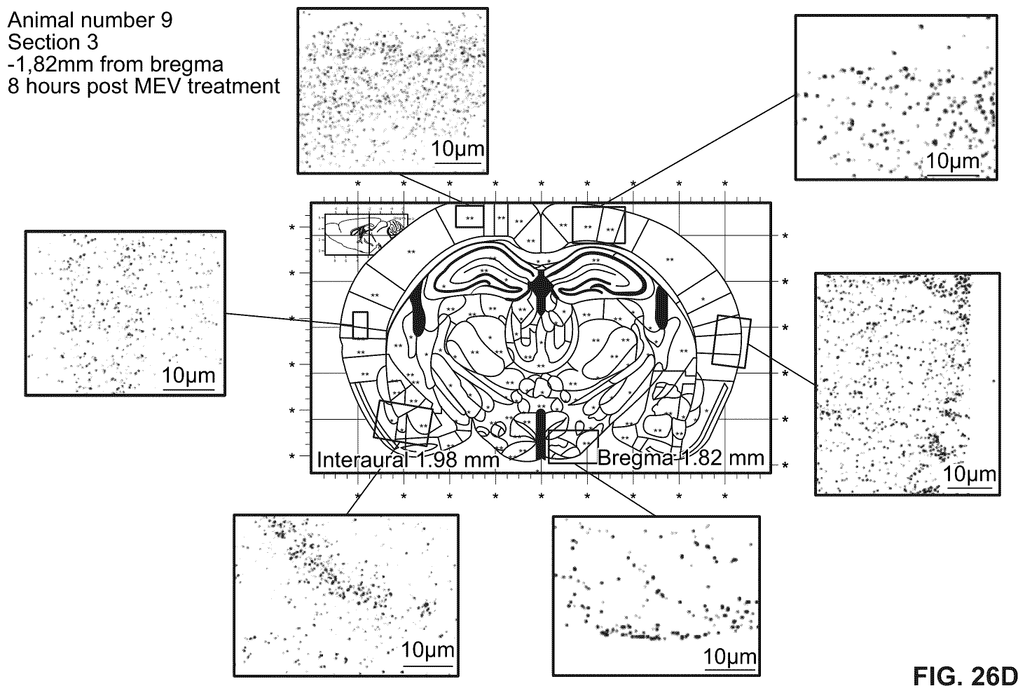

Quantification of MEV-DiR in the brain section 3 (−1.82 mm from bregma). A progressive increase of MEV-DiR normalized intensity and number was observed in the primary somatosensory cortex, auditory cortex, basolateral amygdaloid nucleus, retrosplenial granular cortex, temporal association cortex and the arcuate hypothalamic nucleus from 2-hour post administration. Even if the number of animals per time-point was too small to profile a precise PK curve, the graphical representations suggest the MEVs reached a plateau in the primary somatosensory cortex, auditory cortex, basolateral amygdaloid nucleus and the retrosplenial granular cortex, but not in the temporal association cortex and the arcuate hypothalamic nucleus, at 8 hours post administration (FIGS. 26A-D and 27A-F). These data indicate a progressive diffusion of the MEVs in the described brain regions when administrated by the IN route in mice.

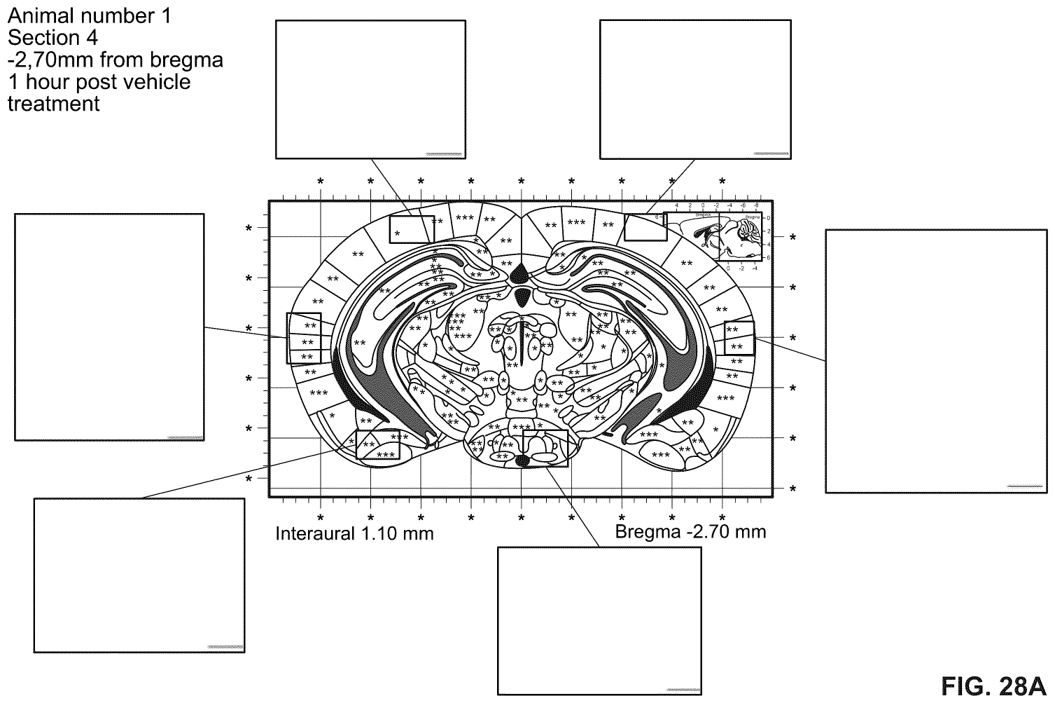

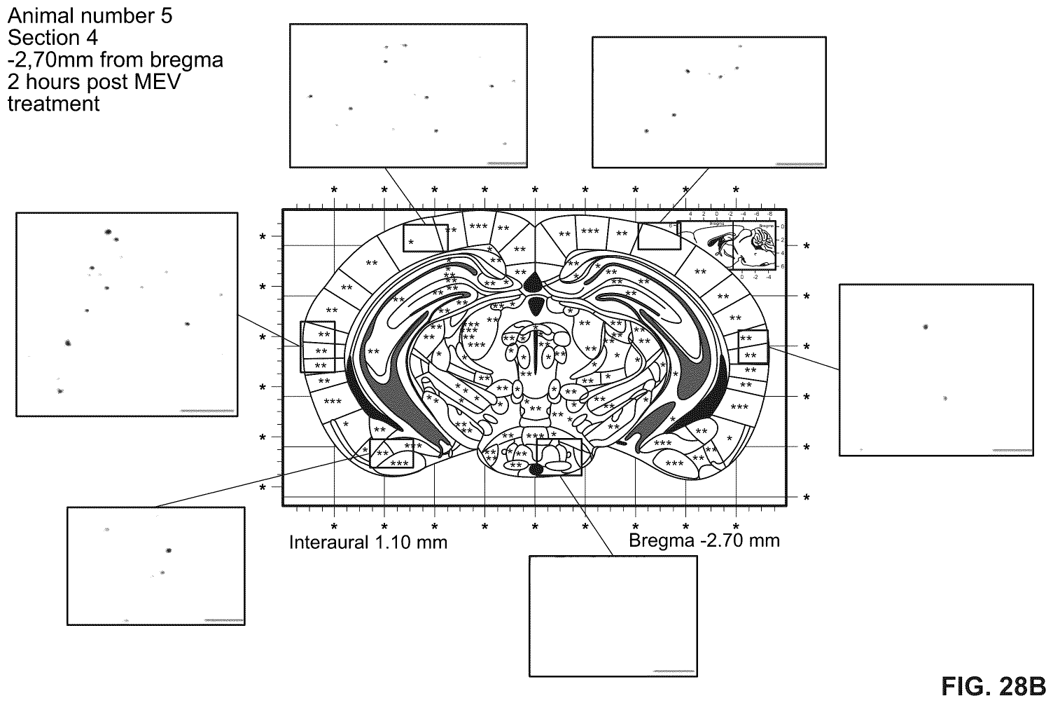

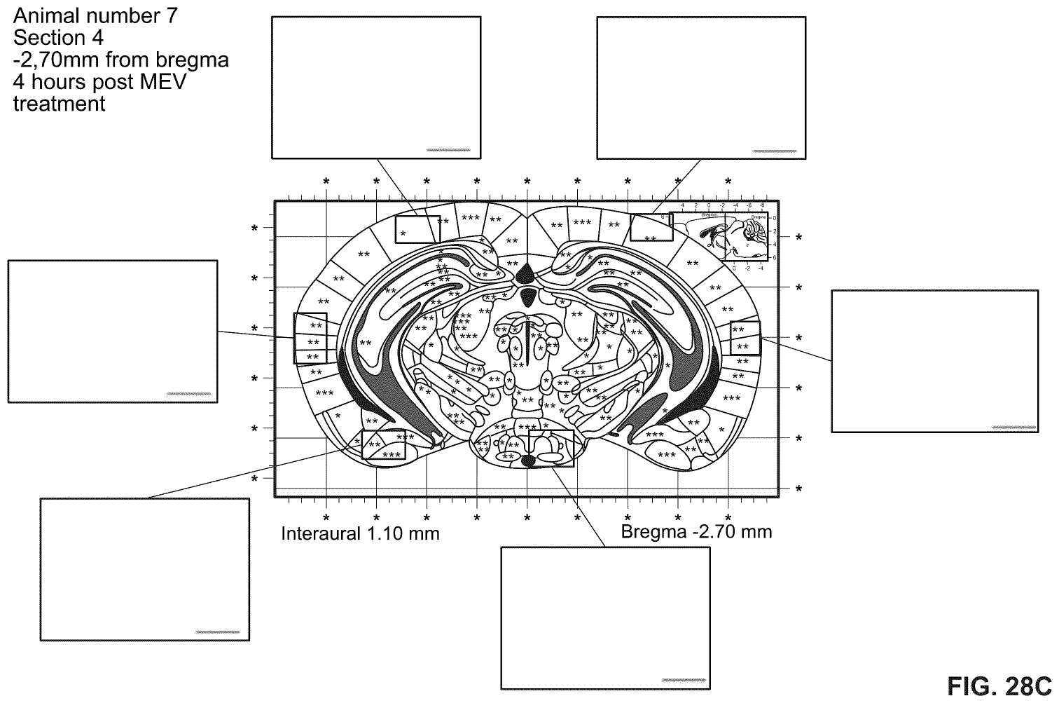

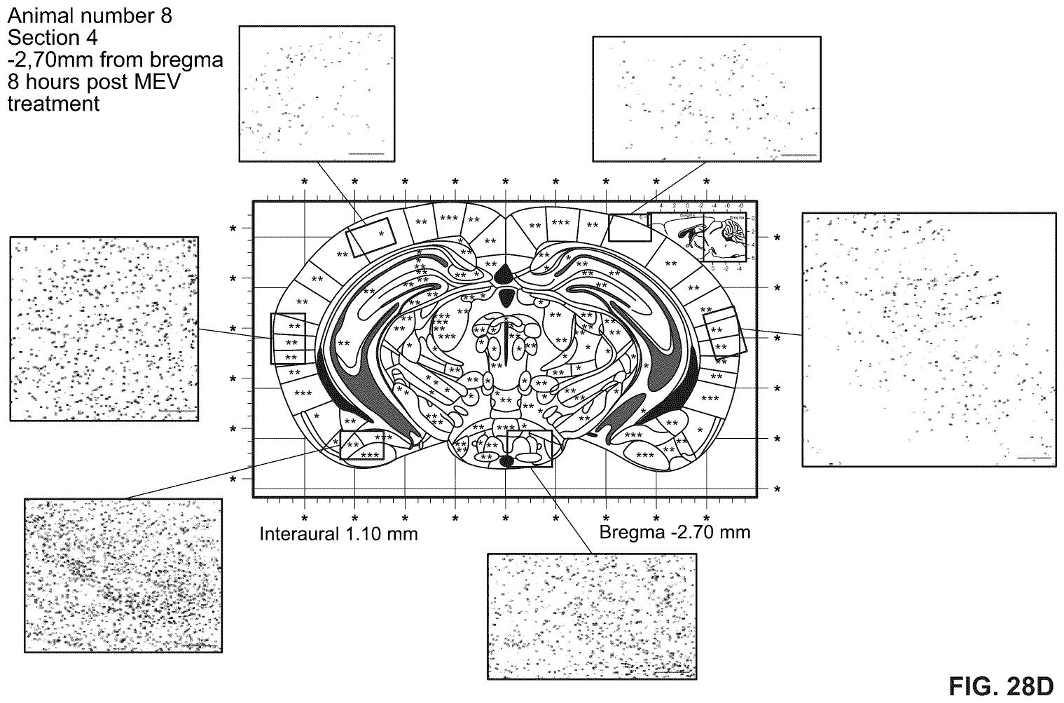

Quantification of MEV-DiR in the brain section 4 (−2.70 mm from bregma). A progressive increase of MEV-DiR normalized intensity and number was observed in the amygdala, left and right auditory cortex, temporal association cortex and ectorhinal cortex, the left and right primary visual cortex and the mammillary nucleus from 4-hour posts administration (FIGS. 28A-D and 29A-D). These data indicate a progressive diffusion of the MEVs in the described caudal brain regions when administrated by the IN route in mice.

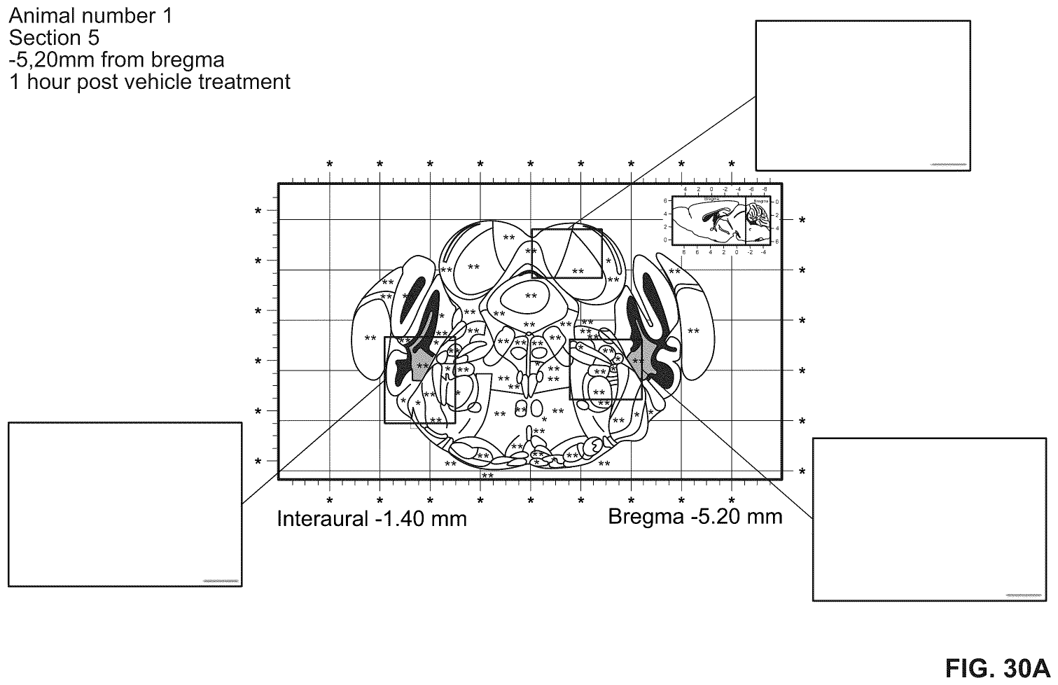

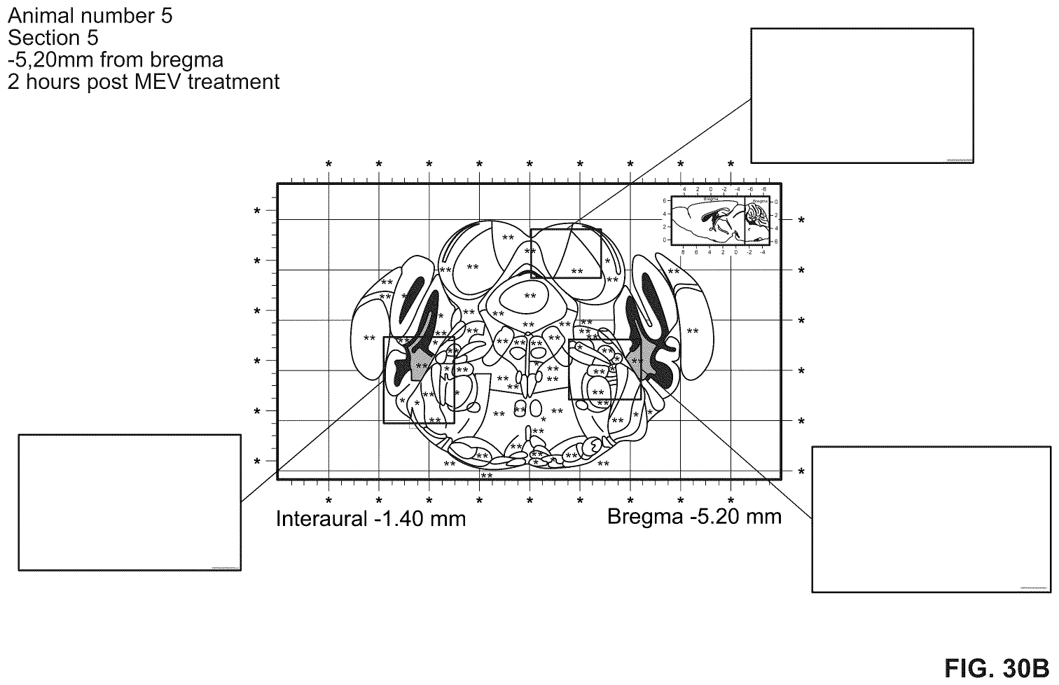

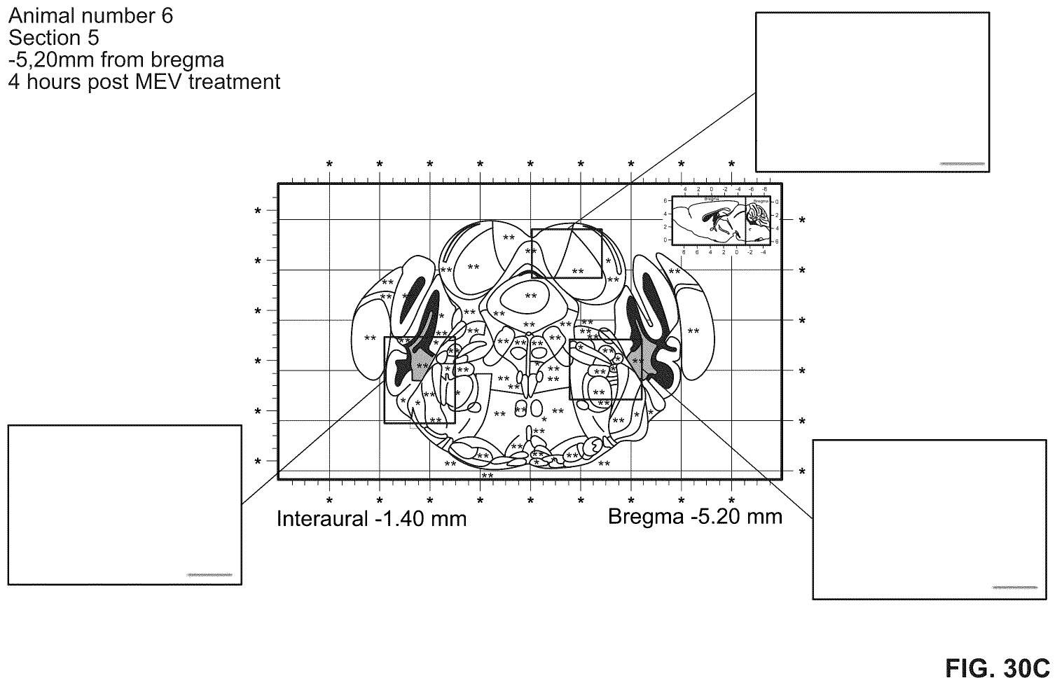

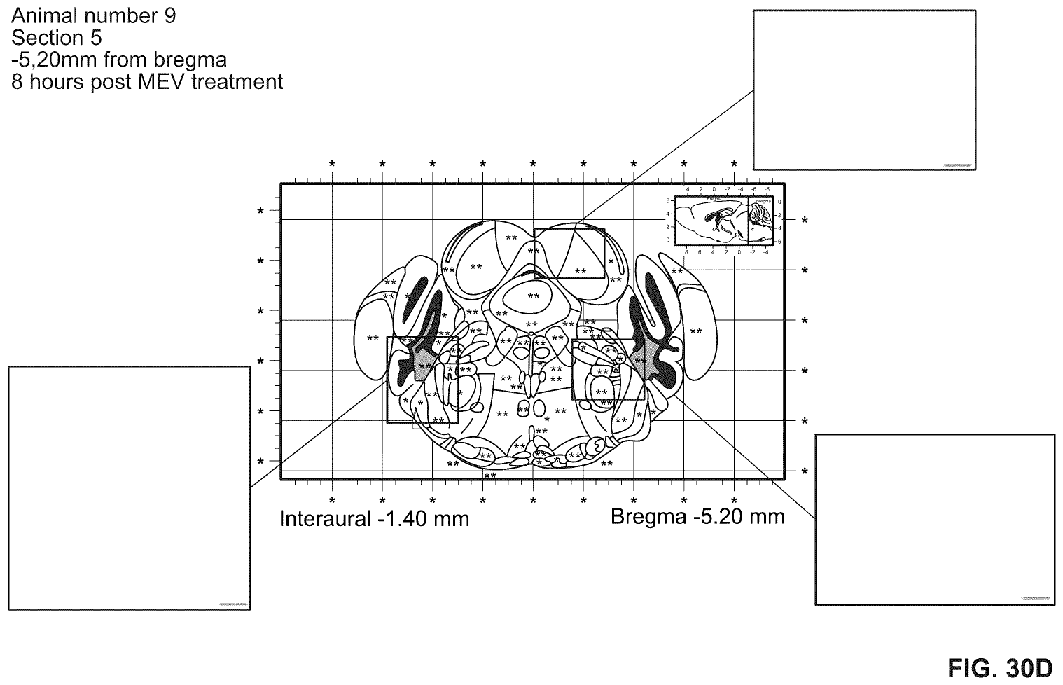

Quantification of the MEV-DiR in the brain section 5 (−5.20 mm from bregma). No MEV-DiR was detected in the principal trigeminal nucleus, inferior colliculus and tegmental nucleus, parabrachial nucleus and cerebellar peduncle and any other caudal area at the analyzed time point. The lack of MEV-DiR can be explained by a lack of diffusion into these brain areas up to 8 hours post administration (FIG. 30A-D).

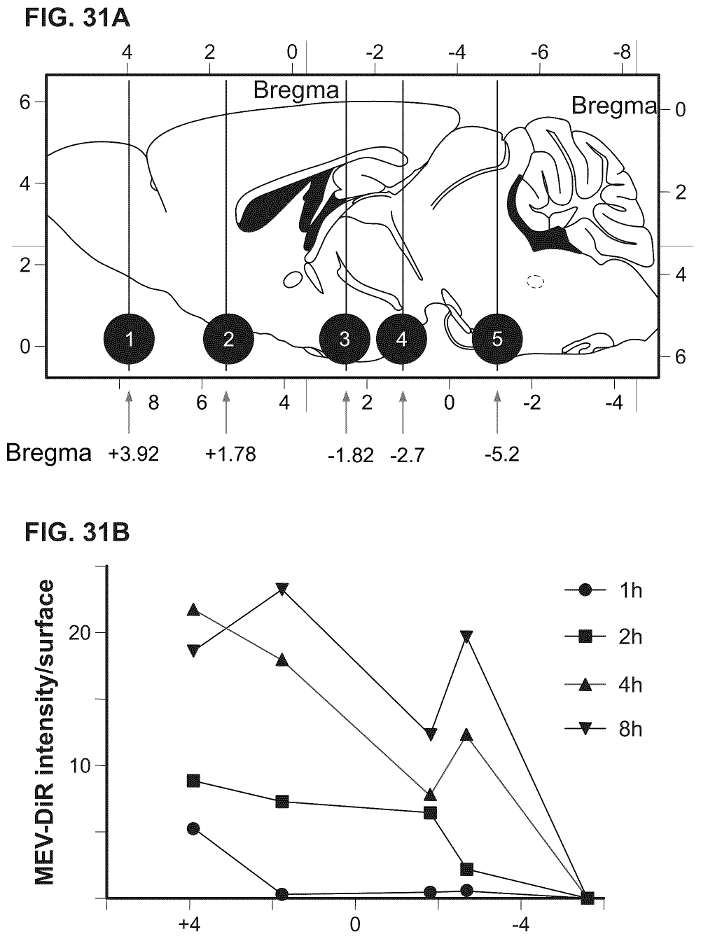

These results show that the intranasal administration of the MEVs leads to a progressive biodistribution of the vesicles in different but specific brain areas. This biodistribution is time-dependent and observed from the rostral to the caudal regions of the mouse brain (FIG. 31A-B). One hour after MEVs administration, the vesicles were specifically detected in the olfactory nerve layer but not in other more caudal regions. At two hours post MEV administration, the vesicles reached cortical regions and reached primary motor cortex, piriform cortex, frontal cortex, agranular insular cortex, primary somatosensory cortex, auditory cortex, retrosplenial granular cortex and temporal association cortex. MEVs also were found in basolateral amygdaloid nucleus, and the arcuate hypothalamic nucleus. No fluorescent signal was detected in the hippocampal regions, caudate putamen or in the nucleus accumbens. At four hours post administration, the MEV migrated up to the caudal brain regions reaching the amygdala, the left and right auditory cortex, the temporal association cortex and the ectorhinal cortex. No labelling was observed in hippocampus or substantia nigra. No vesicles were detected in the most caudal section examined where important structures, such as the trigeminal nucleus, inferior colliculus, tegmental nucleus, and parabrachial nucleus from 1 to 8 hours post administration indicating that the vesicles did not reach the most caudal part of the brain during the analyzed time points.

It is known that direct nose-to-brain transport of drugs and biologics (proteins, oligonucleotides, or viral vectors) is feasible via the olfactory or trigeminal nerve system. Olfactory nerve axons originating in the olfactory bulb (OB) penetrate the cribriform plate and terminate at the apical surface of the olfactory neuroepithelium; it is located at the roof of the nasal cavity. Filaments of the olfactory nerves are present in the anterior and in the posterior parts at the middle turbinate. The respiratory mucosa is densely innervated by sensory and parasympathetic trigeminal nerves and is more extensive than the olfactory nerve. Sensory maxillary branches innervate the deepest nasal segments, including the olfactory cleft. The pathways traversed by the MEVs, as shown herein, however are different.

FIGS. 21A-G provide: (i) a general overview of the experimental design of brain biodistribution studies; and (ii) the positions of the 5 brain sections studied; (FIGS. 21C-G) the regions analyzed to determine the PK and biodistribution of MEVs in each of the 5 brain sections studied. The graphs depict and identify regions of the brain for reference with the FIGS. 22A-D, 24A-D, 26A-D, 28A-D and 30A-D that show MEVs in the brain following IN administration.

### Example 6

In vitro delivery of cargo by the Microalgae Extracellular Vesicles (MEVs) in primary and established cell lines

### A. Cell Culture

Human primary cells (keratinocytes from skin; primary hepatocytes from healthy volunteers or patients) or established cell lines (A459 (lung), PC12 (neuronal), HEK293, and tumoral cell lines) are cultured in specific optimal conditions for each type of cells. The cells are grown in 75 cm² flasks, incubated at 37° C. with 100% humidity and 5% CO₂. The culture medium is changed every 2 days to ensure the growth of cells and to avoid contamination. Once reached sub-confluence, the cells are detached from the bottom of the flask with trypsin (0.25%), centrifuged (1600 g, 4 min), re-suspended in fresh culture medium and seeded at 2×10⁵ cells/plate.

### B. Uptake of Loaded MEVs by Neuronal Cells-Microplate Assay

PC12 neuronal cells are used as a model for in vitro evaluation of neuroprotective effects. Briefly, PC12 cells are seeded into 96-well plate (50,000 cells/well) and cultured for three days. Next, the catalase carrying MEVs (loaded as described above) are stained with DiR (5 μg/mL) and added to the wells in serial dilutions for various times. Following the incubation, the cells are washed three times with ice-cold PBS and solubilized in 1% Triton X-100. The sample fluorescence is measured using a SpectraMax® fluorescence microplate reader (Molecular Devices, USA) with excitation at 750 nm and emission at 780 nm. The amount of MEVs accumulated in neuronal cells is normalized for total protein content and expressed as the number of MEVs per mg of protein. All MEV formulations are prepared at the same level of fluorescence, and a separate calibration curve is used for each MEV formulation.

### C. Uptake of Loaded MEVs by Neuronal Cells-Confocal Microscopy

The catalase loaded MEVs (100 μg/mL total protein) are sonicated, stained with DiR (5 μg/mL) and incubated with PC12 cells grown on chamber slides (1×10⁵ cells/chamber) for various time intervals. Following the incubation, the cells are washed with PBS, fixed in 4% paraformaldehyde and permeabilized with 0.1% Triton X-100. Fluorescent staining is performed using anti-actin antibody (Abcam ab179467, 1:100) with goat anti-rabbit IgG Alexa Fluor 488 (Abcam ab 150077, 1:1000) as the secondary antibody. DAPI (4′,6-diamidino-2-phenylindole) is used as nuclear counterstain prior to the imaging. Accumulation of fluorescently labelled MEVs is visualized by a LSM700 laser scanning confocal microscope (Zeiss) and images are processed with LSM Image Browser.

### D. Neuroprotective Effects of the Loaded MEVs

The protection of PC12 cells against 6-OHDA-induced cytotoxicity is assessed by MTT assay. For this purpose, PC12 cells (1×10⁵ cells/mL) are seeded into a 96-well plate and allowed to attach overnight. Then, the cells are exposed to 200 μM 6-hydroxydopamine (6-OHDA) and different catalase-loaded MEV formulations, or catalase alone, or empty MEVs for four hours. Following the incubation, the cells are washed 3 times with ice-cold PBS, and incubated with the corresponding catalase loaded MEV formulations, or catalase alone, or empty MEVs for another 24 hours. Following the treatment, 20 μL of MTT tetrazolium dye solution (5 mg/mL) is added into each well. After 3 hours of incubation at 37° C., the MTT-containing medium is removed and 100 μL DMSO is added into each well to dissolve the purple formazan precipitate. Absorbance is measured at 570 nm using a SpectraMax® microplate reader (Molecular Devices, USA) and cell viability is expressed as a percentage of viable cells in the treated groups compared to the untreated control group.

### E. MEV Internalization in Human Cancer Cells

HeLa cell line of human cervix epithelioid carcinoma is used to study uptake of GFP-loaded MEVs. For flow cytometry analysis, the cells are seeded in 24-well plates (15,000 cells/well). After 24 h, cells are incubated with the cargo-loaded MEVs for 4 h and subsequently washed with PBS, washed again with acid wash buffer (0.5 M NaCl, 0.2 M acetic acid) to remove membrane-bound MEVs and once more with PBS. Cells are detached with trypsin, fixed in 0.2% paraformaldehyde in PBS and analyzed using LSRII flow cytometer with CellQuest Pro software (BD Biosciences). For confocal microscopy analysis, HeLa cells are seeded in 16-well chamber slides (Lab-Tek) at 4,000 cells/well. After 24 h, cells are incubated with the cargo-loaded MEVs for 4 h and subsequently washed with PBS, washed again with acid wash buffer (0.5 M NaCl, 0.2 M acetic acid) to remove membrane-bound MEVs and once more with PBS. Cells are fixed with 4% paraformaldehyde in PBS at room temperature for 20 min. Slides are then washed with PBS and mounted using Fluorsave (Calbiochem). Confocal fluorescent imaging is performed using a LSM700 laser scanning confocal microscope (Zeiss) and images are processed with LSM Image Browser.

### F. Study of the Internalization of MEVs Loaded with GFP (Green Fluorescent Protein) in Normal Human Hepatocytes

### 1. Effect of MEVs on Primary Human Hepatocytes PHH Viability

PHHs (1×10⁵ cells/mL) are seeded into a 96-well plate and allowed to attach overnight. Then, the cells are exposed to different cargo loaded MEV formulations or empty MEVs for four hours. Following the incubation, 20 μL of MTT tetrazolium dye solution (5 mg/mL) is added into each well. After 3 hours of incubation at 37° C., the MTT-containing medium is removed and 100 μL DMSO is added into each well to dissolve the purple formazan precipitate. Absorbance is measured at 570 nm using a SpectraMax® microplate reader (Molecular Devices, USA) and cell viability is expressed as a percentage of viable cells in the treated groups compared to the untreated control group.

### 2. MEV Internalization in Primary Human Hepatocytes (PHH)

The internalization of the GFP-protein-cargo-loaded MEVs in PHHis determined by flow cytometry. For flow cytometry analysis, cells are seeded in 24-well plates (15,000 cells/well). After 24 h, cells are incubated with the MEVs for 4 h and subsequently washed with PBS, washed again with acid wash buffer (0.5 M NaCl, 0.2 M acetic acid) to remove membrane bound MEVs and once more with PBS. Cells are detached with trypsin, fixed in 0.2% paraformaldehyde in PBS and analyzed using LSRII flow cytometer with CellQuest Pro software (BD Biosciences). For confocal microscopy analysis, cells are seeded in 16-well chamber slides (Lab-Tek) at 4,000 cells/well. After 24 h, cells are incubated with the MEVs for 4 h and subsequently washed with PBS, washed again with acid wash buffer (0.5 M NaCl, 0.2 M acetic acid) to remove membrane bound MEVs and once more with PBS. Cells are fixed with 4% paraformaldehyde in PBS at room temperature for 20 min. Slides are then washed with PBS and mounted using Fluorsave (Calbiochem). Confocal fluorescent imaging is performed using a LSM700 laser scanning confocal microscope (Zeiss) and images are processed with LSM Image Browser.

### G. Delivery and Expression of the GFP mRNA Loaded in MEVs in Human Cells

### 1. mRNA translation studies

The delivery and biological activity in hepatocytes of the GFP-mRNA-cargo-loaded MEVs is determined by total RNA extracted from the cells using the RNeasy Minikit (Qiagen) according to the manufacturer's instructions. Then, 500 ng of total RNA is reverse transcribed into cDNA using a reverse transcriptase (Promega) and real-time PCR is performed using the LightCycler 480 SYBR Green I Master Kit on an LC480 device (both from Roche Diagnostics). The mRNA level is calculated by normalizing the threshold cycle (CT) of target genes to the CT of the 28S ribosomal RNA housekeeping gene. The primers are designed using primer software from Roche Diagnostics and are purchased from Eurogentec.

### 2. Protein Expression Studies

The delivery and biological activity in hepatocytes of the GFP-protein-cargo-loaded MEVs is determined by SDS-PAGE followed by Western blot analysis. Cells are lysed on ice in RIPA buffer supplemented with β-mercaptoethanol (Sigma-Aldrich). Then, 30 μg of proteins are boiled and subjected to SDS-PAGE and transferred to nitrocellulose membranes (Bio-Rad). The blots are blocked with Tris buffered saline (TBS), 0.1% Tween-20 containing 5% BSA and incubated overnight at 4° C. with the following antibodies. Subsequently, the blots are incubated with the relevant HRP-conjugated secondary antibody for 1 h at room temperature. Signals are revealed by chemiluminescence (Thermo Fisher Scientific).

### H. Delivery and Expression of the Payload in Normal Human Keratinocytes Cellular Uptake of GFP-Loaded MEVs

The delivery and expression of the payload in keratinocytes is studied by confocal microscopy. MEVs are loaded with GFP protein as described above, added to the cells at various concentrations, and incubated at 37° C. for 2, 4, 8, and 24 h. The cells subsequently are washed twice with RPMI1640, fixed with 4% paraformaldehyde in PBS (pH 7.4), and treated with Triton X-100 (0.1%) to increase the permeability of the cell membrane. The fixed cells are washed with PBS and then stained with Hoechst blue to visualize the cell nucleus, and with phalloidin-fluorescein isothiocyanate conjugate for staining the actin cytoskeleton. After staining, the cells are washed again with PBS, and allowed to dry overnight. Microscopic images are obtained using an inverted confocal microscope FluoView FV1000 (Olympus) equipped with a 20× UPlanSApo objective. GFP and Hoechs are visualized with a wavelength of excitation/emission of 559/578 nm and 360/460 nm, respectively.

### I. Delivery and Expression of Payload in Human Cancer Cell Lines

### 1. Cellular Uptake of GFP-Loaded MEVs

The internalization, delivery and expression of payload (GFP) in cancer cells is studied by confocal microscopy. The experiments are performed on sub-confluent cells grown on glass slides. MEVs are added to the cells at various concentrations, and incubated at 37° C. for 2, 4, 8, and 24 h. The cells subsequently are washed twice with RPMI1640, fixed with 4% paraformaldehyde in PBS (pH 7.4), and treated with Triton X-100 (0.1%) to increase the permeability of the cell membrane. The fixed cells are washed with PBS and then stained with Hoechst blue to visualize the cell nucleus, and with phalloidin-fluorescein isothiocyanate conjugate for staining the actin cytoskeleton. After staining, the cells are washed again with PBS, and allowed to dry overnight. Microscopic images are obtained using an inverted confocal microscope FluoView FV1000 (Olympus) equipped with a 20× UPlanSApo objective. GFP and Hoechs are visualized with a wavelength of excitation/emission of 559/578 nm and 360/460 nm, respectively.

### 2. RT-PCR Analysis and Gel Electrophoresis

The cells (1×10⁶) are grown in culture plates for 24 h. Next, the loaded MEVs carrying siRNA, or empty MEVs, or siRNA alone, are added to the wells in serial dilutions and incubated for 1-24 hours. Following the incubation, the medium is changed for the normal culture medium. 72 h after the MEV treatment, total RNA is extracted from the cultured cells using Trizol reagent. The RNA precipitate is centrifuged and dissolved in 20 μl of RNase-free water. UV spectrophotometer analysis at 260/280 nm and agarose gel electrophoresis are used to confirm the quality of purified RNA. 1 μg of total RNA is reverse transcribed to synthesize cDNA at 42° C. for 1 h; this cDNA is then subjected to PCR amplification with specific primers in 25-μl reactions. Next, the PCR products are resolved on 2% agarose gel, stained with ethidium bromide, and observed with a UV trans-illuminator. The digital images are quantified with ImageJ analysis software and the results are expressed as the relative expression level of relevant genes over GAPDH.

### 3. Gene Expression Analysis by RT-qPCR

The cells treated with the MEVs and cultured for a total of 72 h are trypsinized and washed with PBS. Total RNA is extracted from the cell pellets using the RNeasy Minikit (Qiagen) according to the manufacturer's instructions. Then, 500 ng of total RNA is reverse transcribed into cDNA using a reverse transcriptase (Promega) and real-time PCR is performed using the LightCycler 480 SYBR Green I Master Kit on an LC480 device (both from Roche Diagnostics). The mRNA level is calculated by normalizing the threshold cycle (CT) of target genes to the CT of the 18S ribosomal RNA housekeeping gene. The primers are designed using primer software from Roche Diagnostics and are purchased from Eurogentec.

### 4. Western Blot Analysis

The cells (2×10⁶) treated with the MEVs and cultured for a total of 72 are trypsinized, washed with PBS and lysed on ice in RIPA buffer supplemented with β-mercaptoethanol (Sigma-Aldrich). Then, 50 μg samples of total protein are subjected to 10% standard sodium dodecyl sulfate-polyacrylamide gel electrophoresis (SDS-PAGE), with pre-stained molecular weight markers run in parallel. Subsequently, the resolved proteins are electro-transferred onto PVDF membranes. After extensive washing, the membranes are incubated with the relevant horseradish peroxidase-conjugated antibody (Sigma-Aldrich) for 1 h at room temperature and developed with a chemiluminescence detection kit (Thermo Fisher Scientific). Membranes probed for the oncoproteins are re-probed for β-actin to normalize for loading and/or quantification errors and to allow comparisons of target protein expression. The protein expression is quantified with a gel analysis system (Bio-Rad). 5. Cell proliferation assay

The cells are seeded into 96-well plate (10,000 cells/well) and allowed to attach for 18 h. Next, the cells are treated with the MEVs as above; at 72 h after the treatment, 10 μl 10 mg/ml MTT is added to the cells in each well. The cells are incubated for 4 h, after which 100 μl DMSO is added, and the cells are allowed to lyse for 15 min. All of the analyses are carried out in triplicate. Optical density at 492 nm is determined with a SpectraMax® microplate reader (Molecular Devices, USA). To determine the inhibitor rate, the absorbance values are then normalized to the values obtained from the blank control cells.

### Example 7

Targeting Neuronal Cells with Microalgae Extracellular Vesicles (MEVs). Biological Activity of Protein- and mRNA-Loaded Microalgae Extracellular Vesicles (MEVs) in Neurons In Vitro

### 1. Overexpression/Rescue in KO Neuronal Mouse Culture by MEV Carrying GABABla mRNA

Pyramidal cells in DIV10-14 hippocampal dissociated cultures from GABABla” mice are analyzed using whole-cell patch clamp technique, recording of spontaneous excitatory activity (sEPSCs). Cultures are either untreated or preincubated with MEVs at different concentrations (from 10⁶ to 5×10¹⁰ particles) or preincubation periods (from 0.5 to 24 hours). For each cell, baseline signal is recorded for 3 mins and compared with post-application baclofen (20 μM) recording to assess rescue efficacy. The analysis includes sEPSCs frequency, amplitude and membrane parameters (Rm, Rs, Cm).

### 2. Expression of GFP Protein in Neuronal Mouse Culture by MEV Carrying eGFP Protein or eGFP mRNA

Pyramidal cells in DIV10-14 hippocampal dissociated cultures from mice are analyzed for the expression of eGFP marker. The experiments are performed on sub-confluent neuronal cells grown on glass slides. MEVs are loaded with GFP protein as described above, added to the cells at various concentrations, and incubated at 37° C. for 2, 4, 8, and 24 h. The cells subsequently are washed twice, fixed with 4% paraformaldehyde in PBS (pH 7.4), and treated with Triton X-100 (0.1%) to increase the permeability of the cell membrane. The fixed cells are washed with PBS and then stained with Hoechst blue to visualize the cell nucleus, and with phalloidin-fluorescein isothiocyanate conjugate for staining the actin cytoskeleton. After staining, the cells are washed again with PBS, and allowed to dry overnight. Microscopic images are obtained using an inverted confocal microscope FluoView FV1000 (Olympus) equipped with a 20× UPlanSApo objective. GFP and Hoechs are visualized with a wavelength of excitation/emission of 559/578 nm and 360/460 nm, respectively.

### Example 8

### A. Evaluation of Toxicity on MEVs in Mice

Experiments were performed to assess the signs of MEV toxicity in vivo using Balb/C mouse model after oral and intratracheal administration.

Analysis of MEV toxicity in vivo:

Toxicity was evaluated in several ways: clinical signs, body weights, hematological analysis, biochemical analysis, histological analysis on main organs (liver, spleen, kidney, lung and brain). The MEV samples were stored at −80° C. and thawed just prior to in vivo administrations. After thawing, each sample was mixed by vigorous vortexing for 1-2 minutes. Male Balb/C mice at 5 weeks of age and with a mass about 20 g each, were used. Animals were acclimatized. Animals were housed in polyethylene cages (<5 animals/cage), in a controlled environment with 12:12 light-dark at the temperature of 24±1° C. (mean±SD) and fed once daily with an adapted pelleted feed. Water was offered ad libitum. The animals were randomly assigned to experimental groups and acclimatized for at least 7 days before the initiation of the designed study.

The experiment was designed to determine the eventual toxicity of the test compound after its administration in mice through exemplary routes including oral, and respiratory tract (intratracheal) routes. As described above, male Balb/C mice, with a mass about 20 g each, were used. Animals were acclimatized. After a test item is administered, all mice are closely monitored for 10 days.

### Clinical Evaluation

Daily clinical examination of all animals included, observation of behavior and signs of suffering (cachexia, weakening, difficulty for moving or feeding, etc.); test item toxicity (hunching, convulsions), and other such parameters. Determination of body weight once a week for each animal, a body weight curve was designed (Mean±SD). Observation of acute reactions was done after administration of the tested compounds.

### Clinical Pathology Investigations

After the end of the in-life phase, all animals were euthanized. Clinical pathology investigations were performed at experiment termination.

### Blood Collection

The blood samples were collected from mice by intracardiac puncture into different vials. Aliquots of blood were collected for various clinical pathology investigations into tubes containing anticoagulants: for hematology analysis with K2 EDTA and for biochemistry analysis with lithium heparin.

### Clinical Chemistry

Plasma was separated after centrifugation of whole blood samples, 45000 rpm for 15 minutes and analyzed for the following parameters at the end of treatment for all animals. Clinical chemistry analysis parameters are indicated as: Alanine, Aminotransferase (ALT), Aspartate Aminotransferase (AST), and Gamma-glutamyl transferase (GGT) in units per liter (U/L); Urea in g/L and Creatinine in mg/L.

### Hematology

The following hematological parameters were determined at the end of treatment of all animals: Hemoglobin (HGB) in g/L; Hematocrit (HCT) in L/L; Mean Corpuscular Volume (MCV) in fL, eosinophils (EO) and MHCH (BASO) as 10⁹/L. When animals are euthanatized, an autopsy is performed, and a careful macroscopic evaluation is performed. If any organs look abnormal, a detailed description and analysis is performed.

### Histological Analysis

Histological analysis is performed for the 5 following organs: Lung, Spleen, Liver, Kidney, and Brain. The organs are collected, weighed, macroscopically observed, fixed in 4% Paraformaldehyde (PFA) and paraffin-embedded, cut into 5-7 μM sections and then observed under fluorescence microscope.

### Histopathological Scoring

Each H&E (hematoxylin- and eosin-stained) section was thoroughly examined histologically, and lesions observed were recorded in an Excel spreadsheet, their severity graded (minimal, mild, moderate, or severe). Their distribution also was characterized (i.e., as focal, multifocal, focally extensive or diffuse), as well as, their localization.

### B. Toxicity Study Results: Clinical Examinations

### Clinical Chemistry

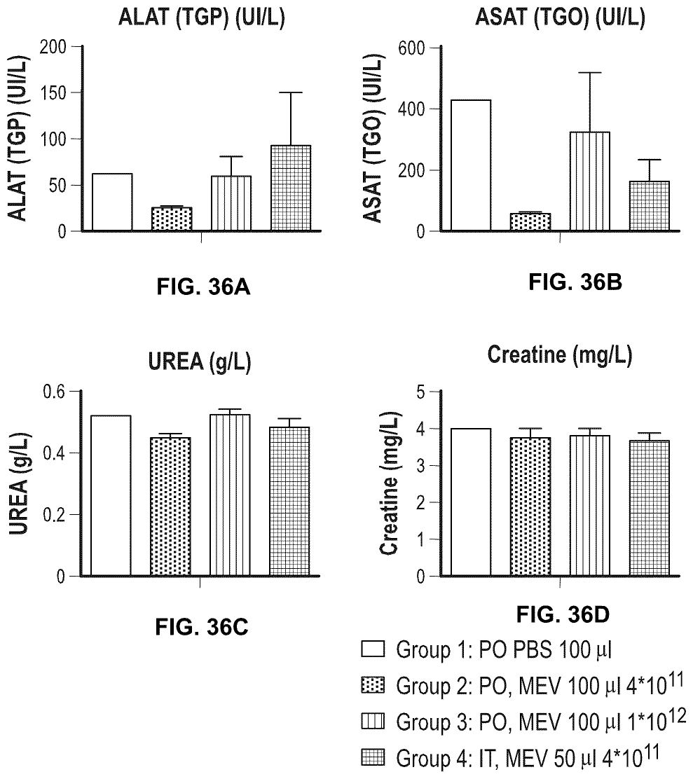

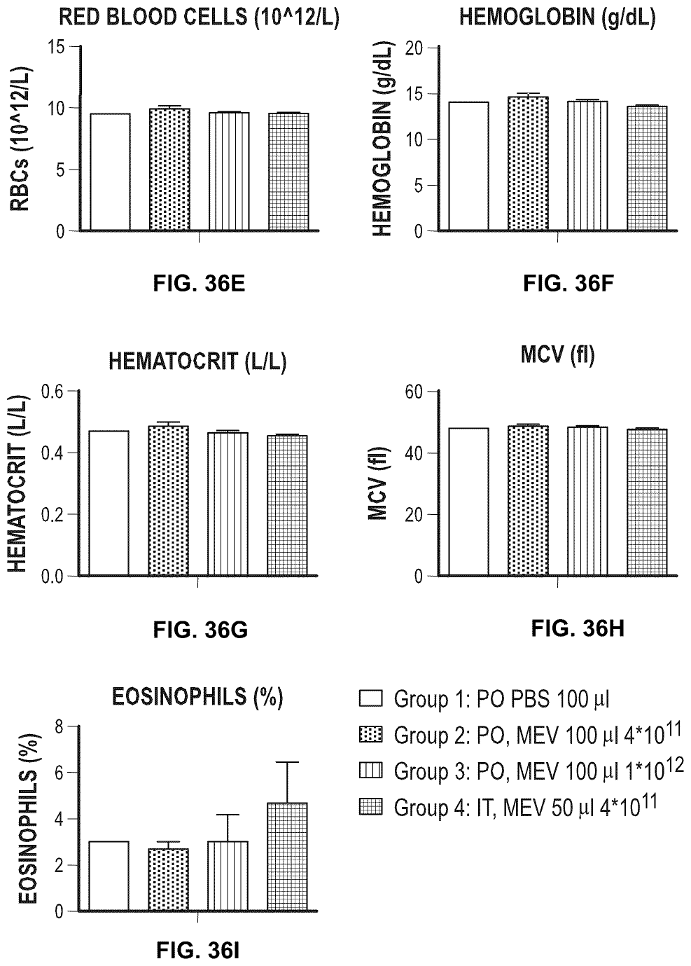

As shown below, two parameters Urea and Creatinine did not change in all treated groups compared to control groups. The enzymes Alanine Aminotransferase (ALT) and Aspartate Aminotransferase (AST) show random non-significant deviations. FIGS. 36A-D present these results. The parameter Gamma-glutamyl transferase value is detected at low level (<6) in all groups indicating a protocol error and are not presented.

The results of the clinical chemistry parameters do not indicate any toxicity at all tested doses in the mice either by oral or intratracheal administration.

### Hematology

The measured parameters were not significantly changed compared to controls. FIGS. 36A-I present the results in which MEV toxicity was evaluated by (1) chemistry parameters: ALAT, ASAT, urea and creatine; and (2) by hematology parameters in four groups of mice: Group 1 mice were administered 100 μl of PBS by PO delivery (white bars); Group 2 mice were administered 100 μl of 4*10¹¹ MEVs/mouse by PO delivery (bar with black and white tiles); Group 3 mice were administered 100 μl of 4*10¹² MEVs/mouse by PO delivery (white bars with vertical black lines); Group 4 mice were administered 100 μl of 4*10¹¹ MEVs/mouse by IT delivery (white bars with black squares).

The results present blood parameters that can be altered when there is one or more of blood-, kidney-, and liver-related toxicity. Hematocrit, hemoglobin, eosinophil levels, RBC count, and volume (MCV) are hematologic parameters; urea and creatine are biochemical markers of kidney injury; ALAT (or ALT) and ASAT (or AST) are biochemical markers of liver injury. Alanine transaminase (ALT), also called alanine aminotransferase (ALAT), is a transaminase enzyme that occurs in plasma and in various body tissues, but most commonly in the liver. Significantly elevated levels of ALT can be related to liver-related problems, such as hepatitis and/or liver damage. Aspartate transaminase (AST), also known as aspartate aminotransferase (ASAT), is another transaminase enzyme important in amino acid metabolism. Beyond liver toxicity, AST can be elevated also in diseases affecting other organs. Serum ALT level, serum AST level, and their ratio (AST/ALT ratio) are used clinically as biomarkers for liver health. In the instant study, no statistically-significant differences between experimental groups in ALT and AST levels were observed.

Eosinophils (eosinophiles) are a variety of white blood cells and one of the immune system components responsible for combating multicellular parasites and certain infections in vertebrates. Eosinophils usually account for less than 7% of the circulating leukocytes. Beyond causes related to infection or parasitic infestation, elevated eosinophil levels (also known as eosinophilia) can be also a sign of allergic or atopic reactions. In the instant study no statistically significant differences between experimental groups in eosinophil levels were observed.

FIGS. 36A-I. depict the clinical chemistry and Hematology results in mice after administration (PO, IT) of MEV.

### Histology

The main organs were collected, fixed in 4% PFA, paraffin-embedded, cut into sections and stained with H&E.

### Lung

Histopathological changes observed in lung sections are primarily limited to minimal to mild focal to multifocal alveolar hemorrhages in 8/10 lung sections examined in animals receiving intratracheal negative control material and test item. Changes were relatively subtle and limited in some of the alveolar spaces. This observation was observed at comparable incidence and severity in negative control and treated animals.

### Kidney

Presence of a few basophilic tubules were observed in 1 animal treated PO with 4×10¹¹ MEV dose. This finding was minimal in severity and often is observed spontaneously at low incidence and severity in laboratory rodent animals. It is therefore considered as incidental in origin in the present study.

### Spleen, Liver, and Brain

No histopathological changes were observed in spleen, brain, or liver in all sections examined.

### SUMMARY

The pulmonary changes observed in most animals receiving the experimental material intra-tracheally were considered not treatment related. There was no treatment-related observed in all organs examined, indicating that MEVs are not toxic to animals under the experimental conditions.

### CONCLUSION

Regarding the clinical aspects, all the animals, at each time of the study, exhibited normal behaviors, body weight, water and food consumption. The clinical findings indicate general tolerance in the mouse model of the test MEV products. Under the experimental conditions, both negative controls (PBS) and MEV at concentration of 4*10¹¹ MEV/mouse or 4*10¹² MEV/mouse by oral administration and at concentration of 4*10¹¹ MEV/mouse by IT administration did not induce any abnormal, toxic effect on animals.

### Example 9

In vitro modulation of human cancer cells by the Microalgae Extracellular Vesicles (MEVs)

### A. Cell Cultures

Human cell lines (ATCC) are grown in DMEM (Dulbecco's modified Eagle's medium) supplemented with 10% fetal bovine serum (FBS), 50 units/ml penicillin, and 50 μg/ml streptomycin. The cells are kept in culture at 37° C. and 5% CO₂, and the medium is changed every three days. To keep the cells at optimal proliferating conditions, they are passaged at 80% confluence and seeded at 20% confluence. The following cell lines are used in the study:
1) MYC oncogene-expressing HT29 (colon cancer), PANC-1 (pancreatic cancer) and MYC-negative LN-18 (malignant glioma);
2) RAS oncogene-expressing SCLC-21H (small-cell lung cancer), MCF-7 (breast cancer) and RAS-negative PC3 (prostate cancer);
3) BCL-2 oncogene-expressing AsPC-1 (pancreatic cancer), U-937 (pro-monocytic myeloid leukemia) and BCL-2-negative LM-MEL-53 (malignant melanoma);
4) PLK-1 oncogene-expressing HepG2 (hepatoma), PC3 (prostate cancer) and PLK-1-negative AsPC-1 (pancreatic cancer).

### B. RT-PCR Analysis and Gel Electrophoresis

The cells (1×10⁶) are grown in culture plates for 24 h. Next, the siRNA carrying MEVs, or empty MEVs, or siRNA alone, are added to the wells in serial dilutions and incubated for 1-24 hours. Following the incubation, the medium is changed for the normal culture medium. 72 h after the MEV treatment, total RNA is extracted from the cultured cells using Trizol reagent. The RNA precipitate is centrifuged and dissolved in 20 μl of RNase-free water. UV spectrophotometer analysis at 260/280 nm and agarose gel electrophoresis are used to confirm the quality of purified RNA. 1 μg of total RNA is reverse transcribed to synthesize cDNA at 42° C. for 1 h; this cDNA is then subjected to PCR amplification with specific primers in 25-μl reactions. Next, the PCR products are resolved on 2% agarose gel, stained with ethidium bromide and observed with a UV trans-illuminator. The digital images are quantified with ImageJ analysis software and the results are expressed as the relative expression level of relevant genes over GAPDH.

### C. Gene Expression Analysis with Quantitative RT-PCR

The cells treated with the MEVs and cultured for a total of 72 h are trypsinized and washed with PBS. Total RNA is extracted from the cell pellets using the RNeasy® Minikit (Qiagen) according to the manufacturer's instructions. Then, 500 ng of total RNA is reverse transcribed into cDNA using a reverse transcriptase (Promega) and real-time PCR is performed using the LightCycler 480 SYBR Green I Master Kit on an LC480 device (both from Roche Diagnostics). The mRNA level is calculated by normalizing the threshold cycle (CT) of target genes to the CT of the 18S ribosomal RNA housekeeping gene. The primers are designed using primer software from Roche Diagnostics and are purchased from Eurogentec.

### D. Western Blot Analysis

The cells (2×10⁶) treated with the MEVs and cultured for a total of 72 h are trypsinized, washed with PBS and lysed on ice in RIPA buffer supplemented with β-mercaptoethanol (Sigma-Aldrich). Then, 50 μg samples of total protein are subjected to 10% standard sodium dodecyl sulfate-polyacrylamide gel electrophoresis (SDS-PAGE), with pre-stained molecular weight markers run in parallel. Subsequently, the resolved proteins are electro-transferred onto PVDF membranes. After extensive washing, the membranes are incubated with the relevant horseradish peroxidase-conjugated antibody (Sigma-Aldrich) for 1 h at room temperature, and developed with a chemiluminescence detection kit (Thermo Fisher Scientific). Membranes probed for the oncoproteins are re-probed for β-actin to normalize for loading and/or quantification errors and to allow comparisons of target protein expression. The protein expression is quantified with a gel analysis system (Bio-Rad).

### E. Cell Proliferation Assay

The cells are seeded into 96-well plate (10,000 cells/well) and allowed to attach for 18 h. Next, the cells are treated with the MEVs as above; at 24, 48, and 72 h after the treatment, 10 μl 10 mg/ml MTT is added to the cells in each well. The cells are incubated for 4 h, after which 100 μl DMSO is added, and the cells are allowed to lyse for 15 min. All of the analyses are carried out in triplicate. Optical density at 492 nm is determined with a SpectraMax® microplate reader (Molecular Devices, USA). To determine the inhibitor rate, the absorbance values are then normalized to the values obtained from the blank control cells.

### 1. Apoptotic Cell Morphology Observation

The cell morphology related to apoptotic cell death is established by staining the cell nuclei with the DNA-binding fluorochrome Hoechst 33258 and assessing the chromatin condensation by fluorescence microscopy. Briefly, the cells are seeded in 24-well plates with glass slides on the bottom of the wells, and treated with the MEVs as above. Afterwards, the slides are gently washed with cold PBS, fixed with 4% paraformaldehyde for 1 h and washed three times with PBS. The slides are stained with 0.5 ml Hoechst 33258 (10 μg/ml) at 37° C. for 10 min in a dark room. In each group, five microscopic fields are randomly selected, and one hundred cells are counted. The apoptotic cell level is then calculated as the percentage of apoptotic cells over the total number of fluorescent cells.

### 2. Fluorescent Analysis of Necrotic/Apoptotic Cell Death

For flow cytometry analysis, the cells are seeded in 24-well plates (15,000 cells/well). After 24 h, cells are treated with the MEVs as above. Subsequently, the cells are washed with PBS and detached with trypsin. Following fixing in 0.2% paraformaldehyde in PBS, the cells are labeled with Annexin V FITC/PI (Thermo Fisher Scientific) and analyzed using LSRII flow cytometer with Cell Quest Pro software (BD Biosciences).

### Example 10A

### In Vivo Biodistribution of Mevs

Experiments were performed to determine the pathway and fate of MEVs when administered via various routes.

### A. MEV Labelling Using DiR Fluorescent

As described in example 2, the fluorescent, lipophilic carbocyanine DiR (1,1-dioctadecyl-3,3,3,3-tetramethylindotricarbocyanine iodide) is weakly fluorescent in water but highly fluorescent and photostable when incorporated into membranes and can be tracked in vivo (see, Example 2 above; ThermoFisher Scientific). The DiR labelled (MEVs) are incubated with human cells. Their uptake by human cells is measured by fluorimetry analysis (fluorescence spectroscopy) on microplate readers. DiR has an excitation of 750 nm and an emission of 780 nm.

### 1. In Vivo Administration of DiR-Labelled MEV for Biodistribution Studies

The methodology for the biodistribution studies is summarized as follows: Chlorella cells were grown in the photobioreactor as described above (120 L per batch), were isolated and labeled with DiR, and labelling efficiency was assessed as described in Examples 1 and 2.

The labeled MEVs were administered to the mouse model animals via one of four routes, via: intranasal, intratracheal, oral, and intravenous administration, to determine the pathways and fate of the MEVs by each route. Treated mice were subjected to full body imaging as a function of time, and at day 3 mice were sacrificed and the organs were imaged ex vivo, and by histology in several mice organs after different routes of application.

MEVs' pharmacokinetics and biodistribution were studied in Specific Pathogen Free (SPF), 7-week old male Balb/cByJ mice (Charles River Laboratories). The animals were labelled with unique ear ID tags and acclimatized for at least 2 days. Background (control) mice were housed individually, while MEV-treated mice were housed collectively in disposable standard cages in ventilated racks at 21±3° C. (temperature recorded and controlled) with a 12 hr-12 hr light/dark cycle. Filtered water and low fluorescence laboratory food for rodents were provided ad libitum. All mice were euthanized at the end of the in vivo experiments.

The DiR-labelled MEVs (prepared as above in Example 2) were used to determine the biodistribution of the MEVs in the whole animal body or in several organs after different routes of application.

Prior to the imaging, the fur of each animal was clipped using an electric clipper in the following areas: abdomen, thorax, head, and whole back. Care was taken in order to clip the fur as homogeneously as possible. Next, DiR-labelled MEV preparations were re-suspended by vortexing before filling the syringes for injection.

The following routes were used to administer the MEVs to the animals: Intravenous (IV): 50 μL of MEV suspension containing 0.6×10¹² MEV/mL were injected in a tail vein by disposable plastic syringe and appropriate needle. Dosage of MEV administered: 3×10¹⁰ MEV/mouse.

Intranasal (IN): Animals were induced and maintained under anesthesia during IN administration by a mixture of isoflurane and oxygen as a carrier gas. A volume of 100 μL of MEV suspension containing 0.3×10¹² MEVs/mL was administered into the nostrils of the mouse using a thin pipette cone. Dosage of MEV administered: 3×10¹⁰ MEV/mouse.
Per os (PO): 100 μL of MEV suspension containing 0.3×10¹² MEVs/mL was administered orally using a disposable plastic syringe and an appropriate feeding probe. Dosage of MEV administered: 3×10¹⁰ MEVs/mouse.
Intratracheal (IT): Animals were induced and maintained under anesthesia during IT administration by a mixture of isoflurane and oxygen as a carrier gas. Once adequate anesthesia was observed, animals were placed on a mouse intubation platform, suspended from the front incisors in the supine position, to maximize view of the trachea. Then, a cold light was placed on the skin near the trachea localization in order to backlight the trachea. If needed, a laryngoscope was inserted to guide the syringe of a Microsprayer® sprayer into the trachea. An injection of 50 μL of MEV suspension containing 0.6×10¹² MEVs/mL was instilled into the trachea. Anesthesia was maintained throughout the procedure. After injection, the mice were maintained in the same position on the intubating platform for at least 30 seconds, before being replaced in their cage. Dosage of MEVs administered: 3×10¹⁰ MEVs/mouse.

### 2. In Vivo Imaging for Biodistribution Studies

Fluorescence acquisitions were performed with the IVIS® Spectrum optical imaging system (acquired from PerkinElmer). Bidimensional (2D) fluorescence imaging was performed by sensitive detection of light emitted by DiR-labelled MEVs. Mice were anesthetized and imaged 1 hour before MEV administration. Next, fluorescence of the material to be administered was confirmed with an in vitro acquisition performed on at least 1 drop of a minimum of 20 μL of MEV suspensions, deposited in 2 different Petri dishes. The fluorescence emission was measured and detected with the selected parameters (see below). Finally, in vivo fluorescence acquisitions were performed on mice administered MEVs. The animals were induced and maintained under anesthesia with a mixture of isoflurane and oxygen. Mice were positioned in dorsal recumbency to obtain ventral images and in ventral recumbency to obtain dorsal images. At least 2 acquisitions/mouse/timepoint were taken and mice were imaged in groups of maximum 3 mice/acquisition. Images were analyzed and fluorescence quantified on 6 organs (liver, spleen, lung, kidney, intestine, and brain).

The following timepoints were used (T0=MEV administration):
Day 0-6 timepoints: T0-1 h±10 min; T0+30 min±5 min; T0+2 h±15 min; T0+4 h±30 min; T0+8 h±30 min; T0+10 h±30 min
Day 1-1 timepoint: T0+24 h±3 h
Day 2-1 timepoint: T0+48 h±3 h
Day 3-1 timepoint: T0+72 h±3 h
   The data were analyzed with the IVIS software (Living Image Software for IVIS).

The following parameters were used for the in vivo and ex-vivo fluorescence acquisitions:

Field of View (FOV) 14×14 cm (FOV C)

Image number: In vivo: For each mouse at each applicable timepoint, at least 2 acquisitions (ventral and dorsal) were performed; 3 mice (at maximum) were imaged in one acquisition.
Ex-vivo: the organs of at least one mouse were imaged in one acquisition
Image format TIFF format
Fluorescent probe DiR
Excitation wavelength 745 nm
Emission wavelength 800 nm
Exposition time Automatic, depending on the fluorescence signal detected
Minimum counts 20000
Binning Between 16 and 1 (depending on the fluorescence signal detected)
F/STOP 2 (in case of fluorescence signal saturation it was automatically increased to 4 or it was decreased to 1 in case of weak signal)
Subject height In vivo: 1.5 cm
   Ex-vivo: 0.5 cm

### 3. Ex Vivo Imaging for Biodistribution Studies

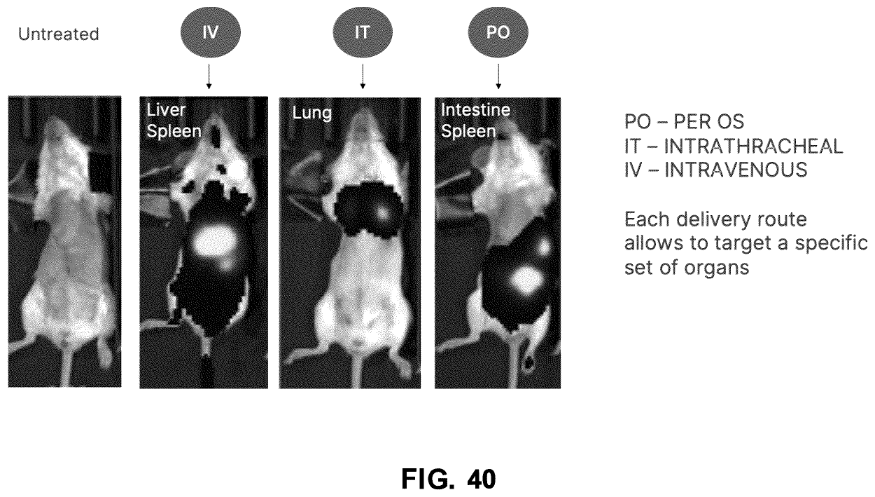

Production, purification, characterization, labelling and administration (by different routes) of MEVs was as described above. On Day 3 after administration, mice were euthanized and the organs of interest of each mouse were sampled and positioned in a Petri dish in order to perform ex vivo acquisitions. The following organs were sampled: liver, spleen, lungs, kidneys, brain, and intestine. The data were analyzed with the IVIS software (Living Image Software for IVIS). FIG. 40 shows representative patterns of biodistribution according to the route of administration, for the Intravenous (IV), Intratracheal (IT), and Per os (PO) routes.

### B. CONCLUSIONS

### Pharmacokinetics

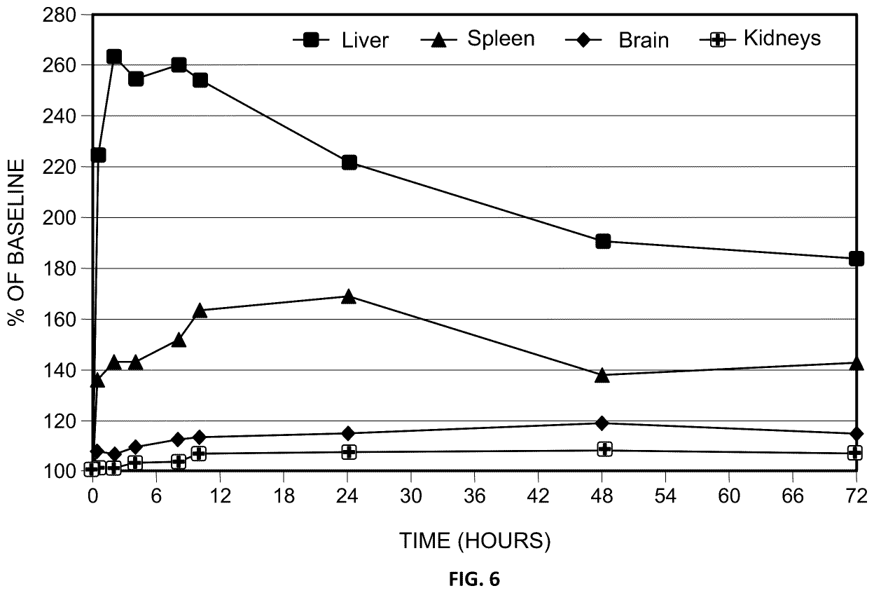

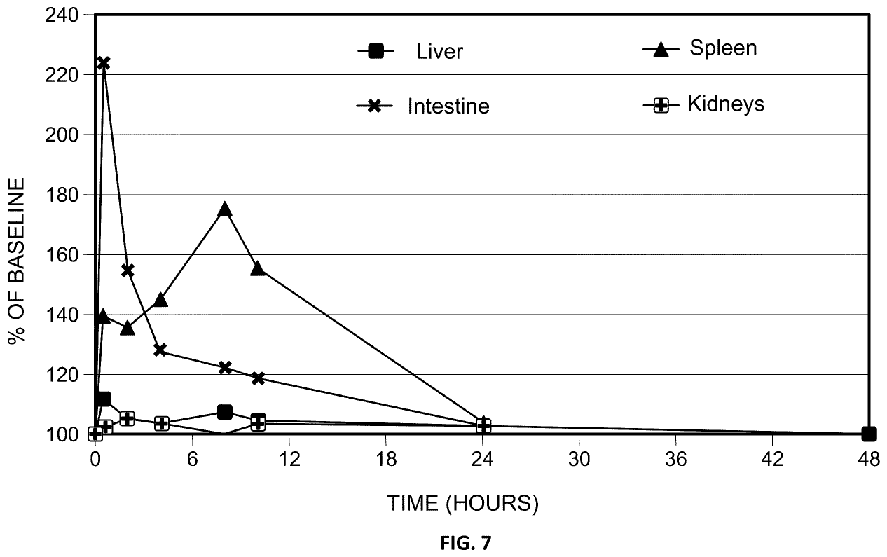

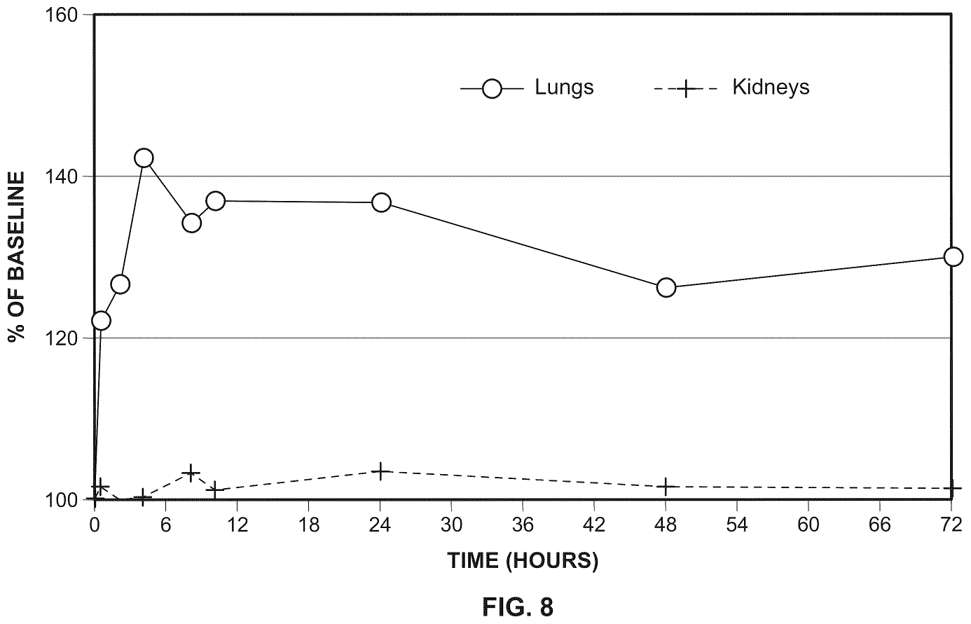

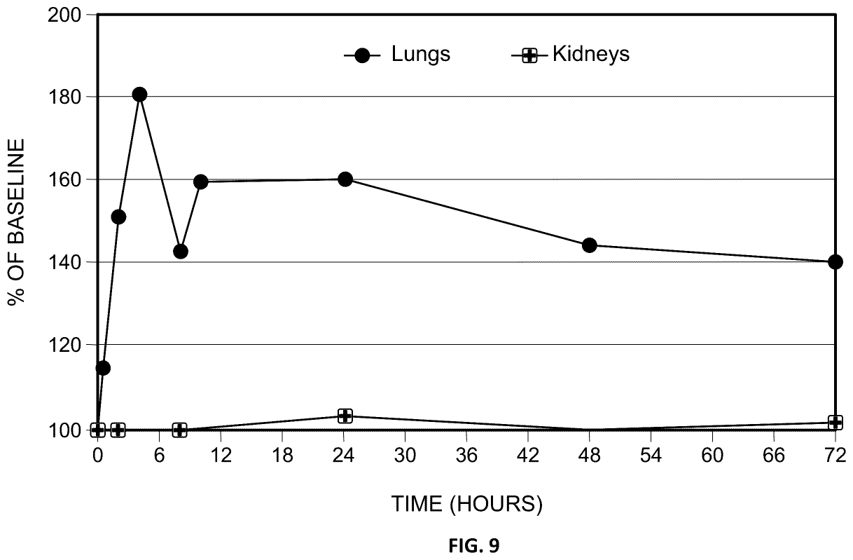

The pharmacokinetic curves demonstrating the accumulation as a percent of baseline over time (hours) after intravenous, Per os, intranasal, and intratracheal administration are set forth in FIGS. 6-9, respectively.

### Biodistribution-Isolated Organs-Ex Vivo Imaging

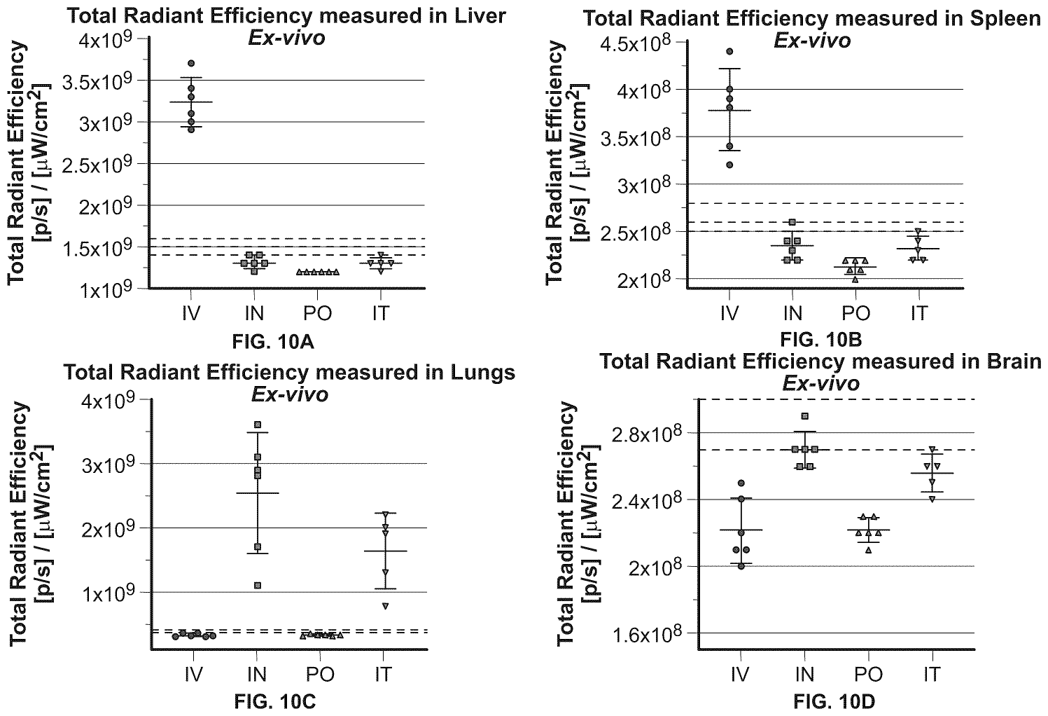

Biodistribution—in vivo full body imaging of a representative animal is depicted in FIG. 40. The biodistribution by ex vivo fluorescence analysis (total radiant efficiency) in liver; spleen; lungs; and brain after intravenous (IV), intranasal (IN), Per os (PO), and intratracheal (IT) administration is set forth in FIGS. 10A-D. Because the amount of MEVs (100 μl) administered was too much, the distribution following intranasal administration is not an accurate reflection of the MEV pathway upon intranasal administration. Subsequent experiments employed 20 μl, and the results show that MEVs, upon intranasal administration, traffic to the brain. This is shown and described in Examples below.

### Intravenous Administration

Organs targeted by unmodified MEVs-liver and spleen
PK parameters:
   Peak/plateau time: 2-24 h (liver), 4-24 h (spleen)
   at t=day 3 compared to peak/plateau approximately 62% (liver), approx. 61% (spleen)
Longer presence or duration compared to mammalian EVs (see, e.g., Kang et al., Biodistribution of extracellular vesicles following administration into animals: A systematic review. J Extracell Vesicles. 2021; 10: e12085. (doi.org/10.1002/jev2.12085)).
No visible signs of toxicity
   Per os (oral) administration
Organs targeted by following oral administration: intestine and spleen
Oral availability:
   MEVs are orally available; they resist the passage through the stomach, reach the intestine and subsequently appear in the spleen.
   Mammalian EVs are not orally available, with the exception of bovine milk EVs.
PK parameters:
   Peak/plateau time: 1-6 h (intestine), 1-10 h (spleen)
   % at t=10 h compared to peak/plateau: approx. 33% (intestine), approx. 44% (spleen)
   % at t=48 h compared to peak/plateau: 0% (intestine), 0% (spleen)
No visible signs of toxicity
   Distribution in organs following intravenous and oral administration.

Liver: Following intravenous administration, the liver is the primary target organ. Following oral administration, the liver is marginally targeted, if at all.

Spleen: The pharmacokinetics profile depends on whether administration is intravenous or oral (per os). Intravenous administration is longer lasting. At day 3, 50-70% of the peak is still remaining. Oral administration is shorter lasting; at 48 hours, 0% remains.

### Intranasal Administration

Organs targeted by the unmodified MEVs appeared to include the lungs, but as demonstrated in Examples below, intranasal administration targets the brain. The volume administered in this experiment was too large and may have been inhaled or directed into the lungs upon administration. Subsequent results and experiment show that intranasal administration results in administration to the brain.
Preliminary PK parameters:
   Peak/plateau time: 4-28 h
   % at t-day 3 compared to peak/plateau: 70-80%
No visible signs of toxicity
   Intratracheal administration
Organs targeted by the MEVs are the lungs
PK parameters:
   Peak/plateau time: 2-72 h
   % at t=day 3 compared to peak/plateau: approx. 80%
No visible signs of toxicity

### Example 10B

In Vivo Biodistribution of Mevs after Per Os (Po) Administration

### a. MEV Labelling Using pKH26 Fluorescent

PKH26 is a lipophilic fluorescent dye used for general membrane labeling. It is used for in vitro studies, as well as long term in vivo experiments due to enhanced stability (in vivo half-life is over 100 days). MEVs were labeled with PKH26 as described above and administered orally to BALB/c mice at a dose of 4×10exp10 MEV particles per animal. Between 0.5 to 24 hours post-administration, the animals were sacrificed, and the intestine (epithelium and GALT), the spleen and the liver were harvested.

### b. Organ Sampling and Processing

The jejunum and ileum were gently rinsed with a cold PBS solution. Then, a piece of approximately 0.5 to 1 cm of jejunum and ileum were snap-frozen and lastly, each piece was longitudinally placed on bottom of the cryomold for OCT inclusion (as described below).

The spleen was snap-frozen, then, the spleen longitudinally plated on bottom of the cryomold for OCT inclusion.

The medial and the left lobes of the liver were cut in two sections of maximum 0.5 cm high. Then, each section was snap-frozen, then plated the cut face on the bottom of the cryomold for OCT inclusion.

### c. Snap-Frozen

A small stainless-steel bowl was placed in the bottom of a container containing dry ice in pellet form and liquid nitrogen. Then, isopentane/2-methylbutane was slowly added in the container. When the dry ice pellets stop bubbling vigorously or/and the isopentane start to become opaque, the isopentane/2-methylbutane is at optimal temperature. Organs were gently immerged in isopentane/2-methylbutane and then placed in cassette (one cassette with each section of liver, the spleen and the piece of jejunum and one cassette with the piece of ileum only). Then, cassettes were stored at ≤−75° C. until OCT inclusion.

### d. OCT Inclusion Protocol

A small stainless-steel bowl was placed in the bottom of a container containing dry ice in pellet form and liquid nitrogen. Some pellets of dry ice were also placed directly in the bowl. Then, isopentane/2-methylbutane was slowly added in the container. When the dry ice pellets stop bubbling vigorously and/or the isopentane starts to become opaque, the isopentane/2-methylbutane is at optimal temperature. The frozen tissue samples were then placed and oriented as mentioned above for each organ in the cryomold. The tissues were gently pushed with forceps to ensure that the bottom of surface of the tissue is placed properly (touching the face of the bottom, center in the mold and properly oriented). The cryomold with frozen tissue samples were placed on the surface of the cold isopentane/2-methylbutane. Optimal cutting temperature compound (OCT compound) was carefully deposited onto the specimen until it is completely covered. After hardening of the OCT compound (between 30 seconds and 1 minute), the OCT embedded block was placed in a bag (such as zip freezer bag). The frozen blocks were temporarily stored in dry ice. Then, slices of 5 μm were performed with a cryostat then glued on untreated slides for the histology and on treated slides for the immunochemistry.

### e. HES Staining

The HES staining allowed the observation of the morphology and the structure of tissues. After fixation in acetone, sections were immersed successively in solutions of Harris hematoxylin, eosin and saffron. After dehydration, sections were mounted between slide and cover slip using Entellan™ mounting medium. Cytoplasm appeared in pink and nuclei in violet blue. Extracellular matrix was stained in yellow to pink.

### f. PKH26 Dye

Accumulation of fluorescent labeled MEVs was visualized by a LSM700 laser scanning confocal microscope (from Zeiss) and images were processed with LSM Image Browser. DAPI (4′,6-diamidino-2-phenylindole) [Invitrogen, Ref. S36938] was used as nuclear counterstain prior to the imaging. Sections were mounted with aqueous medium with DAPI for fluorescent slide scanner observation (maximum 10 days after collection).

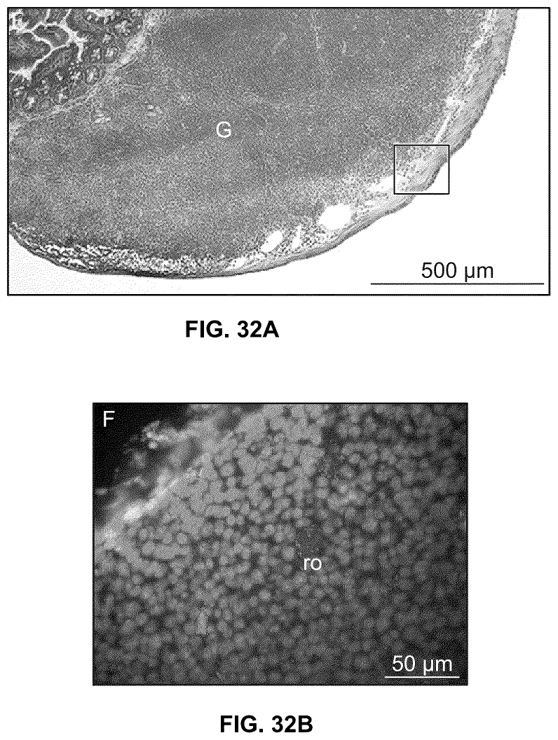

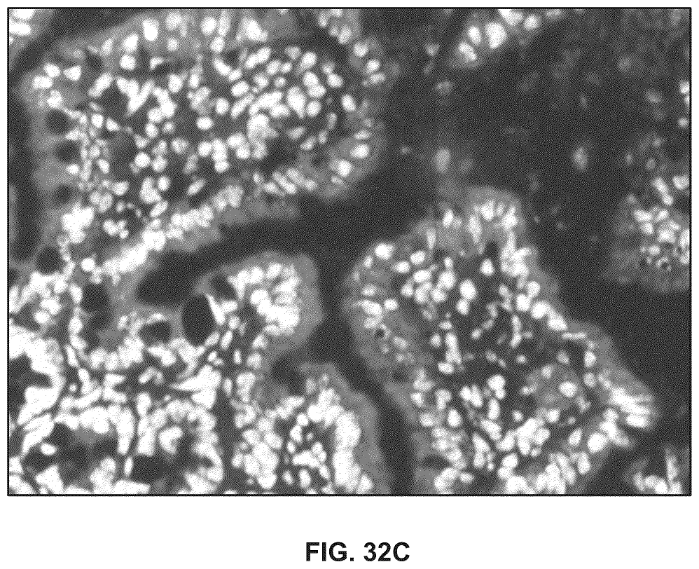

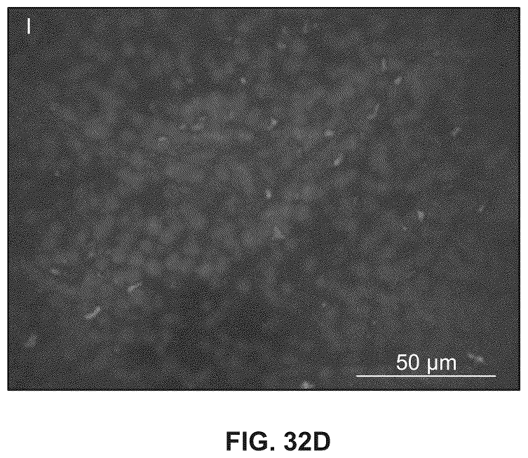

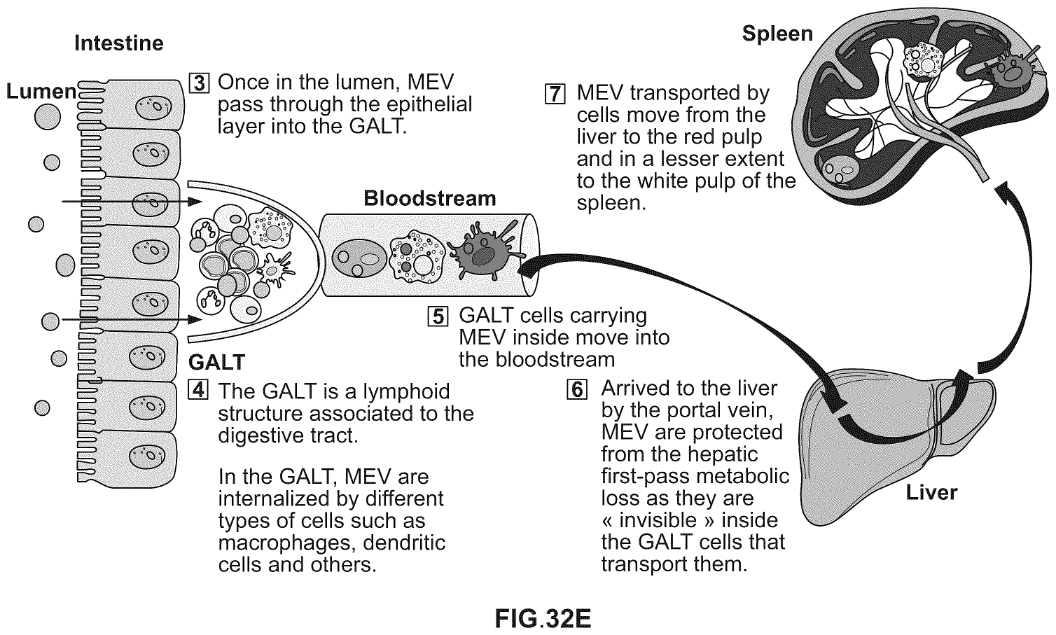

FIG. 32A-E shows a microscopic image of mouse intestinal epithelium 8 hours after PKH26-labeled MEV administration. Fluorescent signal visible in the enterocytes confirms MEV internalization. Cell nuclei were stained with DAPI.

### G. CONCLUSIONS

When MEVs are orally administrated to the mice, they follow through the digestive tract. MEVs first reach the stomach, where they resist the stringent conditions of the gastric juice. In about 30 minutes after administration, they reach the intestine, where they stay for several hours.

Once in the lumen of the intestine, as shown in this Example, MEVs are internalized by the cells of the intestinal epithelium, or enterocytes. MEVs also pass through the epithelial layer into the GALT. The GALT is a lymphoid structure associated to the digestive tract (gut-associated lymphoid tissues) located beneath the intestinal epithelium. It is located at specific spots along the intestine. GALT is a dense tissue composed of germinal centers with B and T lymphocytes, plasmocytes and innate immune cells including dendritic cells and macrophages. In the intestine, fluorescence is observed mostly in the GALT. Fluorescence appears concentrated in discrete spots around nucleus meaning that MEVs are localized in the plasma of cells occupying all the space in the plasma. This allows revealing of the shape of cells. Cells with MEVs inside correspond to histocytes (resident macrophages) or dendritic cells respectively and are located mainly at the periphery of GALT and in the center of the GALT. Representative images are displayed in FIGS. 32A-D. No visible MEVs appear in the liver at all times evaluated (30 min to 24 h). A few hours after administration, dendritic/macrophage cells with MEVs inside move from the GALT to the spleen and stay in the spleen for several hours. MEVs reach principally the red pulp (blood cells and some Th and B cells) and to a lesser extent the white pulp of the spleen (lymphoid cells) (see FIG. 32D).

In the spleen, fluorescence is visible in several areas corresponding to the white and to the red pulps. The intensity of the fluorescence decreases in a gradient from the red pulps to the white pulps. Fluorescence is detected in spots inside the cell's cytoplasm. Cells with MEVs inside are round or reticular and have similar size; they may respectively be histocytes and dendritic cells or macrophages.

Cells can move from the GALT into the bloodstream directly or they can join it after they have first passed through the lymphstream. GALT cells carrying MEVs inside eventually arrived at the liver by the portal vein, MEVs, however, are ‘invisible’ to the liver as they have been internalized by GALT cells that naturally transport them to the spleen (see FIG. 32E). These MEVS, therefore, are not captured by the liver, as are the MEVs that have been administered intravenously.

### Example 11

### In Vitro Delivery of Cargo by the Microalgae Extracellular Vesicles (MEVs) in Primary and Established Cell Lines

### A. Cell culture

Human primary cells (primary hepatocytes from healthy volunteers or patients) or established hepatocytes cell lines (HepG2 and Huh7) or established lung cell lines (A459 and BEAS-2B) are cultured in specific optimal conditions for each type of cells. The cells are grown in 75 cm² flasks, incubated at 37° C. with 100% humidity and 5% CO₂. The culture medium is changed every 2 days to ensure the growth of cells and to avoid contamination. Once reached sub-confluence, the cells are detached from the bottom of the flask with trypsin (0.25%), centrifuged (1600 g, 4 min), re-suspended in fresh culture medium and seeded at 2×10⁵ cells/plate.

### B. Delivery and Expression of the GFP Protein Loaded in MEVs in Human Cells

### 1. Effect on Primary Human Hepatocytes PHH and on Established Hepatocyte Cell Lines Viability

PHHs and hepatocyte cell lines (1×10⁵ cells/mL) are seeded into a 96-well plate and allowed to attach overnight. Then, the cells are exposed to different cargo loaded MEV formulations or empty MEVs for four hours. Following the incubation, 20 μL of MTT tetrazolium dye solution (5 mg/mL) is added into each well. After 3 hours of incubation at 37° C., the MTT-containing medium is removed and 100 μL DMSO is added into each well to dissolve the purple formazan precipitate. Absorbance is measured at 570 nm using a SpectraMax® microplate reader (Molecular Devices, USA) and cell viability is expressed as a percentage of viable cells in the treated groups compared to the untreated control group.

### 2. MEV Internalization in PHH Established Hepatocyte Cell Lines

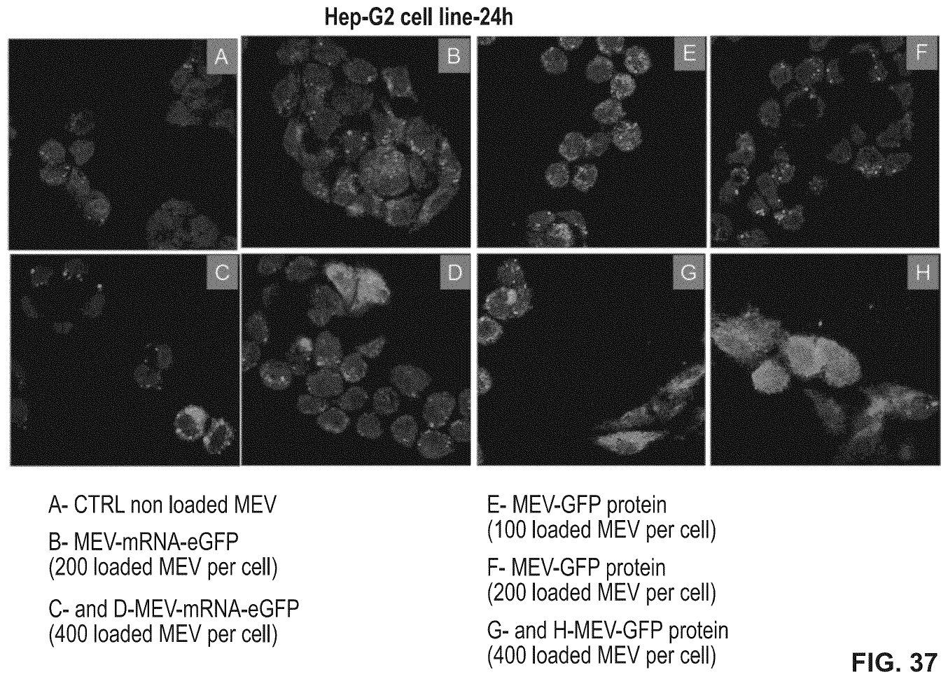

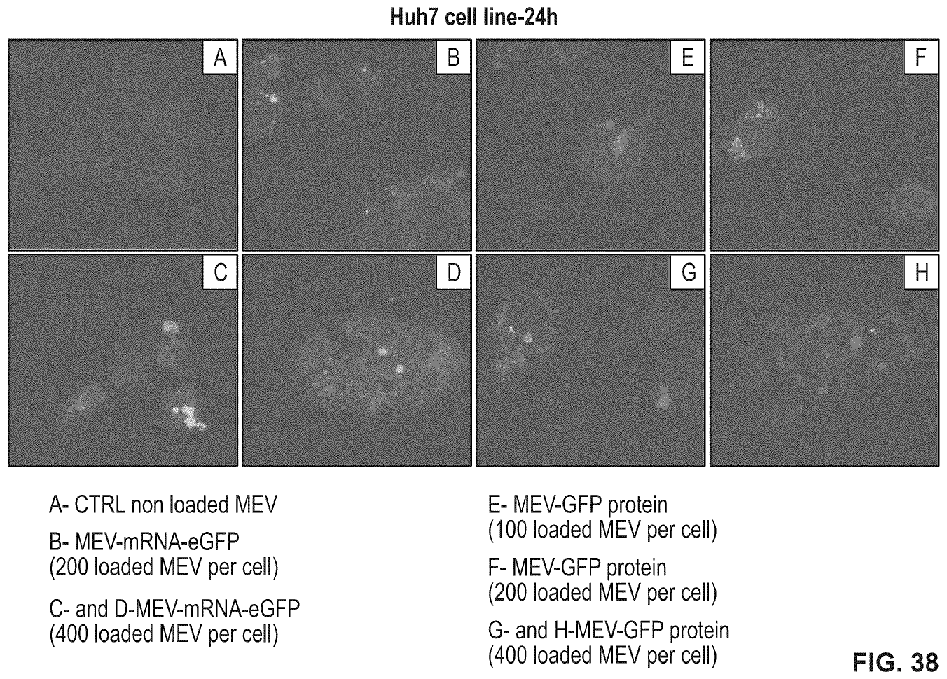

The internalization of the GFP-protein-cargo-loaded MEVs or GFP-mRNA-cargo-loaded MEVs in PHH or in hepatocyte cell lines is studied by confocal microscopy. Cells are seeded in 16-well chamber slides (Lab-Tek) at 1×10⁵ cells/mL. After 24 h, cells are incubated with the MEVs for 24 h and 48 h and subsequently washed with PBS, washed again with acid wash buffer (0.5 M NaCl, 0.2 mM acetic acid) to remove membrane bound MEVs and once more with PBS. Cells are fixed with 4% paraformaldehyde in PBS at room temperature for 20 min. Slides are then washed with PBS and mounted using Fluorsave (Calbiochem). Confocal fluorescent imaging is performed using a LSM700 laser scanning confocal microscope (Zeiss) and images are processed with LSM Image Browser. Results are shown in FIGS. 37 and 38.

### C. MEV-Mediated Delivery and Expression of GFP mRNA in Human Hepatocyte Cells

### 1. mRNA Translation Studies

The delivery and biological activity in hepatocytes of the GFP-mRNA-cargo-loaded MEVs is determined by total RNA extracted from the cells using the RNeasy Minikit (Qiagen) according to the manufacturer's instructions. Then, 500 ng of total RNA is reverse transcribed into cDNA using a reverse transcriptase (Promega) and real-time PCR is performed using the LightCycler 480 SYBR Green I Master Kit on an LC480 device (both from Roche Diagnostics). The mRNA level is calculated by normalizing the threshold cycle (CT) of target genes to the CT of the 28S ribosomal RNA housekeeping gene. The primers are designed using primer software from Roche Diagnostics and are purchased from Eurogentec.

### 2. Protein Expression Studies

The delivery and biological activity in hepatocytes of the GFP-protein-cargo-loaded MEVs is determined by SDS-PAGE followed by Western blot analysis. Cells are lysed on ice in RIPA buffer supplemented with β-mercaptoethanol (Sigma-Aldrich). Then, 30 μg of proteins are boiled and subjected to SDS-PAGE and transferred to nitrocellulose membranes (Bio-Rad). The blots are blocked with Tris buffered saline (TBS), 0.1% Tween-20 containing 5% BSA and incubated overnight at 4° C. with the following antibodies. Subsequently, the blots are incubated with the relevant HRP-conjugated secondary antibody for 1 h at room temperature. Signals are revealed by chemiluminescence (Thermo Fisher Scientific).

### D. MEV-Mediated Delivery and Expression in Normal Human Keratinocytes (NHEK) and in Primary Human Monocytes

### 1. Culture Condition

NHEK and culture medium (KGM-Gold Bullet kit) were purchased from Lonza. Peripheral Blood Mononuclear Cells (PBMC) from healthy volunteers were obtained from 1′Etablissement Français du Sang (EFS). Cell culture medium Macrophage-SFM (M-SFM) was purchased from Gibco.

PBMCs were obtained from healthy blood donor buffy coats by a standard Ficoll-Hypaque gradient method. Monocytes were isolated from PBMCs by adherence to plastic for 2 hours in serum-free medium (M-SFM) optimized for macrophage culture, at 37° C. in a humidified atmosphere containing 5% CO₂. NHEK were cultured in accord with manufacturer's recommendations (Lonza) and seeded in 96-well plates the day before the assay.

Cellular uptake of GFP-loaded MEVs

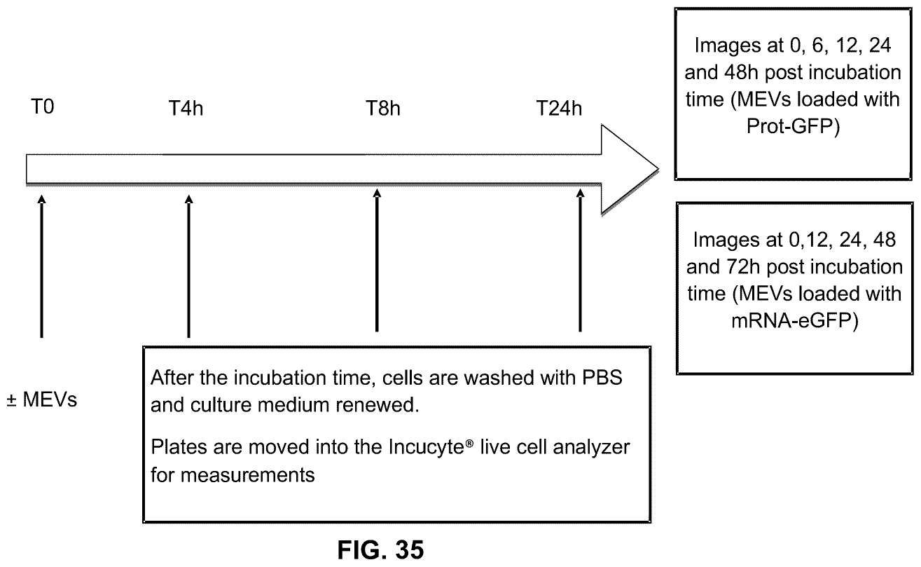

The experimental plan is shown in FIG. 35. GFP-MEVs are incubated with NHEK and monocytes in wells for 4 h, 8 h and 24 h incubation time and then washed with PBS twice. The plates were then placed in the Incucyte apparatus and images were done at 0, 6, 12, 24 and 48 h for Prot-GFP and 0, 12, 24, 48 and 72 h for mRNA-GFP.

Images are analyzed using the Incucyte software after a first quantification of the total number of cells per well. The software then quantified the number of cells that contained green objects in the control wells to determine the autofluorescence threshold level. This threshold is applied to all the images. Then the software calculated the percentage of cells that are green in the treated wells using the following ratio: Ratio=(cells containing green objects/number of cells)×1000

For human monocytes the optimal time of incubation with MEVs is 8 h with ratio of 5 percent of green cells, and for NHEK cells incubation times of 8 h and 24 h show results ratios of 10 and 20 percent of green cells, respectively. The timeline for image analysis is shown in FIG. 35.

### E. MEV-Mediated Delivery and Expression in Human Cancer Cell Lines (A375 (Melanoma), CAMA-1 (Breast Cancer), and HEK 293T (Embryonic Kidney)

### 1. Cell Culture

On the morning, cells are detached with Trypsin (0.05% Trypsin-EDTA (1X); Around 5 min), counted by an automatic cell count (Cellometer® Auto 1000), diluted to a concentration 1.6×10⁶ viable cells/mL. Then, 3 mL of the cell suspension are seeded into a six well plate. On the afternoon of the same day 25 μl of each tested MEVs RNAi against c-myc or k-ras are added on the wells and incubated for 24 h, 48 h, 72 h and 96 h at 37° C. After the indicated time, cells are washed two times with PBS. The cells quantity is split: one for protein extraction, and one for RNA extraction, and placed at −80° C.

### 2. RNA Extraction

Cell pellets are lysed by addition of 350 μL of RLT lysis buffer (99% RLT buffer+1%-mercaptoethanol). RNA is extracted using the RNAeasy mini kit using a protocol provided by the manufacturer.

### 3. Rt-Qpcr

RNA is dosed using a NanoDrop® One (Thermo scientific) and adjusted at 130 ng of RNA per reaction with a volume of water RNAse-free 0.1% DEPC to obtain a final volume of 5.9 μl. Then, 4.1 μl of mix (including SuperScript IV, dNTP mix, Random primers, 0.1 M DTT) are added and incubated at 37° C. for 1 h.

qPCR is realized using the Takyon® No ROX SYBR 2X MasterMix blue dTTP. For one reaction, primers are added at final concentration of 0.66 μM. Then, water qs 5 μL and 1.67 μl of cDNA, diluted at 1/20, are added. LC480 is used to realize the run. Each sample is run in triplicates. The primers used are described below:

c-myc forward primer:
(SEQ ID No. 404)
TACAACACCCGAGCAAGGAC
c-myc reverse primer:
(SEQ ID No. 405)
TTCTCCTCCTCGTCGCAGTA
k-ras forward primer:
(SEQ ID No. 406)
GGTTGCGCTGACCTAGGAAT
k-ras reverse primer:
(SEQ ID NO. 407)
TCCATTTCGGGGCAAACAGT

MEV-RNAi against k-ras is:
1—ineffective for inhibiting k-ras mRNA amount in A375 melanoma cells,
2—effective for inhibiting k-ras mRNA in HEK293T cells (33% at 24 h and 48 h),
3—effective for inhibiting k-ras mRNA in CAMA-1 cells (55% at 24 h).

MEV-RNAi against c-myc is:
1—effective for inhibiting c-myc mRNA in A375 melanoma cells (40% at 24 h),
2—effective for inhibiting c-myc mRNA in HEK293T cells (30% at 24 h),
3—effective for inhibiting c-myc mRNA in CAMA-1 cells (30% at 24 and 48 h).

### 4. Western-Blot

Proteins were extracted using RIPA lysis buffer. Then, after 1 h of incubation, they were centrifuged. The whole manipulation is realized on ice. The supernatant was recovered to be dosed.

Lysates were dosed using the Bradford protein assay and adjusted at 50 μg in each sample. Then, each sample was heated at 95° C. for 5 min, loaded on precast gels (Mini-PROTEAN®, BIO-RAD) to apply a PAGE-SDS. After the ending of electrophoresis, proteins were transferred using the iBlot®2 (from ThermoFisher) with a PVDF Mini Stacks.

C myc, K-ras and B actin (Control) were revealed with a primary antibody and with a near Infrared secondary antibody (references are described below). The near infrared signal was read with Azure 500® (azure biosystems).

MEV-RNAi against k-ras and c-myc are:
1—effective to inhibit k-ras protein in HEK293T cells (90% at 24 h and 50% at 48 h)
2—effective to inhibit c-myc protein in HEK293T cells (40% at 24 h and 30% at 48 h)

### 5. Cytotoxicity Assay

### a. Cells and Treatment with MEVs-RNAi

All cell lines are detached with Trypsin, counted (Cellometer Auto 1000) with Trypan blue solution (Sigma T81154, 0.4%). Viable cells are seeded in 96-well plates at 30000 cells per well in 100 μL per well.

Cells are treated 24 h after seeding. Compounds are directly added in each well containing 180 μL of complete medium. 20 μL of are added in: ten-fold serial dilutions for a total of 5 dilutions. For the mock, PBS was added in each well.

### a. MTT Assay

After 72 hours of MEV exposure, 20 μl of MTT at 5 mg/mL are added to each well and incubate for 2 hours at 37° C.

The supernatant is gently aspirated, without aspirating or disturbing the crystals.

Then, 100 μl of isopropanol-HCl IN were added to each well (900 ml of isopropanol+100 ml of HCl diluted to 1/10). The plate is incubated for 15 minutes and homogenized with multichannel pipette. The plate is read to the luminometer (OD 540 nm-OD 690 nm) and the absorbances are analyzed taking into account of blanks (medium and dissolution MTT solution).

On these cell lines after 72 h of MEV-RNAi exposure, none of tested conditions showed a significant decrease of viability.

### Example 12

Distribution of MEVs in the bulbar region, in the hypothalamic region and in the cerebellar region of the brain following intranasal administration.

A. MEVs produced, purified, characterized, and labelled with DiR as described herein (see, also copending International PCT application No. PCT/EP2022/070371, published Jan. 26, 2023) were administered in C57BL/6 mice by intranasal administration. Mice were euthanatized at 16 h after single intranasal administration (20 mL in each nostril) per animal; the brains were carefully isolated; brains were sectioned, embedded in OCT at max. 30 min post sampling and sliced at different distance of the bregma.

B. 8-week-old Swiss mice were treated by IN administration with up to 20 μl of a MEV suspension. 16 hours after, mice were rapidly anesthetized, perfused with 4% (weight/vol.) paraformaldehyde antigen fix solution (PFA). The brains were taken out from the skull with a special care not to damage the olfactory bulbs, post-fixed in 4% PFA and soaked in 30% sucrose.

C. Fluorescently labeled (DiR) MEVs were detected in the brain by histological examination. Briefly, the brains were cut using a cryostat into free-floating 20-μm coronal sections. Slices were selected in the bulbar region, in the hypothalamic region and in the cerebellar region. The sections were mounted on microscopic slides and analyzed for fluorescence as follows:

DNA was stained with DAPI to visualize the brain tissue by labeling the nuclei of all the cells. MEVs were labelled prior to the study with DiR as described previously and visualized as described in Examples 1 and 2. Positive control slides were prepared using an MEV suspension. The transport of MEVs into the brain was evaluated based on the level of fluorescently labeled MEVs in the analyzed areas of the brain: olfactory bulb, hypothalamus, and cerebellum.

D. Results obtained allowed to identify DiR staining inside the brain of mice after IN administration of DiR stained MEVs. The DiR staining can be detected in different brain regions all along the analyzed part of the brain from Bregma 4 mm to Bregma-1.5 mm, such as the olfactory bulbs, the corpus callosum, the cortex and the internal capsule, the thalamus and at different levels of the hippocampus (fimbria and close to dentate gyrus).

### Example 13

### MEV-Mediated In Vivo Delivery, Expression and Biologic Activity of Luciferase Enzyme and Luciferase-mRNA

### A. Intra-Tracheal Administration of MEVs Loaded with Luciferase mRNA or Luciferase Protein

In vivo bioluminescence was used to study the biodistribution and MEV-mediated delivery and expression of luciferase mRNA and luciferase protein. Eight (8) female BALB/cByJ mice were divided into two groups (4 animals per group) for intra-tracheal (IT) administration. Mice in each group were treated either with MEVs loaded with mRNA encoding luciferase (MEV-luc mRNA) or MEVs loaded with luciferase enzyme (MEV-luc protein). MEVs were loaded and characterized as described above. For the IT administration, the animals were maintained under isoflurane-induced anesthesia and placed on a mouse intubation platform. Then, the trachea was backlit with a cold light and 50 μL/mouse of MEV formulations were administered intra-tracheally using a Microsprayer® aerosolizer (Penn-Century). After administration, mice were maintained in the same position on the intubating platform for at least 30 s before being replaced in their cage. A background mouse also was included in the study; this mouse did not receive any MEV administration and served as a control to measure the background level of the bioluminescence acquisition.

### B. Bioluminescence Acquisition

Prior to imaging, the fur of each animal was shaved on the abdomen and thoracic area using an electric clipper. Each mouse was intraperitoneally (IP) injected with 200 μL of luciferin solution at a concentration of 16.5 mg/mL. Bioluminescence acquisitions were performed 10 minutes after the luciferin injection, with mice anesthetized with a mix of isoflurane/oxygen. Animals were positioned in dorsal recumbency (ventral images) to visualize the mice abdomen and thorax and particularly the lungs, liver, intestine, and spleen. Bioluminescence acquisition in vivo was performed with the IVIS® LUMINA X5 optical imaging system (PerkinElmer). The bioluminescence was measured at 6 timepoints: 1 h before MEV administration and 6, 30, 48, 54 and 72 h post-administration. The bioluminescence signal was visualized and quantified in the lungs, liver, intestine, and spleen. Animals were euthanized after the last bioluminescence acquisition. At each timepoint, bioluminescence acquisitions were first performed on the background mouse to measure the background flux level corresponding to the auto-bioluminescence of mice and the noise emitted by the camera of the optical imaging system. Bioluminescence acquisitions then were performed on the experimental mice.

### C. Delivery of Luciferase mRNA, Protein Expression and Luciferase Activity In Vivo

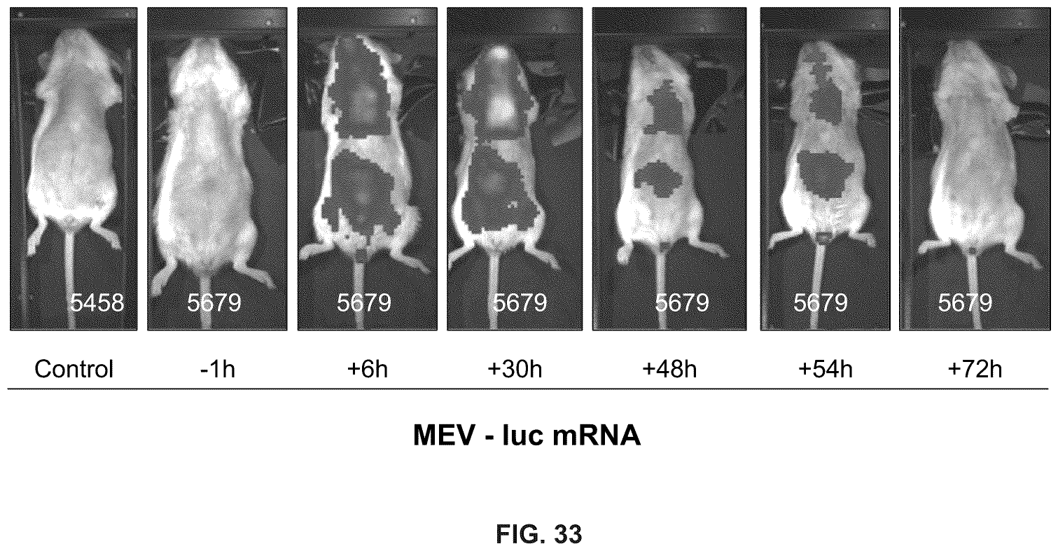

FIG. 33 shows whole-body bioluminescence imaging of a representative animal treated with MEVs loaded with luciferase mRNA. MEV-mediated delivery of mRNA resulted in expression of enzymatically active luciferase in mouse tissues, as evidenced by the luminescence signal detected after the administration of luciferin. The target organs after intratracheal administration include the respiratory system (lungs and naso-buccal epithelium), as well as the gastrointestinal tract (intestinal epithelium) resulting from partial regurgitation of the MEV formulation. Luciferase activity after a single administration of MEVs was detectable starting from 6 hours post-administration and lasted for 2-3 days. The control was a background mouse with no MEV administration.

### D. Luciferase Delivery and Activity In Vivo

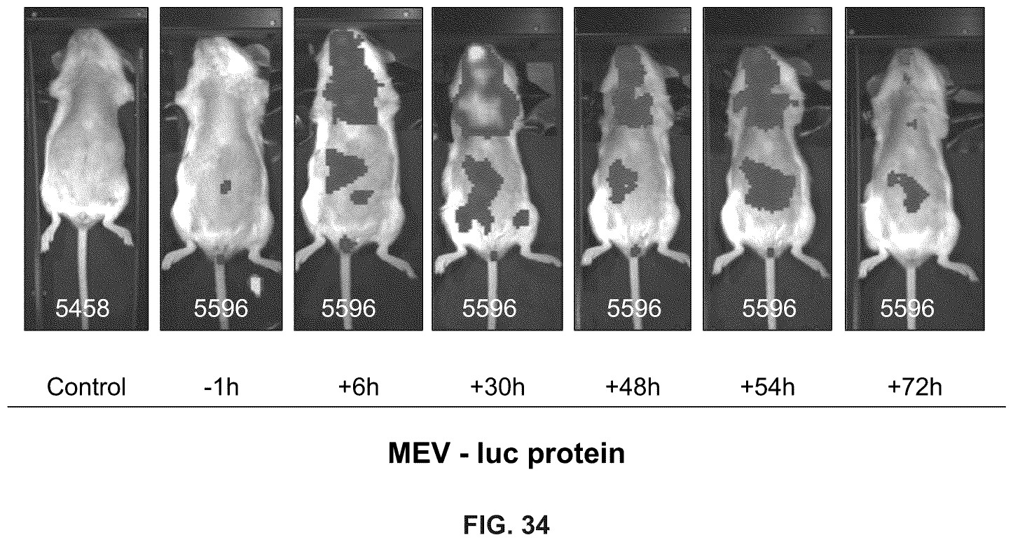

FIG. 34 depicts whole-body bioluminescence imaging of a representative animal treated with MEVs loaded with luciferase enzyme. MEV-mediated delivery of the protein resulted in enzymatic activity of luciferase in mouse tissues, as evidenced by the luminescence signal detected after the administration of luciferin. The target organs after intratracheal administration include the respiratory system (lungs and naso-buccal epithelium), as well as the gastrointestinal tract (intestinal epithelium) resulting from partial regurgitation of the MEV formulation. Luciferase activity after a single administration of MEVs was detectable starting from 6 hours post-administration and lasted for 3-4 days. The control is a background mouse with no MEV administration.

### Example 14

In vitro-Internalization of MEVs by human iPSC-derived neural cells and expression of payload (mRNA, or protein or small molecules).

MEVs can load a wide variety of cargo, such as siRNAs, mRNAs, small molecules, peptides, and proteins. To evaluate MEVs as a delivery system for therapeutic purposes to the central nervous system, the internalization of GFP-loaded MEVs by human brain cells derived from induced pluripotent stem cells was assessed. Human neural stem cells, astrocytes, glial cells and neurons, validated with cell type-specific markers, were used for the internalization assays.
A. MEVs were produced, loaded, and characterized as described above.
B. Culture, differentiation, and characterization of derived cells from human induced pluripotent stem cells (hiPSCs).

Human induced pluripotent stem cells (hiPSCs) are cultured under a neural induction medium to generate neural stem cells (NSCs) according to the protocol described in Yan et al. (2013) NeuroImage 80:246-262. NSCs are expanded in NEM medium (Advanced DMEM/F12 and Neurobasal supplemented with Neural Induction Supplement). NSCs exhibit neural progenitor markers and are negative for pluripotency markers, indicating a commitment to the ectodermal lineage. During brain development, NSCs are capable to give rise to all neural cells (neurons, astrocytes, and oligodendrocytes) and have the capacity for self-renewal. In adulthood, NSCs persist in the hippocampus and in the subventricular zone (SVZ) of the lateral ventricles. These are the most studied regions capable to generate new neural cells and to contribute to neural plasticity. Human neural stem cells exhibited positive staining for Nestin and PAX6, both neural stem cell markers. No OCT4 positive cells were detected in NSCs, which is a pluripotency marker highly expressed in hiPSCs (Casas et al, 2018).

To generate astrocytes, NSCs are cultured under astrocyte differentiation medium (DMEM/F12 supplemented with N₂ and 1% fetal bovine serum) for 21 days according to the protocol described in Yan Y et al., 2013. After the differentiation stage, astrocytes are cultured for 5 weeks under astrocyte medium (DMEM/F12 10% fetal bovine serum) to ensure cell maturation. Mature astrocytes were previously characterized (Ledur et al., 2020 and Trindade et al., 2020) by the expression of main astrocytic markers (as ALDHIL1, GFAP, Vimentin, EAAT1, EAAT2, and S100b) and exhibit functional characteristics (as Human iPSC-derived astrocytes display impairment of [3H]D-aspartate uptake after TNF-α exposure. [3H]D-aspartate is a nonmetabolized analog of glutamate that is commonly used to measure the uptake activity of glutamate transporters and evaluate astrocyte functionality); Ledur et al., 2020 and Trindade et al., 2020, respectively.

NSCs are differentiated in mixed neuronal culture on laminin-coated plates and under neuronal differentiation medium (Neurobasal supplemented with 1X B27, 1X Glutamax, 1X Non-Essential Amino Acids Solution, and 200 nM ascorbic acid) (Yan Y et al., 2013). On day 7 and 14 of the differentiation protocol, cultures are detached with Accutase® and replated in laminin-coated coverslips in neuronal differentiation media containing 10 μM ROCK inhibitor to improve neuronal survival.

Neurons are cultured until day 45 to perform experiments and ensure proper maturation. The characterization uses neuronal markers, such as b-tubulin III (BTUBIII), microtubule-associated protein 2 (MAP2), and presynaptic marker synaptophysin (SYP). Neurons are negative to Nestin, a neural stem cell marker, indicating the mature profile of the culture.

C. Culture and characterization of glial cells.

Primary human glial cells, obtained from Lonza, were isolated. The human glial cells were seeded at a density of 36,000 cells/cm² and cultured in DMEM supplemented with 10% FBS, 2 mmol/l GlutaMAX, 1 mmol/l sodium pyruvate, 100 units/ml penicillin, and 100 μg/ml streptomycin for one week. Glial cells were characterized using specific markers as glial fibrillary acidic protein (GFPA) and S100b, Vimentin and Nestin.

D. Evaluation of the internalization of MEVs in human neuron stem cells, human neurons, human astrocytes and human glial cells.

Cells were incubated with MEVs-mRNA eGFP or GFP (protein) and monitored for up to 24 h for vesicle internalization using a live-cell imaging platform. Hoechst dye is used to stain nuclei. After image acquisition, GFP intensity is measured to estimate the internalization efficiency and the percentage of GFP positive cells is calculated.

E. Internalization, delivery, and analysis of MEVs loaded with mRNA-eGFP or GFP protein to human neural stem cells.

Human NSCs were incubated with MEV-GFP for up to 24 h. Cells were analyzed on a live-cell imaging platform after 30 min of Hoechst incubation for nuclei staining. After image acquisition, GFP intensity was measured and GFP positive cells were calculated. Cells were immuno-stained for PAX6 for representative imaging of each cell type. Calculations made: Mean intensity GFP values and percentage of GFP positive cells.

F. Internalization, delivery, and analysis of MEVs loaded with mRNA-eGFP or GFP protein to human astrocytes.

Human astrocytes were incubated with MEV-GFP for up to 24 h. Cells were analyzed on a live-cell imaging platform after 30 min of Hoechst incubation for nuclei staining. After image acquisition, GFP intensity was measured and GFP positive cells were calculated. Cells were immuno-stained for GFAP for representative imaging of each cell type. Calculations made: Mean intensity GFP values and percentage of GFP positive cells.

G. Internalization, delivery, and analysis of MEVs loaded with mRNA-eGFP or GFP protein to human neurons.

Human neurons were incubated with MEV-GFP for up to 24 h. Cells were analyzed on a live-cell imaging platform after 30 min of Hoechst incubation for nuclei staining. After image acquisition, GFP intensity was measured and GFP positive cells were calculated. Cells were immuno-stained for MAP2 for representative imaging of each cell type. Calculations made: Mean intensity GFP values and percentage of GFP positive cells.

H. Internalization, delivery, and analysis of MEVs loaded with mRNA-eGFP or GFP protein to human microglia cells: human glial cells were incubated with MEV-GFP for up to 24 h. Cells were analyzed on a live-cell imaging platform after 30 min of Hoechst incubation for nuclei staining. After image acquisition, GFP intensity was measured and GFP positive cells were calculated. Cells were immuno-stained for GFAP for representative imaging of each cell type. Calculations made: Mean intensity GFP values and percentage of GFP positive cells.

### Example 15

In Vivo Delivery and Expression of Exogenously Loaded mRNA-Ocular Distribution Following a Single Ocular Topical Administration in Albino Rabbits

A New Zealand White (albino) rabbit is one of the species most used for ocular biodistribution analysis. Eight 3-month males of weight between 2-2.5 kg were used in the ocular topical administration study. All animals were ear-tagged at their arrival and the identification numbers 10 were also marked in ears using indelible ink following the inclusion examination. Animals were daily observed for signs of illness and particular attention was paid to their eyes. Only healthy animals without visible ocular defect were used in the study. Animals were housed individually in standard cages. The temperature was held at 18+/−3° C. and the relative humidity at 45-80%. Rooms were continuously ventilated (15-20 air volumes per hour). Animals were routinely exposed (in-cage) to 10-200 1x light in a 12-hour light (from 7:00 a.m. to 7:00 p.m.) and darkness-controlled cycle. Animals had free access to food (90 g/day) and water available ad libitum from plastic bottles.

A 50-μL single instillation on both eyes was applicated to all animals, using an appropriate micropipette, at a dose of 2λ10⁸ MEVs loaded with mRNA-eGFP. Fluorophotometry was performed as follows: at several time-points (0, 6, 24, 30, 48, 54, and 72-hours post-administration) animals were anesthetized and pupils were dilated; measurements of ocular fluorescence were performed with FM-2 Fluorotron-Master ocular fluorophotometer in both eyes. A series of 148 scans with a step size of 0.25 mm were recorded from the cornea to the retina along the optical axis.

### CONCLUSIONS

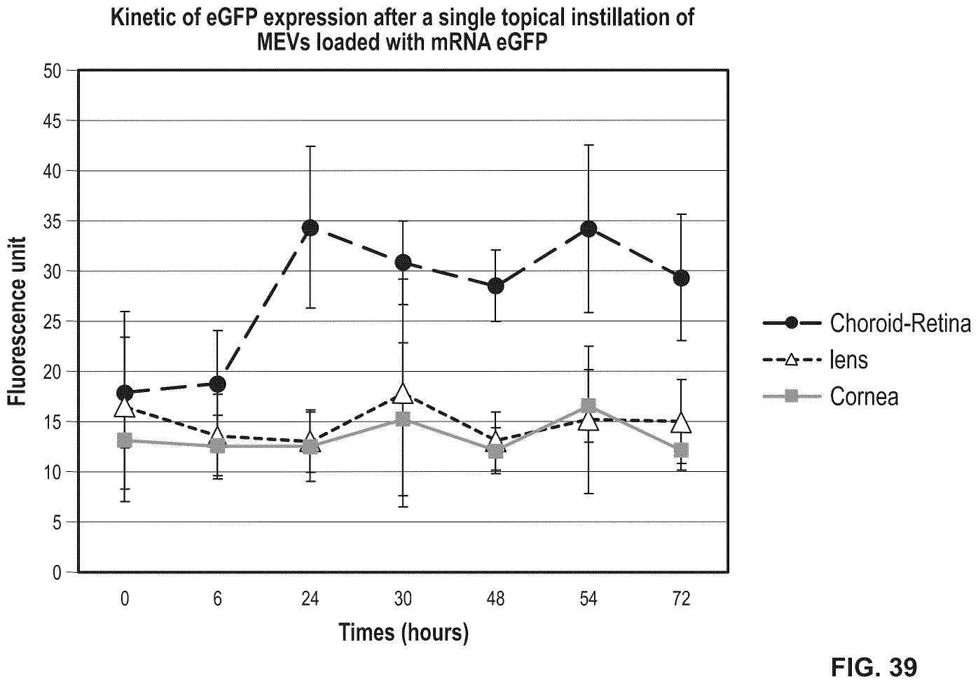

The fluorescence signal in various ocular tissues (choroid/retina, vitreous, lens, anterior chamber, cornea) was obtained at data points that will be 0.25 mm apart along an optical axis by the fluorophotometer. Any detectable fluorescence was measured in the vitreous or the anterior chamber. The results of all animals are summarized in FIG. 39.

Since modifications will be apparent to those of skill in the art, it is intended that this invention be limited only by the scope of the appended claims.

1. A method of preparing microalgae extracellular vesicles (MEVs), comprising:
a) introducing nucleic acid into a microalgae cell, wherein:
the nucleic acid encodes the endogenous cargo, or encodes a biosynthesis pathway that produces the endogenous cargo;
the microalgae is a species of the family Chlorellaceae;
the nucleic acid encodes the endogenous cargo, or encodes a biosynthesis pathway that produces the endogenous cargo, wherein:
the endogenously loaded cargo is heterologous to the microalgae;
the endogenously loaded cargo is encodes a bioactive product or is a bioactive product; and
the endogenously loaded cargo comprises a polypeptide and/or RNA,
with the proviso that the RNA molecule does not comprise RNAi or precursors thereof;
b) culturing the microalgae cells, whereby the encoded cargo is packaged in the resulting MEVs; and
c) isolating the MEVs.

2. An MEV produced by the method of claim 1.

3. The method of claim 1, further comprising formulating the MEVs into a composition for administration to an animal.

4. A composition, comprising an MEV of claim 2, wherein the cargo comprises an immunomodulatory product or an immunizing antigen.

5. The composition of claim 4 that is formulated for oral administration.

6. The composition of claim 5, wherein the cargo comprises a vaccine against a pathogen.

7. The method of claim 1, wherein the endogenous cargo comprises mRNA, a peptide, or a polypeptide.

8. The method of claim 1, wherein the microalgae is a species of Chlorella.

9. The method of claim 1, wherein the microalgae is a species of Chlorella selected from among Chlorella ellipsoidea, Chlorella pyrenoidosa, Chlorella sorokiniana, Chlorella vulgaris, and Chlorella variabilis.

10. The method of claim 9, wherein the Chlorella is Chlorella vulgaris.

11. The method of claim 1, wherein the cargo is for treatment of a disease, disorder, or condition that is a proliferative disorder, or an immune cell disorder, or an infectious disease, or a genetic disease, or a metabolic disease.

12. The method of claim 11, wherein the disease, disorder, or condition is selected from among a disease of or involving one or more of:
the upper respiratory system, and/or the lower respiratory system, and/or respiratory system associated organs and tissues;
the central nervous system, and/or the peripheral nervous system, and/or central nervous system associated organs and tissues;
the skin, dermis, and/or epidermis, or a disease or condition of or involving the exposed epithelia or mucosa;
urogenital mucosa; naso-buccal mucosa; anorectal mucosa; ocular mucosa; or is an ophthalmic disease, disorder, or condition;
the immune system;
the eyes and/or a disease, disorder, or condition of the eyes selected from a macular degeneration, or cataracts, or glaucoma, or conditions resulting from diabetic retinopathy, or genetic diseases affecting the vision, the eyes, and/or associated tissues;
an infectious agent; and
lymphoid tissues, lymph nodes and/or the spleen.

13. The method of claim 12, wherein the disease, disorder, or condition involves lymphoid tissue wherein the lymphoid tissue is selected from among mucosa-associated lymphoid tissues (MALT), gut-associated lymphoid tissues (GALT), bronchus-associated lymphoid tissues (BALT), nasal-associated lymphoid tissues (NALT), conjunctival-associated lymphoid tissues (CALT), larynx-associated lymphoid tissues (LALT), skin-associated lymphoid tissues (SALT), vulval-vaginal-associated lymphoid tissues (VALT), and testis associated lymphoid tissues (TALT).

14. The method of claim 12, wherein the disease, disorder, or condition is a disease of or involving the digestive tract, and the associated organs and tissues.

15. The method of claim 12, wherein the disease, disorder, or condition involves an infectious agent is selected from among a bacterium, a virus, an oomycete, and fungus.

16. The method of claim 1, wherein the cargo comprises an immunostimulatory protein, or an antigen, or encodes an immunostimulatory protein or antigen, whereby the MEV, upon administration is immunostimulating, eliciting an innate or adaptive immune response.

17. The method of claim 1, wherein the endogenous cargo is for treatment, including prevention, of a disease, disorder or condition that is a neurodegenerative disease (such as Parkinson's, or Alzheimer's, or Huntington's, or Creutzfeldt-Jakob disease, or other neurodegenerative disease), or a cognitive disorder (such as dementia, or amnesia, or delirium, or other cognitive disorder), or a brain disorder (such as encephalitis, or seizures, or tumors, or other brain disorder), or a nervous system disorder (such as pain, or seizures, or infections, or other nervous system disorder), or a genetic disease (such as cystic fibrosis, thalassemia, sickle cell anemia, Huntington's Disease, Duchenne's muscular dystrophy, Tay-Sachs disease, or other genetic disease), or a multifactorial disease (such as diabetes, Chronic Obstructive Pulmonary Disease (COPD), or other multifactorial disease), or cancer (such as cancer of the breast, or ovaries, or bowel, or prostate, or skin, or other cancer), or high blood pressure, or high cholesterol, or schizophrenia, or bipolar disorder, or arthritis, or a metabolic disease (such as familial hypercholesterolemia, or Gaucher disease, or Hunter syndrome, or Krabbe disease, or maple syrup urine disease, or metachromatic leukodystrophy, or MELAS (mitochondrial encephalopathy, lactic acidosis, stroke-like episodes), or Niemann-Pick disease, or phenylketonuria (PKU), or porphyria, or Tay-Sachs disease, or Wilson's disease, or other metabolic disease), or a disease of the respiratory system (such as asthma, or Chronic Obstructive Pulmonary Disease (COPD), or chronic bronchitis, or emphysema, or lung cancer, or cystic fibrosis, or bronchiectasis, or pneumonia, or other disease of the respiratory system), or a disease of the digestive tract (such as irritable bowel syndrome (IBS), or inflammatory bowel disease (IBD), or gastroesophageal reflux disease (GERD), or celiac disease, or diverticulitis, or disease of the digestive tract), or a disease of the mucosa (such as Behçet's disease, or burning mouth syndrome, or oral lichen planus, or pemphigus and pemphigoid, or recurrent aphthous stomatitis, or Sjögren's syndrome, or genitourinary infections, or sinusitis, or nasal or sinus polyps, or smell and taste disorders, or allergy, or ocular mucous membrane pemphigoid, or other disease of the mucosa), or a disease of the muscular or neuromuscular tissues (such as amyotrophic lateral sclerosis (ALS), or Charcot-Marie-Tooth disease, or multiple sclerosis, or muscular dystrophy, or myasthenia gravis, or myopathy, or myositis, or peripheral neuropathy, or other muscular or neuromuscular disease), or a disease of the bones (such as cervical spondylosis, or osteoporosis, or metatarsalgia, or polymyalgia rheumatica, or bone cancer, or rheumatoid arthritis, or osteoarthritis, or other bone disease), or a disease of the endocrine tissue (such as acromegaly, or adrenal insufficiency, or Addison's disease, or Cushing's syndrome, or Graves' disease, or Hashimoto's disease, or Creutzfeldt-Jakob disease, or hyperthyroidism, or hypothyroidism, or multiple endocrine neoplasia, or polycystic ovary syndrome (PCOS), or primary hyperparathyroidism, or other disease of the endocrine tissue), or a disease of haemopoietic or lymphoid tissues (such as Fanconi anemia, or thrombocytopenia, or Diamond-Blackfan anemia, or Shwachman-Diamond syndrome, or chronic granulomatous disease, or Gaucher's disease, or myeloid malignancies including myeloproliferative neoplasms, myelodysplastic disorders, chronic myelomonocytic leukemia, acute myeloid leukemia (AML), or lymphoma, or Hodgkin's lymphoma, or Non-Hodgkin's lymphoma, or lymphadenitis, or lymphangitis, or lymphedema, or lymphocytosis).

18. The method of claim 1, comprising formulating the isolated MEVs for oral administration and the endogenously loaded cargo comprises an antigenic polypeptide from a bacterial or viral pathogen.

19. The method of claim 1, comprising formulating the MEVs for parenteral administration, systemic administration, local administration, oral administration, topical administration, mucosal administration, enteral administration, or transdermal administration.

20. The method of claim 1, wherein comprising formulating the MEVs for oral administration as a vaccine.

21. A composition, comprising a microalgae extracellular vesicle (MEV) produced by genetically-modified microalgae, wherein:
the microalgae that produces the MEV is a species of Chlorellaceae;
the MEV comprise the cargo that was endogenously loaded into the MEV by the genetically-modified microalgae cells that produced the MEV; and
the cargo is heterologous to the microalgae, with the proviso that the cargo is not RNAi.

22. The composition of claim 21, wherein the Chlorella is selected from among Chlorella ellipsoidea, Chlorella pyrenoidosa, Chlorella sorokiniana, Chlorella vulgaris, and Chlorella variabilis.

23. The composition of claim 22, wherein the Chlorella is Chlorella vulgaris.

24. The composition of claim 21, wherein the cargo comprises a nucleic acid or protein or a nucleic acid encoding a protein that is a therapeutic product for treatment of cancer, or an infectious disease, or metabolic diseases, or a neurodegenerative disease or other CNS disorder, or aging, or an aging associated disease, or genetic diseases, or ophthalmic disorders, or immunological disorders, or involving internal organs urogenital organs, the cardiovascular system and associated organs and tissues, hematopoietic or lymphoid tissues, sensory organs and tissues, urogenital organs and tissues, muscle tissues, bones, and/or endocrine tissues.

25. The composition of claim 21, wherein the disease, disorder, or condition involves internal organs that comprise the liver, pancreas, spleen, or brain.

26. The composition of claim 21 that is formulated for oral administration, parenteral administration, topical administration, local administration, intratumoral administration, systemic administration, mucosal administration, intravenous administration, subcutaneous administration, intramuscular administration, intraperitoneal administration, transdermal administration, intranasal administration, inhalation, or intratracheal administration.

27. The composition of claim 21, wherein the composition is formulated for oral delivery, or is formulated as an aerosol for intranasal delivery, inhalation, or nebulization.

28. A method of treatment, comprising administering a composition of claim 21, wherein the formulation is selected to target the organ or tissue to be treated.

29. A composition comprising an MEV produced by the method of claim 1, wherein:
the composition is formulated for oral administration; and
the cargo comprises an immunomodulatory product or an immunizing antigen.

30. The composition of claim 29, wherein antigen is a viral or bacterial antigen.

31. A method of immunization, comprising orally administering the composition of claim 30.
